(19) United States
(12) Patent Application Publication
(10) Pub. No.: US 2022/0241430 A1
(43) Pub. Date: Aug. 4, 2022

(54) MODIFIED VIRAL PARTICLES AND USES THEREOF

(71) Applicant: Regeneron Pharmaceuticals, Inc., Tarrytown, NY (US)

(72) Inventors: Leah Sabin, Goldens Bridge, NY (US); Christos Kyratsous, Irvington, NY (US); Sven Moller-Tank, White Plains, NY (US)

(21) Appl. No.: 17/612,669

(22) PCT Filed: May 22, 2020
(86) PCT No.: PCT/US2020/034328

§ 371 Date: Nov. 19, 2021

Related U.S. Application Data
(60) Provisional application No. 62/852,791, filed on May 24, 2019.

(51) Int. Cl. A61K 48/00; C07K 14/005; C07K 14/315; C07K 14/47; C12N 15/86

(52) U.S. Cl. CPC A61K 48/005; C07K 14/005; C07K 14/315; C12N 2750/14145; C12N 15/86; C12N 2750/14122; C12N 2750/14143; C07K 14/47

(57) ABSTRACT

Provided herein are compositions and methods for adapting adeno-associated virus (AAV) particles comprising capsids of non-primate animal AAV, remote AAV, or a combination thereof. AAV adapted accordingly may be a viable gene therapy platform for the treatment of a patient in need thereof, and may be particularly useful in patients excluded from current treatment modalities involving current therapeutic AAV particles due to their high titer of antibodies against the current therapeutic AAV particles.

### REFERENCE TO A SEQUENCE LISTING SUBMITTED AS A TEXT FILE VIA EFS WEB

The Sequence Listing written in file 10364WO01_ST25.txt is 269 kilobytes, was created on May 19, 2020, and is hereby incorporated by reference.

### TECHNICAL FIELDS

The disclosure herein relates to methods of making and using recombinant AAV particles comprising capsid proteins of a non-primate animal AAV and/or a remote AAV.

### BACKGROUND OF THE INVENTION

The delivery of genes into particular target cells has become one of the most important technologies in modern medicine for the potential treatment of a variety of chronic and genetic diseases. So far, progress in the clinical application of gene therapy has been limited by the lack of ideal gene delivery vehicles.

Ideally, a gene delivery vehicle is able to (1) stably introduce genetic material into desired cells, (2) avoid introducing genetic material into non-target cells, and (3) escape neutralization by a patient's immune system, e.g., a patient's antibodies. Although several non-pathogenic vehicles are currently available, the ability of these vehicles to transduce specific cells and remain invisible to host immune responses remains less than ideal.

For example, viral particles based on adeno-associated virus (AAV) isolated from primates, particularly humans e.g., AAV serotypes AAV2, AAV4, AAV6, AAV7, AAV8, and AAV9, have been the focus of much research since AAVs are capable of transducing a wide range of primate species and tissues in vivo with no evidence of toxicity or pathogenicity. (Muzyczka, et al. (1992) Current Topics in Microbiology and Immunology, 158:97-129). Moreover, AAV safely transduces postmitotic tissues. Although the virus can occasionally integrate into host chromosomes, it does so very infrequently into a safe-harbor locus in human chromosome 19, and only when the replication (Rep) proteins are supplied in trans. AAV genomes rapidly circularize and concatemerize in infected cells, and exist in a stable, episomal state in infected cells to provide long-term stable expression of their payloads.

Additionally, manipulating and redirecting primate AAV infection to specific cells has been achieved in recent years. Many of the advances in targeted gene therapy using viral particles may be summarized as non-recombinatorial (non-genetic) or recombinatorial (genetic) modification of the viral particle, which result in the pseudotyping, expanding, and/or retargeting of the natural tropism of the viral particle. (Reviewed in Nicklin and Baker (2002) Curr. Gene Ther. 2:273-93; Verheiji and Rottier (2012) Advances Virol 2012:1-15).

In a direct recombinatorial targeting approach, a targeting ligand is directly inserted into, or coupled to, a viral capsid, i.e., protein viral capsid genes are modified to express capsid proteins comprising a heterologous targeting ligand. The targeting ligand than redirects, e.g., binds, a receptor or marker preferentially or exclusively expressed on a target cell. (Stachler et al. (2006) Gene Ther. 13:926-931; White et al. (2004) Circulation 109:513-519; see also Park et al., (2007) Frontiers in Bioscience 13:2653-59; Girod et al. (1999) Nature Medicine 5:1052-56; Grifman et al. (2001) Molecular Therapy 3:964-75; Shi et al. (2001) Human Gene Therapy 12:1697-1711; Shi and Bartlett (2003) Molecular Therapy 7:515-525).

In indirect recombinatorial approaches, a viral capsid is modified with a heterologous “scaffold”, which then links to an adaptor that includes a targeting ligand. The adaptor binds to the scaffold and the target cell. (Arnold et al. (2006) Mol. Ther. 5:125-132; Ponnazhagen et al. (2002) J. Virol. 76:12900-907; see also WO 97/05266) Scaffolds such as (1) Fc binding molecules (e.g., Fc receptors, Protein A, etc.), which bind to the Fc of antibody adaptors, (2) (strept)avidin, which binds to biotinylated adaptors, (3) biotin, which binds to adaptors fused with (strept)avidin, (4) a detectable label, which is useful for detection and/or isolation of viral particles, bound by a bispecific adaptor able to non-covalently bind the detectable label and target molecule, and recently (5) protein:protein binding pairs that form isopeptide bonds have been described for a variety of viral particles. (See, e.g., Gigout et al. (2005) Molecular Therapy 11:856-865; Stachler et al. (2008) Molecular Therapy 16:1467-1473; Quetglas et al. (2010) Virus Research 153:179-196; Ohno et al. (1997) Nature Biotechnology 15:763-767; Klimstra et al. (2005) Virology 338:9-21).

Despite the advances providing the ability to direct AAV infection, retargeted AAV as a gene delivery vehicle remains less than ideal due to the presence of neutralizing antibodies against the AAV capsids (NAbs). The presence of AAV NAbs in children suggest that infection with an AAV occurs early in life (Calcedo et al. (2011) ASGCT; Huser et al. (2017) J. Virol. 91:e02137-16). It has been shown that antibodies generated by AAV infection early in life can compromise the subsequent use of an AAV gene therapy vector derived from AAV that are recognized and neutralized by those pre-existing antibodies. (Hurlbut et al. (2010) Mol. Ther. 18:1983-94; Jiang et al. (2006) Blood 108:3321-8; Manno et al. (2006) Nat. Med. 12:342-7; Scallan et al. (2006) Blood 107:1810-7; Wang et al. (2010)Mol. Ther. 18:126-34). Moreover, the presence of neutralizing antibody titers against an AAV serotype is clinically significant, as patients with high titers are deemed ineligible for any treatment involving that serotype. (Jeune et al. (2013) Hum Gene Ther Methods 24:59-67).

Thus, there remains a need for viral systems that are non-pathogenic, adaptable for the targeted transfer of nucleic acids of interest to a variety of target cells, and that overcome the hurdle posed by pre-existing antibodies in patients needing treatment.

### SUMMARY OF THE INVENTION

Described herein is a strategy that may simultaneously mitigate several problems associated with previous and current adeno-associated virus (AAV) particle treatment approaches. Without wishing to be bound by theory, it is expected that most humans would lack pre-existing NAbs against an AAV to which previous exposure thereto by humans is low. However, the ability of such AAV serotypes to be successfully manipulated for use as a gene therapy vector capable of targeting and infecting specific cells and/or evading cross-reactivity by any pre-existing antibodies in the human population remained heretofore unknown.

It is shown herein that an AAV capsid protein of a non-primate animal species may be modified to allow for the targeted introduction of a nucleotide of interest into mammalian cells of a different animal species. Moreover, shown herein is evidence that a non-primate animal AAV particle so modified remains less likely than current AAV therapeutic modalities, which are based on well-characterized human AAV serotypes, to be recognized and/or detected by pre-existing antibodies that are found in the human population. Accordingly, described herein are recombinant AAV viral particles that are able to infect a cell of choice and are better able to evade neutralization by pre-existing antibodies.

Described herein are recombinant AAV viral particles comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,

wherein at least one of:
a) the AAV VP1 capsid protein,
b) any portion of the AAV VP1 capsid protein,
c) the AAV VP2 capsid protein,
d) any portion of the AAV VP2 capsid protein,
e) the AAV VP3 capsid protein, and
f) any portion of the AAV VP3 capsid protein,
   comprises an amino acid sequence having significant sequence identity, e.g., at least 95% identity, to the amino acid sequence of a capsid protein of a non-primate animal AAV or portion thereof, or a remote AAV or a portion thereof,

wherein
I. at least one of the AAV VP1, VP2, and VP3 capsid proteins comprises a modification selected from the group consisting of:
   (a) a first member of a protein:protein binding pair, wherein the protein:protein binding pair directs the tropism of the AAV viral particle,
   (b) a detectable label,
   (c) a point mutation, preferably wherein the point mutation reduces the natural tropism of the AAV viral particle and/or creates a detectable label,
   (d) a chimeric amino acid sequence, and
   (e) any combination of (a), (b), (c), and (d), and/or
II. the ITR sequence, or portion thereof, comprises a nucleic acid sequence having significant sequence identity, e.g., at least 95% identity, to the ITR sequence of a second AAV or portion thereof, wherein the second AAV is not the same as the non-primate animal AAV or the remote AAV, and

wherein the recombinant AAV viral particle is capable of infecting a mammalian host, preferably a primate host.

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,

wherein at least one of:
a) the AAV VP1 capsid protein,
b) the AAV VP2 capsid protein, and
c) the AAV VP3 capsid protein,
   comprises an amino acid sequence having significant sequence identity, e.g., at least 95% identity, to the amino acid sequence of a capsid protein of a non-primate animal AAV or a remote AAV,

wherein
I. at least one of the AAV VP1, VP2, and VP3 capsid proteins comprises a modification selected from the group consisting of:
   (a) a first member of a protein:protein binding pair, wherein the protein:protein binding pair directs the tropism of the AAV viral particle,
   (b) a detectable label,
   (c) a point mutation, preferably wherein the point mutation reduces the natural tropism of the AAV viral particle and/or creates a detectable label,
   (d) a chimeric amino acid sequence, and
   (e) any combination of (a), (b), (c), and (d), and/or
II. the ITR sequence, or portion thereof, comprises a nucleic acid sequence having significant sequence identity, e.g., at least 95% identity, to the ITR sequence of a second AAV or portion thereof, wherein the second AAV is not the same as the non-primate animal AAV or the remote AAV, and

wherein the recombinant AAV viral particle is capable of infecting a mammalian host, preferably a primate host.

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,

wherein at least one of:
a. any portion of the AAV VP1 capsid protein,
b. any portion of the AAV VP2 capsid protein, and
c. any portion of the AAV VP3 capsid protein,
   comprises an amino acid sequence having significant sequence identity, e.g., at least 95% identity, to the amino acid sequence of a capsid protein of a non-primate animal AAV or portion thereof, or a remote AAV or a portion thereof,

wherein
I. at least one of the AAV VP1, VP2, and VP3 capsid proteins comprises a modification selected from the group consisting of:
   (a) a first member of a protein:protein binding pair, wherein the protein:protein binding pair directs the tropism of the AAV viral particle,
   (b) a detectable label,
   (c) a point mutation, preferably wherein the point mutation reduces the natural tropism of the AAV viral particle and/or creates a detectable label,
   (d) a chimeric amino acid sequence, and
   (e) any combination of (a), (b), (c), and (d), and/or
II. the ITR sequence, or portion thereof, comprises a nucleic acid sequence having significant sequence identity, e.g., at least 95% identity, to the ITR sequence of a second AAV or portion thereof, wherein the second AAV is not the same as the non-primate animal AAV or the remote AAV, and

wherein the recombinant AAV viral particle is capable of infecting a mammalian host, preferably a primate host

In some recombinant AAV viral particle embodiments, the recombinant viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a non-primate animal AAV or portion thereof, and wherein at least one of the AAV VP1, VP2, and VP3 capsid proteins comprises a modification selected from the group consisting of:
(a) a first member of a protein:protein binding pair, wherein the protein:protein binding pair directs the tropism of the AAV viral particle,
(b) a detectable label,
(c) a point mutation, preferably wherein the point mutation reduces the natural tropism of the AAV viral particle and/or creates a detectable label,
(d) a chimeric amino acid sequence, and
(e) any combination of (a), (b), (c), and (d),

wherein the entire ITR sequence or a portion of the ITR sequence comprises a nucleic acid sequence having significant sequence identity, e.g., at least 95% identity, to the ITR of the non-primate animal AAV, optionally wherein the ITR sequence comprises a chimeric nucleic acid sequence, and wherein a portion of the chimeric nucleic acid sequence having significant sequence identity, e.g., at least 95% identity, to the ITR of the non-primate AAV or portion thereof is operably linked to a portion of the chimeric nucleic acid sequence having significant sequence identity, e.g., at least 95% identity, to the ITR of a second AAV or portion thereof, wherein the second AAV is not the same as the non-primate animal AAV, and wherein the recombinant AAV viral particle is capable of infecting a mammalian host, preferably a primate host.

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a non-primate animal AAV or portion thereof,

wherein the ITR sequence, or portion thereof, comprises a nucleic acid sequence having significant sequence identity, e.g., at least 95% identity, to the ITR sequence of a second AAV or portion thereof, wherein the second AAV is not the same as the non-primate animal AAV,

wherein the recombinant AAV viral particle is capable of infecting a mammalian host preferably a primate host, and

optionally wherein at least one of the AAV VP1, VP2, and VP3 capsid proteins comprises a modification selected from the group consisting of:

(a) a first member of a protein:protein binding pair, wherein the protein:protein binding pair directs the tropism of the AAV viral particle,

(b) a detectable label,

(c) a point mutation, preferably wherein the point mutation reduces the natural tropism of the AAV viral particle and/or creates a detectable label, and

(d) any combination of (a)-(c).

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises a chimeric amino acid sequence comprising (A) an amino acid sequence having significant sequence identity, e.g., at least 95% identity, to the amino acid sequence of a non-primate animal AAV capsid protein, or a portion thereof, operably linked to (B) an amino acid sequence having significant sequence identity, e.g., at least 95% identity, to the amino acid sequence of a second AAV capsid protein, or a portion thereof, wherein the second AAV is not the same as the non-primate animal AAV, wherein the recombinant AAV viral particle is capable of infecting a mammalian host, preferably a primate host, and optionally wherein the at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprising a chimeric amino acid sequence further comprises a modification selected from the group consisting of

(a) a first member of a protein:protein binding pair,

(b) a detectable label, and

(c) a combination of (a) and (b).

In some recombinant AAV viral particle embodiments, the recombinant viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a remote AAV or portion thereof, and wherein at least one of the AAV VP1, VP2, and VP3 capsid proteins comprises a modification selected from the group consisting of:
(a) a first member of a protein:protein binding pair, wherein the protein:protein binding pair directs the tropism of the AAV viral particle,
(b) a detectable label,
(c) a point mutation, preferably wherein the point mutation reduces the natural tropism of the AAV viral particle and/or creates a detectable label,
(d) a chimeric amino acid sequence, and
(e) any combination of (a), (b), (c), and (d),

wherein the entire ITR sequence or a portion of the ITR sequence comprises a nucleic acid sequence having significant sequence identity, e.g., at least 95% identity, to the ITR of the remote AAV, optionally wherein the ITR sequence comprises a chimeric nucleic acid sequence, and wherein a portion of the chimeric nucleic acid sequence having significant sequence identity, e.g., at least 95% identity, to the ITR of the remote AAV or portion thereof is operably linked to a portion of the chimeric nucleic acid sequence having significant sequence identity, e.g., at least 95% identity, to the ITR of a second AAV or portion thereof, wherein the second AAV is not the same as the remote AAV, and wherein the recombinant AAV viral particle is capable of infecting a mammalian host, preferably a primate host.

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a remote AAV or portion thereof,

wherein the ITR sequence, or portion thereof, comprises a nucleic acid sequence having significant sequence identity, e.g., at least 95% identity, to the ITR sequence of a second AAV or portion thereof, wherein the second AAV is not the same as the remote AAV,

wherein the recombinant AAV viral particle is capable of infecting a mammalian host preferably a primate host, and

optionally wherein at least one of the AAV VP1, VP2, and VP3 capsid proteins comprises a modification selected from the group consisting of:

(a) a first member of a protein:protein binding pair, wherein the protein:protein binding pair directs the tropism of the AAV viral particle,

(b) a detectable label,

(c) a point mutation, preferably wherein the point mutation reduces the natural tropism of the AAV viral particle and/or creates a detectable label, and

(d) any combination of (a)-(c).

In some embodiments, a recombinant AAV viral particle comprises (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence, wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises a chimeric amino acid sequence comprising (A) an amino acid sequence having significant sequence identity, e.g., at least 95% identity, to the amino acid sequence of a remote AAV capsid protein, or a portion thereof, operably linked to (B) an amino acid sequence having significant sequence identity, e.g., at least 95% identity, to the amino acid sequence of a second AAV capsid protein, or a portion thereof, wherein the second AAV is not the same as the remote AAV, wherein the recombinant AAV viral particle is capable of infecting a mammalian host, preferably a primate host, and optionally wherein the at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprising a chimeric amino acid sequence further comprises a modification selected from the group consisting of

(a) a first member of a protein:protein binding pair,

(b) a detectable label, and

(c) a combination of (a) and (b).

In some embodiments of the invention, an AAV viral particle comprises an AAV capsid, wherein at least one AAV capsid protein (e.g., an AAV VP1 capsid protein, an AAV VP2 capsid protein, and/or an AAV VP3 capsid protein) of said AAV capsid comprises at least a portion of an amino acid sequence of a capsid protein selected from the group consisting of a capsid protein of a non-primate animal AAV, a capsid protein of a remote AAV, and a combination thereof, and wherein at least one AAV capsid protein of said AAV capsid is modified to comprise (a) at least a first member of a protein:protein binding pair, (b) a detectable label, (c) a point mutation, (d) a chimeric amino acid sequence comprising a portion of an amino acid sequence of an other, e.g., a second, AAV capsid protein that is operably linked to said amino acid sequence of the capsid protein selected from the group consisting of the capsid protein of the non-primate animal AAV, the capsid protein of the remote AAV, or the combination thereof, and (e) any combination of (a), (b), (c), and (d). In some embodiments of the invention, an AAV viral particle comprises an AAV capsid, wherein at least one AAV capsid protein (e.g., an AAV VP1 capsid protein, an AAV VP2 capsid protein, and/or an AAV VP3 capsid protein) of said AAV capsid or portion thereof has significant sequence identity, e.g., at least 95% identity, to a capsid protein selected from the group consisting of a capsid protein of a non-primate animal AAV, a portion of a capsid protein of the non-primate animal AAV, a capsid protein of a remote AAV, a portion of the capsid protein of a remote AAV, and a combination thereof, and wherein the at least one AAV capsid protein of said AAV capsid is modified to comprise (a) at least a first member of a protein:protein binding pair, (b) a detectable label, (c) a point mutation, (d) a chimeric amino acid sequence comprising a portion of an amino acid sequence of an other, e.g., a second, AAV capsid protein that is operably linked to said amino acid sequence of the capsid protein selected from the group consisting of the capsid protein of the non-primate animal AAV, the capsid protein of the remote AAV, or the combination thereof, and (e) any combination of (a), (b), (c), and (d). In some embodiments of the invention, an AAV viral particle comprises an AAV capsid, wherein at least one AAV capsid protein (e.g., an AAV VP1 capsid protein, an AAV VP2 capsid protein, and/or an AAV VP3 capsid protein) of said AAV capsid comprises at least a portion of an amino acid sequence of a capsid protein of a non-primate animal AAV (e.g., wherein at least one AAV capsid protein comprises an amino acid sequence having significant sequence identity, e.g., at least 95% sequence identity, to a capsid protein of a non-primate animal AAV), wherein at least one AAV capsid protein of said AAV capsid is modified to comprise (a) at least a first member of a protein:protein binding pair, (b) a detectable label, (c) a point mutation, (d) a chimeric amino acid sequence comprising a portion of an amino acid sequence of an other, e.g., a second, AAV capsid protein that is operably linked to said amino acid sequence of the capsid of the non-primate animal AAV, and (e) any combination of (a), (b), (c), and (d).

In some embodiments of the invention, an AAV viral particle comprises an AAV capsid, wherein at least one AAV capsid protein (e.g., an AAV VP1 capsid protein, an AAV VP2 capsid protein, and/or an AAV VP3 capsid protein) of said AAV capsid comprises at least a portion of an amino acid sequence of a capsid protein of a remote AAV (e.g., wherein at least one AAV capsid protein comprises an amino acid sequence having significant sequence identity, e.g., at least 95% sequence identity, to a capsid protein of a remote AAV), wherein at least one AAV capsid protein of said AAV capsid is modified to comprise (a) at least a first member of a protein:protein binding pair, (b) a detectable label, (c) a point mutation, (d) a chimeric amino acid sequence comprising a portion of an amino acid sequence of an other, e.g., a second, AAV capsid protein that is operably linked to said amino acid sequence of the capsid protein of the remote AAV, and (e) any combination of (a), (b), (c), and (d).

In some embodiments of the invention, an AAV viral particle comprises (A) at least one AAV capsid protein, e.g., an AAV VP1 capsid protein, an AAV VP2 capsid protein, and/or an AAV VP3 capsid protein, comprising an amino acid sequence identical to or having significant identity, e.g., at least 95% sequence identity, to an amino acid sequence selected from the group consisting of (i) an amino acid sequence of a capsid protein of a non-primate animal AAV, (ii) an amino acid sequence of a capsid protein of a remote primate AAV, and (iii) an amino acid sequence of a combination thereof, and (B) an AAV genome comprising a nucleotide of interest and an AAV ITR comprising at least a portion of an ITR sequence of an other, e.g., a second, AAV, wherein the other AAV is not identical to the non-primate animal AAV and also not identical to the remote primate AAV.

In some embodiments of the invention, an AAV viral particle comprises (A) at least one AAV capsid protein (e.g., an AAV VP1 capsid protein, an AAV VP2 capsid protein, and/or an AAV VP3 capsid protein) comprising an amino acid sequence identical to or having significant identity, e.g., at least 95% sequence identity, to an amino acid sequence of a capsid protein of a non-primate animal AAV, and (B) an AAV genome comprising a nucleotide of interest and an AAV ITR comprising at least a portion of an ITR sequence of an other AAV, e.g., a second AAV, wherein the other AAV is not identical to the non-primate animal AAV.

In some embodiments of the invention, an AAV viral particle comprises (A) at least one AAV capsid protein (e.g., an AAV VP1 capsid protein, an AAV VP2 capsid protein, and/or an AAV VP3 capsid protein) comprising an amino acid sequence identical to or having significant identity, e.g., at least 95% sequence identity, to an amino acid sequence of a capsid protein of a remote AAV, and (B) an AAV genome comprising a nucleotide of interest and an AAV ITR comprising at least a portion of an ITR sequence of an other AAV, e.g., second AAV, wherein the other AAV is not identical to the remote primate AAV.

In some AAV viral particle embodiments of the invention, the capsid protein comprising an amino acid sequence identical to or having significant identity, e.g., at least 95% sequence identity, to an amino acid sequence of the capsid protein of the non-primate animal AAV, the capsid protein of the remote AAV, or the combination thereof, is modified to comprise (a) at least a first member of a protein:protein binding pair, (b) a detectable label, (c) a point mutation.

In some AAV viral particle embodiments of the invention, the capsid protein comprising an amino acid sequence identical to or having significant identity, e.g., at least 95% sequence identity, to an amino acid sequence of the capsid protein the non-primate animal AAV, the capsid protein of the remote AAV, or the combination thereof, comprises an amino acid sequence identical to or having significant identity, e.g., at least 95% sequence identity, to an amino acid sequence of a VP3 capsid protein of the non-primate animal AAV and/or an amino acid sequence of a VP3 capsid protein of the remote AAV. In some embodiments, the capsid protein comprising an amino acid sequence identical to or having significant identity, e.g., at least 95% sequence identity, to the amino acid sequence of the capsid protein of the non-primate animal AAV, the capsid protein of the remote AAV, or the combination thereof, comprises an amino acid sequence identical to or having significant identity, e.g., at least 95% sequence identity, to an amino acid sequence of a VP2 capsid protein of the non-primate animal AAV and/or an amino acid sequence of a VP2 capsid protein of the remote AAV. In some embodiments, the capsid protein comprising an amino acid sequence identical to or having significant identity, e.g., at least 95% sequence identity, to the amino acid sequence of the capsid protein of the non-primate animal AAV, the capsid protein of the remote AAV, or the combination thereof, comprises an amino acid sequence identical to or having significant identity, e.g., at least 95% sequence identity, to an amino acid sequence of a VP1 capsid protein of the non-primate animal AAV and/or an amino acid sequence of a VP1 capsid protein of the remote AAV.

In some AAV viral particle embodiments of the invention, the capsid of said particle comprises (i) a VP1 capsid protein that is either (a) a chimeric AAV VP1 capsid protein, optionally wherein the chimeric VP1 capsid protein comprises a VP1-unique region (VP1-u) of an other, e.g., second, AAV operably linked to a VP1/VP2 common region and a VP3 region of the non-primate AAV or the remote AAV, or (b) a VP1 capsid protein of the non-primate AAV or the remote AAV, (ii) a VP2 capsid protein that is either (a) a chimeric AAV VP2 capsid protein, optionally wherein the chimeric VP2 capsid protein comprises a VP1/VP2 common region of an other, e.g., second, AAV operably linked to a VP3 region of the non-primate AAV or the remote AAV, or (b) a VP2 capsid protein of the non-primate AAV or the remote AAV, and (iii) the VP3 capsid protein of the non-primate AAV or the remote AAV. In some embodiments, the capsid of said particle comprises (i) a chimeric AAV VP1 capsid protein, optionally wherein the chimeric VP1 capsid protein comprises a VP1-unique region (VP1-u) of an other, e.g., second, AAV operably linked to a VP1/VP2 common region and a VP3 region of the non-primate AAV or the remote AAV, (ii) a chimeric AAV VP2 capsid protein, optionally wherein the chimeric VP2 capsid protein comprises a VP1/VP2 common region of an other, e.g., second, AAV operably linked to a VP3 region of the non-primate AAV or the remote AAV, and (iii) the VP3 capsid protein of the non-primate AAV or the remote AAV. In some embodiments, the capsid of said particle comprises (i) a chimeric AAV VP1 capsid protein, optionally wherein the chimeric VP1 capsid protein comprises a VP1-unique region (VP1-u) of an other, e.g., second, AAV operably linked to a VP1/VP2 common region and a VP3 region of the non-primate AAV or the remote AAV, (ii) a VP2 capsid protein of the non-primate AAV or the remote AAV, and (iii) the VP3 capsid protein of the non-primate AAV or the remote AAV. In some embodiments, the capsid comprises (i) a VP1 capsid protein of the non-primate AAV or the remote AAV, (ii) a VP2 capsid protein of the non-primate AAV or the remote AAV, and (iii) a VP3 capsid protein of the non-primate AAV or the remote AAV, and optionally wherein the particle comprises an AAV genome comprising an AAV ITR comprising at least a portion of an ITR sequence of an other, e.g., a second, AAV, within the capsid. In some embodiments, the other AAV is not identical to the non-primate animal AAV.

In some recombinant AAV viral particle embodiments, (i) the VP1 capsid protein comprises either (a) a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of a non-primate animal AAV, or (b) an amino acid sequence having significant sequence identity, e.g., at least 95% identity, to the VP1 capsid protein of the non-primate animal AAV, (ii) the VP2 capsid protein comprises either (a) a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises at least 95% identity to the VP3 region of the non-primate animal AAV, or (b) an amino acid sequence having significant sequence identity, e.g., at least 95% identity, to the VP2 capsid protein of the non-primate animal AAV, and (iii) the VP3 capsid protein comprising an amino acid sequence having significant sequence identity, e.g., at least 95% identity, to an amino acid sequence of the VP3 capsid protein of the non-primate animal AAV. In some embodiments, (i) the VP1 capsid protein comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of a non-primate animal AAV, (ii) the VP2 capsid protein comprises a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises at least 95% identity to the VP3 region of the non-primate animal AAV, and (iii) the VP3 capsid protein comprises an amino acid sequence having significant sequence identity, e.g., at least 95% identity, to an amino acid sequence of the VP3 capsid protein of the non-primate animal AAV. In some embodiments, (i) the AAV VP1 capsid protein comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV, (ii) the VP2 capsid protein comprises an amino acid sequence having significant sequence identity, e.g., at least 95% identity, to an amino acid sequence of the VP2 capsid protein of the non-primate animal AAV, and (iii) the VP3 capsid protein comprises an amino acid sequence having significant sequence identity, e.g., at least 95% identity, to the amino acid sequence of a VP3 capsid protein of the non-primate animal AAV. In some embodiments, (i) the VP1 capsid protein comprises an amino acid sequence having significant sequence identity, e.g., at least 95% identity, to an amino acid sequence of the VP1 capsid protein of the non-primate animal AAV, (ii) the VP2 capsid protein comprises an amino acid sequence having significant sequence identity, e.g., at least 95% identity, to an amino acid sequence of the VP2 capsid protein of the non-primate animal AAV, and (iii) the VP3 capsid protein comprises an amino acid sequence having significant sequence identity, e.g., at least 95% identity, to an amino acid sequence of the VP3 capsid protein of the non-primate animal AAV, and optionally wherein the particle comprises an AAV genome comprising an AAV ITR comprising at least a portion of an ITR sequence of an other, e.g., a second, AAV, within the capsid. In some embodiments, the other AAV is not identical to the non-primate animal AAV.

In some AAV viral particle embodiments of the invention, the other, e.g., second, AAV is a primate AAV or a combination of primate AAVs. In some embodiments, the other AAV is a selected from the group consisting of AAV1, AAV2, AAV3, AAV4, AAV5, AAV6, AAV7, AAV8, AAV9, AAV10, AAV 11, AAV12, AAV13, and a combination thereof. In some embodiments, the other AAV is AAV2.

In some AAV viral particle embodiments of the invention, the non-primate animal AAV is a non-primate AAV listed in Table 2. In some embodiments, the non-primate AAV is an avian AAV (AAAV), a sea lion AAV or a bearded dragon AAV. In some embodiments, the non-primate animal AAV is an AAAV, and optionally an amino acid sequence of an AAAV capsid protein comprises a modification at position I444 or I580 of a VP1 capsid protein of AAAV. In some embodiments, the non-primate animal AAV is a squamate AAV, e.g., a bearded dragon AAV, and optionally an amino acid sequence of a bearded dragon AAV comprises a modification at position I573 or I436 of a VP1 capsid protein of a bearded dragon AAV. In some embodiments, the non-primate animal AAV is a mammalian AAV, e.g., a sea lion AAV, and optionally an amino acid sequence of a sea lion AAV comprises a modification at a position selected from the group consisting of I429, I430, I431, I432, I433, I434, I436, I437, and A565 of a VP1 capsid protein of a sea lion AAV.

In some AAV viral particle embodiments of the invention, the protein:protein binding pair is selected from SpyTag:SpyCatcher, SpyTag:KTag, Isopeptag:pilin C, SnoopTag:SnoopCatcher, and SpyTag002:SpyCatcher002. In some embodiments, the first member of a protein:protein binding pair comprises c-myc comprising a sequence set forth as SEQ ID NO:44. In some embodiments, the detectable label comprises the B1 epitope comprising an amino acid sequence of IGTRYLTR (SEQ ID NO: 45).

In some embodiments, an AAV particle of the invention comprises a VP3 capsid protein of the non-primate animal AAV, the remote AAV, or the combination thereof, wherein the VP3 capsid protein is modified to comprise (a) at least a first member of a protein:protein binding pair, optionally wherein the protein:protein binding pair is selected from the group consisting of SpyTag:SpyCatcher, SpyTag:KTag, Isopeptag:pilin C, SnoopTag:SnoopCatcher, and SpyTag002:SpyCatcher002, (b) a detectable label, optionally wherein the detectable label comprises the amino acid sequence set forth as SEQ ID NO: 44 or the amino acid sequence set forth as SEQ ID NO:45, (c) a point mutation, or (d) any combination of (a), (b), and (c). In some embodiments, the VP3 capsid protein of the non-primate animal AAV, the remote AAV, or the combination thereof is modified to comprise (a) at least SpyTag comprising an amino acid sequence set forth as SEQ ID NO:43 and/or (b) a detectable label comprising amino acid sequence set forth SEQ ID NO:45.

In some embodiments, an AAV particle of the invention comprises a first and/or second linker operably linking a first member of a protein:protein binding pair and/or a detectable label to a capsid protein of the capsid of said AAV particle. In some embodiments, the first and second linker are not identical. In some embodiments, the first and second linker are identical. In some embodiments, the first and/or second linkers is 10 amino acids in length.

In some viral particle embodiments of the invention, at least one of the VP1, VP2, and VP3 capsid proteins, optionally at least the VP3 capsid, is modified to comprise (a) a first member of a protein:protein binding pair, (b) a detectable label, (c) a point mutation, or (d) any combination of (a), (b), and/or (c). In some embodiments, the first member of a protein:protein binding pair and/or the detectable label or the point mutation is placed within a variable region of the capsid protein. In some embodiments, the first member of a protein:protein binding pair or the detectable label is flanked by a first linker and/or a second linker. In some embodiments, the first and/or second linker is 1-10 amino acids in length. In some embodiments, the first and second linker are not identical. In some embodiments, the first and second linker are identical.

In some viral particle embodiments, an AAAV VP3 capsid protein comprises a modification, optionally a first member of a protein:protein binding pair, optionally wherein the modification is at position(s) I444 (e.g., G444) and/or I580 (e.g., K580). In some embodiments, an AAAV VP3 capsid protein comprises a modification, optionally a first member of a protein:protein binding pair, optionally wherein the modification is at position(s) I444 (e.g., G444) and/or I580 (e.g., K580). In some embodiments, a bearded dragon AAV VP3 capsid protein comprises a modification, optionally a first member of a protein:protein binding pair, optionally wherein the modification is at position(s) I573 (e.g., T573) and/or I436 (e.g., G436). In some embodiments, a sea lion VP3 capsid protein comprises a modification, optionally a first member of a protein:protein binding pair, optionally wherein the modification is at a position selected from the group consisting of I429 (e.g., N429), I430 (e.g., P430), I431 (e.g., T431), I432 (e.g., G432), I433 (e.g., S433), I434 (e.g., T434), I436 (e.g., R436), I437 (e.g., D437), and I565 (A565).

In some viral particle embodiments of the invention, at least one capsid protein, optionally at least the VP3 capsid, is modified to comprise a first member of a protein:protein binding pair. In some embodiments, the first member of a protein:protein binding pair comprises a first member of a protein:protein binding pair. In some embodiments, the first member of a protein:protein binding pair comprises a second cognate member of the protein:protein binding pair. In some embodiments, the first and second member of the protein:protein binding pair are bound by a covalent bond, e.g., an isopeptide bond. In some embodiments, the first member of the protein:protein binding pair is SpyTag, and optionally, the second member of the protein:protein binding pair is SpyCatcher or KTag. In some embodiments, the first member of the protein:protein binding pair is KTag, and optionally, the second member of the protein:protein binding pair is SpyTag. In some embodiments, the first member of the protein:protein binding pair is SnoopTag and the second member of the protein:protein binding pair is SnoopCatcher. In some embodiments, the first member of the protein:protein binding pair is isopeptag and the second member of the protein:protein binding pair is Pilin-C. In some embodiments, the first member of the protein:protein binding pair is SpyTag002 and the second member of the protein:protein binding pair is SpyCatcher002. In some embodiments, the second member of the protein:protein binding pair is linked to a targeting ligand, e.g., a binding moiety, e.g., an antibody or a fragment thereof. In some embodiments, the targeting ligand may be fused to a second member of a protein:protein binding pair, e.g., SpyCatcher, optionally via a linker at the C-terminus of the second member, and the linker is fused to SpyCatcher at the linker's C-terminus. In some embodiments, the linker comprises the sequence GSGESG (SEQ ID NO:49). In some embodiments, the first member of a protein:protein binding pair comprises a detectable label. In some embodiments, the first member of a protein:protein binding pair comprises the detectable label c-myc.

In some viral particle embodiments of the invention, at least one capsid protein, optionally at least the VP3 capsid, is modified to comprise a detectable label. In some embodiments the detectable label comprises the AAV B1 epitope, e.g., the amino acid sequence IGTRYLTR (SEQ ID NO: 45).

In some viral particle embodiments of the invention, at least one capsid protein, optionally at least the VP3 capsid, is modified to comprise

(a) a first member of a protein:protein binding pair comprising at least one member of a protein:protein binding pair, optionally wherein the protein:protein binding pair is selected from the group consisting of SpyTag:SpyCatcher, SpyTag:KTag, Isopeptag:pilin-C, SnoopTag:SnoopCatcher, SpyTag002:SpyCatcher002, and c-myc:anti-c-myc antibody.

(b) a detectable label, optionally wherein the detectable label comprises the amino acid sequence set forth as SEQ ID NO:44 or the amino acid sequence set forth as SEQ ID NO:45,

(c) a point mutation, or

(d) any combination of (a), (b), and (c).

In some viral particle embodiments of the invention, at least one capsid protein, optionally at least the VP3 capsid, is modified to comprise

(a) a first member of a protein:protein binding pair comprising at least SpyTag comprising an amino acid sequence set forth as SEQ ID NO:43 and/or

(b) a detectable label comprising amino acid sequence set forth SEQ ID NO:45.

In some embodiments, an AAV particle of the invention comprises a capsid protein comprising an amino acid sequence selected from the group consisting of (a) an amino acid sequence set forth as SEQ ID NO:2, (b) an amino acid sequence set forth as SEQ ID NO:4, (c) an amino acid sequence set forth as SEQ ID NO:6, (d) an amino acid sequence set forth as SEQ ID NO:8, (e) an amino acid sequence set forth as SEQ ID NO:10, (f) an amino acid sequence set forth as SEQ ID NO:12, (g) an amino acid sequence set forth as SEQ ID NO:14, (h) an amino acid sequence set forth as SEQ ID NO:16, (i) an amino acid sequence set forth as SEQ ID NO:18, (j) an amino acid sequence set forth as SEQ ID NO:20, (k) an amino acid sequence set forth as SEQ ID NO:22, (l) an amino acid sequence set forth as SEQ ID NO:24, (m) an amino acid sequence set forth as SEQ ID NO:26, (n) an amino acid sequence set forth as SEQ ID NO:28, (o) an amino acid sequence set forth as SEQ ID NO:30, (p) an amino acid sequence set forth as SEQ ID NO:32, (q) an amino acid sequence set forth as SEQ ID NO:34, (r) an amino acid sequence set forth as SEQ ID NO:36, (s) the amino acid sequence set forth as SEQ ID NO:53, (t) the amino acid sequence set forth as SEQ ID NO:55, (u) the amino acid sequence set forth as SEQ ID NO:57, (v) the amino acid sequence set forth as SEQ ID NO:59, (w) the amino acid sequence set forth as SEQ ID NO:61, (x) the amino acid sequence set forth as SEQ ID NO:63, (y) the amino acid sequence set forth as SEQ ID NO:65, (z) the amino acid sequence set forth as SEQ ID NO:67, (aa) the amino acid sequence set forth as SEQ ID NO:69, (bb) the amino acid sequence set forth as SEQ ID NO:71, (cc) an amino acid sequence having at least 95% identity to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71, and (dd) an amino acid sequence of any VP2 and/or VP3 portions of the amino acid sequences set forth in any of (a)-(cc).

In some viral particle embodiments of the invention, wherein at least one of the AAV VP1, VP2, and VP3 capsid proteins comprises a modification, e.g., a first member of a protein:protein binding pair, the viral particle further comprises a reference capsid protein, optionally a capsid protein corresponding to the at least one of the AAV VP1, VP2, and VP3 capsid proteins except for the modification, such that the capsid is a mosaic capsid. In some embodiments, a mosaic capsid comprises the VP1 capsid protein modified with a first member of a protein:protein binding pair and a reference VP1 capsid protein. In some embodiments, a mosaic capsid comprises the VP2 capsid protein modified with a first member of a protein:protein binding pair and a reference VP2 capsid protein. In some embodiments, a mosaic capsid comprises the VP3 capsid protein modified with a first member of a protein:protein binding pair and a reference VP3 capsid protein.

Also described are viral particles of the invention comprising AAV capsid proteins of the invention. In some embodiments of the invention, an AAV capsid protein of the invention comprises an amino acid sequence identical to or having significant identity, e.g., at least 95% sequence identity, to an amino acid sequence of a capsid protein of a non-primate animal AAV or a remote AAV, wherein the AAV capsid protein is selected from the group consisting of (a) a chimeric AAV VP1 capsid protein, optionally wherein the chimeric animal AAV VP1 capsid protein modified to comprise at least a first member of a protein:protein binding pair, a detectable label, and/or a point mutation, (b) a non-chimeric AAV VP1 capsid protein modified to comprise at least a first member of a protein:protein binding pair and/or a detectable label, (c) a chimeric VP2 capsid protein, optionally wherein the chimeric AAV VP2 capsid protein is modified to comprise at least a first member of a protein:protein binding pair, a detectable label, and/or a point mutation, (d) a non-chimeric AAV VP2 capsid protein modified to comprise at least a first member of a protein:protein binding pair, a detectable label, and/or a point mutation, (e) a chimeric AAV VP3 capsid protein modified to comprise at least a first member of a protein:protein binding pair, a detectable label, and/or a point mutation, and (f) a non-chimeric AAV VP3 capsid protein modified to comprise at least a first member of a protein:protein binding pair, a detectable label, and/or a point mutation.

In some AAV capsid protein embodiments of the invention, the first member of a protein:protein binding pair and/or detectable label is flanked on one or both sides respectively by a first and/or second linker that link(s) the a first member of a protein:protein binding pair and/or detectable label to the capsid protein, wherein the first and/or second linker is each independently at least one amino acid in length. In some embodiments, the first and second linkers are not identical. In some embodiments, the first and second linkers are identical and are 10 amino acids in length.

In some embodiments, an AAV capsid protein of the invention comprises a detectable label, optionally wherein the detectible label comprises a B1 epitope comprising an amino acid sequence set forth as SEQ ID NO:45. In some embodiments, a detectable label comprises c-myc.

In some embodiments, an AAV capsid protein of the invention comprises both a first member and a second cognate member of the protein:protein binding pair, optionally wherein the first and second members are bound by a covalent bond, optionally an isopeptide bond. In some embodiments, the first member of a protein:protein binding pair is SpyTag, and optionally the second cognate member is SpyCatcher or KTag. In some embodiments, the first member is KTag and the second cognate member is SpyTag. In some embodiments, the first member is SnoopTag and the second cognate member is SnoopCatcher. In some embodiments, the first member is isopeptag and the second cognate member is Pilin-C. In some embodiments, the first member is SpyTag002 and the second cognate member is SpyCatcher002. In some embodiments, the first member comprises a detectable label, such as but not limited to c-myc wherein its binding pair is an anti-c-myc antibody or a portion thereof. In some embodiments, the second member is operably linked to a targeting ligand, optionally wherein the targeting ligand is a binding moiety, that optionally targets a cell marker. In some embodiments, the binding moiety is an antibody, or a portion thereof. In some embodiments, the binding moiety is operably linked to a second member of the protein:protein binding pair, optionally via a covalent bond (such as but not limited to an isopeptide bond) or a linker. In some embodiments, the binding moiety is fused to a second member of the protein:protein binding pair via to a linker fused at the C-terminus of the binding moiety, wherein the linker is fused to the second member the linker's C-terminus, optionally wherein the linker comprises a sequence set forth as SEQ ID NO:49 (GSGESG). In some embodiments, the first member of the protein:protein binding pair is at an amino acid position found in VR I, VR II, VR III, VR IV, VR V, VR VI, VR VII, VR VIII VR IX or the HI loop of the capsid protein, optionally the VR VIII or VR IV of the capsid protein

In some AAV capsid protein embodiments of the invention, the non-primate animal AAV is a non-primate AAV listed in Table 2. In some embodiments, the non-primate AAV is an avian AAV (AAAV), a sea lion AAV or a bearded dragon AAV. In some embodiments, the non-primate animal AAV is an AAAV, and optionally an amino acid sequence of an AAAV capsid protein comprises a modification is at position I444 or I580 of a VP1 capsid protein of AAAV. In some embodiments, the non-primate animal AAV is a squamate AAV, e.g., a bearded dragon AAV, and optionally an amino acid sequence of a bearded dragon AAV comprises a modification is at position I573 or I436 of a VP1 capsid protein of a bearded dragon AAV. In some embodiments, the non-primate animal AAV is a mammalian AAV, e.g., a sea lion AAV, and optionally an amino acid sequence of a seal lion AAV comprises a modification at position selected from the group consisting of I429, I430, I431, I432, I433, I434, I436, I437, and A565 of a VP1 capsid protein of a sea lion AAV.

Non-primate animal VP3 capsid proteins of the invention include non-primate VP3 capsids (a) that encapsidate a genome of an other, e.g., second, AAV and/or (b) that are mutated. In some embodiments, the VP3 capsid protein of the non-human animal AAV of the invention encapsidates the genome of a second AAV that is not of the non-primate animal. In some embodiments, a VP3 capsid protein of a non-primate animal AAV of the invention may be operably linked to a first member of a protein:protein binding pair (optionally via a first and/or second linker) and/or comprise a point mutation, e.g., such that the natural tropism of the capsid protein is reduced to abolished and/or such that the capsid protein comprises a detectable label. In some embodiments, a first member of a protein:protein binding pair comprises a detectable label. In some embodiments, a first member of a protein:protein binding pair comprises the detectable label comprises c-myc (SEQ ID NO:44). In some embodiments the first member of a protein:protein binding pair comprises the first member, and optionally the second member, of a protein:protein binding pair that forms a covalent bond. In some embodiments the protein:protein binding pair is selected from the group consisting of (a) SpyTag:SpyCatcher, (b) SpyTag:KTag, (c) Isopeptag:pilin C, (d) SnoopTag:SnoopCatcher, and I SpyTag002:SpyCatcher002. In some embodiments, a VP3 capsid protein ofa non-primate animal AAV may comprise (a) a B1 epitope (SEQ ID NO:45), (b) SpyTag, (c) SpyCatcher, or any combination of (a)-(c).

In some embodiments, a VP3 capsid protein of a non-primate animal AAV of the invention comprises a first member of a protein:protein binding pair operably linked thereto, optionally via a first or second linker. In some embodiments a first member of a protein:protein binding pair is operably linked to a VP3 capsid of a non-primate animal AAV at an amino acid position found in a variable region (VR) or portion thereof of the VP3 capsid protein, optionally wherein the first member of a protein:protein binding pair is linked to the VP3 capsid via a first and/or second linker. In some embodiments, a first member of a protein:protein binding pair is operably linked to a VP3 capsid of a non-primate animal AAV at an amino acid position found in VR I, VR II, VR III, VR IV, VR V, VR VI, VR VII, VR VIII VR IX or the HI loop of the VP3 capsid protein, optionally wherein the first member of a protein:protein binding pair is linked to the VP3 capsid via a first and/or second linker. In some embodiments, a first member of a protein:protein binding pair is operably linked to a VP3 capsid of a non-primate animal AAV at an amino acid position found in VR VIII or VR IV of the VP3 capsid protein, optionally wherein the first member of a protein:protein binding pair is linked to the VP3 capsid via a first and/or second linker. In some embodiments, a VP3 capsid protein of a non-primate animal AAV is a VP3 capsid protein of a non-primate animal AAV selected from the non-primate animal AAV provided in Table 2. In some embodiments, a VP3 capsid protein of a non-primate animal AAV is VP3 capsid protein of an avian AAV (AAAV). In some embodiments, a VP3 capsid protein of AAAV comprises a first member of a protein:protein binding pair (e.g., SpyTag) operably linked, optionally via a first and/or second linker, at position I444 or I580. In some embodiments, a VP3 capsid protein of a non-primate animal AAV is VP3 capsid protein of a bearded dragon AAV. In some embodiments, a VP3 capsid protein of a bearded dragon AAV comprises a first member of a protein:protein binding pair (e.g., SpyTag) operably linked, optionally via a first and/or second linker, at position I573 or I436. In some embodiments, a VP3 capsid protein of a non-primate animal AAV is VP3 capsid protein of a sea lion AAV. In some embodiments, a VP3 capsid protein of a sea lion AAV comprises a first member of a protein:protein binding pair (e.g., SpyTag) operably linked, optionally via a first and/or second linker, at a position selected from the group consisting of I429, I430, I431, I432, I433, I434, I436, I437, and I565, and optionally at a position selected from the group consisting of I429, I430, I431, I432, I433, I436 and I437; optionally at I432.

Non-primate animal VP2 capsid proteins of the invention include non-primate VP2 capsids (a) that encapsidate a genome of an other, e.g., second, AAV and/or (b) that are mutated. In some embodiments, a VP2 capsid protein of a non-primate animal AAV of the invention encapsidates the genome of an other, e.g., second, AAV. In some embodiments, a VP2 capsid protein of a non-primate animal AAV of the invention may be operably linked to a first member of a protein:protein binding pair and/or comprise a point mutation, e.g., such that the natural tropism of the capsid protein is reduced to abolished and/or such that the capsid protein comprises a detectable label. In some embodiments, a first member of a protein:protein binding pair comprises a detectable label. In some embodiments, a first member of a protein:protein binding pair comprises a detectable label comprising c-myc (SEQ ID NO:44). In some embodiments the first member of a protein:protein binding pair comprises the first member, and optionally the second member, of a protein:protein binding pair that forms a covalent bond. In some embodiments the protein:protein binding pair is selected from the group consisting of (a) SpyTag:SpyCatcher, (b) SpyTag:KTag, (c) Isopeptag:pilin C, (d) SnoopTag:SnoopCatcher, and (e) SpyTag002:SpyCatcher002. In some embodiments, a VP2 capsid protein of a non-primate animal AAV may comprise (a) a B1 epitope (SEQ ID NO:45), (b) SpyTag, (c) SpyCatcher, or any combination of (a)-(c).

In some embodiments, a VP2 capsid protein of a non-primate animal AAV of the invention comprises a first member of a protein:protein binding pair operably linked thereto, optionally via a first and second linker. In some embodiments a first member of a protein:protein binding pair is operably linked to a VP2 capsid of a non-primate animal AAV at an amino acid position found in a variable region (VR) or portion thereof of the VP2 capsid protein, optionally wherein the first member of a protein:protein binding pair is linked to the VP2 capsid via a first and/or second linker. In some embodiments, a first member of a protein:protein binding pair is operably linked to a VP2 capsid of a non-primate animal AAV at an amino acid position found in VR I, VR II, VR III, VR IV, VR V, VR VI, VR VII, VR VIII VR IX or the HI loop of the VP2 capsid protein, optionally wherein the first member of a protein:protein binding pair is linked to the VP2 capsid via a first and/or second linker. In some embodiments, a first member of a protein:protein binding pair is operably linked to a VP2 capsid of a non-primate animal AAV at an amino acid position found in VR VIII or VR IV of the VP2 capsid protein, optionally wherein the first member of a protein:protein binding pair is linked to the VP2 capsid via a first and/or second linker. In some embodiments, a VP2 capsid protein of a non-primate animal AAV is a VP2 capsid protein of a non-primate animal AAV selected from the non-primate animal AAV provided in Table 2. In some embodiments, a VP2 capsid protein of a non-primate animal AAV is VP2 capsid protein of an avian AAV (AAAV). In some embodiments, a VP2 capsid protein of AAAV comprises a first member of a protein:protein binding pair (e.g., SpyTag) operably linked, optionally via a first and/or second linker, at position I444 or I580. In some embodiments, a VP2 capsid protein of a non-primate animal AAV is VP2 capsid protein of a bearded dragon AAV. In some embodiments, a VP2 capsid protein of a bearded dragon AAV comprises a first member of a protein:protein binding pair (e.g., SpyTag) operably linked, optionally via a first and/or second linker, at position I573 or I436. In some embodiments, a VP2 capsid protein of a non-primate animal AAV is VP2 capsid protein of a sea lion AAV. In some embodiments, a VP2 capsid protein of a sea lion AAV comprises a first member of a protein:protein binding pair (e.g., SpyTag) operably linked, optionally via a first and/or second linker, at a position selected from the group consisting of I429, I430, I431, I432, I433, I434, I436, I437, and I565; optionally at a position selected from the group consisting of I429, I430, I431, I432, I433, I436 and I437; optionally at I431.

In some embodiments, a VP2 capsid protein of the invention may be a chimeric VP2 capsid protein that comprises in operable linkage a portion of a VP2 capsid protein of the non-primate animal AAV and a portion of a VP2 capsid protein of an other, e.g., second, AAV. In some embodiments, a chimeric VP2 capsid protein comprises from N-terminus to C-terminus (a) a portion of the VP2 capsid protein of an other, e.g., second, AAV operably linked to (b) a portion of the VP2 capsid of the non-primate animal AAV that comprises at least the amino acid sequence of the VP3 capsid protein of the non-primate animal AAV. In some embodiments, a chimeric VP2 capsid protein may comprise from N-terminus to C-terminus (a) an amino acid sequence of the VP1/VP2 common region of the other AAV operably linked to (b) an amino acid sequence of the VP3 capsid protein of the non-primate animal AAV. In some embodiments the other AAV is a non-primate animal AAV. In some other embodiments, the other AAV is a primate AAV.

In some embodiments, a chimeric VP2 capsid protein of the invention comprises (a) a portion of the VP2 capsid protein of a primate AAV operably linked to (b) a portion of the VP2 capsid of a non-primate animal AAV that comprises at least the amino acid sequence of the VP3 capsid protein of a non-primate animal AAV. In some embodiments, a chimeric VP2 capsid protein may comprise from N-terminus to C-terminus (a) an amino acid sequence of the VP1/VP2 common region of a primate AAV operably linked to (b) an amino acid sequence of a VP3 capsid protein of a non-primate animal AAV. In some embodiments, the primate AAV is AAV1. In some embodiments, the primate AAV is AAV2. In some embodiments, the primate AAV is AAV3. In some embodiments, the primate AAV is AAV4. In some embodiments, the primate AAV is AAV5. In some embodiments, the primate AAV is AAV6. In some embodiments, the primate AAV is AAV7. In some embodiments, the primate AAV is AAV8. In some embodiments, the primate AAV is AAV9. In some embodiments, the non-primate animal AAV is selected from the group of non-primate animal AAV provided in Table 2. In some embodiments, the non-primate animal AAV is an avian AAV, a bearded dragon AAV, or a sea lion AAV.

In some embodiments, a chimeric VP2 capsid protein of the invention comprises (a) a portion of the VP2 capsid protein of AAV2 operably linked to (b) a portion of the VP2 capsid of a non-primate animal AAV that comprises at least the amino acid sequence of the VP3 capsid protein of the non-primate animal AAV. In some embodiments, a chimeric VP2 capsid protein may comprise from N-terminus to C-terminus (a) an amino acid sequence of the VP1/VP2 common region of AAV2 operably linked to (b) an amino acid sequence of a VP3 capsid protein of a non-primate animal AAV.

In some embodiments, a chimeric AAV2/AAAV VP2 capsid protein of the invention comprises (a) a portion of the VP2 capsid protein of AAV2 operably linked to (b) a portion of the VP2 capsid protein of an avian AAV (AAAV) that comprises at least the amino acid sequence of the VP3 capsid protein of the AAAV. In some embodiments, a chimeric AAV2/AAAV VP2 capsid protein may comprise from N-terminus to C-terminus (a) an amino acid sequence of the VP1/VP2 common region of AAV2 operably linked to (b) an amino acid sequence of a VP3 capsid protein of the AAAV.

In some embodiments, a chimeric AAV2/sea lion AAV VP2 capsid protein of the invention comprises (a) a portion of the VP2 capsid protein of AAV2 operably linked to (b) a portion of the VP2 capsid protein of a sea lion AAV that comprises at least the amino acid sequence of the VP3 capsid protein of the sea lion AAV. In some embodiments, a chimeric AAV2/sea lion AAV VP2 capsid protein may comprise from N-terminus to C-terminus (a) an amino acid sequence of the VP1/VP2 common region of AAV2 operably linked to (b) an amino acid sequence of a VP3 capsid protein of the sea lion AAV.

In some embodiments, a chimeric AAV2/bearded dragon AAV VP2 capsid protein of the invention comprises (a) a portion of the VP2 capsid protein of AAV2 operably linked to (b) a portion of the VP2 capsid protein of a bearded dragon AAV that comprises at least the amino acid sequence of the VP3 capsid protein of the bearded dragon AAV. In some embodiments, a chimeric AAV2/bearded dragon AAV VP2 capsid protein may comprise from N-terminus to C-terminus (a) an amino acid sequence of the VP1/VP2 common region of AAV2 operably linked to (b) an amino acid sequence of a VP3 capsid protein of a bearded dragon AAV.

In some embodiments, a chimeric VP2 capsid protein of the invention may be operably linked to a first member of a protein:protein binding pair and/or comprise a point mutation, e.g., such that the natural tropism of the capsid protein is reduced to abolished and/or such that the capsid protein comprises a detectable label. In some embodiments, a first member of a protein:protein binding pair comprises a detectable label. In some embodiments, a first member of a protein:protein binding pair comprises the detectable label comprises c-myc (SEQ ID NO:44). In some embodiments the first member of a protein:protein binding pair comprises the first member, and optionally the second member, of a protein:protein binding pair that forms a covalent bond. In some embodiments the protein:protein binding pair is selected from the group consisting of (a) SpyTag:SpyCatcher, (b) SpyTag:KTag, (c) Isopeptag:pilin C, (d) SnoopTag:SnoopCatcher, and (e) SpyTag002:SpyCatcher002. In some embodiments, a chimeric VP2 capsid protein may comprise (a) a B1 epitope (SEQ ID NO:45), (b) SpyTag, (c) SpyCatcher, or any combination of (a)-(c).

In some embodiments, a chimeric primate/non-primate animal VP2 capsid protein of the invention (e.g., a chimeric AAV2/AAAV VP2 capsid protein, a chimeric AAV2/sea lion AAV VP2 capsid protein, a chimeric AAV2/bearded dragon AAV VP2 capsid protein, etc.) comprises a first member of a protein:protein binding pair operably linked thereto, optionally via a first or second linker. In some embodiments a first member of a protein:protein binding pair is operably linked to a chimeric primate/non-primate animal VP2 capsid protein (e.g., a chimeric AAV2/AAAV VP2 capsid protein, a chimeric AAV2/sea lion AAV VP2 capsid protein, a chimeric AAV2/bearded dragon AAV VP2 capsid protein, etc.) at an amino acid position found in a variable region (VR) or portion thereof of the chimeric primate/non-primate VP2 capsid protein, optionally wherein the first member of a protein:protein binding pair is linked to the chimeric primate/non-primate VP2 capsid protein via a first and/or second linker. In some embodiments, a first member of a protein:protein binding pair is operably linked to a chimeric primate/non-primate VP2 capsid protein at an amino acid position found in VR I, VR II, VR III, VR IV, VR V, VR VI, VR VII, VR VIII VR IX or the HI loop of the chimeric primate/non-primate VP2 capsid protein, optionally wherein the first member of a protein:protein binding pair is linked to the VP2 capsid via a first and/or second linker. In some embodiments, a first member of a protein:protein binding pair is operably linked to a chimeric primate/non-primate VP2 capsid protein (e.g., a chimeric AAV2/AAAV VP2 capsid protein, a chimeric AAV2/sea lion AAV VP2 capsid protein, a chimeric AAV2/bearded dragon AAV VP2 capsid protein, etc.) at an amino acid position found in VR VIII or VR IV of the chimeric primate/non-primate VP2 capsid protein, optionally wherein the first member of a protein:protein binding pair is linked to the chimeric primate/non-primate VP2 capsid protein via a first and/or second linker. In some embodiments, a chimeric AAV2/AAAV VP2 capsid protein comprises a first member of a protein:protein binding pair (e.g., SpyTag) operably linked, optionally via a first and/or second linker, at position I444 or I580. In some embodiments, a chimeric AAV2/bearded dragon AAV VP2 capsid protein comprises a first member of a protein:protein binding pair (e.g., SpyTag) operably linked, optionally via a first and/or second linker, at position I573 or I436. In some embodiments, a chimeric AAV2/sea lion AAV VP2 capsid protein comprises a first member of a protein:protein binding pair (e.g., SpyTag) operably linked, optionally via a first and/or second linker, at a position selected from the group consisting of I429, I430, I431, I432, I433, I434, I436, I437, and I565; optionally at a position selected from the group consisting of I429, I430, I431, I432, I433, I436 and I437; optionally at I431.

Non-primate animal VP1 capsid proteins of the invention include non-primate VP1 capsids (a) that encapsidate a genome of an other, e.g., second, AAV and/or (b) that are mutated. In some embodiments, a VP1 capsid protein of a non-primate animal AAV of the invention encapsidates a genome of an other, e.g., second, AAV. In some embodiments, a VP1 capsid protein of a non-primate animal of the invention may be operably linked to a first member of a protein:protein binding pair and/or comprise a point mutation, e.g., such that the natural tropism of the capsid protein is reduced to abolished and/or such that the capsid protein comprises a detectable label. In some embodiments, a first member of a protein:protein binding pair comprises a detectable label. In some embodiments, a first member of a protein:protein binding pair comprises the detectable label comprises c-myc (SEQ ID NO:44). In some embodiments the first member of a protein:protein binding pair comprises the first member, and optionally the second member, of a protein:protein binding pair that forms a covalent bond. In some embodiments the protein:protein binding pair is selected from the group consisting of (a) SpyTag:SpyCatcher, (b) SpyTag:KTag, (c) Isopeptag:pilin C, (d) SnoopTag:SnoopCatcher, and (e) SpyTag002:SpyCatcher002. In some embodiments, a VP3 capsid protein ofa non-primate animal AAV may comprise (a) a B1 epitope (SEQ ID NO:45), (b) SpyTag, (c) SpyCatcher, or any combination of (a)-(c).

In some embodiments a first member of a protein:protein binding pair is operably linked to a VP1 capsid of a non-primate animal AAV of the invention at an amino acid position found in a variable region (VR) or portion thereof of the VP1 capsid protein, optionally wherein the first member of a protein:protein binding pair is linked to the VP1 capsid via a first and/or second linker. In some embodiments, a first member of a protein:protein binding pair is operably linked to a VP1 capsid of a non-primate animal AAV at an amino acid position found in VR I, VR II, VR III, VR IV, VR V, VR VI, VR VII, VR VIII VR IX or the HI loop of the VP1 capsid protein, optionally wherein the first member of a protein:protein binding pair is linked to the VP1 capsid via a first and/or second linker. In some embodiments, a first member of a protein:protein binding pair is operably linked to a VP1 capsid of a non-primate animal AAV at an amino acid position found in VR VIII or VR IV of the VP1 capsid protein, optionally wherein the first member of a protein:protein binding pair is linked to the VP1 capsid via a first and/or second linker. In some embodiments, a VP1 capsid protein of a non-primate animal AAV is a VP1 capsid protein of a non-primate AAV selected from the group of non-primate AAVs provided in Table 2. In some embodiments, a VP1 capsid protein of a non-primate animal AAV is VP1 capsid protein of an avian AAV (AAAV). In some embodiments, a VP1 capsid protein of AAAV comprises a first member of a protein:protein binding pair (e.g., SpyTag) operably linked, optionally via a first and/or second linker, at position I444 or I580. In some embodiments, a VP1 capsid protein of a non-primate animal AAV is VP31 capsid protein of a bearded dragon AAV. In some embodiments, a VP1 capsid protein of a bearded dragon AAV comprises a first member of a protein:protein binding pair (e.g., SpyTag) operably linked, optionally via a first and/or second linker, at position I573 or I436. In some embodiments, a VP1 capsid protein of a non-primate animal AAV is VP1 capsid protein of a sea lion AAV. In some embodiments, a VP1 capsid protein of a sea lion AAV comprises a first member of a protein:protein binding pair (e.g., SpyTag) operably linked, optionally via a first and/or second linker, at position selected from the group consisting I429, I430, I431, I432, I433, I434, I436, I437, and I565; optionally at a position selected from the group consisting of I429, I430, I431, I432, I433, I436 and I437; optionally at I431.

In some other embodiments, a VP1 capsid protein of the invention may be a chimeric VP1 capsid protein that comprises in operably linkage a portion of a VP1 capsid protein of the non-primate animal AAV and a portion of the VP1 capsid protein of an other AAV, wherein the other AAV is not the non-primate animal AAV. In some embodiments, a chimeric VP1 capsid protein comprises from N-terminus to C-terminus (a) a portion of the VP1 capsid protein of the other AAV that comprises at least a PLA₂ domain of the other AAV and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) a portion of the VP1 capsid protein of the other AAV that comprises at least a VP1-u domain of the other AAV and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) an amino acid sequence of a VP1-u domain and a VP1/VP2 common region of an other, e.g., second, AAV and (b) an amino acid sequence of the VP3 capsid protein of the non-primate animal AAV. In some embodiments the other AAV is a non-primate animal AAV. In some other embodiments, the other AAV is a primate AAV.

In some embodiments, a chimeric VP1 capsid protein comprises from N-terminus to C-terminus (a) a portion of the VP1 capsid protein of a primate AAV that comprises at least a PLA₂ domain of the primate AAV and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) a portion of the VP1 capsid protein of a primate AAV that comprises at least a VP1-u domain of the primate AAV and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) an amino acid sequence of a VP1-u domain and a VP1/VP2 common region of a primate AAV and (b) an amino acid sequence of the VP3 capsid protein of the non-primate animal AAV. In some embodiments, the primate AAV is AAV1. In some embodiments, the primate AAV is AAV2. In some embodiments, the primate AAV is AAV3. In some embodiments, the primate AAV is AAV4. In some embodiments, the primate AAV is AAV5. In some embodiments, the primate AAV is AAV6. In some embodiments, the primate AAV is AAV7. In some embodiments, the primate AAV is AAV8. In some embodiments, the primate AAV is AAV9. In some embodiments, the non-primate animal AAV is selected from the group of non-primate animal AAV provided in Table 2. In some embodiments, the non-primate animal AAV is an avian AAV, a bearded dragon AAV, or a sea lion AAV.

In some embodiments, a chimeric VP1 capsid protein comprises from N-terminus to C-terminus (a) a portion of the VP1 capsid protein of AAV2 that comprises at least a PLA₂ domain of AAV2 and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) a portion of the VP1 capsid protein of AAV2 that comprises at least a VP1-u domain of AAV2 and (b) a portion of the VP1 capsid of the non-primate animal AAV that comprises at least the amino acid sequence of the VP3 capsid protein of the non-primate animal AAV. In some embodiments, a chimeric VP1 capsid protein comprises from N-terminus to C-terminus (a) an amino acid sequence of a VP1-u domain and a VP1/VP2 common region of AAV2 and (b) an amino acid sequence of the VP3 capsid protein of the non-primate animal.

In some embodiments, a chimeric AAV2/AAAV VP1 capsid protein comprises from N-terminus to C-terminus (a) a portion of the VP1 capsid protein of AAV2 that comprises at least a PLA₂ domain of AAV2 and (b) a portion of the VP1 capsid of an avian AAV (AAAV) that comprises at least the amino acid sequence of the VP3 capsid protein of the AAAV. In some embodiments, a chimeric AAV2/AAAV VP1 capsid protein comprises from N-terminus to C-terminus (a) a portion of the VP1 capsid protein of AAV2 that comprises at least a VP1-u domain of AAV2 and (b) a portion of the VP1 capsid of an AAAV that comprises at least the amino acid sequence of the VP3 capsid protein of the AAAV. In some embodiments, a chimeric AAV2/AAAV VP1 capsid protein comprises from N-terminus to C-terminus (a) an amino acid sequence of a VP1-u domain and a VP1/VP2 common region of AAV2 and (b) an amino acid sequence of the VP3 capsid protein of an AAAV. In some embodiments, a chimeric AAV2/AAAV VP1 capsid protein comprises the amino acid sequence set forth as SEQ ID NO:2.

In some embodiments, a chimeric AAV2/sea lion VP1 capsid protein comprises from N-terminus to C-terminus (a) a portion of the VP1 capsid protein of AAV2 that comprises at least a PLA₂ domain of AAV2 and (b) a portion of the VP1 capsid of a sea lion AAV that comprises at least the amino acid sequence of the VP3 capsid protein of the sea lion. In some embodiments, a chimeric AAV2/sea lion VP1 capsid protein comprises from N-terminus to C-terminus (a) a portion of the VP1 capsid protein of AAV2 that comprises at least a VP1-u domain of AAV2 and (b) a portion of the VP1 capsid of a sea lion that comprises at least the amino acid sequence of the VP3 capsid protein of the sea lion. In some embodiments, a chimeric AAV2/sea lion VP1 capsid protein comprises from N-terminus to C-terminus (a) an amino acid sequence of a VP1-u domain and a VP1/VP2 common region of AAV2 and (b) an amino acid sequence of the VP3 capsid protein of a sea lion. In some embodiments, a chimeric AAV2/sea lion VP1 capsid protein comprises the amino acid sequence set forth as SEQ ID NO:4.

In some embodiments, a chimeric AAV2/bearded dragon VP1 capsid protein comprises from N-terminus to C-terminus (a) a portion of the VP1 capsid protein of AAV2 that comprises at least a PLA₂ domain of AAV2 and (b) a portion of the VP1 capsid of a bearded dragon AAV that comprises at least the amino acid sequence of the VP3 capsid protein of the bearded dragon. In some embodiments, a chimeric AAV2/bearded dragon VP1 capsid protein comprises from N-terminus to C-terminus (a) a portion of the VP1 capsid protein of AAV2 that comprises at least a VP1-u domain of AAV2 and (b) a portion of the VP1 capsid of a bearded dragon that comprises at least the amino acid sequence of the VP3 capsid protein of the bearded dragon. In some embodiments, a chimeric AAV2/bearded dragon VP1 capsid protein comprises from N-terminus to C-terminus (a) an amino acid sequence of a VP1-u domain and a VP1/VP2 common region of AAV2 and (b) an amino acid sequence of the VP3 capsid protein of a bearded dragon. In some embodiments, a chimeric AAV2/bearded dragon VP1 capsid protein comprises the amino acid sequence set forth as SEQ ID NO:6.

In some embodiments, a chimeric VP1 capsid protein may be operably linked to a first member of a protein:protein binding pair and/or comprise a point mutation, e.g., such that the natural tropism of the capsid protein is reduced to abolished and/or such that the capsid protein comprises a detectable label. In some embodiments, a first member of a protein:protein binding pair comprises a detectable label. In some embodiments, a first member of a protein:protein binding pair comprises the detectable label comprises c-myc (SEQ ID NO:44). In some embodiments the first member of a protein:protein binding pair comprises the first member, and optionally the second member, of a protein:protein binding pair that forms a covalent bond. In some embodiments the protein:protein binding pair is selected from the group consisting of (a) SpyTag:SpyCatcher, (b) SpyTag:KTag, (c) Isopeptag:pilin C, (d) SnoopTag:SnoopCatcher, and (e) SpyTag002:SpyCatcher002. In some embodiments, a chimeric VP1 capsid protein may comprise (a) a B1 epitope (SEQ ID NO:45), (b) SpyTag, (c) SpyCatcher, and any combination of (a)-(c).

In some embodiments, a chimeric primate/non-primate animal VP1 capsid protein (e.g., a chimeric AAV2/AAAV VP1 capsid protein, a chimeric AAV2/sea lion AAV VP1 capsid protein, a chimeric AAV2/bearded dragon AAV VP1 capsid protein, etc.) comprises a first member of a protein:protein binding pair operably linked thereto, optionally via a first or second linker. In some embodiments a first member of a protein:protein binding pair is operably linked to a chimeric primate/non-primate animal VP1 capsid protein (e.g., a chimeric AAV2/AAAV VP1 capsid protein, a chimeric AAV2/sea lion AAV VP1 capsid protein, a chimeric AAV2/bearded dragon AAV VP1 capsid protein, etc.) at an amino acid position found in a variable region (VR) or portion thereof of the chimeric primate/non-primate VP1 capsid protein, optionally wherein the first member of a protein:protein binding pair is linked to the chimeric primate/non-primate VP1 capsid protein via a first and/or second linker. In some embodiments, a first member of a protein:protein binding pair is operably linked to a VP1 capsid of a chimeric primate/non-primate animal VP1 capsid protein at an amino acid position found in VR I, VR II, VR III, VR IV, VR V, VR VI, VR VII, VR VIII VR IX or the HI loop of the chimeric primate/non-primate animal VP1 capsid protein, optionally wherein the first member of a protein:protein binding pair is linked to the chimeric primate/non-primate animal VP1 capsid protein capsid via a first and/or second linker. In some embodiments, a first member of a protein:protein binding pair is operably linked to a chimeric primate/non-primate VP1 capsid protein (e.g., a chimeric AAV2/AAAV VP1 capsid protein, a chimeric AAV2/sea lion AAV VP1 capsid protein, a chimeric AAV2/bearded dragon AAV VP1 capsid protein, etc.) at an amino acid position found in VR VIII or VR IV of the chimeric primate/non-primate VP1 capsid protein, optionally wherein the first member of a protein:protein binding pair is linked to the chimeric primate/non-primate VP1 capsid protein via a first and/or second linker.

In some embodiments, a chimeric AAV2/AAAV VP1 capsid protein comprises a first member of a protein:protein binding pair operably linked, optionally via a first and/or second linker, at position I444 or I580. In some embodiments, a chimeric AAV2/AAAV VP1 capsid protein comprises SpyTag operably linked, optionally via a first and second linker sequence, at position I444. In some embodiments, a chimeric AAV2/AAAV VP1 capsid protein comprises an amino acid sequence set forth as SEQ ID NO:8. In some embodiments, a chimeric AAV2/AAAV VP1 capsid protein comprises SpyTag operably linked, optionally via a first and second linker sequence, at position I580. In some embodiments, a chimeric AAV2/AAAV VP1 capsid protein comprises an amino acid sequence set forth as SEQ ID NO:10.

In some embodiments, a chimeric AAV2/sea lion AAV VP1 capsid protein comprises a first member of a protein:protein binding pair operably linked, optionally via a first and/or second linker, at a position selected from the group consisting of I429, I430, I431, I432, I433, I434, I436, I437, and I565; optionally at a position selected from the group consisting of I429, I430, I431, I432, I433, I436 and I437; optionally at I432. In some embodiments, a chimeric AAV2/sea lion AAV VP1 capsid protein comprises SpyTag operably linked, optionally via a first and second linker sequence, at position I432. In some embodiments, a chimeric AAV2/sea lion AAV VP1 capsid protein comprises an amino acid sequence set forth as SEQ ID NO:12. In some embodiments, a chimeric AAV2/sea lion AAV VP1 capsid protein comprises SpyTag operably linked, optionally via a first and second linker sequence, at position I565. In some embodiments, a chimeric AAV2/sea lion AAV VP1 capsid protein comprises an amino acid sequence set forth as SEQ ID NO:14. In some embodiments, a chimeric AAV2/sea lion AAV VP1 capsid protein comprises SpyTag operably linked, optionally via a first and second linker sequence, at position I429. In some embodiments, a chimeric AAV2/sea lion AAV VP1 capsid protein comprises an amino acid sequence set forth as SEQ ID NO:16. In some embodiments, a chimeric AAV2/sea lion AAV VP1 capsid protein comprises SpyTag operably linked, optionally via a first and second linker sequence, at position I430. In some embodiments, a chimeric AAV2/sea lion AAV VP1 capsid protein comprises an amino acid sequence set forth as SEQ ID NO:18. In some embodiments, a chimeric AAV2/sea lion AAV VP1 capsid protein comprises SpyTag operably, optionally linked via a first and second linker sequence, at position I431. In some embodiments, a chimeric AAV2/sea lion AAV VP1 capsid protein comprises an amino acid sequence set forth as SEQ ID NO:20. In some embodiments, a chimeric AAV2/sea lion AAV VP1 capsid protein comprises SpyTag operably linked, optionally via a first and second linker sequence, at position I433. In some embodiments, a chimeric AAV2/sea lion AAV VP1 capsid protein comprises an amino acid sequence set forth as SEQ ID NO:22. In some embodiments, a chimeric AAV2/sea lion AAV VP1 capsid protein comprises SpyTag operably linked, optionally via a first and second linker sequence, at position I434. In some embodiments, a chimeric AAV2/sea lion AAV VP1 capsid protein comprises an amino acid sequence set forth as SEQ ID NO:24. In some embodiments, a chimeric AAV2/sea lion AAV VP1 capsid protein comprises SpyTag operably linked, optionally via a first and second linker sequence, at position I435. In some embodiments, a chimeric AAV2/sea lion AAV VP1 capsid protein comprises an amino acid sequence set forth as SEQ ID NO:26. In some embodiments, a chimeric AAV2/sea lion AAV VP1 capsid protein comprises SpyTag operably, optionally linked via a first and second linker sequence, at position I436. In some embodiments, a chimeric AAV2/sea lion AAV VP1 capsid protein comprises an amino acid sequence set forth as SEQ ID NO:28. In some embodiments, a chimeric AAV2/sea lion AAV VP1 capsid protein comprises SpyTag operably linked, optionally via a first and second linker sequence, at position I437. In some embodiments, a chimeric AAV2/sea lion AAV VP1 capsid protein comprises an amino acid sequence set forth as SEQ ID NO:30. In some embodiments, a chimeric AAV2/sea lion AAV VP1 capsid protein comprises SpyTag operably linked, optionally via a first and second linker sequence, at position I432. In some embodiments, a chimeric AAV2/sea lion AAV VP1 capsid protein comprises an amino acid sequence set forth as SEQ ID NO:32. In some embodiments, a chimeric AAV2/sea lion AAV VP1 capsid protein comprises an amino acid sequence set forth as SEQ ID NO:53. In some embodiments, a chimeric AAV2/sea lion AAV VP1 capsid protein comprises an amino acid sequence set forth as SEQ ID NO:55. In some embodiments, a chimeric AAV2/sea lion AAV VP1 capsid protein comprises an amino acid sequence set forth as SEQ ID NO:57. In some embodiments, a chimeric AAV2/sea lion AAV VP1 capsid protein comprises an amino acid sequence set forth as SEQ ID NO:59. In some embodiments, a chimeric AAV2/sea lion AAV VP1 capsid protein comprises an amino acid sequence set forth as SEQ ID NO:61. In some embodiments, a chimeric AAV2/sea lion AAV VP1 capsid protein comprises an amino acid sequence set forth as SEQ ID NO:63. In some embodiments, a chimeric AAV2/sea lion AAV VP1 capsid protein comprises an amino acid sequence set forth as SEQ ID NO:65. In some embodiments, a chimeric AAV2/sea lion AAV VP1 capsid protein comprises an amino acid sequence set forth as SEQ ID NO:67. In some embodiments, a chimeric AAV2/sea lion AAV VP1 capsid protein comprises an amino acid sequence set forth as SEQ ID NO:69. In some embodiments, a chimeric AAV2/sea lion AAV VP1 capsid protein comprises an amino acid sequence set forth as SEQ ID NO:71.

In some embodiments, a chimeric AAV2/bearded dragon AAV VP1 capsid protein comprises a first member of a protein:protein binding pair operably linked, optionally via a first and/or second linker, at position I436 or I573. In some embodiments, a chimeric AAV2/bearded dragon AAV VP1 capsid protein comprises SpyTag operably linked, optionally via a first and second linker sequence, at position I436. In some embodiments, a chimeric AAV2/bearded dragon AAV VP1 capsid protein comprises an amino acid sequence set forth as SEQ ID NO:34. In some embodiments, a chimeric AAV2/bearded dragon AAV VP1 capsid protein comprises SpyTag operably linked, optionally via a first and second linker sequence, at position I573. In some embodiments, a chimeric AAV2/bearded dragon AAV VP1 capsid protein comprises an amino acid sequence set forth as SEQ ID NO:36.

In some embodiments, a capsid protein of the invention further comprises a first member and a second member of a protein:protein binding pair, optionally wherein the second member is operably linked to a targeting ligand, optionally wherein the targeting ligand is a binding moiety. In some embodiments, the binding moiety is an antibody, or a portion thereof. In some embodiments, the antibody, or portion thereof, is fused to the second member, e.g., SpyCatcher. In some embodiments, the antibody or portion thereof is fused at its C-terminus to a linker, optionally a linker comprising a sequence set forth as SEQ ID NO:49 (GSGESG), and the linker is fused to the second member, e.g., SpyCatcher, at the linker's C-terminus.

In some embodiments, a capsid protein of the invention may comprise a detectable label, which detectable label may optionally act as a first member of a protein:protein binding pair and/or for detection and/or isolation of the capsid protein. In some embodiments, the detectable label is c-myc. In some embodiments the detectable label comprises the AAV B1 epitope, e.g., the amino acid sequence IGTRYLTR (SEQ ID NO: 45).

An AAV capsid protein of the invention may comprise an amino acid sequence selected from the group consisting of (a) an amino acid sequence set forth as SEQ ID NO:2, (b) an amino acid sequence set forth as SEQ ID NO:4, (c) an amino acid sequence set forth as SEQ ID NO:6, (d) an amino acid sequence set forth as SEQ ID NO:8, (e) an amino acid sequence set forth as SEQ ID NO:10, (f) an amino acid sequence set forth as SEQ ID NO:12, (g) an amino acid sequence set forth as SEQ ID NO:14, (h) an amino acid sequence set forth as SEQ ID NO:16, (i) an amino acid sequence set forth as SEQ ID NO:18, (j) an amino acid sequence set forth as SEQ ID NO:20, (k) an amino acid sequence set forth as SEQ ID NO:22, (l) an amino acid sequence set forth as SEQ ID NO:24, (m) an amino acid sequence set forth as SEQ ID NO:26, (n) an amino acid sequence set forth as SEQ ID NO:28, (o) an amino acid sequence set forth as SEQ ID NO:30, (p) an amino acid sequence set forth as SEQ ID NO:32, (q) an amino acid sequence set forth as SEQ ID NO:34, (r) an amino acid sequence set forth as SEQ ID NO:36, (s) the amino acid sequence set forth as SEQ ID NO:53, (t) the amino acid sequence set forth as SEQ ID NO:55, (u) the amino acid sequence set forth as SEQ ID NO:57, (v) the amino acid sequence set forth as SEQ ID NO:59, (w) the amino acid sequence set forth as SEQ ID NO:61, (x) the amino acid sequence set forth as SEQ ID NO:63, (y) the amino acid sequence set forth as SEQ ID NO:65, (z) the amino acid sequence set forth as SEQ ID NO:67, (aa) the amino acid sequence set forth as SEQ ID NO:69, (bb) the amino acid sequence set forth as SEQ ID NO:71, (cc) an amino acid sequence having significant sequence identity, e.g., at least 95% identity, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34,SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71 and (dd) an amino acid sequence of any VP2 and/or VP3 portions of the amino acid sequences set forth in any of (a)-(cc).

In some embodiments a capsid protein of the invention is encoded by a nucleic acid molecule of the invention. Also provided herein are nucleic acid molecules encoding the capsid proteins of the invention.

Described herein are nucleic acid molecules comprising an AAV cap gene that encodes an AAV VP1 capsid protein, an AAV VP2 capsid protein and/or an AAV VP3 capsid protein, wherein the AAV cap gene, or portion thereof, comprises a nucleic acid sequence having significant sequence identity, e.g., at least 95% identity, to the nucleic acid sequence of a cap gene of a non-primate animal AAV or portion thereof, or a remote AAV or a portion thereof, and wherein the AAV cap gene is further modified to comprise (a) a nucleotide sequence encoding a first member of a protein:protein binding pair, (b) a nucleotide sequence encoding a detectable label, (c) a point mutation, (d) a chimeric nucleotide sequence, or (e) any combination of (a), (b), (c), and (d). In some nucleic acid molecule embodiments, the nucleic acid comprises an AAV rep gene and an AAV cap gene, wherein the entire AAV cap gene comprises a first nucleic acid sequence having significant sequence identity, e.g., at least 95% identity, to the nucleic acid sequence of a cap gene of a non-primate animal AAV or a remote AAV, and wherein the AAV rep gene, or portion thereof, comprises a second nucleic acid sequence having significant sequence identity, e.g., at least 95% identity, to the nucleic acid sequence of a rep gene of a second AAV, or portion thereof, wherein the non-primate animal AAV is not identical to the second AAV. In some embodiments, a nucleic acid molecule of the invention comprises an AAV cap gene that encodes an AAV capsid protein, wherein the AAV cap gene comprises a nucleotide sequence identical to or having significant identity, e.g., at least 95% sequence identity, to at least a portion of the nucleotide sequence of a cap gene selected from the group consisting of (i) a cap gene of a non-primate animal AAV, (ii) a cap gene of a remote AAV, or (iii) a combination thereof, wherein said AAV cap gene is further modified to comprise (a) a nucleotide sequence encoding a first member of a protein:protein binding pair, (b) a nucleotide sequence encoding a detectable label, (c) a point mutation, (d) a chimeric nucleotide sequence comprising a portion of a nucleotide sequence of an other, e.g., second, AAV cap gene that is operably linked to said nucleotide sequence of the AAV cap gene selected from the group consisting of the cap gene non-primate animal AAV, a remote AAV, or a combination thereof, (e) any combination of (a), (b), (c), and (d).

In some embodiments, a nucleic acid molecule of the invention comprises an AAV cap gene that encodes an AAV capsid protein, wherein the AAV cap gene comprises a nucleotide sequence identical to or having significant identity, e.g., at least 95% sequence identity, to at least a portion of the nucleotide sequence of a cap gene of a non-primate animal AAV, wherein said AAV cap gene is further modified to comprise (a) a nucleotide sequence encoding a first member of a protein:protein binding pair, (b) a nucleotide sequence encoding a detectable label, (c) a point mutation, (d) a chimeric nucleotide sequence comprising a portion of a nucleotide sequence of an other, e.g., second, AAV cap gene that is operably linked to said nucleotide sequence of the cap gene non-primate animal AAV, (e) any combination of (a), (b), (c), and (d).

In some embodiments, a nucleic acid molecule of the invention comprises an AAV cap gene that encodes an AAV capsid protein, wherein the AAV cap gene comprises a nucleotide sequence identical to or having significant identity, e.g., at least 95% sequence identity, to at least a portion of the nucleotide sequence of a cap gene of a remote animal AAV, wherein said AAV cap gene is further modified to comprise (a) a nucleotide sequence encoding a first member of a protein:protein binding pair, (b) a nucleotide sequence encoding a detectable label, (c) a point mutation, (d) a chimeric nucleotide sequence comprising a portion of a nucleotide sequence of an other AAV cap gene that is operably linked to said nucleotide sequence of the cap gene remote animal AAV, (e) any combination of (a), (b), (c), and (d).

In some embodiments, a nucleic acid molecule of the invention comprises an AAV rep gene and an AAV cap gene, wherein the AAV cap gene comprises a first nucleotide sequence identical to or having significant identity, e.g., at least 95% sequence identity, to a nucleotide sequence of a cap gene selected from the group consisting of (i) a cap gene of a non-primate animal AAV, (ii) a cap gene of a remote primate AAV, and (iv) a combination thereof, wherein the AAV rep gene comprises a second nucleotide sequence of an AAV rep gene of an other, e.g., second AAV.

In some embodiments, a nucleic acid molecule of the invention comprises an AAV rep gene and an AAV cap gene, wherein the AAV cap gene comprises a first nucleotide sequence a nucleotide sequence identical to or having significant identity, e.g., at least 95% sequence identity, to a nucleotide sequence of a cap gene of a non-primate animal AAV, wherein the AAV rep gene comprises a second nucleotide sequence a nucleotide sequence identical to or having significant identity, e.g., at least 95% sequence identity, to a nucleotide sequence of an AAV rep gene of an other, e.g., second AAV.

In some embodiments, a nucleic acid molecule of the invention comprises an AAV rep gene and an AAV cap gene, wherein the AAV cap gene comprises a nucleotide sequence identical to or having significant identity, e.g., at least 95% sequence identity, to a first nucleotide sequence of a cap gene of a remote animal AAV, wherein the AAV rep gene comprises a nucleotide sequence identical to or having significant identity, e.g., at least 95% sequence identity, to a second nucleotide sequence of an AAV rep gene of an other, e.g., second AAV.

In some nucleic acid molecule embodiments of the invention, a nucleotide sequence of cap gene comprising a nucleotide sequence identical to or having significant identity, e.g., at least 95% sequence identity, to a nucleotide sequence of the non-primate animal AAV, the cap gene a nucleotide sequence identical to or having significant identity, e.g., at least 95% sequence identity, to a nucleotide sequence of the remote AAV, or the combination thereof, is modified to comprise (a) a nucleotide sequence encoding at least a first member of a protein:protein binding pair, (b) a nucleotide sequence encoding a detectable label, and/or (c) a nucleotide sequence encoding a point mutation.

In some nucleic acid embodiments, the protein:protein binding pair is selected from SpyTag:SpyCatcher, SpyTag:KTag, Isopeptag:pilin-C, SnoopTag:SnoopCatcher, and SpyTag002:SpyCatcher002. In some embodiments, the first member of a protein:protein binding pair comprises a detectable label, e.g., c-myc comprising a sequence set forth as SEQ ID NO:44.

In some nucleic acid embodiments, a nucleotide sequence of a cap gene of the non-primate animal AAV, the cap gene of the remote AAV, or the combination thereof, is modified to comprise the B1 epitope comprising an amino acid sequence of IGTRYLTR (SEQ ID NO: 45).

In some nucleic acid molecule embodiments of the invention, a nucleotide sequence of a cap gene of the non-primate animal AAV, the cap gene of the remote AAV, or the combination thereof, comprises a nucleotide sequence encoding a VP3 capsid protein, or portion thereof, comprising an amino acid sequence identical to or having significant identity, e.g., at least 95% sequence identity, to an amino acid sequence of a VP3 capsid protein of the non-primate animal AAV and/or an amino acid sequence of a VP3 capsid protein of the remote AAV. In some embodiments, a nucleotide sequence of a cap gene of the non-primate animal AAV the cap gene of the remote AAV, or the combination thereof, comprises a nucleotide sequence encoding a VP2 capsid protein, or portion thereof, comprising an amino acid sequence identical to or having significant identity, e.g., at least 95% sequence identity, to an amino acid sequence of a VP2 capsid protein of the non-primate animal AAV and/or an amino acid sequence of a VP2 capsid protein of the remote AAV. In some embodiments, a nucleotide sequence of a cap gene of the non-primate animal AAV, the cap gene of the remote AAV, or the combination thereof, comprises a nucleotide sequence encoding a VP1 capsid protein, or portion thereof, comprising an amino acid sequence identical to or having significant identity, e.g., at least 95% sequence identity, to an amino acid sequence of a VP1 capsid protein of the non-primate animal AAV and/or an amino acid sequence of a VP1 capsid protein of the remote AAV.

In some embodiments, a nucleic acid molecule of the invention comprises a nucleotide sequence encoding a non-primate animal VP3 capsid protein of the invention. In some embodiments, a nucleic acid molecule of the invention comprises a nucleotide sequence encoding a non-primate animal VP3 capsid protein of the invention and a non-primate animal VP2 capsid protein of the invention. In some embodiments, a nucleic acid molecule of the invention comprises a nucleotide sequence encoding a non-primate animal VP3 capsid protein of the invention, a VP2 capsid protein of the invention and a VP1 capsid protein of the invention.

In some embodiments, cap gene of a nucleic acid molecule of the invention encodes (i) a VP1 capsid protein that is either (a) a chimeric AAV VP1 capsid protein, optionally wherein the chimeric VP1 capsid protein comprises a VP1-unique region (VP1-u) comprising an amino acid sequence identical to or having significant identity, e.g., at least 95% sequence identity, to an amino acid sequence of an other, e.g., second, AAV operably linked to a VP1/VP2 common region and a VP3 region comprising an amino acid sequence identical to or having significant identity, e.g., at least 95% sequence identity, to an amino acid sequence of the non-primate AAV or the remote AAV, or (b) a VP1 capsid protein comprising an amino acid sequence identical to or having significant identity, e.g., at least 95% sequence identity, to an amino acid sequence of a VP1 capsid protein of the non-primate AAV or the remote AAV, (ii) a VP2 capsid protein that is either (a) a chimeric AAV VP2 capsid protein, optionally wherein the chimeric VP2 capsid protein comprises a VP1/VP2 common region comprising an amino acid sequence identical to or having significant identity, e.g., at least 95% sequence identity, to an amino acid sequence of an other, e.g., second, AAV operably linked to a VP3 region comprising an amino acid sequence identical to or having significant identity, e.g., at least 95% sequence identity, to an amino acid sequence of the non-primate AAV or (b) a VP2 capsid protein comprising an amino acid sequence identical to or having significant identity, e.g., at least 95% sequence identity, to an amino acid sequence of a VP2 capsid protein the non-primate AAV and/or (iii) a VP3 capsid protein comprising an amino acid sequence identical to or having significant identity, e.g., at least 95% sequence identity, to an amino acid sequence of a VP3 capsid protein of the non-primate AAV or the remote AAV.

In some embodiments, a cap gene of a nucleic acid molecule of the invention encodes (i) a chimeric AAV VP1 capsid protein, optionally wherein the chimeric VP1 capsid protein comprises a VP1-unique region (VP1-u) comprising an amino acid sequence identical to or having significant identity, e.g., at least 95% sequence identity, to an amino acid sequence of an other, e.g., AAV operably linked to a VP1/VP2 common region and a VP3 region comprising an amino acid sequence identical to or having significant identity, e.g., at least 95% sequence identity, to an amino acid sequence of the non-primate AAV or the remote AAV, (ii) a chimeric AAV VP2 capsid protein, optionally wherein the chimeric VP2 capsid protein comprises a VP1/VP2 common region comprising an amino acid sequence identical to or having significant identity, e.g., at least 95% sequence identity, to an amino acid sequence of an other, e.g., AAV operably linked to a VP3 region of the non-primate AAV or the remote AAV, and/or (iii) the VP3 capsid protein comprising an amino acid sequence identical to or having significant identity, e.g., at least 95% sequence identity, to an amino acid sequence of the non-primate AAV or the remote AAV.

In some embodiments, cap gene of a nucleic acid molecule of the invention encodes (i) a chimeric AAV VP1 capsid protein, optionally wherein the chimeric VP1 capsid protein comprises a VP1-unique region (VP1-u) comprising an amino acid sequence identical to or having significant identity, e.g., at least 95% sequence identity, to an amino acid sequence of an other AAV operably linked to a VP1/VP2 common region and a VP3 region comprising an amino acid sequence identical to or having significant identity, e.g., at least 95% sequence identity, to an amino acid sequence of the non-primate AAV or the remote AAV, (ii) a VP2 capsid protein comprising an amino acid sequence identical to or having significant identity, e.g., at least 95% sequence identity, to an amino acid sequence of the non-primate AAV or the remote AAV, and (iii) the VP3 capsid protein comprising an amino acid sequence identical to or having significant identity, e.g., at least 95% sequence identity, to an amino acid sequence of the non-primate AAV or the remote AAV.

In some embodiments, cap gene of a nucleic acid molecule of the invention encodes (i) a VP1 capsid protein comprising an amino acid sequence identical to or having significant identity, e.g., at least 95% sequence identity, to an amino acid sequence of the non-primate AAV or the remote AAV, (ii) a VP2 capsid protein comprising an amino acid sequence identical to or having significant identity, e.g., at least 95% sequence identity, to an amino acid sequence of the non-primate AAV or the remote AAV, and/or (iii) a VP3 capsid protein comprising an amino acid sequence identical to or having significant identity, e.g., at least 95% sequence identity, to an amino acid sequence of the non-primate AAV or the remote AAV.

In some nucleic acid molecule embodiments of the invention, the other, second, AAV is a primate AAV or a combination of primate AAVs. In some embodiments, the other AAV is a selected from the group consisting of AAV1, AAV2, AAV3, AAV4, AAV5, AAV6, AAV7, AAV8, AAV9, AAV10, AAV 11, AAV12, AAV13, and a combination thereof. In some embodiments, the other AAV is AAV2.

In some nucleic acid molecule embodiments of the invention, the non-primate animal AAV is a non-primate AAV listed in Table 2. In some embodiments, the non-primate AAV is an avian AAV (AAAV), a sea lion AAV or a bearded dragon AAV. In some embodiments, the non-primate animal AAV is an AAAV, and optionally a nucleotide sequence of an AAAV capsid protein comprises a modification is at position I444 or I580 of a VP1 capsid protein of AAAV. In some embodiments, the non-primate animal AAV is a squamate AAV, e.g., a bearded dragon AAV, and optionally a nucleotide sequence of a bearded dragon AAV comprises a modification is at position I573 or I436 of a VP1 capsid protein of a bearded dragon AAV. In some embodiments, the non-primate animal AAV is a mammalian AAV, e.g., a sea lion AAV, and optionally a nucleotide sequence of a sea lion AAV comprises a modification at position selected from the group consisting of I429, I430, I431, I432, I433, I434, I436, I437, and A565 of a VP1 capsid protein of a sea lion AAV.

Nucleic acid molecule embodiments of the invention may comprise a nucleotide sequence selected from the group consisting of (a) the nucleotide sequence set forth as SEQ ID NO:1, (b) the nucleotide sequence set forth as SEQ ID NO:3, (c) the nucleotide sequence set forth as SEQ ID NO:5, (d) the nucleotide sequence set forth as SEQ ID NO:7, (e) the nucleotide sequence set forth as SEQ ID NO:9, (f) the nucleotide sequence set forth as SEQ ID NO:11, (g) the nucleotide sequence set forth as SEQ ID NO:13, (h) the nucleotide sequence set forth as SEQ ID NO:15, (i) the nucleotide sequence set forth as SEQ ID NO:17, (j) the nucleotide sequence set forth as SEQ ID NO:19, (k) the nucleotide sequence set forth as SEQ ID NO:21, (l) the nucleotide sequence set forth as SEQ ID NO:23, (m) the nucleotide sequence set forth as SEQ ID NO:25, (n) the nucleotide sequence set forth as SEQ ID NO:27, (o) the nucleotide sequence set forth as SEQ ID NO:29, (p) the nucleotide sequence set forth as SEQ ID NO:31, (q) the nucleotide sequence set forth as SEQ ID NO:33, (r) the nucleotide sequence set forth as SEQ ID NO:35, (s) the nucleotide sequence set forth as SEQ ID NO:52, (t) the nucleotide sequence set forth as SEQ ID NO:54, (u) the nucleotide sequence set forth as SEQ ID NO:56, (v) the nucleotide sequence set forth as SEQ ID NO:58, (w) the nucleotide sequence set forth as SEQ ID NO:60, (x) the nucleotide sequence set forth as SEQ ID NO:62, (y) the nucleotide sequence set forth as SEQ ID NO:64, (z) the nucleotide sequence set forth as SEQ ID NO:66, (aa) the nucleotide sequence set forth as SEQ ID NO:68, (bb) the nucleotide sequence set forth as SEQ ID NO:70, (cc) a nucleotide sequence having at least 95% identity to the nucleotide sequence set forth as SEQ ID NO: 1, 3, 5, 7, 9, 11, 13, 15, 17, 19, 21, 23, 25, 27, 29, 31, 33, 35, 52, 54, 56, 58, 60, 62, 64, 66, 68, or 70, (dd) any portion of the nucleotide sequence of (a)-(cc) encoding a VP2 capsid protein and/or a VP3 capsid protein.

In some embodiments a nucleic acid molecule comprises a nucleotide sequence set forth as SEQ ID NO:1, a portion thereof that encodes a VP2 and/or VP3 capsid protein, and degenerate variants thereof. In some embodiments a nucleic acid molecule comprises a nucleotide sequence set forth as SEQ ID NO:3, a portion thereof that encodes a VP2 and/or VP3 capsid protein, and degenerate variants thereof. In some embodiments a nucleic acid molecule comprises a nucleotide sequence set forth as SEQ ID NO:5, a portion thereof that encodes a VP2 and/or VP3 capsid protein, and degenerate variants thereof. In some embodiments a nucleic acid molecule comprises a nucleotide sequence set forth as SEQ ID NO:7, a portion thereof that encodes a VP2 and/or VP3 capsid protein, and degenerate variants thereof. In some embodiments a nucleic acid molecule comprises a nucleotide sequence set forth as SEQ ID NO:9, a portion thereof that encodes a VP2 and/or VP3 capsid protein, and degenerate variants thereof. In some embodiments a nucleic acid molecule comprises a nucleotide sequence set forth as SEQ ID NO:11, a portion thereof that encodes a VP2 and/or VP3 capsid protein, and degenerate variants thereof. In some embodiments a nucleic acid molecule comprises a nucleotide sequence set forth as SEQ ID NO:13, a portion thereof that encodes a VP2 and/or VP3 capsid protein, and degenerate variants thereof. In some embodiments a nucleic acid molecule comprises a nucleotide sequence set forth as SEQ ID NO:15, a portion thereof that encodes a VP2 and/or VP3 capsid protein, and degenerate variants thereof. In some embodiments a nucleic acid molecule comprises a nucleotide sequence set forth as SEQ ID NO:17, a portion thereof that encodes a VP2 and/or VP3 capsid protein, and degenerate variants thereof. In some embodiments a nucleic acid molecule comprises a nucleotide sequence set forth as SEQ ID NO:19, a portion thereof that encodes a VP2 and/or VP3 capsid protein, and degenerate variants thereof. In some embodiments a nucleic acid molecule comprises a nucleotide sequence set forth as SEQ ID NO:21, a portion thereof that encodes a VP2 and/or VP3 capsid protein, and degenerate variants thereof. In some embodiments a nucleic acid molecule comprises a nucleotide sequence set forth as SEQ ID NO:23, a portion thereof that encodes a VP2 and/or VP3 capsid protein, and degenerate variants thereof. In some embodiments a nucleic acid molecule comprises a nucleotide sequence set forth as SEQ ID NO:25, a portion thereof that encodes a VP2 and/or VP3 capsid protein, and degenerate variants thereof. In some embodiments a nucleic acid molecule comprises a nucleotide sequence set forth as SEQ ID NO:27, a portion thereof that encodes a VP2 and/or VP3 capsid protein, and degenerate variants thereof. In some embodiments a nucleic acid molecule comprises a nucleotide sequence set forth as SEQ ID NO:29, a portion thereof that encodes a VP2 and/or VP3 capsid protein, and degenerate variants thereof. In some embodiments a nucleic acid molecule comprises a nucleotide sequence set forth as SEQ ID NO:31, a portion thereof that encodes a VP2 and/or VP3 capsid protein, and degenerate variants thereof. In some embodiments a nucleic acid molecule comprises a nucleotide sequence set forth as SEQ ID NO:33, a portion thereof that encodes a VP2 and/or VP3 capsid protein, and degenerate variants thereof. In some embodiments a nucleic acid molecule comprises a nucleotide sequence set forth as SEQ ID NO:35, a portion thereof that encodes a VP2 and/or VP3 capsid protein, and degenerate variants thereof. In some embodiments a nucleic acid molecule comprises a nucleotide sequence set forth as SEQ ID NO:52, a portion thereof that encodes a VP2 and/or VP3 capsid protein, and degenerate variants thereof. In some embodiments a nucleic acid molecule comprises a nucleotide sequence set forth as SEQ ID NO:54, a portion thereof that encodes a VP2 and/or VP3 capsid protein, and degenerate variants thereof. In some embodiments a nucleic acid molecule comprises a nucleotide sequence set forth as SEQ ID NO:56, a portion thereof that encodes a VP2 and/or VP3 capsid protein, and degenerate variants thereof. In some embodiments a nucleic acid molecule comprises a nucleotide sequence set forth as SEQ ID NO:58, a portion thereof that encodes a VP2 and/or VP3 capsid protein, and degenerate variants thereof. In some embodiments a nucleic acid molecule comprises a nucleotide sequence set forth as SEQ ID NO:60, a portion thereof that encodes a VP2 and/or VP3 capsid protein, and degenerate variants thereof. In some embodiments a nucleic acid molecule comprises a nucleotide sequence set forth as SEQ ID NO:62, a portion thereof that encodes a VP2 and/or VP3 capsid protein, and degenerate variants thereof. In some embodiments a nucleic acid molecule comprises a nucleotide sequence set forth as SEQ ID NO:64, a portion thereof that encodes a VP2 and/or VP3 capsid protein, and degenerate variants thereof. In some embodiments a nucleic acid molecule comprises a nucleotide sequence set forth as SEQ ID NO:66, a portion thereof that encodes a VP2 and/or VP3 capsid protein, and degenerate variants thereof. In some embodiments a nucleic acid molecule comprises a nucleotide sequence set forth as SEQ ID NO:68, a portion thereof that encodes a VP2 and/or VP3 capsid protein, and degenerate variants thereof. In some embodiments a nucleic acid molecule comprises a nucleotide sequence set forth as SEQ ID NO:70, a portion thereof that encodes a VP2 and/or VP3 capsid protein, and degenerate variants thereof.

In some embodiments, the nucleotide sequence encoding a VP1 capsid protein of the invention and optionally a VP2 and/or VP3 capsid protein of the invention is operably linked to a promoter. In some embodiments, the promoter is selected from a viral promoter, a bacterial promoter, a mammalian promoter (e.g., human or non-human), an avian promoter, a fish promoter, an insect promoter, and any combination thereof. In some embodiments, the promoter is selected from p40, SV40, EF, CMV, B19p6, and CAG. In some embodiments, the promoter is an AAV p40 promoter. In some embodiments, the wherein the promoter directs the expression of the capsid protein(s) in a packaging cell.

In some embodiments, the cap gene of a nucleic acid molecule of the invention comprises is operably linked to a promoter. In some embodiments, promoter directs the expression of the capsid protein(s) in a packaging cell. In some embodiments, the promoter is selected from p40, SV40, EF, e.g., EF1α CMV, B19p6, and CAG.

In some embodiments, a nucleic acid molecule of the invention further comprises a second nucleotide sequence encoding one or more AAV Rep proteins, optionally wherein the second nucleotide sequence is operably linked to a promoter. In some embodiments, the one or more Rep proteins are primate animal AAV Rep proteins. In some embodiments, the one or more Rep proteins are non-primate animal AAV Rep proteins. In some embodiments, the one or more Rep proteins are selected from Rep78, Rep68, Rep52 and Rep40, optionally the one or more Rep proteins comprises Rep78. In some embodiments, the promoter operably linked to the second nucleotide sequence encoding one or more AAV Rep proteins is selected from a viral promoter, a bacterial promoter, a mammalian promoter (e.g., human or non-human), an avian promoter, a fish promoter, an insect promoter, and any combination thereof. In some embodiments, the promoter operably linked to the second nucleotide sequence encoding one or more AAV Rep proteins is selected from p19, p5, p40, SV40, EF, e.g., EF1α CMV, B19p6, and CAG. In some embodiments, the promoter directs the expression of the capsid protein(s) in a packaging cell. In some embodiments, the promoter operably linked to the second nucleotide sequence encoding one or more AAV Rep proteins is selected from p19, and/or p5.

Compositions and packaging cells comprising, and capsid proteins encoded from the nucleic acid molecules of the invention, are also part of the invention. Viral particles expressed by packaging cells of the invention are also described.

Compositions and packaging cells for producing AAV viral particles of the invention, in some embodiments, comprise nucleic acid molecule of the invention, e.g., comprising a cap gene of the invention encoding an AAV capsid protein of the invention. In some embodiments, a composition and/or packaging cell of the invention comprises a nucleic acid molecule of the invention. In some embodiments, the cap gene comprises a nucleotide sequence selected from the group consisting of the nucleotide sequence set forth as SEQ ID NO:1, nucleotide sequence set forth as SEQ ID NO:3, nucleotide sequence set forth as SEQ ID NO:5, nucleotide sequence set forth as SEQ ID NO:7, nucleotide sequence set forth as SEQ ID NO:9, nucleotide sequence set forth as SEQ ID NO:11, nucleotide sequence set forth as SEQ ID NO:13, nucleotide sequence set forth as SEQ ID NO:15, nucleotide sequence set forth as SEQ ID NO:17, nucleotide sequence set forth as SEQ ID NO:19, nucleotide sequence set forth as SEQ ID NO:21, nucleotide sequence set forth as SEQ ID NO:23, nucleotide sequence set forth as SEQ ID NO:25, nucleotide sequence set forth as SEQ ID NO:27, nucleotide sequence set forth as SEQ ID NO:29, nucleotide sequence set forth as SEQ ID NO:31, nucleotide sequence set forth as SEQ ID NO:33, nucleotide sequence set forth as SEQ ID NO:35, nucleotide sequence set forth as SEQ ID NO:52, nucleotide sequence set forth as SEQ ID NO:54, nucleotide sequence set forth as SEQ ID NO:56, nucleotide sequence set forth as SEQ ID NO:58, nucleotide sequence set forth as SEQ ID NO:60, nucleotide sequence set forth as SEQ ID NO:62, nucleotide sequence set forth as SEQ ID NO:64, nucleotide sequence set forth as SEQ ID NO:66, nucleotide sequence set forth as SEQ ID NO:68, nucleotide sequence set forth as SEQ ID NO:70, any portion of the nucleotide sequence set forth as SEQ ID NOs: 1, 3, 5, 7, 9, 11, 13, 15, 17, 19, 21, 23, 25, 27, 29, 31, 33, 35, 52, 54, 56, 58, 60, 62, 64, 66, 68, or 70, encoding a VP2 capsid, any portion of the nucleotide sequence set forth as SEQ ID NOs: 1, 3, 5, 7, 9, 11, 13, 15, 17, 19, 21, 23, 25, 27, 29, 31, 33, 35, 52, 54, 56, 58, 60, 62, 64, 66, 68, or70 encoding a VP3 capsid, and any combination thereof.

In some embodiments, compositions and packaging cells further comprise a nucleic acid molecule comprising a rep gene encoding one or more AAV Rep proteins, wherein said rep gene is operably linked to a promoter, optionally wherein the rep gene and the cap gene are of two different AAV. In some embodiments, the promoter operably linked to the rep gene directs the expression of the Rep protein(s) in the packaging cell, e.g., the promoter is selected from p5, p19 SV40, EF, CMV, B19p6, and CAG. In some embodiments, the one or more Rep proteins are selected from Rep78, Rep68, Rep52 and Rep40, optionally the one or more Rep proteins comprises Rep78. In some embodiments, the one or more Rep proteins are primate animal AAV Rep proteins. In some other embodiments, the one or more Rep proteins are non-primate animal AAV Rep proteins. In some embodiments, the one or more Rep proteins comprise both.

In some embodiments, compositions and packaging cells of the invention further comprise a nucleic acid molecule comprising a nucleotide sequence of a nucleotide of interest flanked on at least one side by at least one AAV inverted terminal repeat (ITR) that is recognized by the one or more Rep proteins. In some embodiments, the nucleotide of interest is flanked on the other side by a second ITR of the same AAV as the at least one ITR. In some embodiments, the nucleotide of interest is flanked on the other side by a second ITR, wherein the second ITR and the at least one ITR are of different AAV.

In some embodiments, a composition and/or packaging cell of the invention further comprises a nucleic acid molecule comprising a nucleotide of interest (e.g., nucleotide sequence of a transgene) flanked by 5′ and 3′ AAV inverted terminal repeats (ITRs) such that the viral particles of the interest further comprise a genome that comprises the nucleotide of interest flanked by the 5′ and 3′ AAV ITRs.

In some embodiments, a composition and/or packaging cell of the invention further comprises a nucleotide sequence encoding a reference capsid protein.

Accordingly, in some embodiments, a composition, packaging cell, and/or viral particle of the invention further comprises a genome comprising from 5′ to 3′: a 5′ ITR, a nucleotide of interest, and a 3′ITR. In some embodiments, the genome further comprises a promoter operably linked to the nucleotide of interest. In some embodiments, the 5′ and 3′ ITR are from AAVs of the same species. In some embodiments, the 5′ and 3′ ITR are from AAVs of two different species.

In some embodiments, the nucleotide of interest is a reporter gene. In some embodiments, the reporter gene encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

In some embodiments, the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA.

Described herein is a method of making an AAV viral particle of the invention, the method comprising culturing a packaging cell under conditions sufficient for the production of viral particles, the packaging cell comprising (1) at least one nucleotide sequence encoding one or more AAV Rep proteins, e.g., a rep gene, and (2) a first nucleotide sequence encoding an AAV VP1 capsid protein, optionally a second nucleotide sequence encoding an AAV VP2 capsid protein, and a third nucleotide sequence encoding a non-primate animal AAV VP3 capsid protein, (e.g., a first, second, and third nucleotide molecules of the invention) and optionally (3) a nucleotide of interest flanked by a first and/or second ITR of a second AAV, wherein the one or more AAV Rep proteins recognize a recognition site of the first and/or second ITR of the second AAV, wherein the third nucleotide sequence encodes a non-primate animal AAV VP3 capsid protein of the invention, and optionally wherein the first nucleotide sequence encodes an AAV VP1 capsid protein of the invention and/or the second nucleotide sequence encodes an AAV VP2 capsid protein of the invention. In some embodiments, a single cap gene of the invention comprises the first, second and third nucleotide sequences. In some embodiments a single packaging plasmid comprises the at least one nucleotide sequence encoding one or more AAV Rep proteins and any combination of the first, second and third nucleotides respectively encoding an AAV VP1 capsid protein, an AAV VP2 capsid protein, and an AAV VP3 capsid protein of a non-primate animal.

In some embodiments, the method comprises culturing a packaging cell of the invention. In some embodiments, the method comprises culturing a packaging cell of the invention comprising a nucleic acid molecule of the invention, wherein the packaging cell optionally further comprises a helper plasmid and/or a transfer plasmid comprising a nucleotide of interest.

Some method embodiments further comprise isolating self-complementary adeno-associated viral particles from culture supernatant and/or the cell lysate. Some method embodiments further comprise lysing the packaging cell and isolating single-stranded adeno-associated viral particles from culture supernatant and/or the cell lysate. Some embodiments further comprise a. clearing cell debris, b. treating the supernatant containing viral particles with Benzonase or DNase I and MgCl2, c. concentrating viral particles, d. purifying the viral particles, and e. any combination of a.-d.

In some embodiments, mosaic viral particles are generated by transfecting mixtures of a first cap gene encoding a VP capsid protein comprising a first member of a protein:protein binding pair and at least one reference cap gene encoding a reference VP capsid protein into packaging cells at certain ratio. In some embodiments, a mosaic viral particle of the invention may be generated with mixtures of a modified cap gene:reference cap gene(s). In some embodiments, the modified cap gene encodes at least one of the AAV VP1, VP2, and VP3 capsid proteins that comprises a modification, e.g., a first member of a protein:protein binding pair, and reference cap gene encodes a reference capsid protein corresponding to the at least one of the modified AAV VP1, VP2, and VP3 capsid proteins except for the modification. In some embodiments, a modified cap gene encodes a VP1 capsid protein modified with a first member of a protein:protein binding pair and a reference cap gene encodes a reference VP1 capsid protein lacking the modification. In some embodiments, a modified cap gene encodes a VP2 capsid protein modified with a first member of a protein:protein binding pair and a reference cap gene encodes a reference VP2 capsid protein lacking the modification. In some embodiments, a modified cap gene encodes a VP3 capsid protein modified with a first member of a protein:protein binding pair and a reference cap gene encodes a reference VP3 capsid protein lacking the modification.

Generally, viral particles as described herein comprise a viral capsid comprising a viral capsid protein as described herein, including mosaic viral capsids, wherein the viral capsid encapsidates a nucleotide of interest. In some embodiments, the nucleotide of interest is under the control of a promoter selected from the group consisting of a viral promoter, a bacterial promoter, a mammalian promoter, an avian promoter, a fish promoter, an insect promoter, and any combination thereof. In some embodiments, the nucleotide of interest is under the control of a non-human promoter. In some embodiments, the promoter is a cytomegalovirus (CMV) promoter. In some embodiments, the promoter is an EF, e.g., EF1α, promoter. In some embodiments, the promoter is a CAG promoter. In some embodiments, the promoter is a Ubiquitin C (UbC) promoter.

Generally, a nucleotide of interest may be one or more genes, which may encode a detectable marker, e.g., reporter, or a therapeutic polypeptide. In some embodiments, the nucleotide of interest is a reporter gene. In some embodiments, the nucleotide of interest is a reporter gene that encodes a detectable marker selected from the group consisting of green fluorescent protein, luciferase, β-galactosidase, etc. In some embodiments, the detectable marker is green fluorescent protein. In other embodiments, the nucleotide of interest is selected from the group consisting of a suicide gene, a nucleotide encoding an antibody or fragment thereof, a nucleotide encoding a CRISPR/Cas system or portion(s) thereof, a nucleotide encoding antisense RNA, a nucleotide encoding siRNA, a secreted enzyme, a gene encoding a therapeutic protein, etc. In one embodiment, the nucleotide of interest encodes a multidomain therapeutic, e.g., a protein that comprises at least two domains providing two distinct functions.

Compositions described herein generally comprise a viral particle that comprises a viral capsid protein as described herein, e.g., comprises a capsid comprising the viral capsid protein (including a mosaic capsid), wherein the capsid encapsidates a nucleotide of interest. In some embodiments, a composition described herein comprises (1) a viral particle having a capsid comprising a viral capsid protein described herein, and (2) a pharmaceutically acceptable carrier.

Also described herein are methods of using the viral capsid proteins, viral particles comprising same, compositions, etc. In some embodiments, the methods comprise contacting a target cell (which may be in vitro (e.g., ex vivo) or in vivo, e.g., in a human) with a viral particle comprising a viral capsid protein as described herein, wherein the viral capsid or viral particle comprises a targeting ligand that specifically binds a protein expressed on the surface the target cell.

Viral particles as described herein are particularly suited for the targeted introduction of a nucleotide of interest to a specific cell since the viral capsid protein(s) described herein comprise a first member of a protein:protein binding pair to its cognate second member, optionally linked to a targeting ligand. In some embodiments, the targeting ligand is operably linked to the protein (second member), e.g., fused to the protein, optionally via a linker. In some embodiments, a targeting ligand may be a binding moiety, e.g., a natural ligand, antibody, a multispecific binding molecule, etc. In some embodiments, the targeting ligand is an antibody or portion thereof. In some embodiments, the targeting ligand is an antibody comprising a variable domain that binds a cell surface protein on a target cell and a heavy chain constant domain. In some embodiments, the targeting ligand is an antibody comprising a variable domain that binds a cell surface protein on a target cell and an IgG heavy chain constant domain. In some embodiments, the targeting ligand is an antibody comprising a variable domain that binds a cell surface protein on a target cell and an IgG heavy chain constant domain, wherein the IgG heavy chain constant domain is operably linked, e.g., via a linker, to a protein (e.g., second member of a protein:protein binding pair) that forms an isopeptide covalent bond with the first member. In some embodiments, a capsid protein described herein comprises a first member comprising SpyTag operably linked to the viral capsid protein, and covalently linked to the SpyTag, an second member comprising SpyCatcher linked to a targeting ligand comprising an antibody variable domain and an IgG heavy chain domain, wherein SpyCatcher and the IgG heavy chain domain are linked via an amino acid linker, e.g., GSGESG (SEQ ID NO:49). In some embodiments, the second member comprises the sequence set forth as SEQ ID NO:47, which comprises a portion of a human IgG4 heavy chain, said IgG4 portion having a sequence set forth as SEQ ID NO:51, linked via linker (SEQ ID NO:49) to SpyCatcher (SEQ ID NO:43).

Generally, a targeting ligand specifically binds a cell surface molecule, e.g., an oligosaccharide, a receptor, cell surface marker, etc., expressed on the surface of a mammalian (e.g., human) eukaryotic cell, e.g., a target cell.

### BRIEF DESCRIPTION OF THE DRAWINGS

FIG. 1 provides an illustrative (not to scale), non-limiting, and exemplary embodiment of a Rep-Cap expression plasmid of the invention that may be used to generate an AAV chimeric viral particle. Primate AAV sequences are shown as unfilled boxes and non-primate animal AAV sequences are shown in filled boxes. Also depicted for illustrative purposes only are exemplary non-limiting positions (not to scale) for insertion of (1) a first member of a protein:protein binding pair for directing the tropism of the assembled capsid comprising the encoded VP1, VP2, and VP3 proteins (solid line within capsid sequences of the non-primate animal AAV) and (2) a detectable label for detection of the encoded VP1, VP2, and VP3 proteins (dotted line within capsid sequences of the non-primate animal AAV). A Rep-Cap expression plasmid as shown in FIG. 1 may be used to produce AAV viral particles comprising a nucleotide of interest that is flanked by the 5′ and 3′ inverted terminal repeat (ITR) sequences of the primate AAV.

FIG. 2 provides western blots using B1 antibody, which recognizes a B1 epitope engineered into a chimeric primate/non-primate animal AAV cap gene (see FIG. 1), to analyze the resulting chimeric primate/non-primate animal AAV VP1 protein, non-primate animal AAV VP2 protein, and non-primate animal AAV VP3 protein. The primate AAV was AAV2 and the non-primate animal AAV was (A) Avian AAV, (B) Sea Lion AAV or (C) Bearded Dragon AAV. Western blots analyzed protein samples collected during various steps of purification of AAV particles via affinity chromatography, including the input sample, the flow-through (FT) fraction, and the elution fraction from the affinity chromatography column.

FIG. 3A provides a non-limiting predicted avian AAV VP3 ribbon structure highlighting K580 and G444 as non-limiting insertion sites for a first member of a protein:protein binding pair. FIG. 3B provides qPCR quantification of the virus titer achieved from crude virus preparations of a panel of chimeric AAV2/Avian AAV viral particles, either having no SpyTag or comprising a SpyTag insertion at the indicated position, or mosaic particles comprised of AAV2/Avian AAV particles having no SpyTag mixed with AAV2/Avian AAV particles bearing a SpyTag peptide insertion at the indicated position. FIG. 3C provides a western blot using B1 antibody, which recognizes a linear epitope engineered into chimeric AAV2/Avian AAV VP1, VP2 and VP3 capsid proteins, analyzing the reaction between an anti-ASGR1 antibody fused with SpyCatcher “SpyC-anti-ASGR1 mAb”, and a panel of chimeric AAV2/Avian AAV viral particles either lacking or bearing a SpyTag insertion at the indicated position, or mosaic particles comprising chimeric AAV2/Avian AAV particles lacking SpyTag mixed with chimeric AAV2/Avian AAV particles bearing a SpyTag peptide insertion at the indicated position.

FIG. 4A provides a non-limiting predicted sea lion AAV VP3 ribbon structure highlighting A565 and G432 as non-limiting insertion sites for a first member of a protein:protein binding pair. FIG. 4B provides qPCR quantification of the virus titer achieved from crude virus preparations of a panel of chimeric AAV2/sea lion AAV viral particles, either having no SpyTag or comprising a SpyTag insertion at the indicated position, or mosaic particles comprised of AAV2/sea lion AAV particles having no SpyTag mixed with AAV2/sea lion AAV particles bearing a SpyTag peptide insertion at the indicated position. FIG. 4C provides a western blot using B1 antibody, which recognizes a linear epitope engineered into chimeric AAV2/Sea Lion AAV VP1, VP2 and VP3 capsid proteins, analyzing the reaction between an anti-HER2 antibody (HERCEPTIN®) fused with SpyCatcher “SpyC-anti-HER2 mAb”, and a panel of AAV2/sea lion AAV viral particles either lacking or bearing a SpyTag insertion at the indicated position, or mosaic AAV2/sea lion AAV particles comprising chimeric AAV2/sea lion AAV particles lacking SpyTag mixed with chimeric AAV2/sea lion AAV particles bearing a SpyTag peptide insertion at the indicated position.

FIG. 5 provides (A) qPCR quantification of the virus titer achieved from crude virus preparations of a panel of AAV2/Sea Lion AAV viral particles, either having no SpyTag or bearing a SpyTag insertion at the indicated position and (B) a western blot using B1 antibody, which recognizes a linear epitope engineered into chimeric AAV2/Sea Lion AAV VP1, VP2 and VP3 capsid proteins, analyzing the reaction between an anti-HER2 antibody (HERCEPTIN®) fused with SpyCatcher “SpyC-mAb”, and a panel of Sea Lion AAV viral particles, either having no SpyTag or bearing a SpyTag insertion at the indicated position.

FIG. 6A provides a non-limiting predicted bearded dragon AAV VP3 ribbon structure highlighting T573 and G436 as non-limiting insertion sites for a first member of a protein:protein binding pair. FIG. 6B provides qPCR quantification of the virus titer achieved from crude virus preparations of a panel of chimeric AAV2/bearded dragon AAV viral particles, either having no SpyTag or comprising a SpyTag insertion at the indicated position, or mosaic particles comprised of AAV2/bearded dragon AAV particles having no SpyTag mixed with AAV2/bearded dragon AAV particles bearing a SpyTag peptide insertion at the indicated position. FIG. 6C provides a western blot using B1 antibody, which recognizes a linear epitope engineered into chimeric AAV2/bearded dragon AAV VP1, VP2 and VP3 capsid proteins, analyzing the reaction between an anti-HER2 antibody (HERCEPTIN®) fused with SpyCatcher “SpyC-anti-HER2 mAb”, and a panel of AAV2/bearded dragon AAV viral particles either lacking or bearing a SpyTag insertion at the indicated position, or mosaic AAV2/bearded dragon AAV particles comprising chimeric AAV2/bearded dragon AAV particles lacking SpyTag mixed with chimeric AAV2/bearded dragon AAV particles bearing a SpyTag peptide insertion at the indicated position.

FIG. 7A provides scatter plots obtained from flow cytometry evaluating green fluorescent protein (GFP) expression by HER2-positive (+) 293 hErbB2 cells infected with chimeric AAV2/AAAV particles having no SpyTag, chimeric AAV2/AAAV G444 Linker6 SpyTag particles, or chimeric AAV2/AAAV K580 Linker6 SpyTag particles. The chimeric AAV2/AAAV G444 Linker6 SpyTag particles and the chimeric AAV2/AAAV K580 Linker6 SpyTag particles were conjugated to either an irrelevant isotype control antibody against GLP1R or an anti-HER2 antibody (HERCEPTIN®) fused with SpyCatcher (SEQ ID NO:43) via SpyTag (SEQ ID NO:42). Viruses express GFP as a marker of transduction. FIG. 7B provides scatter plots obtained from flow cytometry evaluating green fluorescent protein (GFP) expression by parental ASGR1-negative (−) 293 cells or ASGR1-positive (+) 293 hASGR1 cells infected with chimeric AAV2/AAAV particles having no SpyTag or chimeric AAV2/AAAV K580 Linker6 SpyTag particles. The chimeric AAV2/AAAV K580 Linker6 SpyTag particles were conjugated to either an irrelevant isotype control antibody against GLP1R fused with SpyCatcher via SpyTag, or conjugated to a SpyCatcher-fused antibody that specifically binds ASGR1 via SpyTag. Viruses express GFP as a marker of transduction.

FIG. 8A provides scatter plots obtained from flow cytometry evaluating green fluorescent protein (GFP) expression by HER2-positive (+) 293 hErbB2 or HER2-negative (−) 293 parental cells infected with chimeric AAV2/Sea Lion particles having no SpyTag or chimeric AAV2/Sea Lion G432 Linker6 SpyTag particles. The chimeric AAV2/Sea Lion G432 Linker6 SpyTag particles were conjugated to either an irrelevant isotype control antibody against GLP1R fused with SpyCatcher via SpyTag, or conjugated to an anti-HER2 antibody (HERCEPTIN®) fused with SpyCatcher (SEQ ID NO:43) via SpyTag. Viruses express GFP as a marker of transduction. FIG. 8B provides scatter plots obtained from flow cytometry evaluating green fluorescent protein (GFP) expression by ASGR1-positive (+) 293 hASGR1 or ASGR1-negative (−) 293 parental cells infected with chimeric AAV2/Sea Lion particles having no SpyTag or cells infected with chimeric AAV2/Sea Lion G432 Linker6 SpyTag particles. The chimeric AAV2/Sea Lion G432 Linker6 SpyTag particles were conjugated to either an irrelevant isotype control antibody against GLP1R fused with SpyCatcher via SpyTag, or conjugated to a SpyCatcher-fused antibody that specifically binds ASGR1 via SpyTag. Viruses express GFP as a marker of transduction.

FIG. 9 provides scatter plots obtained from flow cytometry evaluating green fluorescent protein (GFP) expression by HER2-positive (+) 293 infected with a panel of chimeric AAV2/Sea Lion AAV viral particles, either AAV2/Sea Lion AAV particles with no SpyTag or comprising a SpyTag insertion at the indicated position. The SpyTag inserted into chimeric AAV2/Sea Lion particles were conjugated to an anti-HER2 antibody (HERCEPTIN®) fused with SpyCatcher (SEQ ID NO:43) via SpyTag. Viruses express GFP as a marker of transduction.

FIG. 10A provides scatter plots obtained from flow cytometry evaluating green fluorescent protein (GFP) expression by HER2-positive (+) 293 hErbB2 cells either “Uninfected” or infected with chimeric AAV2/bearded dragon AAV particles lacking SpyTag, chimeric AAV2/bearded dragon T573 Linker6 SpyTag Mosaic particles, or chimeric AAV2/bearded dragon G436 Linker6 SpyTag Mosaic particles. The chimeric AAV2/bearded dragon T573 Linker6 SpyTag Mosaic particles and chimeric AAV2/bearded dragon G436 Linker6 SpyTag Mosaic particles were conjugated to an anti-HER2 antibody (HERCEPTIN®) fused with SpyCatcher (SEQ ID NO: 43) via SpyTag. Viruses express GFP as a marker of transduction. FIG. 10B provides scatter plots obtained from flow cytometry evaluating green fluorescent protein (GFP) expression by ASGR1-positive (+) 293 hASGR1 or ASGR1-negative (−) 293 parental cells infected with chimeric AAV2/bearded dragon AAV particles having no SpyTag, chimeric AAV2/bearded dragon T573 Linker6 SpyTag particles, or chimeric AAV2/bearded dragon T573 Linker6 SpyTag anti-ASGR1 particles. The chimeric AAV2/bearded dragon T573 Linker6 SpyTag anti-ASGR1 particles were conjugated to a SpyCatcher-fused antibody that specifically binds ASGR1 via SpyTag. Viruses express GFP as a marker of transduction.

FIG. 11A provides the results of a Nanoluc luciferase assay evaluating Nanoluc reporter expression by cells positive (+) for hASGR1 after infection with “AAV2 anti-ASGR1” particles, chimeric AAV2/AAAV anti-ASGR1 particles, or chimeric AAV2/Sea Lion AAV anti-ASGR1 particles in the presence of the indicated concentration of purified humanIgG. All particles were conjugated to a SpyCatcher-fused antibody that specifically binds ASGR1 via SpyTag. Viruses express Nanoluc as a marker of transduction. FIG. 11B provides a quantification of the graphs in FIG. 11A, but normalized to the “PBS only” condition. FIG. 11C provides a table of IC50 values for concentration of IgG required to neutralize the indicated viruses by 50%.

FIG. 12 provides (A) luminescence images of genetically modified mice that express human ASGR1 on liver cells (ASGR1 Humanized mice) 33 days post intravenous injection with phosphate buffered saline (PBS) or with 5.0×10¹¹ viral genomes (vg)/animal of SpyTagged chimeric AAV2/AAAV particles carrying a firefly luciferase nucleotide of interest and modified by (1) SpyCatcher-anti-human ASGR1 antibody or (2) SpyCatcher-anti-human GLP1R antibody (control mAb). Viruses express Firefly luciferase as a marker of transduction. Mice were anesthetized using isoflurane, injected with a Luciferin substrate and imaged 10 minutes later using the IVIS Spectrum In Vivo Imaging System (PerkinElmer); (B) a quantification of the average radiance of the individual animals within the luminescence images depicted in Panel A; and (C) a quantification of the average radiance of dissected organs (liver and lung) from the animals depicted in Panel A.

FIG. 13 provides (A) luminescence images of genetically modified mice that express human ASGR1 on liver cells (ASGR1 Humanized mice) 33 days post intravenous injection with phosphate buffered saline (PBS) or with 5.0×10¹¹ viral genomes (vg)/animal of SpyTagged chimeric AAV2/Sea Lion AAV particles carrying a firefly luciferase nucleotide of interest and modified by (1) SpyCatcher-anti-human ASGR1 antibody or (2) SpyCatcher-anti-human GLP1R antibody (control mAb). Viruses express a Firefly luciferase as a marker of transduction. Mice were anesthetized using isoflurane, injected with a Luciferin substrate and imaged 10 minutes later using the IVIS Spectrum In Vivo Imaging System (PerkinElmer); (B) quantification of the average radiance of the individual animals within the luminescence images depicted in panel A; (C) quantification of the average radiance of dissected organs (liver and lung) from the animals depicted in panel A.

FIG. 14 provides an immunofluorescence image of a neonatal mouse inner ear organ of corti explant culture 3 days after infection with chimeric AAV2/Sea Lion AAV particles lacking SpyTag. Virus expresses GFP as a marker of transduction (green), and hair cells are labeled with an antibody that detects Myo7a (red).

FIG. 15A provides an alignment of C-terminal 16 amino acids of AAV2 or AAV2/Sea Lion chimeric sequences comprising a modification of the B1 epitope to replace it entirely or only at residue 730 with homologous Sea Lion AAV sequence. The B1 monoclonal antibody epitope is illustrated. FIG. 15B provides qPCR quantification of the virus titer achieved from purified virus preparations of chimeric AAV2/Sea Lion AAV particles having no SpyTag or having no SpyTag and comprising a modification of the B1 epitope to replace it entirely or only at residue 730 with homologous Sea Lion AAV sequence. FIG. 15C provides a protein gel stain using SYPRO Ruby analyzing the expression of VP1, VP2, and VP3 capsid proteins of chimeric AAV2/Sea Lion AAV particles having no SpyTag or having no SpyTag and comprising a modification of the B1 epitope to replace it entirely or only at residue 730 with homologous Sea Lion AAV sequence. FIG. 15D provides an XY-plot obtained from luminescence evaluation of NanoLuc Luciferase expression from HEK293T cell lysates infected at various multiplicities of infection (MOIs) with chimeric AAV2/Sea Lion AAV particles having no SpyTag or having no SpyTag and comprising a modification of the B1 epitope to replace it entirely or only at residue 730 with homologous Sea Lion AAV sequence. Viruses express NanoLuc Luciferase as a marker of transduction.

FIG. 16 provides quantification of average radiance of dissected organs from mice 34 days post intravenous injection with phosphate buffered saline (PBS) or 5.0×10¹¹ viral genomes (vg)/animal of chimeric AAV2/Sea Lion AAV particles carrying a firefly luciferase nucleotide of interest and modified by having no SpyTag or having no SpyTag and comprising a modification of the B1 epitope to replace it entirely or only at residue 730 with homologous Sea Lion AAV sequence. Viruses express a Firefly luciferase as a marker of transduction. Mice were anesthetized using isoflurane, injected with a Luciferin substrate and imaged 10 minutes later using the IVIS Spectrum In Vivo Imaging System (PerkinElmer).

FIG. 17 provides an illustrative (not to scale), non-limiting, and exemplary embodiment of a Rep-Cap expression plasmid of the invention that may be used to generate an AAV chimeric viral particle. Primate AAV sequences are shown as unfilled boxes and Sea Lion AAV sequences are shown in filled boxes. Also depicted for illustrative purposes only are exemplary non-limiting positions (not to scale) for (1) alternate interface locations between primate and Sea Lion capsid sequences (dotted black lines within capsid sequence of the primate animal AAV) and (2) a detectable label for detection of the encoded VP1, VP2, and VP3 proteins (dotted line within capsid sequences of the non-primate animal AAV). A Rep-Cap expression plasmid as shown in FIG. 17 may be used to produce AAV viral particles comprising a nucleotide of interest that is flanked by the 5′ and 3′ inverted terminal repeat (ITR) sequences of the primate AAV.

FIG. 18A provides qPCR quantification of the virus titer achieved from purified virus preparations of chimeric AAV2/Sea Lion AAV particles having no SpyTag and comprising of alternate interface locations between AAV2 and Sea Lion capsid sequences. Preparations were purified from cell Lysate (v2-v4) or both cell Lysate and Media (v5). FIG. 18B and FIG. 18C provide XY-plots obtained from luminescence evaluation of NanoLuc Luciferase expression from HEK293T cell lysates infected at various multiplicities of infection (MOIs) with AAV2/Sea Lion AAV particles having no SpyTag and alternative interface locations between AAV2 and Sea Lion capsid sequences. A historical dataset for AAV2/Sea Lion AAV No SpyTag is used as a reference. Viruses express NanoLuc Luciferase as a marker of transduction. FIG. 18D provides quantification of average radiance of dissected organs from mice 56 days post intravenous injection with phosphate buffered saline (PBS) or 5.0×10¹¹ viral genomes (vg)/animal of AAV2/Sea Lion AAV particles carrying a firefly luciferase nucleotide of interest and modified by having no SpyTag or having no SpyTag and the B1 epitope replaced entirely with homologous Sea Lion AAV sequence with or without alternative interface locations between AAV2 and Sea Lion capsid sequences. Viruses express Firefly luciferase as a marker of transduction. Mice were anesthetized using isoflurane, injected with a Luciferin substrate and imaged 10 minutes later using the IVIS Spectrum In Vivo Imaging System (PerkinElmer).

### DETAILED DESCRIPTION

Although recombinatorial approaches for targeting specific cells via AAV gene therapy has seen improvement in recent years, current recombinatorial approaches used to develop AAV that escape detection and/or neutralization by pre-existing antibodies, that presumably developed, early in life, remain problematic. Described herein is an approach that harnesses (1) the natural abilities of non-primate animal AAV or remote AAV to infect primate cells, (2) the lack of NAbs in humans to non-primate animal AAV capsid proteins, and if desired or necessary (3) the adaptability of a first member of a protein:protein binding pair engineered into the AAV capsid proteins for the production of AAV viral particles useful for directed gene therapy, e.g., the introduction of a nucleotide of interest to a specific cell of interest. Described herein is a nucleotide molecule that comprises at least an AAV cap gene harnessed according to their desired function. A nucleotide molecule of the invention comprises a cap gene or portion thereof from a non-primate animal and/or remote AAV (for the production of virus capsids that are not readily recognized by pre-existing NAbs). The cap gene or portion thereof of a non-primate AAV may be modified with a first member of a protein:protein binding pair to which a second member comprising a targeting ligand may bind and direct the tropism of the resulting AAV viral particles.

For those non-primate animal AAV or remote AAV that are unable to infect primate cells, the cap gene may be designed as a chimeric cap gene that encodes at least the phospholipase A₂ (PLA₂) domain of a primate AAV VP1 capsid protein and at least a portion of the VP3 capsid protein of a non-human primate AAV or remote AAV. The PLA₂ domain is carried by a VP1 capsid protein (more particularly the VP1-unique (VP1-u) region of the VP1 capsid) and is thought to be important during AAV infection by mediating transfer of the viral genome from late endosomes/lysosomes to the nucleus to initiate replication (Zadori et al., 2001, Dev Cell, 1(2):291-302). The VP3 capsid protein is the major surface capsid protein of an AAV virus particle, and as such, a virus capsid comprising at least a portion of a VP3 capsid protein of a non-primate animal AAV or a remote AAV is unlikely to be recognized by NAbs raised against AAV serovars isolated from primates during the course of infection with the primate AAV.

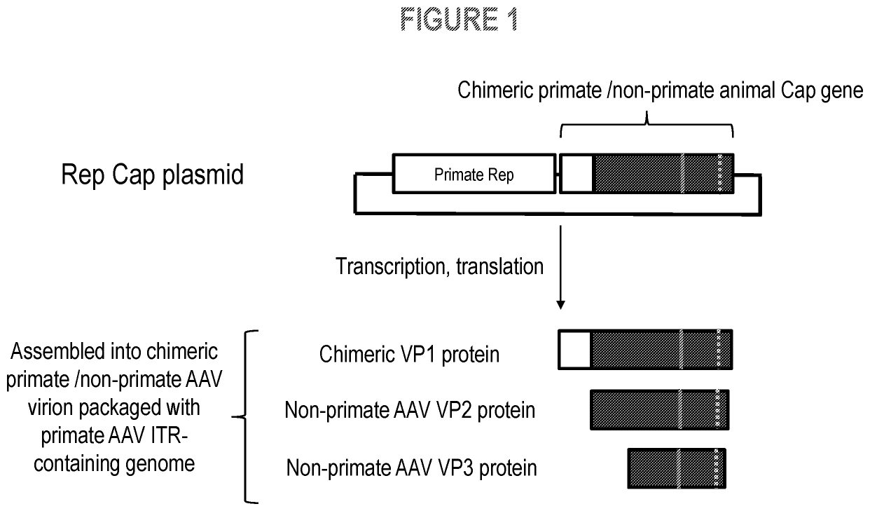

A non-limiting depiction of a nucleic acid molecule comprising a rep gene of a primate AAV and a chimeric cap gene is provided in FIG. 1. For those non-primate human AAV remote AAV that are able to infect primate cells, the rep gene of a primate AAV may be operably linked to a chimeric cap gene as described herein or a cap gene of a non-primate AAV. The examples herein show that such a nucleic acid molecule, when expressed in a packaging cell line with a helper plasmid and a primate AAV genome carrying a nucleotide of interest, is able to encode the appropriate replication and capsid proteins that function to replicate and encapsidate, respectively, the primate AAV genome into a viral particle capable of infecting cells in vivo. Moreover, the examples show that the tropism of such a AAV viral particle is easily adapted via the use of first and second members of a protein:protein binding pair, and moreover, that insertion of a first and second member of a protein:protein binding pair does not increase the likelihood of recognition from NAbs raised against primate AAV infection. As such, provided herein are the genetically modified viral particles, compositions comprising same, and methods of making and using same.

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The “percent (%) identity” or the like may be readily determined for amino acid or nucleotide sequences, over the full-length of a protein, or a portion thereof. A portion may be at least about 5 amino acids or 24 nucleotides, respectively, in length, and may be up to about 700 amino acids or 2100 nucleotides, respectively. Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Alignments may be performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using FASTA™, a program in GCG Version 6.1. Fasta™ provides alignments and percent sequence identity of the regions of the best overlap between the query and search sequences. For instance, percent sequence identity between nucleic acid sequences can be determined using FASTA™ with its default parameters (a word size of 6 and the NOPAM factor for the scoring matrix) as provided in GCG Version 6.1, herein incorporated by reference.

“Significant identity” encompasses amino acid or nucleic acid sequences alignments that are at least 90%, e.g., at least 93%, e.g., at least 95%, e.g., at least 96%, e.g., at least 97%, e.g., at least 98%, e.g., at least 99%, or e.g., at least 100% identical.

The term “chimeric” encompasses a functional gene or polypeptide comprising nucleic acid sequences or amino acid sequences, respectively, from at least two different organisms, e.g., portions of a gene or polypeptide of at least a first and second AAV, wherein the at least first and second portions are operably linked. Unless specified as chimeric, nucleotide sequences, genes, polypeptides, and amino acids are considered non-chimeric, e.g., comprising a nucleic acid sequence or amino acid sequence of only a single organism, e.g., a single AAV.

The term “antibody” includes immunoglobulin molecules comprising four polypeptide chains, two heavy (H) chains and two light (L) chains inter-connected by disulfide bonds. Each heavy chain comprises a heavy chain variable domain (V_{H}) and a heavy chain constant region (C_{H}). The heavy chain constant region comprises at least three domains, C_{H}1, C_{H}2, C_{H}3 and optionally CH₄. Each light chain comprises a light chain variable domain (C_{H}) and a light chain constant region (C_{L}). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. Typical tetrameric antibody structures comprise two identical antigen-binding domains, each of which formed by association of the V_{H} and V_{L} domains, and each of which together with respective C_{H} and C_{L} domains form the antibody Fv region. Single domain antibodies comprise a single antigen-binding domain, e.g., a V_{H} or a V_{L}. The antigen-binding domain of an antibody, e.g., the part of an antibody that recognizes and binds to the first member of a specific binding pair of an antigen, is also referred to as a “paratope.” It is a small region (of 5 to 10 amino acids) of an antibody's Fv region, part of the fragment antigen-binding (Fab region), and may contain parts of the antibody's heavy and/or light chains. A paratope specifically binds a first member of a specific binding pair when the paratope binds the first member of a specific binding pair with a high affinity. The term “high affinity” antibody refers to an antibody that has a K_{D} with respect to its target first member of a specific binding pair about of 10⁹ M or lower (e.g., about 1×10⁻⁹ M, 1×10⁻⁰ M, 1×10⁻¹¹ M, or about 1×10⁻¹² M). In one embodiment, K_{D} is measured by surface plasmon resonance, e.g., BIACORE™; in another embodiment, K_{D} is measured by ELISA.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germ line sequence or a rearranged or unrearranged sequence, and, for example, by a naive or a mature B cell or a T cell. A CDR can be somatically mutated (e.g., vary from a sequence encoded in an animal's germ line), humanized, and/or modified with amino acid substitutions, additions, or deletions. In some circumstances (e.g., for a CDR3), CDRs can be encoded by two or more sequences (e.g., germ line sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

The phrase “light chain” includes an immunoglobulin light chain sequence from any organism, and unless otherwise specified includes human K and k light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises V_{L} and J_{L} segments, derived from a repertoire of V and J segments present in the germ line. Sequences, locations and nomenclature for V and J light chain segments for various organisms can be found in IMGT database, www.imgt.org. Light chains include those, e.g., that do not selectively bind either a first or a second first member of a specific binding pair selectively bound by the first member of a specific binding pair-binding protein in which they appear. Light chains also include those that bind and recognize, or assist the heavy chain or another light chain with binding and recognizing, one or more first member of a specific binding pairs selectively bound by the first member of a specific binding pair-binding protein in which they appear. Common or universal light chains include those derived from a human Vκ1-39Jκ gene or a human Vκ3-20Jκ gene, and include somatically mutated (e.g., affinity matured) versions of the same. Exemplary human V_{L} segments include a human Vκ1-39 gene segment, a human Vκ3-20 gene segment, a human Vλ1-40 gene segment, a human Vλ1-44 gene segment, a human Vλ2-8 gene segment, a human Vλ2-14 gene segment, and human Vλ3-21 gene segment, and include somatically mutated (e.g., affinity matured) versions of the same. Light chains can be made that comprise a variable domain from one organism (e.g., human or rodent, e.g., rat or mouse; or bird, e.g., chicken) and a constant region from the same or a different organism (e.g., human or rodent, e.g., rat or mouse; or bird, e.g., chicken).

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an first member of a specific binding pair (e.g., recognizing the first member of a specific binding pair with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “heavy chain only antibody,” “heavy chain only antigen binding protein,” “single domain antigen binding protein,” “single domain binding protein” or the like refers to a monomeric or homodimeric immunoglobulin molecule comprising an immunoglobulin-like chain comprising a variable domain operably linked to a heavy chain constant region, that is unable to associate with a light chain because the heavy chain constant region typically lacks a functional C_{H}1 domain. Accordingly, the term “heavy chain only antibody,” “heavy chain only antigen binding protein,” “single domain antigen binding protein,” “single domain binding protein” or the like encompasses a both (i) a monomeric single domain antigen binding protein comprising one of the immunoglobulin-like chain comprising a variable domain operably linked to a heavy chain constant region lacking a functional C_{H}1 domain, or (ii) a homodimeric single domain antigen binding protein comprising two immunoglobulin-like chains, each of which comprising a variable domain operably linked to a heavy chain constant region lacking a functional C_{H}1 domain. In various aspects, a homodimeric single domain antigen binding protein comprises two identical immunoglobulin-like chains, each of which comprising an identical variable domain operably linked to an identical heavy chain constant region lacking a functional C_{H}1 domain. Additionally, each immunoglobulin-like chain of a single domain antigen binding protein comprises a variable domain, which may be derived from heavy chain variable region gene segments (e.g., V_{H}, D_{H}, J_{H}), light chain gene segments (e.g., V_{L}, J_{L}), or a combination thereof, linked to a heavy chain constant region (CH) gene sequence comprising a deletion or inactivating mutation in a C_{H}1 encoding sequence (and, optionally, a hinge region) of a heavy chain constant region gene, e.g., IgG, IgA, IgE, IgD, or a combination thereof. A single domain antigen binding protein comprising a variable domain derived from heavy chain gene segments may be referred to as a “V_{H}-single domain antibody” or “V_{H}-single domain antigen binding protein”, see, e.g., U.S. Pat. No. 8,754,287; U.S. Patent Publication Nos. 20140289876; 20150197553; 20150197554; 20150197555; 20150196015; 20150197556 and 20150197557, each of which is incorporated in its entirety by reference. A single domain antigen binding protein comprising a variable domain derived from light chain gene segments may be referred to as a or “V_{L}-single domain antigen binding protein,” see, e.g., U.S. Publication No. 20150289489, incorporated in its entirety by reference.

The phrase “light chain” includes an immunoglobulin light chain sequence from any organism, and unless otherwise specified includes human kappa (x) and lambda (k) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IMGT database, which is accessible via the internet on the world wide web (www) at the URL “imgt.org.” Light chains include those, e.g., that do not selectively bind either a first or a second first member of a specific binding pair selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Light chains also include those that bind and recognize, or assist the heavy chain with binding and recognizing, one or more first member of a specific binding pairs selectively bound by the first member of a specific binding pair-binding protein in which they appear. Common or universal light chains include those derived from a human Vκ1-39Jκ5 gene or a human Vκ3-20Jκ1 gene, and include somatically mutated (e.g., affinity matured) versions of the same.

The phrase “operably linked”, as used herein, includes a physical juxtaposition (e.g., in three-dimensional space) of components or elements that interact, directly or indirectly with one another, or otherwise coordinate with each other to participate in a biological event, which juxtaposition achieves or permits such interaction and/or coordination. To give but one example, a control sequence (e.g., an expression control sequence) in a nucleic acid is said to be “operably linked” to a coding sequence when it is located relative to the coding sequence such that its presence or absence impacts expression and/or activity of the coding sequence. In many embodiments, “operable linkage” involves covalent linkage of relevant components or elements with one another. Those skilled in the art will readily appreciate that, in some embodiments, covalent linkage is not required to achieve effective operable linkage. For example, in some embodiments, nucleic acid control sequences that are operably linked with coding sequences that they control are contiguous with the nucleotide of interest. Alternatively or additionally, in some embodiments, one or more such control sequences acts in trans or at a distance to control a coding sequence of interest. In some embodiments, the term “expression control sequence” as used herein refers to polynucleotide sequences which are necessary and/or sufficient to effect the expression and processing of coding sequences to which they are ligated. In some embodiments, expression control sequences may be or comprise appropriate transcription initiation, termination, promoter and/or enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and/or, in some embodiments, sequences that enhance protein secretion. In some embodiments, one or more control sequences are preferentially or exclusively active in a particular host cell or organism, or type thereof. To give but one example, in prokaryotes, control sequences typically include promoter, ribosomal binding site, and transcription termination sequence; in eukaryotes, in many embodiments, control sequences typically include promoters, enhancers, and/or transcription termination sequences. Those of ordinary skill in the art will appreciate from context that, in many embodiments, the term “control sequences” refers to components whose presence is essential for expression and processing, and in some embodiments includes components whose presence is advantageous for expression (including, for example, leader sequences, targeting sequences, and/or fusion partner sequences).

“Retargeting” or “redirecting” may include a scenario in which the wildtype particle targets several cells within a tissue and/or several organs within an organism, and general targeting of the tissue or organs is reduced or abolished by insertion of the heterologous amino acid, and retargeting to more a specific cell in the tissue or a specific organ in the organism is achieved with the targeting ligand (e.g., via a targeting ligand) that binds a marker expressed by the specific cell. Such retargeting or redirecting may also include a scenario in which the wildtype particle targets a tissue, and targeting of the tissue is reduced to or abolished by insertion of the heterologous amino acid, and retargeting to a completely different tissue is achieved with the targeting ligand.

“Specific binding pair,” “protein:protein binding pair” and the like includes two proteins (e.g., a first member (e.g., a first polypeptide) and a second cognate member (e.g., a second polypeptide)) that interact to form a bond (e.g., a non-covalent bond between a first member epitope and a second member antigen-binding portion of an antibody that recognizes the epitope) or a covalent isopeptide bond under conditions that enable or facilitate bond formation. In some embodiments, the term “cognate” refers to components that function together. Epitopes and cognate antibodies thereto, particularly epitopes that may also act as a detectable label (e.g., c-myc) are well-known in the art. Specific protein:protein binding pairs capable of interacting to form a covalent isopeptide bond are reviewed in Veggiani et al. (2014) Trends Biotechnol. 32:506, and include peptide:peptide binding pairs such as SpyTag:SpyCatcher, SpyTag002:SpyCatcher002; SpyTag:KTag; isopeptag:pilin C, SnoopTag:SnoopCatcher, etc. Generally, a first member of a protein:protein binding pair refers to member of a protein:protein binding pair, which is generally less than 30 amino acids in length, and which forms a covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e. between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 and Zaveri et al. (2012) PNAS 109:E690-E697, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO:42) which forms an amide bond to its cognate protein “SpyCatcher,” an 112 amino acid polypeptide having the amino acid sequence set forth in SEQ ID NO:43. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181) SpyLigase was engineered by excising the R strand from SpyCatcher that contains a reactive lysine, resulting in KTag, 10-residue first member of a protein:protein binding pair having the amino acid sequence ATHIKFSKRD (SEQ ID NO:72). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK, set forth as SEQ ID NO:73, and binds SpyCatcher002.

The SnoopTag:SnoopCatcher system is described in Veggiani (2016) PNAS 113:1202-07. The D4 Ig-like domain of RrgA, an adhesion from Streptococcus pneumoniae, was split to form SnoopTag (residues 734-745) and SnoopCatcher (residues 749-860). Incubation of SnoopTag and SnoopCatcher results in a spontaneous isopeptide bond that is specific between the complementary proteins. Veggiani (2016)), supra.

The isopeptag:pilin-C specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE, set forth as SEQ ID NO:75, and binds pilin-C (residues 18-299 of Spy0128). Incubation of SnoopTag and SnoopCatcher results in a spontaneous isopeptide bond that is specific between the complementary proteins. Zakeir and Howarth (2010), supra.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag, FLAG tag, Strep II tag, streptavidin-binding peptide (SBP) tag, calmodulin-binding peptide (CEP), glutathione S-transferase (GST), maltose-binding protein (MBP), S-tag, HA tag, and c-myc (SEQ ID NO:44). (Reviewed in Zhao et al. (2013) J Analytical Meth. Chem. 1-8; incorporated herein by reference). A common detectable label for primate AAV is the B1 epitope (SEQ ID NO:45). Non-primate AAV capsid proteins of the invention, which do not naturally comprise the B1 epitope, may be modified herein to comprise a B1 epitope. Generally, non-primate AAV capsid proteins may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

The term “target cells” includes any cells in which expression of a nucleotide of interest is desired. Preferably, target cells exhibit a receptor on their surface that allows the cell to be targeted with a targeting ligand, as described below.

The term “transduction” or “infection” or the like refers to the introduction of a nucleic acid into a target cell nucleus by a viral particle. The term efficiency in relation to transduction or the like, e.g., “transduction efficiency” refers to the fraction (e.g., percentage) of cells expressing a nucleotide of interest after incubation with a set number of viral particles comprising the nucleotide of interest. Well-known methods of determining transduction efficiency include flow cytometry of cells transduced with a fluorescent reporter gene, RT-PCR for expression of the nucleotide of interest, etc.

Generally “reference” viral capsid protein/capsid/particle are identical to test viral capsid protein/capsid/particle but for the change for which the effect is to be tested. For example, to determine the effect, e.g., on transduction efficiency, of inserting a first member of a specific binding pair into a test viral particle, the transduction efficiencies of the test viral particle (in the absence or presence of an appropriate targeting ligand) can be compared to the transduction efficiencies of a reference viral particle (in the absence or presence of an appropriate targeting ligand if necessary) which is identical to the test viral particle in every instance (e.g., additional point mutations, nucleotide of interest, numbers of viral particles and target cells, etc.) except for the presence of a first member of a specific binding pair. In some embodiments, a reference viral capsid protein is one that is able to form a capsid with a second viral capsid protein modified to comprise at least a first member of a protein:protein binding pair, where the reference viral capsid protein does not comprise the first member of a protein:protein binding pair, preferably wherein the capsid formed by the reference viral capsid protein and the modified viral capsid protein is a mosaic capsid.

Adeno-Associated Viruses (AAV)

“AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 2 herein provides sequences of various non-primate AAV.

“AAV” encompasses all subtypes and both naturally occurring and modified forms, except where required otherwise. AAV includes primate AAV (e.g., AAV type 1 (AAV1), primate AAV type 2 (AAV2), primate AAV type 3 (AAV3), primate AAV type 4 (AAV4), primate AAV type 5 (AAV5), primate AAV type 6 (AAV6), primate AAV type 7 (AAV7), primate AAV type 8 (AAV8), non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc., and remote AAV etc. “Primate AAV” refers to AAV generally isolated from primates. Similarly, “non-primate animal AAV” refers to AAV isolated from non-primate animals. “Remote AAV” as used herein encompasses:

AAV isolated from primate or non-primate animals generally having limited contact with the general human population,

AAV isolated from primate animals, e.g., a primate AAV, comprising a wildtype VP1 capsid protein that comprises an amino acid sequence with less than 99%, e.g., less than 95%, e.g., less than 90%, e.g., less than 85%, amino acid sequence identity to each of the following: the VP1 capsid protein of AAV1, the VP1 capsid protein of AAV2, the VP1 capsid protein of AAV3, the VP1 capsid protein of AAV4, the VP1 capsid protein of AAV5, the VP1 capsid protein of AAV6, the VP1 capsid protein of AAV7, the VP1 capsid protein of AAV8, the VP1 capsid protein of AAV9, the VP1 capsid protein of AAV10, the VP1 capsid protein of AAV11, the VP1 capsid protein of AAV12, and the VP1 capsid protein of AAV13 and/or

AAV isolated from non-primate animals, e.g., a non-primate animal AAV, comprising a wildtype capsid protein that comprises an amino acid sequence with less than 99%, e.g., less than 95%, e.g., less than 90%, e.g., less than 85% amino acid sequence identity to each of the AAV listed in Table 2.

Seropositivity may be assessed using well-known methods. For example, the absence of presence of IgG may be performed by enzyme-linked immunosorbent assay (ELISA) or other well-known immune based assays. To detect neutralizing antibodies, a neutralization assay can be conducted in which AAV particles are incubated with increasing amounts (serial dilutions) of (i) serum of a specific subject or mixed serum of multiple subjects or (ii) purified immunoglobulins (IVIG or IgG) prepared from an individual sample or pooled serum samples (e.g., from tens to thousands of donors representing a cross-section of immunoglobulins in a given population), followed by cell infection detection, e.g., by following a reporter gene expression (e.g., a luciferase gene, GFP, etc.). The level of infection is then compared to the level in a control sample not exposed to serum/IVIG/IgG. See, e.g., Example 8. The neutralizing titer can be defined, e.g., as the concentration of IVIG/IgG or the highest dilution factor of serum that results in 50% or greater inhibition of the reporter gene expression as compared to the control. In one embodiment, a serum dilution in which more than 70% reduction in the number of infected cells is observed compared with the control is considered to be positive for neutralizing activity. Interactions with specific known neutralizing antibodies can be studied using, e.g., an immunoblot assay.

As used herein, “of a [specified] AAV” in relation to a gene (e.g., rep, cap, etc.), capsid protein (e.g., a VP1 capsid protein, a VP2 capsid protein, a VP3 capsid protein, etc.), region of a capsid protein of a specified AAV (e.g., PLA₂ region, VP1-u region, VP1/VP2 common region, VP3 region), nucleotide sequence (e.g., ITR sequence), e.g., a cap gene or capsid protein of AAV2 etc., encompasses, in addition to the gene or the polypeptide respectively comprising a nucleic acid sequence or amino acid sequence set forth herein for the specified AAV, also variants of the gene or polypeptide, including variants comprising the least number of nucleotides or amino acids required to retain one or more biological functions. As used herein, a variant gene or a variant polypeptide comprises a nucleic acid sequence or amino acid sequence that differs from the nucleic acid sequence or amino acid sequence set forth herein for the gene or polypeptide of a specified AAV, wherein the difference(s) does not generally alter at least one biological function of the gene or polypeptide, and/or the phylogenetic characterization of the gene or polypeptide, e.g., where the difference(s) may be due to degeneracy of the genetic code, isolate variations, length of the sequence, etc. For example, rep gene and the cap gene as used here may encompass rep and cap genes that differ from the wildtype gene in that the genes may encode one or more Rep proteins and Cap proteins, respectively. In some embodiments, a Rep gene encodes at least Rep78 and/or Rep68. In some embodiments, cap gene includes those may differ from the wildtype in that one or more alternative start codons or sequences between one or more alternative start codons are removed such that the cap gene encodes only a single Cap protein, e.g., wherein the VP2 and/or VP3 start codons are removed or substituted such that the cap gene encodes a functional VP1 capsid protein but not a VP2 capsid protein or a VP3 capsid protein. Accordingly, as used herein, a rep gene encompasses any sequence that encodes a functional Rep protein. A cap gene encompasses any sequence that encodes at least one functional cap gene.

It is well-known that the wildtype cap gene expresses all three VP1, VP2, and VP3 capsid proteins from a single open reading frame of the cap gene under control of the p40 promoter found in the rep ORF. The term “capsid protein,” “Cap protein” and the like includes a protein that is part of the capsid of the virus. For adeno-associated viruses, the capsid proteins are generally referred to as VP1, VP2 and/or VP3, and may be encoded by the single cap gene. For AAV, the three AAV capsid proteins are produced in nature an overlapping fashion from the cap ORF alternative translational start codon usage, although all three proteins use a common stop codon. The ORF of a wildtype cap gene encodes from 5′ to 3′ three alternative start codons: “the VP1 start codon,” “the VP2 start codon,” and “the VP3 start codon”; and one “common stop codon”. The largest viral protein, VP1, is generally encoded from the VP1 start codon to the “common stop codon.” VP2 is generally encoded from the VP2 start codon to the common stop codon. VP3 is generally encoded from the VP3 start codon to the common stop codon. Accordingly, VP1 comprises at its N-terminus sequence that it does not share with the VP2 or VP3, referred to as the VP1-unique region (VP1-u). The VP1-u region is generally encoded by the sequence of a wildtype cap gene starting from the VP1 start codon to the “VP2 start codon.” VP1-u comprises a phospholipase A2 domain (PLA₂), which may be important for infection, as well as nuclear localization signals which may aid the virus in targeting to the nucleus for uncoating and genome release. The VP1, VP2, and VP3 capsid proteins share the same C-terminal sequence that makes up the entirety of VP3, which may also be referred to herein as the VP3 region. The VP3 region is encoded from the VP3 start codon to the common stop codon. VP2 has an additional ˜60 amino acids that it shares with the VP1. This region is called the VP1/VP2 common region.

In some embodiments, one or more of the Cap proteins of the invention may be encoded by one or more cap genes having one or more ORFs. In some embodiments, the VP proteins of the invention may be expressed from more than one ORF comprising nucleotide sequence encoding any combination of VP1, VP2, and/or VP3 by use of separate nucleotide sequences operably linked to at least one expression control sequence for expression in packaging cell, each producing one or more of VP1, VP2, and/or VP3 capsid proteins of the invention. In some embodiments, a VP capsid protein of the invention may be expressed individually from an ORF comprising nucleotide sequence encoding any one of VP1, VP2, or VP3 by use of separate nucleotide sequences operably linked to one expression control sequence for expression in a viral replication cell, each producing only one of VP1, VP2, or VP3 capsid protein. In another embodiment, VP proteins may be expressed from one ORF comprising nucleotide sequences encoding VP1, VP2, and VP3 capsid proteins operably linked to at least one expression control sequence for expression in a viral replication cell, each producing VP1, VP2, and VP3 capsid protein. Accordingly, although amino acid positions provided herein may be provided in relation to the VP1 capsid protein of the referenced AAV, a skilled artisan would be able to respectively and readily determine the position of that same amino acid within the VP2 and/or VP3 capsid protein of the AAV, and the corresponding position of amino acids among different AAV.

The phrase “Inverted terminal repeat” or “ITR” includes symmetrical nucleic acid sequences in the genome of adeno-associated viruses required for efficient replication. ITR sequences are located at each end of the AAV DNA genome. The ITRs serve as the origins of replication for viral DNA synthesis and are essential cis components for generating AAV particles, e.g., packaging into AAV particles.

AAV ITR comprise recognition sites for replication proteins Rep78 or Rep68. A “D” region of the ITR comprises the DNA nick site where DNA replication initiates and provides directionality to the nucleic acid replication step. An AAV replicating in a mammalian cell typically comprises two ITR sequences.

A single ITR may be engineered with Rep binding sites on both strands of the “A” regions and two symmetrical D regions on each side of the ITR palindrome. Such an engineered construct on a double-stranded circular DNA template allows Rep78 or Rep68 initiated nucleic acid replication that proceeds in both directions. A single ITR is sufficient for AAV replication of a circular particle. In methods of producing an AAV viral particle of the invention, the rep encoding sequence encodes a Rep protein or Rep protein equivalent that is capable of binding an ITR comprised on the transfer plasmid.

The Cap proteins of the invention, when expressed with appropriate Rep proteins by a packaging cell, may encapsidate a transfer plasmid comprising a nucleotide of interest and an even number of two or more ITR sequences. In some embodiments, a transfer plasmid comprises one ITR sequence. In some embodiments, a transfer plasmid comprises two ITR sequences.

Either Rep78 and/or Rep68 bind to unique and known sites on the sequence of the ITR hairpin, and act to break and unwind the hairpin structures on the end of an AAV genome, thereby providing access to replication machinery of the viral replication cell. As is well-known, Rep proteins may be expressed from more than one ORF comprising nucleotide sequence encoding any combination of Rep78, Rep68, Rep 52 and/or Rep40 by use of separate nucleotide sequences operably linked to at least one expression control sequence for expression in a viral replication cell, each producing one or more of Rep78, Rep68, Rep 52 and/or Rep40 Rep proteins. Alternatively, Rep proteins may be expressed individually from an ORF comprising a nucleotide sequence encoding any one of Rep78, Rep68, Rep 52, or Rep40 by use of separate nucleotide sequences operably linked to one expression control sequence for expression in a packaging cell, each producing only one Rep78, Rep68, Rep 52, or Rep40 Rep protein. In another embodiment, Rep proteins may be expressed from one ORF comprising nucleotide sequences encoding Rep78 and Rep52 Rep proteins operably linked to at least one expression control sequence for expression in a viral replication cell each producing Rep78 and Rep52 Rep protein.

In a method of producing an AAV virion, e.g., viral particle, of the invention, a rep encoding sequence and a cap gene of the invention may be provided a single packaging plasmid (see, e.g., FIG. 1). However, a skilled artisan will recognize that such proviso is not necessary. Such viral particles may or may not include a genome.

A “chimeric AAV capsid protein” includes an AAV capsid protein that comprises amino acid sequences, e.g., portions, from two or more different AAV and that is capable of forming and/or forms an AAV viral capsid/viral particle. A chimeric AAV capsid protein is encoded by a chimeric AAV capsid gene, e.g., a chimeric nucleotide comprising a plurality, e.g., at least two, nucleic acid sequences, each of which plurality is identical to a portion of a capsid gene encoding a capsid protein of distinct AAV, and which plurality together encodes a functional chimeric AAV capsid protein. Association of a chimeric capsid protein to a specific AAV indicates that the capsid protein comprises one or more portions from a capsid protein of that AAV and one or more portions from a capsid protein of a different AAV. For example, a chimeric AAV2 capsid protein includes a capsid protein comprising one or more portions of a VP1, VP2, and/or VP3 capsid protein of AAV2 and one or more portions of a VP1, VP2, and/or VP3 capsid protein of a different AAV.

The term “portion” refers to at least 5 amino acids or at least 15 nucleotides, but less than the full-length polypeptide or nucleic acid molecule, with 100% identity to a sequence from which the portion is derived, see Penzes (2015) J. General Virol. 2769. A “portion” encompasses any contiguous segment of amino acids or nucleotides sufficient to determine that the polypeptide or nucleic acid molecule form which the portion is derived is “of a [specified] AAV” or has “significant identity” to a particular AAV, e.g., a non-primate animal AAV or remote AAV. In some embodiments, a portion comprises at least 5 amino acids or 15 nucleotides with 100% identity to a sequence associated with the specified AAV. In some embodiments, a portion comprises at least 10 amino acids or 30 nucleotides with 100% identity to a sequence associated with the specified AAV. In some embodiments, a portion comprises at least 15 amino acids or 45 nucleotides with 100% identity to a sequence associated with the specified AAV. In some embodiments, a portion comprises at least 20 amino acids or 60 nucleotides with 100% identity to a sequence associated with the specified AAV. In some embodiments, a portion comprises at least 25 amino acids or 75 nucleotides with 100% identity to a sequence associated with the specified AAV. In some embodiments, a portion comprises at least 30 amino acids or 90 nucleotides with 100% identity to a sequence associated with the specified AAV. In some embodiments, a portion comprises at least 35 amino acids or 105 nucleotides with 100% identity to a sequence associated with the specified AAV. In some embodiments, a portion comprises at least 40 amino acids or 120 nucleotides with 100% identity to a sequence associated with the specified AAV. In some embodiments, a portion comprises at least 45 amino acids or 135 nucleotides with 100% identity to a sequence associated with the specified AAV. In some embodiments, a portion comprises at least 50 amino acids or 150 nucleotides with 100% identity to a sequence associated with the specified AAV. In some embodiments, a portion comprises at least 60 amino acids or 180 nucleotides with 100% identity to a sequence associated with the specified AAV. In some embodiments, a portion comprises at least 70 amino acids or 210 nucleotides with 100% identity to a sequence associated with the specified AAV. In some embodiments, a portion comprises at least 80 amino acids or 240 nucleotides with 100% identity to a sequence associated with the specified AAV. In some embodiments, a portion comprises at least 90 amino acids or 270 nucleotides with 100% identity to a sequence associated with the specified AAV. In some embodiments, a portion comprises at least 100 amino acids or 300 nucleotides with 100% identity to a sequence associated with the specified AAV.

Modified Virus Capsid Proteins, Viral Particles, Nucleic Acids

In some embodiments, a Cap protein of the invention, e.g., a VP1 capsid protein as described herein, a VP2 capsid protein as described herein, and/or a VP3 capsid protein as described herein, is modified to comprise e.g., a first member of a protein:protein binding pair, a detectable label, point mutation, etc.

Chimerism is a type of modification as described herein. Generally, modification of gene or a polypeptide of a specified AAV, or variants thereof, results in nucleic acid sequence or an amino acid sequence that differs from the nucleic acid sequence or amino acid sequence set forth herein for the specified AAV, wherein the modification alters, confers, or removes one or more biological functions, but does not change the phylogenetic characterization of, the gene or polypeptide. A modification may include an insertion of, e.g., a first member of a protein:protein binding pair and a point mutation, e.g., such that the natural tropism of the capsid protein is reduced to abolished and/or such that the capsid protein comprises a detectable label. Preferred modifications include those that do not alter and preferably decrease the low to no recognition of the modified capsid by pre-existing antibodies found in the general population that were produced during the course of infection with another AAV, e.g., infection with serotypes such as AAV1, AAV2, AAV3, AAV4, AAV5, AAV6, AAV7, AAV8, AAV9, AAV10, AAV11, AAV12, AAV13, AAVDJ, Anc80L65, AAV2G9, AAV-LK03, virions based on such serotypes, virions from currently used AAV gene therapy modalities, or a combination thereof. Other modifications as described herein include modification of a capsid protein such that it comprises a first member of a protein:protein binding pair, a detectable label, etc., which modifications generally result from modifications at the genetic level, e.g., via modification of a cap gene.

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a protein:protein binding pair and a second corresponding viral capsid protein lacking the first member of a protein:protein binding pair. In relation to a mosaic capsid, the second viral capsid protein lacking the first member of a protein:protein binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a protein:protein binding pair, except that the reference capsid protein lacks the first member of a protein:protein binding pair. In some mosaic capsid embodiments, a VP1, VP2, and/or VP3 reference capsid protein corresponds to the viral VP1, VP2, and/or VP3 capsid protein modified with a first member of a protein:protein binding pair, except that the reference capsid protein lacks the first member of a protein:protein binding pair. In some embodiments, a VP1 reference capsid protein corresponds to the viral VP1 capsid protein modified with a first member of a protein:protein binding pair, except that the reference capsid protein lacks the first member of a protein:protein binding pair. In some embodiments, a VP2 reference capsid protein corresponds to the viral VP2 capsid protein modified with a first member of a protein:protein binding pair, except that the reference capsid protein lacks the first member of a protein:protein binding pair. In some embodiments, a VP3 reference capsid protein corresponds to the viral VP3 capsid protein modified with a first member of a protein:protein binding pair, except that the reference capsid protein lacks the first member of a protein:protein binding pair. In some mosaic capsid embodiments comprising a chimeric VP1, VP2, and/or VP3 capsid protein further modified to comprise a first member of a protein:protein binding pair, a reference protein may be a corresponding capsid protein from which portions thereof form part of the chimeric capsid protein. As a non-limiting example in some embodiments, mosaic capsid comprising a chimeric AAV2/AAAV VP1 capsid protein modified to comprise a first member of a protein:protein binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a protein:protein binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the protein:protein binding pair and is able to form a capsid with the first capsid protein modified with the first member of a protein:protein binding pair.

Generally mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a protein:protein binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1. In some mosaic viral particle embodiments, the protein subunit is at least about 1:1 (e.g., the mosaic viral particle comprises about 30 modified capsid proteins and about 30 reference capsid protein). In some mosaic viral particle embodiments, the protein subunit ratio is at least about 1:2 (e.g., the mosaic viral particle comprises about 20 modified capsid proteins and about 40 reference capsid proteins). In some mosaic viral particle embodiments, the protein subunit ratio is at least about 3:5. In some mosaic viral particle embodiments, the protein subunit ratio is at least about 1:3 (e.g., the mosaic viral particle comprises about 15 modified capsid proteins and about 45 reference capsid proteins). In some mosaic viral particle embodiments, the protein subunit ratio is at least about 1:4 (e.g., the mosaic viral particle comprises about 12 modified capsid proteins and 48 reference capsid proteins). In some mosaic viral particle embodiments, the protein subunit ratio is at least about 1:5 (e.g., the mosaic viral particle comprises 10 modified capsid proteins and 50 reference capsid proteins). In some mosaic viral particle embodiments, the protein subunit ratio is at least about 1:6. In some mosaic viral particle embodiments, the protein subunit ratio is at least about 1:7. In some mosaic viral particle embodiments, the protein subunit ratio is at least about 1:8. In some mosaic viral particle embodiments, the protein subunit ratio is at least about 1:9 (e.g., the mosaic viral particle comprises about 6 modified capsid proteins and about 54 reference capsid proteins). In some mosaic viral particle embodiments, the protein subunit ratio is at least about 1:10. In some mosaic viral particle embodiments, the protein subunit ratio is at least about 1:11 (e.g., the mosaic viral particle comprises about 5 modified capsid proteins and about 55 reference capsid proteins). In some mosaic viral particle embodiments, the protein subunit ratio is at least about 1:12. In some mosaic viral particle embodiments, the protein subunit ratio is at least about 1:13. In some mosaic viral particle embodiments, the protein subunit ratio is at least about 1:14 (e.g., the mosaic viral particle comprises about 4 modified capsid proteins and about 56 reference capsid proteins). In some mosaic viral particle embodiments, the protein subunit ratio is at least about 1:15. In some mosaic viral particle embodiments, the protein subunit ratio is at least about 1:19 (e.g., the mosaic viral particle comprises about 3 modified capsid proteins and about 57 reference capsid proteins). In some mosaic viral particle embodiments, the protein subunit ratio is at least about 1:29 (e.g., the mosaic viral particle comprises about 2 modified capsid proteins and about 58 reference capsid proteins). In some mosaic viral particle embodiments, the protein subunit ratio is at least about 1:59. In some mosaic viral particle embodiments, the protein subunit ratio is at least about 2:1 (e.g., the mosaic viral particle comprises about 40 modified capsid proteins and about 20 reference capsid proteins). In some mosaic viral particle embodiments, the protein subunit ratio is at least about 5:3. In some mosaic viral particle embodiments, the protein subunit ratio is at least about 3:1 (e.g., the mosaic viral particle comprises about 45 modified capsid proteins and about 15 reference capsid proteins). In some mosaic viral particle embodiments, the protein subunit ratio is at least about 4:1 (e.g., the mosaic viral particle comprises about 48 modified capsid proteins and 12 reference capsid proteins). In some mosaic viral particle embodiments, the protein subunit ratio is at least about 5:1 (e.g., the mosaic viral particle comprises 50 modified capsid proteins and 10 reference capsid proteins). In some mosaic viral particle embodiments, the protein subunit ratio is at least about 6:1. In some mosaic viral particle embodiments, the protein subunit ratio is at least about 7:1. In some mosaic viral particle embodiments, the protein subunit ratio is at least about 8:1. In some mosaic viral particle embodiments, the protein subunit ratio is at least about 9:1 (e.g., the mosaic viral particle comprises about 54 modified capsid proteins and about 6 reference capsid proteins). In some mosaic viral particle embodiments, the protein subunit ratio is at least about 10:1. In some mosaic viral particle embodiments, the protein subunit ratio is at least about 11:1 (e.g., the mosaic viral particle comprises about 55 modified capsid proteins and about 5 reference capsid proteins). In some mosaic viral particle embodiments, the protein subunit ratio is at least about 12:1. In some mosaic viral particle embodiments, the protein subunit ratio is at least about 13:1. In some mosaic viral particle embodiments, the protein subunit ratio is at least about 14:1 (e.g., the mosaic viral particle comprises about 56 modified capsid proteins and about 4 reference capsid proteins). In some mosaic viral particle embodiments, the protein subunit ratio is at least about 15:1. In some mosaic viral particle embodiments, the protein subunit ratio is at least about 19:1 (e.g., the mosaic viral particle comprises about 57 modified capsid proteins and about 3 reference capsid proteins). In some mosaic viral particle embodiments, the protein subunit ratio is at least about 29:1 (e.g., the mosaic viral particle comprises about 58 modified capsid proteins and about 2 reference capsid proteins). In some mosaic viral particle embodiments, the protein subunit ratio is at least about 59:1.

In some non-mosaic viral particle embodiments, the protein subunit ratio may be 1:0 wherein each capsid protein of the non-mosaic viral particle is modified with a first member of a protein:protein binding pair. In some non-mosaic viral particle embodiments, the protein subunit ratio may be 0:1 wherein each capsid protein of the non-mosaic viral particle is not modified with a first member of a protein:protein binding pair.

In some embodiments, a capsid protein of the invention is modified to comprise a detectable label. Many detectable labels are known in the art. (See, e.g.: Nilsson et al. (1997) “Affinity fusion strategies for detection, purification, and immobilization of modified proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein). Detectable labels include, but are not limited to, a polyhistidine detectable labels (e.g., a His-6, His-8, or His-10) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high detectable label that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary detectable label is a SNAP-tag, commercially available from Covalys (www.covalys.com). In some embodiments, a detectable label disclosed herein comprises a detectable label recognized only by an antibody paratope. In some embodiments, a detectable label disclosed herein comprises a detectable label recognized by an antibody paratope and other specific binding pairs.

In some embodiments, the detectable label forms a binding pair with an immunoglobulin constant domain. In some embodiments, the detectable label and/or detectable label does form a binding pair with a metal ion, e.g., Ni²⁺, Co²⁺, Cu²⁺, Zn²⁺, Fe³⁺, etc. In some embodiments, the detectable label is selected from the group consisting of Streptavidin, Strep II, HA, L14, 4C-RGD, LH, and Protein A.

In some embodiments, the detectable label is selected from the group consisting of FLAG, HA and c-myc (EQKLISEEDL; SEQ ID NO:44). In some embodiments, the detectable label is c-myc (SEQ ID NO:44).

In some embodiments, a detectable label is a B cell epitope, e.g., is between about 1 amino acid and about 35 amino acids in length, and forms a binding pair with an antibody paratope, e.g., an immunoglobulin variable domain. In some embodiments, the detectable label comprises a B1 epitope (SEQ ID NO:45). In some embodiments, a capsid protein is modified to comprise a B1 epitope in the VP3 region.

In some embodiments, a capsid protein of the invention comprises at least a first member of a peptide:peptide binding pair.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc (SEQ ID NO:44). Use of a detectable label as a first member of a protein:protein binding pair is described in, e.g., WO2019006043, incorporated herein in its entirety by reference.

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from the group consisting of SpyTag:SpyCatcher, SpyTag002:SpyCatcher002, SpyTag:KTag, Isopeptag:pilin-C, and SnoopTag:SnoopCatcher. In some embodiments, wherein the first member is SpyTag (or a biologically active portion thereof) and the protein (second cognate member) is SpyCatcher (or a biologically active portion thereof). In some embodiments, wherein the first member is SpyTag (or a biologically active portion thereof) and the protein (second cognate member) is KTag (or a biologically active portion thereof). In some embodiments, wherein the first member is KTag (or a biologically active portion thereof) and the protein (second cognate member) is SpyTag (or a biologically active portion thereof). In some embodiments, wherein the first member is SnoopTag (or a biologically active portion thereof) and the protein (second cognate member) is SnoopCatcher (or a biologically active portion thereof). In some embodiments, wherein the first member is Isopeptag (or a biologically active portion thereof) and the protein (second cognate member) is Pilin-C (or a biologically active portion thereof). In some embodiments, wherein the first member is SpyTag002 (or a biologically active portion thereof) and the protein (second cognate member) is SpyCatcher002 (or a biologically active portion thereof). In some embodiments, a Cap protein of the invention comprises a SpyTag. Use of a first member of a protein:protein binding pair is described in WO2019006046, incorporated herein in its entirety.

In some embodiments, a first member of a protein:protein binding pair and/or detectable label is operably linked to (translated in frame with, chemically attached to, and/or displayed by) a Cap protein of the invention via a first or second linker, e.g., an amino acid spacer that is at least one amino acid in length. In some embodiments, the first member of a protein:protein binding pair is flanked by a first and/or second linker, e.g., a first and/or second amino acid spacer, each of which spacer is at least one amino acid in length.

In some embodiments, the first and/or second linkers are not identical. In some embodiments, the first and/or second linker is each independently one or two amino acids in length. In some embodiments, the first and/or second linker is each independently one, two or three amino acids in length. In some embodiments, the first and/or second linker is each independently one, two, three, or four amino acids in length. In some embodiments, the first and/or second linker is each independently one, two, three, four, or five amino acids in length. In some embodiments, the first and/or second linker are each independently one, two, three, four, or five amino acids in length. In some embodiments, the first and/or second linker is each independently one, two, three, four, five, or six amino acids in length. In some embodiments, the first and/or second linker is each independently one, two, three, four, five, six, or seven amino acids in length. In some embodiments, the first and/or second linker is each independently one, two, three, four, five, six, seven, or eight amino acids in length. In some embodiments, the first and/or second linker is each independently one, two, three, four, five, six, seven, eight or nine amino acids in length. In some embodiments, the first and or second linker is each independently one, two, three, four, five, six, seven, eight, nine, or ten amino acids in length. In some embodiments, the first and or second linker is each independently one, two, three, four, five, six, seven, eight, nine, ten, or more amino acids in length.

In some embodiments, the first and second linkers are identical in sequence and/or in length and are each one amino acid in length. In some embodiments, the first and second linkers are identical in length, and are each one amino acid in length. In some embodiments, the first and second linkers are identical in length, and are each two amino acids in length. In some embodiments, the first and second linkers are identical in length, and are each three amino acids in length. In some embodiments, the first and second linkers are identical in length, and are each four amino acids in length, e.g., the linker is GLSG (SEQ ID NO:37). In some embodiments, the first and second linkers are identical in length, and are each five amino acids in length. In some embodiments, the first and second linkers are identical in length, and are each six amino acids in length, e.g., the first and second linkers each comprise a sequence of GLSGSG (SEQ ID NO:38). In some embodiments, the first and second linkers are identical in length, and are each seven amino acids in length. In some embodiments, the first and second linkers are identical in length, and are each eight amino acids in length, e.g., the first and second linkers each comprise a sequence of GLSGLSGS (SEQ ID NO:39). In some embodiments, the first and second linkers are identical in length, and are each nine amino acids in length. In some embodiments, the first and second linkers are identical in length, and are each ten amino acids in length, e.g., the first and second linkers each comprise a sequence of GLSGLSGLSG (SEQ ID NO:40) or GLSGGSGLSG (SEQ ID NO:41). In some embodiments, the first and second linkers are identical in length, and are each more than ten amino acids in length.

Generally, a first member of a protein:protein binding pair amino acid sequence as described herein, e.g., comprising a first member of a specific binding pair by itself or in combination with one or more linkers, is between about 5 amino acids to about 50 amino acids in length. In some embodiments, the first member of a protein:protein binding pair amino acid sequence is at least 5 amino acids in length. In some embodiments, the first member of a protein:protein binding pair amino acid sequence is 6 amino acids in length. In some embodiments, the first member of a protein:protein binding pair amino acid sequence is 7 amino acids in length. In some embodiments, the first member of a protein:protein binding pair amino acid sequence is 8 amino acids in length. In some embodiments, the first member of a protein:protein binding pair amino acid sequence is 9 amino acids in length. In some embodiments, the first member of a protein:protein binding pair amino acid sequence is 10 amino acids in length. In some embodiments, the first member of a protein:protein binding pair amino acid sequence is 11 amino acids in length. In some embodiments, the first member of a protein:protein binding pair amino acid sequence is 12 amino acids in length. In some embodiments, the first member of a protein:protein binding pair amino acid sequence is 13 amino acids in length. In some embodiments, the first member of a protein:protein binding pair amino acid sequence is 14 amino acids in length. In some embodiments, the first member of a protein:protein binding pair amino acid sequence is 15 amino acids in length. In some embodiments, the first member of a protein:protein binding pair amino acid sequence is 16 amino acids in length. In some embodiments, the first member of a protein:protein binding pair amino acid sequence is 17 amino acids in length. In some embodiments, the first member of a protein:protein binding pair amino acid sequence is 18 amino acids in length. In some embodiments, the first member of a protein:protein binding pair amino acid sequence is 19 amino acids in length. In some embodiments, the first member of a protein:protein binding pair amino acid sequence is 20 amino acids in length. In some embodiments, the first member of a protein:protein binding pair amino acid sequence is 21 amino acids in length. In some embodiments, the first member of a protein:protein binding pair amino acid sequence is 22 amino acids in length. In some embodiments, the first member of a protein:protein binding pair amino acid sequence is 23 amino acids in length. In some embodiments, the first member of a protein:protein binding pair amino acid sequence is 24 amino acids in length. In some embodiments, the first member of a protein:protein binding pair amino acid sequence is 25 amino acids in length. In some embodiments, the first member of a protein:protein binding pair amino acid sequence is 26 amino acids in length. In some embodiments, the first member of a protein:protein binding pair amino acid sequence is 27 amino acids in length. In some embodiments, the first member of a protein:protein binding pair amino acid sequence is 28 amino acids in length. In some embodiments, the first member of a protein:protein binding pair amino acid sequence is 29 amino acids in length. In some embodiments, the first member of a protein:protein binding pair amino acid sequence is 30 amino acids in length. In some embodiments, the first member of a protein:protein binding pair amino acid sequence is 31 amino acids in length. In some embodiments, the first member of a protein:protein binding pair amino acid sequence is 32 amino acids in length. In some embodiments, the first member of a protein:protein binding pair amino acid sequence is 33 amino acids in length. In some embodiments, the first member of a protein:protein binding pair amino acid sequence is 34 amino acids in length. In some embodiments, the first member of a protein:protein binding pair amino acid sequence is 35 amino acids in length. In some embodiments, the first member of a protein:protein binding pair amino acid sequence is 36 amino acids in length. In some embodiments, the first member of a protein:protein binding pair amino acid sequence is 37 amino acids in length. In some embodiments, the first member of a protein:protein binding pair amino acid sequence is 38 amino acids in length. In some embodiments, the first member of a protein:protein binding pair amino acid sequence is 39 amino acids in length. In some embodiments, the first member of a protein:protein binding pair amino acid sequence is 40 amino acids in length. In some embodiments, the first member of a protein:protein binding pair amino acid sequence is 41 amino acids in length. In some embodiments, the first member of a protein:protein binding pair amino acid sequence is 42 amino acids in length. In some embodiments, the first member of a protein:protein binding pair amino acid sequence is 43 amino acids in length. In some embodiments, the first member of a protein:protein binding pair amino acid sequence is 44 amino acids in length. In some embodiments, the first member of a protein:protein binding pair amino acid sequence is 45 amino acids in length. In some embodiments, the first member of a protein:protein binding pair amino acid sequence is 46 amino acids in length. In some embodiments, the first member of a protein:protein binding pair amino acid sequence is 47 amino acids in length. In some embodiments, the first member of a protein:protein binding pair amino acid sequence is 48 amino acids in length. In some embodiments, the first member of a protein:protein binding pair amino acid sequence is 49 amino acids in length. In some embodiments, the first member of a protein:protein binding pair amino acid sequence is 50 amino acids in length.

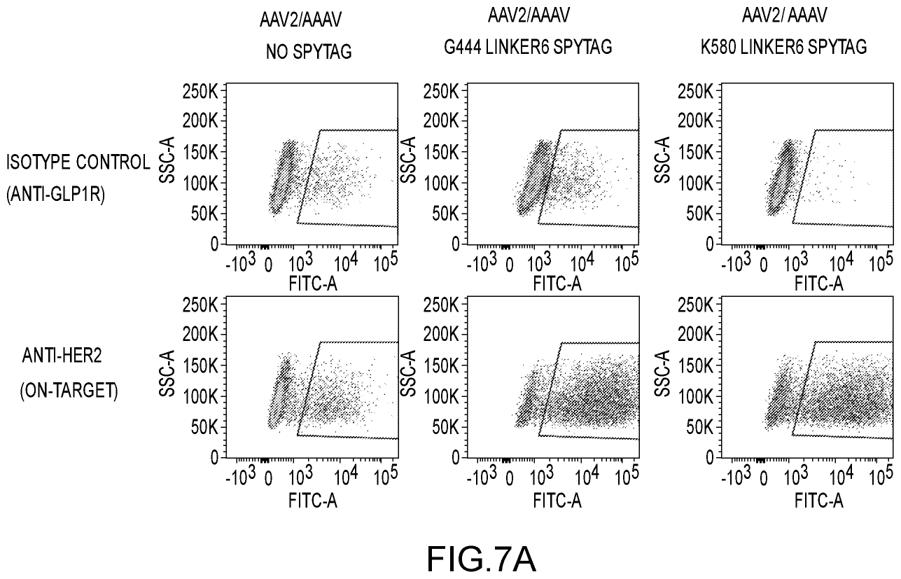

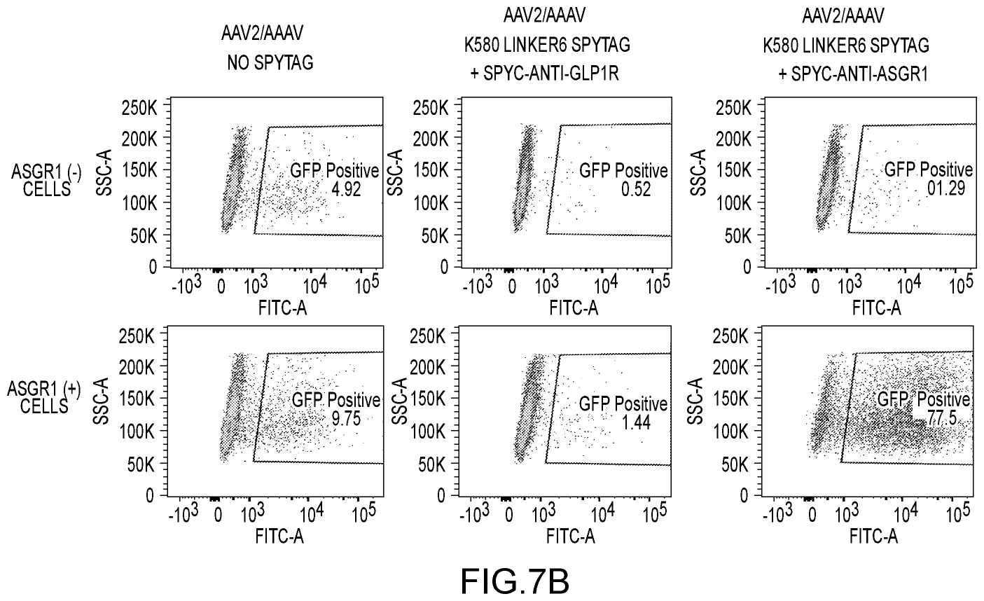

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of a first member of a protein:protein binding pair and/or detectable label.

Accordingly, in some embodiments, the first member of a protein:protein binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a protein:protein binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713 and I-716 of the VP1 capsid protein of AAV2 (Wu et al. (2000) J. Virol. 74:8635-8647). A modified virus capsid protein as described herein may be a non-primate animal capsid protein comprising a first member of a protein:protein binding pair and/or detectable label inserted into a position corresponding with a position of an AAV2 capsid protein selected from the group consisting of I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites of a non-primate animal AAV that include those corresponding to I-587 of AAV1, I-589 of AAV1, I-585 of AAV3, I-585 of AAV4, and I-585 of AAV5. In some embodiments, a modified virus capsid protein as described herein may be a non-primate animal capsid protein comprising a first member of a protein:protein binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a protein:protein binding pair and/or detectable label is inserted in a VP1 capsid protein of a non-primate animal AAV after an amino acid position corresponding with an amino acid position selected from the group consisting of I444 of an avian AAV capsid protein VP1, 1580 of an avian AAV capsid protein VP1, 1573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and I565 of a sea lion AAV capsid protein VP1.

The nomenclature I-###, I# or the like herein refers to the insertion site (I) with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Also provided herein are nucleic acids that encode a VP3 capsid protein of the invention. AAV capsid proteins may be, but are not necessarily, encoded by overlapping reading frames of the same gene with staggered start codons. In some embodiments, a nucleic acid that encodes a VP3 capsid protein of the invention does not also encode a VP2 capsid protein or VP1 capsid protein of the invention. In some embodiments, a nucleic acid that encodes a VP3 capsid protein of the invention may also encode a VP2 capsid protein of the invention but does not also encode a VP1 capsid of the invention. In some embodiments, a nucleic acid that encodes a VP3 capsid protein of the invention may also encode a VP2 capsid protein of the invention and a VP1 capsid of the invention.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid.

In some embodiments, a viral capsid comprising the modified viral capsid protein comprising the first and second members of a protein:protein binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a protein:protein binding pair, e.g., comprises a control capsid protein. In some embodiments, a viral capsid comprising a modified viral capsid protein as described herein bound to the first and second members of a protein:protein binding pair linked to a targeting ligand exhibits a detectable transduction efficiency compared to the undetectable transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 10% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 20% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 30% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 40% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 50% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 60% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 70% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 75% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 80% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 85% greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is 90% greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is 95% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is 99% greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 1.5-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 2-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 3-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 4-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 5-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 6-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 7-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 8-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 9-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 10-fold greater than the transduction efficiency of a control viral capsid. In some embodiments, a viral capsid comprising a modified viral capsid protein as described herein bound to an appropriate the first and second members of a protein:protein binding pair linked to a targeting ligand exhibits a transduction efficiency that is at least 20-fold greater than the transduction efficiency of a control capsid. In some embodiments, a viral capsid comprising a modified viral capsid protein as described herein bound to an appropriate the first and second members of a protein:protein binding pair linked to a targeting ligand exhibits a transduction efficiency that is at least 30-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 40-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 50-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 60-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 70-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 80-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 90-fold greater than the transduction efficiency of a control viral capsid. In some embodiments, a viral capsid comprising a modified viral capsid protein as described herein bound to the first and second members of a protein:protein binding pair linked to a targeting ligand exhibits a transduction efficiency that is at least 100-fold greater than the transduction efficiency of a control viral capsid In some embodiments, a viral particle of the invention comprising a viral capsid protein comprising an amino acid sequence of a capsid protein of a non-primate animal AAV, a remote AAV, or a combination thereof, and optionally comprising a first and second members of a protein:protein binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a protein:protein binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC50 value that is at least 2-fold that of a control virus particle).

In some embodiments, a viral particle of the invention comprising a viral capsid protein comprising an amino acid sequence of a capsid protein of a non-primate animal AAV, a remote AAV, or a combination thereof requires at least 3-fold more total IVIG or IgG for neutralization (e.g., 50% or more infection inhibition) compared to an appropriate control viral particle, e.g., (e.g., a viral particle of the invention has an IC50 value that is at least 3-fold that of a control virus particle). In some embodiments, a viral particle of the invention comprising a viral capsid protein comprising an amino acid sequence of a capsid protein of a non-primate animal AAV, a remote AAV, or a combination thereof requires at least 4-fold more total IVIG or IgG for neutralization (e.g., 50% or more infection inhibition) compared to an appropriate control viral particle, e.g., (e.g., a viral particle of the invention has an IC50 value that is at least 4-fold that of a control virus particle). In some embodiments, a viral particle of the invention comprising a viral capsid protein comprising an amino acid sequence of a capsid protein of a non-primate animal AAV, a remote AAV, or a combination thereof requires at least 5-fold more total IVIG or IgG for neutralization (e.g., 50% or more infection inhibition) compared to an appropriate control viral particle, e.g., (e.g., a viral particle of the invention has an IC50 value that is at least 5-fold that of a control virus particle). In some embodiments, a viral particle of the invention comprising a viral capsid protein comprising an amino acid sequence of a capsid protein of a non-primate animal AAV, a remote AAV, or a combination thereof requires at least 6-fold more total IVIG or IgG for neutralization (e.g., 50% or more infection inhibition) compared to an appropriate control viral particle, e.g., (e.g., a viral particle of the invention has an IC50 value that is at least 6-fold that of a control virus particle). In some embodiments, a viral particle of the invention comprising a viral capsid protein comprising an amino acid sequence of a capsid protein of a non-primate animal AAV, a remote AAV, or a combination thereof requires at least 7-fold more total IVIG or IgG for neutralization (e.g., 50% or more infection inhibition) compared to an appropriate control viral particle, e.g., (e.g., a viral particle of the invention has an IC50 value that is at least 7-fold that of a control virus particle. In some embodiments, a viral particle of the invention comprising a viral capsid protein comprising an amino acid sequence of a capsid protein of a non-primate animal AAV, a remote AAV, or a combination thereof requires at least 8-fold more total IVIG or IgG for neutralization (e.g., 50% or more infection inhibition) compared to an appropriate control viral particle, e.g., (e.g., a viral particle of the invention has an IC50 value that is at least 8-fold that of a control virus particle). In some embodiments, a viral particle of the invention comprising a viral capsid protein comprising an amino acid sequence of a capsid protein of a non-primate animal AAV, a remote AAV, or a combination thereof requires at least 9-fold more total IVIG or IgG for neutralization (e.g., 50% or more infection inhibition) compared to an appropriate control viral particle, e.g., (e.g., a viral particle of the invention has an IC50 value that is at least 9-fold that of a control virus particle). In some embodiments, a viral particle of the invention comprising a viral capsid protein comprising an amino acid sequence of a capsid protein of a non-primate animal AAV, a remote AAV, or a combination thereof requires at least 10-fold more total IVIG or IgG for neutralization (e.g., 50% or more infection inhibition) compared to an appropriate control viral particle, e.g., (e.g., a viral particle of the invention has an IC50 value that is at least 10-fold that of a control virus particle). In some embodiments, a viral particle of the invention comprising a viral capsid protein comprising an amino acid sequence of a capsid protein of a non-primate animal AAV, a remote AAV, or a combination thereof requires at least 20-fold more total IVIG or IgG for neutralization (e.g., 50% or more infection inhibition) compared to an appropriate control viral particle, e.g., (e.g., a viral particle of the invention has an IC50 value that is at least 20-fold that of a control virus particle). In some embodiments, a viral particle of the invention comprising a viral capsid protein comprising an amino acid sequence of a capsid protein of a non-primate animal AAV, a remote AAV, or a combination thereof requires at least 30-fold more total IVIG or IgG for neutralization (e.g., 50% or more infection inhibition) compared to an appropriate control viral particle, e.g., (e.g., a viral particle of the invention has an IC50 value that is at least 30-fold that of a control virus particle). In some embodiments, a viral particle of the invention comprising a viral capsid protein comprising an amino acid sequence of a capsid protein of a non-primate animal AAV, a remote AAV, or a combination thereof requires at least 40-fold more total IVIG or IgG for neutralization (e.g., 50% or more infection inhibition) compared to an appropriate control viral particle, e.g., (e.g., a viral particle of the invention has an IC50 value that is at least 40-fold that of a control virus particle). In some embodiments, a viral particle of the invention comprising a viral capsid protein comprising an amino acid sequence of a capsid protein of a non-primate animal AAV, a remote AAV, or a combination thereof requires at least 50-fold more total IVIG or IgG for neutralization (e.g., 50% or more infection inhibition) compared to an appropriate control viral particle, e.g., (e.g., a viral particle of the invention has an IC50 value that is at least 50-fold that of a control virus particle). In some embodiments, a viral particle of the invention comprising a viral capsid protein comprising an amino acid sequence of a capsid protein of a non-primate animal AAV, a remote AAV, or a combination thereof requires at least 60-fold more total IVIG or IgG for neutralization (e.g., 50% or more infection inhibition) compared to an appropriate control viral particle, e.g., (e.g., a viral particle of the invention has an IC50 value that is at least 60-fold that of a control virus particle). In some embodiments, a viral particle of the invention comprising a viral capsid protein comprising an amino acid sequence of a capsid protein of a non-primate animal AAV, a remote AAV, or a combination thereof requires at least 70-fold more total IVIG or IgG for neutralization (e.g., 50% or more infection inhibition) compared to an appropriate control viral particle, e.g., (e.g., a viral particle of the invention has an IC50 value that is at least 70-fold that of a control virus particle). In some embodiments, a viral particle of the invention comprising a viral capsid protein comprising an amino acid sequence of a capsid protein of a non-primate animal AAV, a remote AAV, or a combination thereof requires at least 80-fold more total IVIG or IgG for neutralization (e.g., 50% or more infection inhibition) compared to an appropriate control viral particle, e.g., (e.g., a viral particle of the invention has an IC50 value that is at least 80-fold that of a control virus particle). In some embodiments, a viral particle of the invention comprising a viral capsid protein comprising an amino acid sequence of a capsid protein of a non-primate animal AAV, a remote AAV, or a combination thereof requires at least 90-fold more total IVIG or IgG for neutralization (e.g., 50% or more infection inhibition) compared to an appropriate control viral particle, e.g., (e.g., a viral particle of the invention has an IC50 value that is at least 90-fold that of a control virus particle). In some embodiments, a viral particle of the invention comprising a viral capsid protein comprising an amino acid sequence of a capsid protein of a non-primate animal AAV, a remote AAV, or a combination thereof requires at least 100-fold more total IVIG or IgG for neutralization (e.g., 50% or more infection inhibition) compared to an appropriate control viral particle, e.g., (e.g., a viral particle of the invention has an IC50 value that is at least 100-fold that of a control virus particle). In some embodiments, a viral particle of the invention comprising a viral capsid protein comprising an amino acid sequence of a capsid protein of a non-primate animal AAV, a remote AAV, or a combination thereof has an undetectable IC50 when incubated with pooled human serum Ig from at least 100; 10,000; 20,000; 30,000; 40,000; 50,000; or more human donors.

Targeting Ligands

A viral particle described herein may further comprise a targeting ligand.

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule comprising (i) an antibody paratope that specifically binds the detectable label and (ii) a second binding domain that specifically binds a receptor targets the viral particle. Such “targeting” or “directing” may include a scenario in which the wildtype viral particle targets several cells within a tissue and/or several organs within an organism, which broad targeting of the tissue or organs is reduced to abolished by insertion of the detectable label, and which retargeting to more specific cells in the tissue or more specific organ in the organism is achieved with the multispecific binding molecule. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule. An antibody paratope as described herein generally comprises at a minimum a complementarity determining region (CDR) that specifically recognizes the detectable label, e.g., a CDR3 region of a heavy and/or light chain variable domain. In some embodiments, a multispecific binding molecule comprises an antibody (or portion thereof) that comprises the antibody paratope that specifically binds the detectable label. For example, a multispecific binding molecule may comprise a single domain heavy chain variable region or a single domain light chain variable region, wherein the single domain heavy chain variable region or single domain light chain variable region comprises an antibody paratope that specifically binds the detectable label. In some embodiments, a multispecific binding molecule may comprise an Fv region, e.g., a multispecific binding molecule may comprise an scFv, that comprises an antibody paratope that specifically binds the detectable label. In some embodiments, a multispecific binding molecule as described herein comprises an antibody paratope that specifically binds c-myc (SEQ ID NO:44).

One embodiment of the present invention is a multimeric structure comprising a modified viral capsid protein of the present invention. A multimeric structure comprises at least 5, preferably at least 10, more preferably at least 30, most preferably at least 60 modified viral capsid proteins comprising a first member of a specific binding pair as described herein. They can form regular viral capsids (empty viral particles) or viral particles (capsids encapsidating a nucleotide of interest). The formation of viral particles comprising a viral genome is a highly preferred feature for use of the modified viral capsids described herein.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

Methods of Use and Making

A further embodiment of the modified viral capsid proteins described herein is their use for delivering a nucleotide of interest, e.g., a reporter gene or a therapeutic gene, to a target cell. Generally, a nucleotide of interest may be a transfer plasmid, which may generally comprise 5′ and 3′ inverted terminal repeat (ITR) sequences flanking the reporter gene(s) or therapeutic gene(s) (which may be under the control of a viral or non-viral promoter, when encompassed within an AAV particle. In one embodiment, a nucleotide of interest is a transfer plasmid comprising from 5′ to 3′: a 5′ ITR, a promoter, a gene (e.g., a reporter and/or therapeutic gene) and a 3′ITR.

Non-limiting examples of useful promoters include, e.g., cytomegalovirus (CMV)-promoter, the spleen focus forming virus (SFFV)-promoter, the elongation factor 1 alpha (EF 1a)-promoter (the 1.2 kb EF1a-promoter or the 0.2 kb EF1a-promoter), the chimeric EF 1 a/IF4-promoter, and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, the CMV enhancer can be used in combination with the chicken β-actin promoter.

A variety of reporter genes (or detectable moieties) can be encapsidated in a multimeric structure comprising the modified viral capsid proteins described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting particles that employ the use of a reporter gene that encodes green fluorescent protein, however, persons of skill upon reading this disclosure will understand that the viral capsids described herein can be generated in the absence of a reporter gene or with any reporter gene known in the art.

A variety of therapeutic genes can also be encapsidated in a multimeric structure comprising the modified viral capsid proteins described herein, e.g., as part of a transfer particle. Non-limiting examples of a therapeutic gene include those that encode a toxin (e.g., a suicide gene), a therapeutic antibody or fragment thereof, a CRISPR/Cas system or portion(s) thereof, antisense RNA, siRNA, shRNA, etc.

A further embodiment of the present invention is a process for the preparation of a modified capsid protein, the method comprising the steps of:
a) expressing a nucleic acid encoding the modified capsid protein under suitable conditions, and
b) isolating the expressed capsid protein of step a).

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. A method for making such a mosaic viral particle comprises
a) expressing a nucleic acid encoding the modified capsid protein and a nucleotide encoding a reference capsid protein at a ratio (wt/wt) of at least about 60:1 to about 1:60, e.g., 2:1, 1:1, 3:5, 1:2, 1:3, etc. under suitable conditions, and
b) isolating the expressed capsid protein of step a).

In some embodiments, a composition described herein comprises, or a method described herein combines, a modified cap gene: reference cap gene (or combination of reference cap genes) at a ratio that ranges from at least about 1:60 to about 60:1, e.g., 2:1, 1:1, 3:5, 1:2, 1:3, etc. In some embodiments, the ratio is at least about 1:2. In some embodiments, the ratio is at least about 1:3. In some embodiments, the ratio is at least about 1:4. In some embodiments, the ratio is at least about 1:5. In some embodiments, the ratio is at least about 1:6. In some embodiments, the ratio is at least about 1:7. In some embodiments, the ratio is at least about 1:8. In some embodiments, the ratio is at least about 1:9. In some embodiments, the ratio is at least about 1:10. In some embodiments, the ratio is at least about 1:11. In some embodiments, the ratio is at least about 1:12. In some embodiments, the ratio is at least about 1:13. In some embodiments, the ratio is at least about 1:14. In some embodiments, the ratio is at least about 1:15. In some embodiments, the ratio is at least about 1:16. In some embodiments, the ratio is at least about 1:17. In some embodiments, the ratio is at least about 1:18. In some embodiments, the ratio is at least about 1:19. In some embodiments, the ratio is at least about 1:20. In some embodiments, the ratio is at least about 1:25. In some embodiments, the ratio is at least about 1:30. In some embodiments, the ratio is at least about 1:35. In some embodiments, the ratio is at least about 1:40. In some embodiments, the ratio is at least about 1:45. In some embodiments, the ratio is at least about 1:50. In some embodiments, the ratio is at least about 1:55. In some embodiments, the ratio is at least about 1:60. In some embodiments, the ratio is at least about 2:1. In some embodiments, the ratio is at least about 3:1. In some embodiments, the ratio is at least about 4:1. In some embodiments, the ratio is at least about 5:1. In some embodiments, the ratio is at least about 6:1. In some embodiments, the ratio is at least about 7:1. In some embodiments, the ratio is at least about 8:1. In some embodiments, the ratio is at least about 9:1. In some embodiments, the ratio is at least about 10:1. In some embodiments, the ratio is at least about 11:1. In some embodiments, the ratio is at least about 12:1. In some embodiments, the ratio is at least about 13:1. In some embodiments, the ratio is at least about 14:1. In some embodiments, the ratio is at least about 15:1. In some embodiments, the ratio is at least about 16:1. In some embodiments, the ratio is at least about 17:1. In some embodiments, the ratio is at least about 18:1. In some embodiments, the ratio is at least about 19:1. In some embodiments, the ratio is at least about 20:1. In some embodiments, the ratio is at least about 25:1. In some embodiments, the ratio is at least about 30:1. In some embodiments, the ratio is at least about 35:1. In some embodiments, the ratio is at least about 40:1. In some embodiments, the ratio is at least about 45:1. In some embodiments, the ratio is at least about 50:1. In some embodiments, the ratio is at least about 55:1. In some embodiments, the ratio is at least about 60:1.

In some embodiments, VP protein subunit ratios in the mosaic viral particle may, but do not necessarily, stoichiometrically reflect the ratios of modified cap gene:reference cap gene. As a non-limiting exemplary embodiment, a mosaic capsid formed according to the method may be considered to, but does not necessarily, have a modified capsid protein:reference capsid protein ratio similar to the ratio (wt:wt) of nucleic acids encoding same used to produce the mosaic capsid. In some embodiments, a mosaic capsid comprises a protein subunit ratio of about 1:59 to about 59:1.

Further embodiments of the present invention is a method for altering the tropism of a virus, the method comprising the steps of: (a) inserting a nucleic acid encoding an amino acid sequence into a nucleic acid sequence encoding an viral capsid protein to form a nucleotide sequence encoding a genetically modified capsid protein comprising the amino acid sequence and/or (b) culturing a packaging cell in conditions sufficient for the production of viral particles, wherein the packaging cell comprises the nucleic acid. A further embodiment of the present invention is a method for displaying a targeting ligand on the surface of a capsid protein, the method comprising the steps of: (a) expressing a nucleic acid encoding a modified viral capsid protein as described herein (and optionally with a nucleotide encoding a reference capsid protein) under suitable conditions, wherein the nucleic acid encodes a capsid protein comprising a first member of a specific binding pair, (b) isolating the expressed capsid protein comprising a first member of a specific binding pair of step (a) or capsid comprising same, and (c) incubating the capsid protein or capsid with a second cognate member of the specific binding pair under conditions suitable for allowing the formation of an isopeptide bond between the first and second member, wherein the second cognate member of the specific binding pair is fused with a targeting ligand.

In some embodiments, the packaging cell further comprises a helper plasmid and/or a transfer plasmid comprising a nucleotide of interest. In some embodiments, the methods further comprise isolating self-complementary adeno-associated viral particles from culture supernatant. In some embodiments, the methods further comprise lysing the packaging cell and isolating single-stranded adeno-associated viral particles from the cell lysate. In some embodiments, the methods further comprise (a) clearing cell debris, (b) treating the supernatant containing viral particles with nucleases, e.g., DNase I and MgCl₂, (c) concentrating viral particles, (d) purifying the viral particles, and (e) any combination of (a)-(d).

Packaging cells useful for production of the viral particles described herein include, e.g., animal cells permissive for the virus, or cells modified to be permissive for the virus; or the packaging cell construct, for example, with the use of a transformation agent such as calcium phosphate. Non-limiting examples of packaging cell lines useful for producing viral particles described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raji (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J Virol 69, 7430-7436, NSO (murine myeloma) cells, human amniocytic cells (e.g., CAP, CAP-T), yeast cells (including, but not limited to, S. cerevisiae, Pichia pastoris), plant cells (including, but not limited to, Tobacco NT1, BY-2), insect cells (including but not limited to SF9, S2, SF21, Tni (e.g. High 5)) or bacterial cells (including, but not limited to, E. coli).

For additional packaging cells and systems, packaging techniques and particles for packaging the nucleic acid genome into the pseudotyped viral particle see, for example, Polo, et al, Proc Natl Acad Sci USA, (1999) 96:4598-4603. Methods of packaging include using packaging cells that permanently express the viral components, or by transiently transfecting cells with plasmids.

Further embodiments include methods of redirecting a virus and/or delivering a reporter or therapeutic gene to a target cell, the method comprising a method for transducing cells in vitro (e.g., ex vivo) or in vivo, the method comprising the steps of: contacting the target cell with a viral particle comprising a capsid described herein, wherein the capsid comprises a targeting ligand that specifically binds a receptor expressed by the target cell. In some embodiments, the target cell is in vitro (e.g., ex vivo). In other embodiments, the target cell is in vivo in a subject, e.g., a human.

Target Cells

A wide variety of cells may be targeted in order to deliver a nucleotide of interest using a modified viral particle as disclosed herein. The target cells will generally be chosen based upon the nucleotide of interest and the desired effect.

In some embodiments, a nucleotide of interest may be delivered to enable a target cell to produce a protein that makes up for a deficiency in an organism, such as an enzymatic deficiency, or immune deficiency, such as X-linked severe combined immunodeficiency. Thus, in some embodiments, cells that would normally produce the protein in the animal are targeted. In other embodiments, cells in the area in which a protein would be most beneficial are targeted.

In other embodiments, a nucleotide of interest, such as a gene encoding an siRNA, may inhibit expression of a particular gene in a target cell. The nucleotide of interest may, for example, inhibit expression of a gene involved in a pathogen life cycle. Thus, cells susceptible to infection from the pathogen or infected with the pathogen may be targeted. In other embodiments, a nucleotide of interest may inhibit expression of a gene that is responsible for production of a toxin in a target cell.

In other embodiments a nucleotide of interest may encode a toxic protein that kills cells in which it is expressed. In this case, tumor cells or other unwanted cells may be targeted.

In still other embodiments a nucleotide of interest that encodes a therapeutic protein.

Once a particular population of target cells is identified in which expression of a nucleotide of interest is desired, a target receptor is selected that is specifically expressed on that population of target cells. The target receptor may be expressed exclusively on that population of cells or to a greater extent on that population of cells than on other populations of cells. The more specific the expression, the more specifically delivery can be directed to the target cells. Depending on the context, the desired amount of specificity of the marker (and thus of the gene delivery) may vary. For example, for introduction of a toxic gene, a high specificity is most preferred to avoid killing non-targeted cells. For expression of a protein for harvest, or expression of a secreted product where a global impact is desired, less marker specificity may be needed.

As discussed above, the target receptor may be any receptor for which a targeting ligand can be identified or created. Preferably the target receptor is a peptide or polypeptide, such as a receptor. However, in other embodiments the target receptor may be a carbohydrate or other molecule that can be recognized by a binding partner. If a binding partner, e.g., ligand, for the target receptor is already known, it may be used as the affinity molecule. However, if a binding molecule is not known, antibodies to the target receptor may be generated using standard procedures. The antibodies can then be used as a targeting ligand.

Thus, target cells may be chosen based on a variety of factors, including, for example, (1) the application (e.g., therapy, expression of a protein to be collected, and conferring disease resistance) and (2) expression of a marker with the desired amount of specificity.

Target cells are not limited in any way and include both germline cells and cell lines and somatic cells and cell lines. Target cells can be stem cells derived from either origin. When the target cells are germline cells, the target cells are preferably selected from the group consisting of single-cell embryos and embryonic stem cells (ES).

Pharmaceutical Compositions, Dosage Forms and Administration

A further embodiment provides a medicament comprising at least one modified viral capsid protein and appropriate targeting ligand according to this invention and/or a nucleic acid according to this invention. Preferably such medicament is useful as a gene transfer particle.

Also disclosed herein are pharmaceutical compositions comprising the viral particles described herein and a pharmaceutically acceptable carrier and/or excipient. In addition, disclosed herein are pharmaceutical dosage forms comprising the viral particle described herein.

As discussed herein, the viral particles described herein can be used for various therapeutic applications (in vivo and ex vivo) and as research tools.

Pharmaceutical compositions based on the viral particles disclosed herein can be formulated in any conventional manner using one or more physiologically acceptable carriers and/or excipients. The viral particles may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the viral particles described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered viral particles described herein on a daily or weekly basis for a time period or on a monthly, bi-yearly or yearly basis depending on need or exposure to a pathogenic organism or to a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the viral particles and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal or anti-bacterial agents or anti-tumor agents may be needed for compositions described.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Any method known to one skilled in the art maybe used for large scale production of viral particles, packaging cells and particle constructs described herein. For example, master and working seed stocks may be prepared under GMP conditions in qualified primary CEFs or by other methods. Packaging cells may be plated on large surface area flasks, grown to near confluence and viral particles purified. Cells may be harvested and viral particles released into the culture media isolated and purified, or intracellular viral particles released by mechanical disruption (cell debris can be removed by large-pore depth filtration and host cell DNA digested with endonuclease). Virus particles may be subsequently purified and concentrated by tangential-flow filtration, followed by diafiltration. The resulting concentrated bulk maybe formulated by dilution with a buffer containing stabilizers, filled into vials, and lyophilized. Compositions and formulations may be stored for later use. For use, lyophilized viral particles may be reconstituted by addition of diluent.

Certain additional agents used in the combination therapies can be formulated and administered by any means known in the art.

Compositions as disclosed herein can also include adjuvants such as aluminum salts and other mineral adjuvants, tensoactive agents, bacterial derivatives, vehicles and cytokines. Adjuvants can also have antagonizing immunomodulating properties. For example, adjuvants can stimulate Th1 or Th2 immunity. Compositions and methods as disclosed herein can also include adjuvant therapy.

Non-limiting and exemplary embodiments are listed below.
Embodiment 1. An AAV viral particle comprising an AAV capsid,
   wherein at least one AAV capsid protein of said AAV capsid comprises at least a portion of an amino acid sequence of a capsid protein selected from the group consisting of
      a capsid protein of a non-primate animal AAV,
      a capsid protein of a remote AAV, and
      a combination thereof, and
   wherein at least one AAV capsid protein of said AAV capsid is modified to comprise
      (a) at least a first member of a protein:protein binding pair,
      (b) a detectable label,
      (c) a point mutation,
      (d) a chimeric amino acid sequence comprising a portion of an amino acid sequence of an other AAV capsid protein that is operably linked to said amino acid sequence of the capsid protein selected from the group consisting of the capsid protein of the non-primate animal AAV, the capsid protein of the remote AAV, or the combination thereof, and (e) any combination of (a), (b), (c), and (d).
Embodiment 2. An AAV viral particle comprising an AAV capsid,
wherein at least one AAV capsid protein of said AAV capsid comprises at least a portion of an amino acid sequence of a capsid protein of a non-primate animal AAV,
wherein at least one AAV capsid protein of said AAV capsid is modified to comprise
(a) at least a first member of a protein:protein binding pair,
(b) a detectable label,
(c) a point mutation,
(d) a chimeric amino acid sequence comprising a portion of an amino acid sequence of an other AAV capsid protein that is operably linked to said amino acid sequence of the capsid of the non-primate animal AAV, and
(e) any combination of (a), (b), (c), and (d).
Embodiment 3. An AAV viral particle comprising an AAV capsid,
wherein at least one AAV capsid protein of said AAV capsid comprises at least a portion of an amino acid sequence of a capsid protein of a remote AAV,
wherein at least one AAV capsid protein of said AAV capsid is modified to comprise
(a) at least a first member of a protein:protein binding pair,
(b) a detectable label,
(c) a point mutation,
(d) a chimeric amino acid sequence comprising a portion of an amino acid sequence of an other AAV capsid protein that is operably linked to said amino acid sequence of the capsid protein of the remote AAV, and
(e) any combination of (a), (b), (c), and (d).
Embodiment 4. An AAV viral particle comprising
(A) at least one AAV capsid protein comprising an amino acid sequence selected from the group consisting of
(i) an amino acid sequence of a capsid protein of a non-primate animal AAV,
(ii) an amino acid sequence of a capsid protein of a remote AAV, and
(iii) an amino acid sequence of a combination thereof, and
(B) an AAV genome comprising a nucleotide of interest and an AAV ITR comprising at least a portion of an ITR sequence of an other AAV.
Embodiment 5. An AAV viral particle comprising
(A) at least one AAV capsid protein comprising an amino acid sequence of a capsid protein of a non-primate animal AAV, and
(B) an AAV genome comprising a nucleotide of interest and an AAV ITR comprising at least a portion of an ITR sequence of an other AAV.
Embodiment 6. An AAV viral particle comprising
(A) at least one AAV capsid protein comprising an amino acid sequence of a capsid protein of a remote AAV, and
(B) an AAV genome comprising a nucleotide of interest and an AAV ITR comprising at least a portion of an ITR sequence of an other AAV.
Embodiment 7. The AAV viral particle of any of the preceding embodiments,
wherein said amino acid sequence of the capsid protein of the non-primate animal AAV, the capsid protein of the remote AAV, or the combination thereof, is modified to comprise
(a) at least a first member of a protein:protein binding pair,
(b) a detectable label,
(c) a point mutation.
Embodiment 8. The AAV particle of embodiment 7, wherein the protein:protein binding pair is selected from SpyTag:SpyCatcher, SpyTag:KTag, Isopeptag:pilin-C, SnoopTag:SnoopCatcher, and SpyTag002:SpyCatcher002.
Embodiment 9. The AAV particle of embodiment 7, wherein the first member of a protein:protein binding pair comprises c-myc comprising a sequence set forth as SEQ ID NO:44.
Embodiment 10. The AAV particle of any one of the preceding embodiments wherein the detectable label comprises the B1 epitope comprising an amino acid sequence of IGTRYLTR (SEQ ID NO: 45).
Embodiment 11. The AAV viral particle of any of the preceding embodiments, wherein said amino acid sequence of the capsid protein the non-primate animal AAV, the capsid protein of the remote AAV, or the combination thereof, comprises an amino acid sequence of a VP3 capsid protein of the non-primate animal AAV and/or an amino acid sequence of a VP3 capsid protein of the remote AAV.
Embodiment 12. The AAV viral particle of any of the preceding embodiments, wherein said amino acid sequence of the capsid protein the capsid protein of the non-primate animal AAV, the capsid protein of the remote AAV, or the combination thereof, comprises an amino acid sequence of a VP2 capsid protein of the non-primate animal AAV and/or an amino acid sequence of a VP2 capsid protein of the remote AAV.
Embodiment 13. The AAV viral particle of any of the preceding embodiments, wherein said amino acid sequence of the capsid protein of the capsid protein of the non-primate animal AAV the capsid protein of the remote AAV, or the combination thereof, comprises an amino acid sequence of a VP1 capsid protein of the non-primate animal AAV and/or an amino acid sequence of a VP1 capsid protein of the remote AAV.
Embodiment 14. The AAV viral particle of any of the preceding embodiments, wherein the capsid of said particle comprises
(i) a VP1 capsid protein that is either
   a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV, or
a VP1 capsid protein of the non-primate AAV or the remote AAV,
(ii) a VP2 capsid protein that is either a chimeric AAV VP2 capsid protein, optionally wherein the chimeric VP2 capsid protein comprises a VP1/VP2 common region of an other AAV operably linked to a VP3 region of the non-primate AAV or the remote AAV, or a VP2 capsid protein of the non-primate AAV or the remote AAV, and
(iii) the VP3 capsid protein of the non-primate AAV or the remote AAV.
Embodiment 15. The AAV viral particle of any of the preceding embodiments, wherein the capsid of said particle comprises
(i) a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV,
(ii) a chimeric AAV VP2 capsid protein, optionally wherein the chimeric VP2 capsid protein comprises a VP1/VP2 common region of an other AAV operably linked to a VP3 region of the non-primate AAV or the remote AAV, and
(iii) the VP3 capsid protein of the non-primate AAV or the remote AAV.
Embodiment 16. The AAV viral particle of any of the preceding embodiments, wherein the capsid of said particle comprises
(i) a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV,
(ii) a VP2 capsid protein of the non-primate AAV or the remote AAV, and
(iii) the VP3 capsid protein of the non-primate AAV or the remote AAV.
Embodiment 17. The AAV viral particle of any of the preceding embodiments, wherein the capsid comprises
(i) a VP1 capsid protein of the non-primate AAV or the remote AAV,
(ii) a VP2 capsid protein of the non-primate AAV or the remote AAV, and
(iii) a VP3 capsid protein of the non-primate AAV or the remote AAV.
Embodiment 18. The AAV viral particle of any of the preceding embodiments, wherein said other AAV is a primate AAV or a combination of primate AAV.
Embodiment 19. The AAV viral particle of any of the preceding embodiments, wherein said other AAV is a selected from the group consisting of AAV1, AAV2, AAV3, AAV4, AAV5, AAV6, AAV7, AAV8, AAV9, and a combination thereof.
Embodiment 20. The AAV viral particle of any of the preceding embodiments, wherein said other AAV is AAV2.
Embodiment 21. The AAV viral particle of any of any one of embodiments 1-2, 4-5, and 7-20, wherein said non-primate animal AAV is a non-primate AAV listed in Table 2.
Embodiment 22. The AAV viral particle of any of any one of embodiments 1-2, 4-5, and 7-21, wherein the non-primate AAV is an avian AAV, a sea lion AAV or a bearded dragon AAV.
Embodiment 23. The AAV viral particle of any of any one of embodiments 1-2, 4-5, and 7-22, wherein the non-primate animal AAV is an AAAV.
Embodiment 24. The AAV viral particle of any of any one of embodiments 1-2, 4-5, and 7-23, wherein the modification is at position I444 or I580 of a VP1 capsid protein of AAAV.
Embodiment 25. The AAV viral particle of any of any one of embodiments 1-2, 4-5, and 7-22, wherein the non-primate animal AAV is a squamate AAV.
Embodiment 26. The AAV viral particle of any of any one of embodiments 1-2, 4-5, 7-22, and 25, wherein the squamate AAV is a bearded dragon AAV.
Embodiment 27. The AAV viral particle of any of any one of embodiments 1-2, 4-5, 7-22, and 25-26, wherein the modification is at position I573 or I436 of a VP1 capsid protein of a bearded dragon AAV.
Embodiment 28. The AAV viral particle of any of any one of embodiments 1-2, 4-5, and 7-22, wherein the non-primate animal AAV is a mammalian AAV.
Embodiment 29. The AAV viral particle of any of any one of embodiments 1-2, 4-5, 7-22, and 28, wherein the mammalian AAV is a sea lion AAV.
Embodiment 30. The AAV viral particle of any of any one of embodiments 1-2, 4-5, 7-22, and 28-29, wherein modification is at position selected from the group consisting of I429, I430, I431, I432, I433, I434, I436, I437, and A565 of a VP1 of a sea lion AAV.
Embodiment 31. The AAV particle of any of the preceding embodiments, comprising a VP3 capsid protein of the non-primate animal AAV, the remote AAV, or the combination thereof, wherein the VP3 capsid protein is modified to comprise
(a) at least a first member of a protein:protein binding pair, optionally wherein the protein:protein binding pair is selected from the group consisting of SpyTag:SpyCatcher, SpyTag:KTag, Isopeptag:pilin-C, SnoopTag:SnoopCatcher, and SpyTag002:SpyCatcher002
(b) a detectable label, optionally wherein the detectable label comprises the amino acid sequence set forth as SEQ ID NO: 44 or the amino acid sequence set forth as SEQ ID NO:45,
(c) a point mutation, or
(d) any combination of (a), (b), and (c).
Embodiment 32. The AAV particle of embodiment 31, wherein the VP3 capsid protein of the non-primate animal AAV, the remote AAV, or the combination thereof is modified to comprise
   (a) at least SpyTag comprising an amino acid sequence set forth as SEQ ID NO:42 and/or
   (b) a detectable label comprising amino acid sequence set forth SEQ ID NO:45.
Embodiment 33. The AAV particle of any of the preceding embodiments, comprising a first and/or second linker operably linking a first member of a protein:protein binding pair and/or a detectable label to a capsid protein of the capsid of said AAV particle.
Embodiment 34. The AAV particle of embodiment 33, wherein the first and second linker are not identical.
Embodiment 35. The AAV particle of embodiment 33 or embodiment 34, wherein the first and second linker are identical.
Embodiment 36. The AAV particle of any one of embodiments 33-35, wherein the first and/or second linkers is 10 amino acids in length.
Embodiment 37. The AAV particle of any of the preceding embodiments, comprising a first member of a protein:protein binding pair and/or a detectable label operably linked to a variable region of a capsid protein of the capsid of said AAV particle.
Embodiment 38. The AAV particle of embodiment 1 or embodiment 2, comprising a capsid protein comprising an amino acid sequence selected from the group consisting of
(a) an amino acid sequence set forth as SEQ ID NO:2,
(b) an amino acid sequence set forth as SEQ ID NO:4,
(c) an amino acid sequence set forth as SEQ ID NO:6,
(d) an amino acid sequence set forth as SEQ ID NO:8,
(e) an amino acid sequence set forth as SEQ ID NO:10,
(f) an amino acid sequence set forth as SEQ ID NO:12,
(g) an amino acid sequence set forth as SEQ ID NO:14,
(h) an amino acid sequence set forth as SEQ ID NO:16,
(i) an amino acid sequence set forth as SEQ ID NO:18,
(j) an amino acid sequence set forth as SEQ ID NO:20,
(k) an amino acid sequence set forth as SEQ ID NO:22,
(l) an amino acid sequence set forth as SEQ ID NO:24,
(m) an amino acid sequence set forth as SEQ ID NO:26,
(n) an amino acid sequence set forth as SEQ ID NO:28,
(o) an amino acid sequence set forth as SEQ ID NO:30,
(p) an amino acid sequence set forth as SEQ ID NO:32,
(q) an amino acid sequence set forth as SEQ ID NO:34,
(r) an amino acid sequence set forth as SEQ ID NO:36,
(s) the amino acid sequence set forth as SEQ ID NO:53,
(t) the amino acid sequence set forth as SEQ ID NO:55,
(u) the amino acid sequence set forth as SEQ ID NO:57,
(v) the amino acid sequence set forth as SEQ ID NO:59,
(w) the amino acid sequence set forth as SEQ ID NO:61,
(x) the amino acid sequence set forth as SEQ ID NO:63,
(y) the amino acid sequence set forth as SEQ ID NO:65,
(z) the amino acid sequence set forth as SEQ ID NO:67,
(aa) the amino acid sequence set forth as SEQ ID NO:69,
(bb) the amino acid sequence set forth as SEQ ID NO:71,
(cc) an amino acid sequence having significant sequence identity, e.g., at least 95% identity, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71, and
(dd) an amino acid sequence of any VP2 and/or VP3 portions of the amino acid sequences set forth in any of (a)-(cc).
Embodiment 39. The AAV particle of any one of the preceding embodiments, further comprising a reference capsid protein such that the capsid is a mosaic capsid.
Embodiment 40. The AAV particle of any one of the preceding embodiments, comprising a mosaic capsid that comprises the VP3 capsid protein modified with a first member of a protein:protein binding pair and a reference VP3 capsid protein.
Embodiment 41. An adeno-associated virus (AAV) capsid protein comprising an amino acid sequence of a capsid protein of a non-primate animal AAV or a remote AAV, wherein the AAV capsid protein is selected from the group consisting of
   (a) a chimeric AAV VP1 capsid protein, optionally wherein the chimeric animal AAV VP1 capsid protein modified to comprise at least a first member of a protein:protein binding pair, a detectable label, and/or a point mutation,
   (b) a non-chimeric AAV VP1 capsid protein modified to comprise at least a first member of a protein:protein binding pair and/or a detectable label,
   (c) a chimeric VP2 capsid protein, optionally wherein the chimeric AAV VP2 capsid protein is modified to comprise at least a first member of a protein:protein binding pair, a detectable label, and/or a point mutation,
   (d) a non-chimeric AAV VP2 capsid protein modified to comprise at least a first member of a protein:protein binding pair, a detectable label, and/or a point mutation,
   (e) a chimeric AAV VP3 capsid protein modified to comprise at least a first member of a protein:protein binding pair, a detectable label, and/or a point mutation, and
   (f) a non-chimeric AAV VP3 capsid protein modified to comprise at least a first member of a protein:protein binding pair, a detectable label, and/or a point mutation.
Embodiment 42. The AAV capsid protein of embodiment 41, wherein the first member of a protein:protein binding pair is flanked by a first and/or second linker that link(s) the a first member of a protein:protein binding pair to the capsid protein, and wherein the first and/or second linker is each independently at least one amino acid in length.
Embodiment 43. The AAV capsid protein of embodiment 42, wherein the first and second linkers are not identical.
Embodiment 44. The AAV capsid protein of embodiment 42, wherein the first and second linkers are identical and are 10 amino acids in length.
Embodiment 45. The AAV capsid protein of any one of embodiments 41-44, wherein the capsid protein further comprises a second cognate member of the protein:protein binding pair, optionally wherein the first and second members are bound by a covalent bond, optionally an isopeptide bond.
Embodiment 46. The AAV capsid protein of any one of embodiments 41-45, wherein the first member of a protein:protein binding pair comprises SpyTag.
Embodiment 47. The AAV capsid protein of embodiment 45 or embodiment 46, wherein the second cognate member comprises SpyCatcher.
Embodiment 48. The AAV capsid protein of any one of embodiments 45-47, wherein the second cognate member comprises KTag.
Embodiment 49. The AAV capsid protein of embodiment 45, wherein the first member is KTag and the second cognate member comprises SpyTag.
Embodiment 50. The AAV capsid protein of embodiment 45, wherein the first member is SnoopTag and the second cognate member comprises SnoopCatcher.
Embodiment 51. The AAV capsid protein of embodiment 45, wherein the first member is isopeptag and the second cognate member comprises Pilin-C.
Embodiment 52. The AAV capsid protein of embodiment 45, wherein the first member is SpyTag002 and the second cognate member comprises SpyCatcher002.
Embodiment 53. The AAV capsid protein of any one of embodiments 45-52, wherein the second member is operably linked to a targeting ligand, optionally wherein the targeting ligand is a binding moiety.
Embodiment 54. The AAV capsid protein of embodiment 53, wherein the binding moiety is an antibody, or a portion thereof.
Embodiment 55. The AAV capsid protein of embodiment 54, wherein the antibody, or portion thereof, is fused to SpyCatcher.
Embodiment 56. The AAV capsid protein of embodiment 54 or embodiment 55, wherein the antibody, or portion thereof, is fused to a linker at the C-terminus, and the linker is fused to SpyCatcher at the linker's C-terminus.
Embodiment 57. The AAV capsid protein of embodiment 56, wherein the linker comprises a sequence set forth as SEQ ID NO:49 (GSGESG).
Embodiment 58. The AAV capsid protein of any one of embodiments 41-57, wherein the detectable label comprises a B1 epitope comprising an amino acid sequence set forth as SEQ ID NO:45.
Embodiment 59. The AAV capsid protein of any one of embodiments 41-58, wherein said non-primate animal AAV is a non-primate AAV listed in Table 2.
Embodiment 60. The AAV capsid protein of any one of embodiments 41-59, wherein the non-primate AAV is an avian AAV, a sea lion AAV or a bearded dragon AAV.
Embodiment 61. The AAV capsid protein of any one of embodiments 41-60, wherein the non-primate animal AAV is an AAAV.
Embodiment 62. The AAV capsid protein of any one of embodiments 41-61, wherein the modification is at position I444 or I580 of a VP1 capsid protein of AAAV.
Embodiment 63. The AAV capsid protein of any one of embodiments 41-60, wherein the non-primate animal AAV is a squamate AAV.
Embodiment 64. The AAV capsid protein of any one of embodiments 41-60 and 63, wherein the squamate AAV is a bearded dragon AAV.
Embodiment 65. The AAV capsid protein of any one of embodiments 41-60, and 63-64, wherein the modification is at position I573 or I436 of a VP1 capsid protein of a bearded dragon AAV.
Embodiment 66. The AAV capsid protein of any one of embodiments 41-60, wherein the non-primate animal AAV is a mammalian AAV.
Embodiment 67. The AAV capsid protein of any one of embodiments 41-60 and 66, wherein the mammalian AAV is a sea lion AAV.
Embodiment 68. The AAV capsid protein of any one of embodiments 41-60 and 66-67, wherein modification is at position selected from the group consisting of I429, I430, I431, I432, I433, I434, I436, I437, and A565 of a VP1 of a sea lion AAV.
Embodiment 69. The AAV capsid protein of any one of embodiments 41-68, comprising an amino acid sequence selected from the group consisting of
(a) an amino acid sequence set forth as SEQ ID NO:2,
(b) an amino acid sequence set forth as SEQ ID NO:4,
(c) an amino acid sequence set forth as SEQ ID NO:6,
(d) an amino acid sequence set forth as SEQ ID NO:8,
(e) an amino acid sequence set forth as SEQ ID NO:10,
(f) an amino acid sequence set forth as SEQ ID NO:12,
(g) an amino acid sequence set forth as SEQ ID NO:14,
(h) an amino acid sequence set forth as SEQ ID NO:16,
(i) an amino acid sequence set forth as SEQ ID NO:18,
(j) an amino acid sequence set forth as SEQ ID NO:20,
(k) an amino acid sequence set forth as SEQ ID NO:22,
(l) an amino acid sequence set forth as SEQ ID NO:24,
(m) an amino acid sequence set forth as SEQ ID NO:26,
(n) an amino acid sequence set forth as SEQ ID NO:28,
(o) an amino acid sequence set forth as SEQ ID NO:30,
(p) an amino acid sequence set forth as SEQ ID NO:32,
(q) an amino acid sequence set forth as SEQ ID NO:34,
(r) an amino acid sequence set forth as SEQ ID NO:36,
(s) the amino acid sequence set forth as SEQ ID NO:53,
(t) the amino acid sequence set forth as SEQ ID NO:55,
(u) the amino acid sequence set forth as SEQ ID NO:57,
(v) the amino acid sequence set forth as SEQ ID NO:59,
(w) the amino acid sequence set forth as SEQ ID NO:61,
(x) the amino acid sequence set forth as SEQ ID NO:63,
(y) the amino acid sequence set forth as SEQ ID NO:65,
(z) the amino acid sequence set forth as SEQ ID NO:67,
(aa) the amino acid sequence set forth as SEQ ID NO:69,
(bb) the amino acid sequence set forth as SEQ ID NO:71,
(cc) an amino acid sequence having significant sequence identity, e.g., at least 95% identity, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71, and
(dd) an amino acid sequence of any VP2 and/or VP3 portions of the amino acid sequences set forth in any of (a)-(cc).
Embodiment 70. The AAV capsid protein of any one of 41-69, wherein the first member of a protein:protein binding pair comprises a detectable label.
Embodiment 71. The AAV capsid protein of embodiment 70, wherein the detectable label comprises c-myc (SEQ ID NO:44).
Embodiment 72. An AAV particle comprising the AAV capsid protein of any one of embodiments 41-71.
Embodiment 73. A nucleic acid comprising a cap gene encoding the AAV capsid protein of any one of embodiments 41-71.
Embodiment 74. A nucleic acid molecule comprising an AAV cap gene that encodes an AAV capsid protein,
wherein the AAV cap gene comprises at least a portion of the nucleotide sequence of a cap gene selected from the group consisting of
(i) a cap gene of a non-primate animal AAV,
(ii) a cap gene of a remote AAV, or
(iii) a combination thereof,
wherein said AAV cap gene is further modified to comprise
(a) a nucleotide sequence encoding a first member of a protein:protein binding pair,
(b) a nucleotide sequence encoding a detectable label,
(c) a point mutation,
(d) a chimeric nucleotide sequence comprising a portion of a nucleotide sequence of an other AAV cap gene that is operably linked to said nucleotide sequence of the AAV cap gene selected from the group consisting of the cap gene non-primate animal AAV, a remote AAV, or a combination thereof,
(e) any combination of (a), (b), (c), and (d).
Embodiment 75. A nucleic acid molecule comprising an AAV cap gene that encodes an AAV capsid protein,
wherein the AAV cap gene comprises at least a portion of the nucleotide sequence of a cap gene of a non-primate animal AAV
wherein said AAV cap gene is further modified to comprise
(a) a nucleotide sequence encoding a first member of a protein:protein binding pair,
(b) a nucleotide sequence encoding a detectable label,
(c) a point mutation,
(d) a chimeric nucleotide sequence comprising a portion of a nucleotide sequence of an other AAV cap gene that is operably linked to said nucleotide sequence of the cap gene non-primate animal AAV,
(e) any combination of (a), (b), (c), and (d).
Embodiment 76. A nucleic acid molecule comprising an AAV cap gene that encodes an AAV capsid protein,
wherein the AAV cap gene comprises at least a portion of the nucleotide sequence of a cap gene of a remote animal AAV
wherein said AAV cap gene is further modified to comprise
(a) a nucleotide sequence encoding a first member of a protein:protein binding pair,
(b) a nucleotide sequence encoding a detectable label,
(c) a point mutation,
(d) a chimeric nucleotide sequence comprising a portion of a nucleotide sequence of an other AAV cap gene that is operably linked to said nucleotide sequence of the cap gene remote animal AAV,
(e) any combination of (a), (b), (c), and (d).
Embodiment 77. A nucleic acid molecule comprising an AAV rep gene and an AAV cap gene,
wherein the AAV cap gene comprises a first nucleotide sequence of a cap gene selected from the group consisting of
(i) a cap gene of a non-primate animal AAV,
(ii) a cap gene of a remote AAV, and
(iii) a combination thereof,
wherein the AAV rep gene comprises a second nucleotide sequence of an AAV rep gene of an other AAV.
Embodiment 78. A nucleic acid molecule comprising an AAV rep gene and an AAV cap gene,
wherein the AAV cap gene comprises a first nucleotide sequence of a cap gene of a non-primate animal AAV,
wherein the AAV rep gene comprises a second nucleotide sequence of an AAV rep gene of an other AAV.
Embodiment 79. A nucleic acid molecule comprising an AAV rep gene and an AAV cap gene,
wherein the AAV cap gene comprises a first nucleotide sequence of a cap gene of a remote animal AAV,
wherein the AAV rep gene comprises a second nucleotide sequence of an AAV rep gene of an other AAV.
Embodiment 80. The nucleic acid molecule of any one of embodiments 73-79, wherein the cap gene is operably linked to a promoter.
Embodiment 81. The nucleic acid molecule of embodiment 80, wherein said promoter directs the expression of the capsid protein(s) in a packaging cell.
Embodiment 82. The nucleic acid molecule of embodiment 80 or embodiment 81, wherein the promoter is selected from p40, SV40, EF, CMV, B19p6, and CAG.
Embodiment 83. The nucleic acid molecule of any one of embodiments 73-82, wherein said nucleotide sequence of cap gene of the non-primate animal AAV, the cap gene of the remote AAV, or the combination thereof, is modified to comprise
(a) a nucleotide sequence encoding at least a first member of a protein:protein binding pair,
(b) a nucleotide sequence encoding a detectable label, and/or
(c) a nucleotide sequence encoding a point mutation.
Embodiment 84. The nucleic acid molecule of embodiment 83, wherein the protein:protein binding pair is selected from SpyTag:SpyCatcher, SpyTag:KTag, Isopeptag:pilin-C, SnoopTag:SnoopCatcher, and SpyTag002:SpyCatcher002.
Embodiment 85. The nucleic acid molecule of embodiment 83, wherein the first member of a protein:protein binding pair comprises c-myc comprising a sequence set forth as SEQ ID NO:44.
Embodiment 86. The nucleic acid molecule of any one of embodiments 83-85, wherein the detectable label comprises the B1 epitope comprising an amino acid sequence of IGTRYLTR (SEQ ID NO: 45).
Embodiment 87. The nucleic acid molecule of any one of embodiments 73-86 wherein said nucleotide sequence of the cap gene the non-primate animal AAV, the cap gene of the remote AAV, or the combination thereof, comprises a nucleotide sequence encoding an amino acid sequence of a VP3 capsid protein of the non-primate animal AAV and/or an amino acid sequence of a VP3 capsid protein of the remote AAV.
Embodiment 88. The nucleic acid molecule of any one of embodiments 73-87, wherein said nucleotide sequence of the cap gene the non-primate animal AAV, the cap gene of the remote AAV, or the combination thereof, comprises a nucleotide sequence encoding an amino acid sequence of a VP2 capsid protein of the non-primate animal AAV and/or an amino acid sequence of a VP2 capsid protein of the remote AAV.
Embodiment 89. The nucleic acid molecule of any one of embodiments 73-87, wherein said nucleotide sequence of the cap gene the non-primate animal AAV, the cap gene of the remote AAV, or the combination thereof, comprises a nucleotide sequence encoding an amino acid sequence of a VP1 capsid protein of the non-primate animal AAV and/or an amino acid sequence of a VP1 capsid protein of the remote AAV.
Embodiment 90. The nucleic acid molecule of any one of embodiments 73-89, wherein cap gene encodes
(i) a VP1 capsid protein that is either
   a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV, or
a VP1 capsid protein of the non-primate AAV or the remote AAV,
(ii) a VP2 capsid protein that is either
a chimeric AAV VP2 capsid protein, optionally wherein the chimeric VP2 capsid protein comprises a VP1/VP2 common region of an other AAV operably linked to a VP3 region of the non-primate AAV or the remote AAV, or
a VP2 capsid protein of the non-primate AAV or the remote AAV, and/or
(iii) the VP3 capsid protein of the non-primate AAV or the remote AAV.
Embodiment 91. The nucleic acid molecule of any one of embodiments 73-90, wherein cap gene encodes
(i) a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV,
(ii) a chimeric AAV VP2 capsid protein, optionally wherein the chimeric VP2 capsid protein comprises a VP1/VP2 common region of an other AAV operably linked to a VP3 region of the non-primate AAV or the remote AAV, and/or
(iii) the VP3 capsid protein of the non-primate AAV or the remote AAV.
Embodiment 92. The nucleic acid molecule of any one of embodiments 73-90. wherein cap gene encodes
(i) a chimeric AAV VP1 capsid protein, optionally wherein the chimeric VP1 capsid protein comprises a VP1-unique region (VP1-u) of an other AAV operably linked to a VP1/VP2 common region and a VP3 region of the non-primate AAV or the remote AAV,
(ii) a VP2 capsid protein of the non-primate AAV or the remote AAV, and
(iii) the VP3 capsid protein of the non-primate AAV or the remote AAV.
Embodiment 93. The nucleic acid molecule of any one of embodiments 73-90 wherein cap gene encodes
(i) a VP1 capsid protein of the non-primate AAV or the remote AAV,
(ii) a VP2 capsid protein of the non-primate AAV or the remote AAV, and/or
(iii) a VP3 capsid protein of the non-primate AAV or the remote AAV.
Embodiment 94. The nucleic acid molecule of any one of embodiments 73-93, wherein said other AAV is a primate AAV or a combination of primate AAV.
Embodiment 95 The nucleic acid molecule of any one of embodiments 73-94, wherein said other AAV is a selected from the group consisting of AAV1, AAV2, AAV3, AAV4, AAV5, AAV6, AAV7, AAV8, AAV9, and a combination thereof.
Embodiment 96. The nucleic acid molecule of any one of embodiments 73-95, wherein said other AAV is AAV2.
Embodiment 97. The nucleic acid molecule of any one of embodiments 73-75, 77-78, and 80-96, wherein said non-primate animal AAV is a non-primate AAV listed in Table 2.
Embodiment 98. The nucleic acid molecule of any one of embodiments 73-75, 77-78, and 80-97, wherein the non-primate AAV is an avian AAV, a sea lion AAV or a bearded dragon AAV.
Embodiment 99. The nucleic acid molecule of any one of embodiments 73-75, 77-78, and 80-98, wherein the non-primate animal AAV is an AAAV.
Embodiment 100. The nucleic acid molecule of any one of embodiments 73-75, 77-78, and 80-99, wherein the modification is at a codon encoding position I444 or I580 of a VP1 capsid protein of AAAV.
Embodiment 101. The nucleic acid molecule of any one of embodiments 73-75, 77-78, and 80-98, wherein the non-primate animal AAV is a squamate AAV.
Embodiment 102. The nucleic acid molecule of any one of embodiments 73-75, 77-78, 80-98, and 101, wherein the squamate AAV is a bearded dragon AAV.
Embodiment 103. The nucleic acid molecule of any one of embodiments 73-75, 76-77, and 80-98, and 101-102, wherein the modification is at a codon encoding position I573 or I436 of a VP1 capsid protein of a bearded dragon AAV.
Embodiment 104. The nucleic acid molecule of any one of embodiments 73-75, 76-77, and 80-98, wherein the non-primate animal AAV is a mammalian AAV.
Embodiment 105. The nucleic acid molecule of any one of embodiments 73-75, 77-78, 80-98, and 104 wherein the mammalian AAV is a sea lion AAV.
Embodiment 106. The nucleic acid molecule of any one of embodiments 73-75, 77-78, 80-98, and 105, wherein modification is at position selected from the group consisting of I429, I430, I431, I432, I433, I434, I436, I437, and A565 of a VP1 of a sea lion AAV.
Embodiment 107. The nucleic acid molecule of any one of embodiments 73-106, comprising a nucleotide sequence selected from the group consisting of
(a) the nucleotide sequence set forth as SEQ ID NO:1,
(b) the nucleotide sequence set forth as SEQ ID NO:3,
(c) the nucleotide sequence set forth as SEQ ID NO:5,
(d) the nucleotide sequence set forth as SEQ ID NO:7,
(e) the nucleotide sequence set forth as SEQ ID NO:9,
(f) the nucleotide sequence set forth as SEQ ID NO:11,
(g) the nucleotide sequence set forth as SEQ ID NO:13,
(h) the nucleotide sequence set forth as SEQ ID NO:15,
(i) the nucleotide sequence set forth as SEQ ID NO:17,
(j) the nucleotide sequence set forth as SEQ ID NO:19,
(k) the nucleotide sequence set forth as SEQ ID NO:21,
(l) the nucleotide sequence set forth as SEQ ID NO:23,
(m) the nucleotide sequence set forth as SEQ ID NO:25,
(n) the nucleotide sequence set forth as SEQ ID NO:27,
(o) the nucleotide sequence set forth as SEQ ID NO:29,
(p) the nucleotide sequence set forth as SEQ ID NO:31,
(q) the nucleotide sequence set forth as SEQ ID NO:33,
(r) the nucleotide sequence set forth as SEQ ID NO:35,
(s) the nucleotide sequence set forth as SEQ ID NO:52,
(t) the nucleotide sequence set forth as SEQ ID NO:54
(u) the nucleotide sequence set forth as SEQ ID NO:56
(v) the nucleotide sequence set forth as SEQ ID NO:58
(w) the nucleotide sequence set forth as SEQ ID NO:60
(x) the nucleotide sequence set forth as SEQ ID NO:62
(y) the nucleotide sequence set forth as SEQ ID NO:64
(z) the nucleotide sequence set forth as SEQ ID NO:66
(aa) the nucleotide sequence set forth as SEQ ID NO:68
(bb) the nucleotide sequence set forth as SEQ ID NO:70
(cc) a nucleotide sequence having significant sequence identity, e.g., at least 95% identity, to the nucleotide sequence set forth as SEQ ID NO: 1, 3, 5, 7, 9, 11, 13, 15, 17, 19, 21, 23, 25, 27, 29, 31, 33, 35, 52, 54, 56, 58, 60, 62, 64, 66, 68, 70, or any thereof encoding a VP2 capsid,
(dd) any portion of the nucleotide sequence of (a)-(s) encoding a VP2 capsid protein and/or a VP3 capsid protein.
Embodiment 108. The nucleic acid molecule of any one of embodiments 73-107, further comprising an AAV rep gene that encodes one or more AAV Rep proteins and is operably linked to a promoter.
Embodiment 109. The nucleic acid molecule of embodiment 108, wherein the promoter is selected from the group consisting of p5, p19 SV40, EF, CMV, B19p6, and CAG.
Embodiment 110. The nucleic acid molecule of embodiment 108 or embodiment 109, wherein the one or more Rep proteins are selected from Rep78, Rep68, Rep52 and Rep40.
Embodiment 111. The nucleic acid molecule of any one of embodiments 108-110, wherein the one or more Rep proteins comprises Rep78.
Embodiment 112. The nucleic acid molecule of any one of embodiments 108-110, wherein the one or more Rep proteins are primate animal AAV Rep proteins.
Embodiment 113. An AAV capsid protein comprising an amino acid sequence of any one of the nucleic acid molecules of embodiments 73-112.
Embodiment 114. An AAV particle comprising the capsid protein of embodiment 113.
Embodiment 115. A packaging cell for producing AAV particles comprising the nucleic acid molecule that comprises a cap gene according to any one of embodiments 73-112.
Embodiment 116. The packaging cell of embodiment 115, further comprising a nucleic acid molecule comprising a rep gene encoding one or more AAV Rep proteins, wherein said rep gene is operably linked to a promoter, optionally wherein the rep gene and the cap gene are of two different AAV.
Embodiment 117. The packaging cell of embodiment 116, wherein the promoter operably linked to the rep gene directs the expression of the Rep protein(s) in the packaging cell.
Embodiment 118. The packaging cell of embodiment 116 or embodiment 117, wherein the promoter is selected from p5, p19 SV40, EF, CMV, B19p6, and CAG.
Embodiment 119. The packaging cell of any one of embodiments 116-118, wherein the one or more Rep proteins are selected from Rep78, Rep68, Rep52 and Rep40.
Embodiment 120. The packaging cell of any one of embodiments 116-119, wherein the one or more Rep proteins comprises Rep78.
Embodiment 121. The packaging cell of any one of embodiments 116-120, wherein the one or more Rep proteins are primate animal AAV Rep proteins.
Embodiment 122. The packaging cell of any one of embodiments 116-120, wherein the one or more Rep proteins are non-primate animal AAV Rep proteins.
Embodiment 123. The packaging cell of any one of embodiments 116-122, further comprising a nucleic acid molecule comprising a nucleotide sequence of a nucleotide of interest flanked on at least one side by at least one AAV inverted terminal repeat (ITR) that is recognized by the one or more Rep proteins.
Embodiment 124. The packaging cell of embodiment 123, wherein the nucleotide is flanked on the other side by a second ITR of the same AAV as the at least one ITR.
Embodiment 125. The packaging cell of embodiment 124, wherein the nucleotide is flanked on the other side by a second ITR, wherein the second ITR and the at least one ITR are of different AAV.
Embodiment 126. The packaging cell of any one of embodiments 123-125, wherein the nucleotide of interest is a reporter gene.
Embodiment 127. The packaging cell of embodiment 126, wherein the reporter gene encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.
Embodiment 128. The packaging cell of any one of embodiments 123-125, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.
Embodiment 129. The packaging cell of any one of embodiments 115-133, further comprising a nucleotide sequence encoding a reference capsid protein.
Embodiment 130. A method of producing a viral particle comprising culturing a packaging cell according to any one of embodiments 115-129 in conditions sufficient for the production of viral particles.
Embodiment 131. The method of embodiment 130, wherein the packaging cell further comprises a helper plasmid and/or a transfer plasmid comprising a nucleotide of interest.
Embodiment 132. The method of embodiment 130 or embodiment 131, further comprising one or more of the following steps:
a. clearing cell debris,
b. treating the supernatant containing viral particles with Benzonase or DNase I and MgCl2,
c. concentrating viral particles,
d. purifying the viral particles, and
e. any combination of a.-d,
optionally wherein the viral particles are self-complementary adeno-associated viral particles and/or isolated from culture supernatant.
Embodiment 133. An AAV particle made according to the method of any one of embodiments 130-132.
Embodiment 134. An AAV particle according to any one of embodiments 1-40, 72, 114, and 133, further comprising a nucleotide of interest.
Embodiment 135. The AAV particle of embodiment 134, wherein the nucleotide of interest is a reporter gene.
Embodiment 136. The AAV particle of embodiment 135, wherein the reporter gene encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.
Embodiment 137. The AAV particle of embodiment 134, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.
Embodiment 138. A pharmaceutical composition comprising (a) an AAV particle according to any one of embodiments 1-40, 72, 114, and 133, an AAV particle comprising an AAV capsid protein according to any one of embodiments 41-71 and 113, or an AAV particle made according to the method of any one of embodiments 130-132, and (b) a pharmaceutically acceptable carrier or excipient.
Embodiment 139. A method of delivering a nucleotide of interest to a target cell comprising contacting the target cell with (a) the AAV particle according to any one of embodiments 1-40, 72, 114, and 133, or (b) the composition of embodiment 138.
Embodiment 140. The method of embodiment 139, wherein the capsid of the AAV particle comprises a targeting ligand that specifically binds a protein expressed on the surface the target cell.
Embodiment 141. The method of embodiment 139 or embodiment 140, wherein the contacting is performed ex vivo.
Embodiment 142. The method of embodiment 139 or embodiment 140, wherein the target cell is in a subject.
Embodiment 143. The method of embodiment 142, wherein the subject is a human.
Embodiment 144. The method of any one of embodiments 139-143, wherein the target cell is a human cell.
Embodiment 145. The method of any one of embodiments 139-144, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

TABLE 2

| | | | | |
|---|---|---|---|---|
| | | | Region | |
| | | | encoding the | |
| | | | non- | Region |
| | | | structural/rep | encoding the |
| | | | protein | capsid protein |
| | | | (Accession | (Accession |
| | Accession | | Number/ | Number/ |
| | Number/ | | GI Number of | GI Number of |
| Description | GI Number | ITR | protein) | protein) |

Avian adeno-associated virus

| | | | | |
|---|---|---|---|---|
| Avian Adeno-associated | GQ368252. 1/ | | 243-2237 | 2255-4471 |
| virus isolate YZ-1, | GI: | | (ACU30841.1/ | (ACU30842.1/ |
| complete genome | 255110032 | | GI:25571003) | GI:255710034) |
| Avian adeno-assoeialed | KF937791.1/ | 1 -142 | 244-2235 | 2253-44690 |
| virus isolate ZNI, | GI: | 4540-4682 | (AHK22792.1/ | (AHK22793.1/ |
| complete genome | 588284633 | | GI:588284634 | GI:588284635 |

Avian adeno-associated virus ATCC VR-865

| | | | | |
|---|---|---|---|---|
| Avian adeno-associated | AY186198.1/ | 1-142 | 244-2232 | 2250-4481 |
| virus ATCC VR-865 | GI:3141477 | 4552-469 | (AAO32086.1/ | (AA032087.1/ |
| complete genome | | | GI:31414778 | GI:31414779 |
| Avian adeno-associated | NC...004828.1 | 1-142 | 244-2232 | 2250-4481 |
| virus ATCC VR-865 | GI:31543922 | 4552-4694 | (NP...852780.1/ | (NP...852781.1/ |
| complete genome | | | GI:48996103) | GI:31543994) |
| Avian adeno-associated | AY629581.1/ | 1-142 | 244-2235 | 2253-4469 |
| virus ATCC VR-865 | GI:48996102 | 4552-4694 | (AAT48612.1/ | (ATT48613.1/ |
| complete genome | | | GI:48996103) | GI:48996104) |

Avianadeno-associated virus strain DA-1

| | | | | |
|---|---|---|---|---|
| Avian adeno-associated | AY629583.1/ | 1-142 | 244-2235 | 2253-4469 |
| virus strain DA-1 | GI: 48996105 | 4540-4682 | (ATT48614.1/ | (AAT48615.1/ |
| complete genome | | | GI:51949969 | GI:48996107) |
| Avian adeno-associated | NC_006263.1 | 1-142 | 244-2235 | 2253-4469 |
| virus strain DA-1 | /GI: | 4540-4682 | (YP_077182.1/ | (YP_077183.1/ |
| complete genome | 51949968 | | GI:51949969) | GI:51949970 |

Bat Adeno-associated virus YNM

| | | | |
|---|---|---|---|
| Bat adeno-associaded | GU226971.1/ | 200-2041 | 2059-4233 |
| virus YNM, complete | /GI:30442291 | ADD17085.1/ | (ADD17086.1/ |
| sequence | 8 | GI:289719009 | GI:289719010 |
| Bat adeno-associaded | NC...014468.1 | 200-2041 | 2059-4233 |
| virus YNM, complete | /GI:30442291 | (YP_00385857 | (YP_00385857 |
| genome | 6 | 1.1/GI: | 2.1/GI: |
| | | 304422917 | 304422918 |

Bovine adeno-associated virus

| | | | |
|---|---|---|---|
| Bovine adeno- | AY388617.1/ | 368-2200 | 2216-4426 |
| associated virus | GI:38679253 | (AAR26464.1/ | AAR26465.1/ |
| compleed genome | | GI:38679254 | GI:38679255 |
| Bovine adeno- | NC_005889.1 | 368-2200 | 2216-4426 |
| associated virus, | / | (YP_024970.1/ | YP_024971.1/ |
| completed genome | GI:48696557 | GI:48696558 | GI:48696559 |

Bovine parvovirus-2

| | | | |
|---|---|---|---|
| Bovine parvovirus-2 | AF406966.1/ | 306-1919 | 2268-5384 |
| putative non-structural | | (AAL0967.1/ | (AAL09672.1/ |
| protein and putative | | GI:15825371) | GI:15825372) |
| capsid protien genes, | | | |
| complete cds. | | | |
| Bovine parvovirus-2 | NC_006259.1 | 306-1919 | 2268-5384 |
| complete genome | /GI:51949957 | (YP_077175.1/ | (YP_077176.1/ |
| | | GI:51949958) | GI:51949959) |

California sea lion adeno-associated virus 1

| | | | |
|---|---|---|---|
| California sea lion | JN420371.1 | 306-1919 | 2268-5384 |
| adeno-associated virus | GI:34345896 | (AEM37641.1/ | (AEM37642.1/ |
| 1 isolate 1136 rep 78 | 6 | (GI:343458967 | GI:343458968 |
| and VPI genes, | | | |
| complete cds, | | | |
| California sea lion | JN420372.1/ | 291-2093 | 2124-4280 |
| adeno-associated virus | GI:34345896 | (AEM37643.1/ | (AEM37644.1/ |
| 1 isolate 1187 rep 78 | 9 | GI:343458970) | GI:343458971) |
| and VPI genes, | | | |
| complete cds, | | | |

Goose parvovirus

| | | | | |
|---|---|---|---|---|
| Goose parvovirus | (U25749.1/GI | 1-444 | 537-2420 | 2439-4637 |
| virulent B strain | :1113795 | | (AAA83229.1/ | (AAA83230.1/ |
| Complete genome | | 4663-5106 | GI:1113796) | GI:1113797) |
| | | | | 2874-4637 |
| | | | | (AAA83231.1/ |
| | | | | GI:1113798) |
| | | | | 3033-4637 |
| | | | | (AAA83232.1/ |
| | | | | GI:1113799) |
| Goose parvovirus | (NC_001701 | 1-444 | 537-2420 | 2439-4637 |
| Complete genome | 1/GI:9628649 | | (NP_043514.1/ | (NP_043515.1/ |
| | ) | 4663-5106 | GI:9628650) | GI:9628651) |
| | | | | 2874-4637 |
| | | | | (NP_043516.1/ |
| | | | | GI:9628652) |
| Goose parvovirus strain | (EU583389.1/ | 1-381 | 474-2357 | 2376-4574 |
| 82-0321V, complete | GI:19088818 | | (ACE95848.1/ | (ACE95849.1/ |
| genome | 8) | 4600-4980 | GI:190888189) | GI:190888190) |
| Goose parvovirus strain | (EU583390.1/ | 1-416 | 509-2395 | 2411-4609 |
| 82-0321,complete | GI:19088819 | | (ACE95850.1/ | (ACE95851.1/ |
| genome | 1) | 4635-5050 | GI:190888192) | GI:190888192) |
| Goose parvovirus strain | (EU583391.1/ | 1-418 | 511-2394 | 2413-4611 |
| 06-0329, complete | GI:19088819 | | (ACE598521.1 | ACE95853.1/ |
| geonme | 4) | 4637-5054 | GI:190888195) | GI:190888196) |
| Goose Parvovirus strain | (EU583392.1/ | 1-443 | 536-2419 | 2438-4636 |
| VG32/1, complete | GI:19088819 | | (ACE95854.1/ | (ACE95855.1/ |
| genome | 7) | 4662-5104 | GI:190888198 | GI:190888199) |
| Goose parvovirus strain | (JF333590.1/ | 1-444 | 537-2420 | 2439-4637 |
| SH, complete genome | GI:34311361 | | (AEL87777.1/ | (AEL87778.1/ |
| | 8) | 4663-5106 | GI:343113619) | GI:343113620) |
| | | | | 2874-4637 |
| | | | | (AEL87779.1/ |
| | | | | GI:343113621) |
| | | | | 3033-4637 |
| | | | | (AEL87780.1/ |
| | | | | GI:343113622 |
| Goose parvovirus strain | (HQ891825.1/ | 1-444 | 537-2420 | 2439-4637 |
| GDaGPV, complete | GI:35984330 | 4663-5106 | (AEV89789.1/ | (AEV89790.1/ |
| genome | 7) | | GI:359843308) | GI:359843309) |
| | | | | 2874-4637 |
| | | | | (AEV89791.1/ |
| | | | | 3033-4637 |
| | | | | (AEV89792.1 |
| | | | | GI:3598-43311) |
| Goose parvovirus strain | (KC478066.1/ | 1-416 | 509-2392 | 2411-4609 |
| SHFX1201, complete | GI:45936051 | | (AGG56527.1/ | (AGG56528.1/ |
| genome | 4) | | GI:459360515 | GI:459360516) |
| Goose parvovirus strain | (KC178571.1/ | 1-444 | 537-2420 | 2439-4637 |
| Y, complete genome | GI:51312976 | | (AGO17637.1/ | (AG017638.1/ |
| | 1) | | | |
| Goose parvovirus strain | (KC184133.1/ | 1-443 | 536-2419 | 2438-4636 |
| E, complete genome | GI:51312976 | | (AG017639.1/ | (AGO17640.1/ |
| | 4) | | GI:513129765) | GI:51312766) |
| Goose parvovirus strain | (KC996729.1/ | 1-442 | 535-2418 | 2437-4635 |
| SYG61v, complete | GI:53199721 | | (AGT62580.1 | (AGT62581.1/ |
| | 7) | | GI:531997218) | GI:531997219) |
| Goose parvovirus strain | (KC996730.1/ | 1-414 | 507-2390 | 2409-4607 |
| YZ99-6 complete | GI:53199722 | | (ACT62582.1 | (AGT30253.1/ |
| | 6) | | GI:531997221 | GI:531997222) |
| Goose parvovirus | (AF416726.1/ | | 1-1884 | 1903-4101 |
| nonstructural protein | GI:17226299 | | (AAL37721.1/ | (AAL37722.1/ |
| NS (ns) and capsid | | | GI:17226300) | GI:17226301) |
| protein VP (vp) genes | | | 529-1884 | |
| complete cds. | | | (ABP93843.1/ | |
| | | | GI:14569447) | |
| Goose parvovirus strain | (EF515837.1/ | | 1-884 | 1903-4101 |
| DY NS1 (NS1), NS2 | GI:14569444 | | (ABP93842.1/ | (ABP82770.1/ |
| (NS2, VP1 (vp1), | 5) | | GI:145694446 | GI:145573169) |
| (VP3) genes, complete | | | | 2338-4101 |
| cds. | | | | (ABP93844.1/G |
| | | | | I:145694448 |
| | | | | 2497-4101 |
| | | | | (ABP938441.1/G |
| | | | | I:145694448) |
| | | | | 2497-4101 |
| | | | | (ABP93845.1/ |
| | | | | GI:145694449) |
| Goose parvovirus strain | (JF926695.1/ | | 9-1892 | 1911-4109 |
| PT NS1 protien and | GI:35446316 | | (AER25356.1/ | (AER25357.1/ |
| VP1 protien genes | 2) | | GI:354463163 | GI:354463164 |
| complete cds. | | | | |
| Goose parvovirus strain | (JF926696.1/ | | 6-1889 | 1908-4106 |
| D NS1 protein and VP1 | GI:35446316 | | (AER25358.1/ | (AER25359.1/ |
| protein genes, complete | 5) | | GI:354463166 | GI:354463167) |
| cds. | | | | |

Mouse adeno-associated virus 1

| | | | |
|---|---|---|---|
| Mouse adeno- | DQ100362.1/ | 1-327 | 344-2485 |
| associated virus 1 rep | GI:73665994 | (AAZ79671.1/ | (AAZ79672.1/ |
| gene, partial cds; and | | GI:73665995) | GI73665996) |
| VP1 capsid, VP2 | | | 731-2485 |
| capsid, and VP3 capsid | | | (AAZ79673.1/ |
| genes, complete cds. | | | GI:73665997) |
| | | | 911-2485 |
| | | | (AAZ79674.1/ |
| | | | GI:73665998) |

Muscovy duck parvovirus

| | | | | |
|---|---|---|---|---|
| Barbarie duck | U22967.1/GI: | 1-457 | 548-2431 | 2450-4648 |
| parvovirus REP protien | 1113784 | | (AAA83224.1/ | (AAA83225.1/ |
| | | 4678-5132 | GI:1113785) | GI:1113786) |
| | | | | 2885-4648 |
| | | | | (AAA83226.1/ |
| | | | | GI:1113787) |
| | | | | 3044-4648 |
| | | | | (AAA83227.1/ |
| | | | | GI:1113788) |
| Muscovy duck | NC_006147.2/ | 1-457 | 548-2431 | 2450-4648 |
| parvovirus, complete | GI:51593841 | 4678-5132 | (YP_068410.1/ | (YP_068411.1/ |
| genome | | | GI:51593842) | GI:51593843) |
| | | | | 2885-4648 |
| | | | | (YP_068412.1/ |
| | | | | GI:51593844) |
| | | | | (YP_068413.1/ |
| | | | | GI:51593845) |
| Muscovy duck | JF926697.1/ | | 61-1944 | 1963-4161 |
| parvovirus strain P REP | GI:35446316 | | (AER25360.1/ | (AER25361.1/ |
| protien and CP1 protien | 8 | | GI:354463169) | GI:354463170) |
| Genes, complete cds. | | | | |
| Muscovy duck | JF926698.1/ | | 61-1944 | 1963-4161 |
| parvovirus strain P1 | GI:35446317 | | (AER25362.1/ | (AER25363.1/ |
| REP protein and VP1 | 1 | | GI:354463172) | GI:354463173) |
| protien genes, complete | | | | |
| cds. | | | | |
| Muscovy duck | KC171936.1/ | 1-381 | 512-2395 | 2414-4612 |
| parvovirus isolate | GI:45925686 | | (AGG53766.1/ | (AGG53768.1/ |
| SAAS-SHNH, | 7 | | GI:459256868) | GI:459256870) |
| complete genome | | | | 2849-4612 |
| | | | | (AGG53769.1/ |
| | | | | GI:459256871) |
| | | | | (AGG53767.1/ |
| | | | | GI:459256869) |

Sepentine adeno-associaed virus 2

| | | | |
|---|---|---|---|
| Sepentine adeno- | EU87249.1/ | 1-642 | 661-1297 |
| associaed virus 2 non- | GI:21540198 | (ACJ66590.1/ | (ACJ66591.1/ |
| structural protein 1 and | 1 | GI:215401982 | GI:215401983 |
| capsid protein genes, | | | |
| partial cds | | | |

Snake parvovirus 1

| | | | |
|---|---|---|---|
| Snake parvovirus 1 | AY349010.1/ | 324-2012 | 2030-4210 |
| non-structural protein 1 | GI:38017148 | (AAR07954.1/ | (AAR07955.1/ |
| (NS1) and capsid | | GI:38017149 | GI:38017150) |
| protein (VP1) genes, | | | |
| complete cds | | | |
| Snake parvovirus 1 | NC_006148.1/ | 324-2012 | 2030-4210 |
| complete genome | GI:51555744 | (YP_068093.1/ | (YP_068094.1/ |
| | | GI:51555745) | GI:51555746) |

### EXAMPLES

The following examples are provided for illustrative purposes only and are not intended to limit the scope of the invention.

Materials and Methods

Cell Lines and Antibodies

All 293 and 293T cell lines were maintained in DMEM supplemented with 10% FBS, 1% Pen/Strep, and 1% L-glutamine. 293 hErbB2 and 293hASGR1/2 cell lines were generated by lentiviral transduction of the parental 293 cell line with a particle expressing the corresponding cDNA. All cell lines were obtained from the Regeneron TC core facility. The B1 antibody recognizes a linear epitope shared by AAV VP1, VP2 and VP3.

AAV Capsid Protein Constructs

GeneBlocks encoding the desired AAV capsid sequences or primers for polymerase chain reaction amplification of desired AAV capsid sequences, including SpyTag insertions, flanking linker amino acids, and additional point mutations were purchased from IDT and cloned into pAAV R2C2 using Gibson Assembly according to the manufacturer's protocol (NEB).

Fusion of SpyCatcher to Antibodies

GeneBlocks encoding SpyCatcher were purchased from IDT, and Gibson Assembly was used to clone the coding sequence in-frame into expression plasmids for antibody heavy chains at the C terminus of each construct, separated by a flexible amino acid linker GSGESG (SEQ ID NO:49).

Preparation of AAV Viral Particles

Virus was generated by transfecting 293T packaging cells using PEI Pro or PEI Max with the following plasmids: pAd Helper, an AAV2 ITR-containing genome plasmid encoding a reporter protein, and a pAAV-CAP plasmid encoding AAV Rep and Cap genes, either with or without additional plasmids encoding either an scFv or the heavy and light chains of an antibody. The scFv and antibody heavy chain constructs are all fused to SpyCatcher at their C terminus as described above. Transfection was performed in OptiMEM, and media was changed to DMEM supplemented with 10% FBS, 1% Pen/Strep, and 1% L-Glut after 8 hours. An alternate transfection protocol was performed in 150 mM NaCl without exchanging media after transfection.

Transfected packaging cells were incubated for 3 days at 37° C., then virus was collected from cell lysates using a standard freeze-thaw protocol. In brief, packaging cells were lifted by scraping and pelleted. Supernatant was removed, and cells were resuspended in a solution of 50 mM Tris-HCl; 150 mM NaCl; and 2 mM MgCl₂ [pH 8.0] or 25 mM Tris-HCl; 100 mM NaCl; 1 mM MgCl₂; 2.5 mM KCl; 0.001% Pluronic F68 [pH 7.4]. Intracellular virus particles were released by inducing cell lysis via three consecutive freeze-thaw cycles, consisting of shuttling cell suspension between dry ice/ethanol bath and 37° C. water bath with vigorous vortexing. Viscosity was reduced by treating lysate with EMD Millipore Benzonase (50 U/ml of cell lysate) for 60 min at 37° C., with occasional mixing. Debris was then pelleted by centrifugation, and the resulting supernatant was processed for crude lysate preparation or for further iodixanol-gradient purification. For crude lysate preparation, the supernatant was filtered through a 0.22 μm PVDF Millex-GV Filter, directly into the upper chamber of an Amicon Ultra-15 Centrifugal Filter Unit with Ultracel-100 membrane (100 KDa MWCO) filter cartridge. The filter unit was centrifuged at 5-10 minute intervals until desired volume was reached in the upper chamber, then concentrated crude virus was pipetted into a low-protein-binding tube and stored at 4° C. For iodixanol-gradient purification, the lysate was filtered through a 0.2 μm PES Nalgene Rapid-Flow filter. AAV containing medium was separately concentrated by tangential flow filtration and diafiltration with 1×PBS supplemented with 0.001% Pluronic F-68. Clarified lysates or concentrated medium were loaded onto a discontinuous iodixanol gradient and centrifuged at 29,600 rpm for 16 to 18 hours at 10° C. using a SW 32 Ti rotor. Virus fractions were removed from the interface between the 40 and 60 percent iodixanol solutions and exchanged into 1×PBS supplemented with 0.001% Pluronic F-68 using Amicon Ultra Centrifugal filters with 100 KDa nominal molecular weight limit. Titer (vector genomes per milliliter vg/mL) was determined by qPCR using a standard curve of a virus of known concentration.

Cell Infection/Transduction and Flow Cytometric/Luminescence Analysis

To infect cells, viral particles were added directly to the media of cells in culture, and the mixture was incubated overnight at 37° C. For transductions with crude lysate, the media in each well was replaced 24 hours later, and cells were incubated for 3-5 days. For assessment of GFP expression, on day 3, 4 or 5 post-infection, cells were trypsinized, resuspended in PBS with 2% FBS, and the percentage of GFP+ cells was collected on a BD FACSCanto flow cytometer and analyzed using FlowJo software. For assessment of NanoLuc expression, on day 2 or 3 post-infection, cells were lysed in 1× Passive Lysis Buffer (Promega) and incubated with Nano-Glo Luciferase Assay Reagent. Luminescence was assessed using a SpectraMax plate reader.

Neutralization Assay in the Presence of IgG

Viral particles were mixed with increasing concentrations of purified human IgG prepared in PBS, and the mixture of viral particles and IgG was incubated at 37° C. for 30 minutes to allow binding. To infect cells, viral particles were added directly to the media of cells in culture, and the mixture was incubated at 37° C. for 2 days. On day 2 post-infection, Nanoluc expression was measured using a Nanoglo luciferase assay (Promega) and RLU data collected on a plate reader (PerkinElmer).

Western Blot Analysis

The reaction between SpyTagged AAV proteins VP1, VP2 and VP3 and SpyCatcher-tagged antibodies or scFvs was monitored by Western blot analysis. Novex® Tris-Glycine SDS Sample Buffer with Reducing Agent was added to equal volumes of crude virus preparations, and samples were heated to 85° C. for 5 minutes, then cooled to room temperature and loaded onto a pre-cast 4-12% Tris-Glycine gel (Invitrogen). Proteins were separated by reducing SDS-PAGE and blotted onto PVDF via a wet transfer. Membranes were blocked with 5% milk and probed with the mouse monocolonal B1 antibody (ARP American Research Products, Inc.) diluted 1:100 in TBST overnight at 4° C. Blots were washed in TBST, probed with an anti-mouse HRP-conjugated antibody, and detected using chemiluminescent detection reagents on a Bio-Rad ChemiDoc MP imager.

Protein Stain Analysis

The relative expression of AAV proteins VP1, VP2, and VP3 was monitored by protein stain analysis of SDS-PAGE gels. AAV samples were prepared using NuPAGE LDS sample buffer and Reducing agent (Invitrogen) according to manufacturer's instructions. Samples were heated to 95° C. for 10 minutes, then cooled to room temperature and loaded onto a precast 4-12% NuPAGE Bis-Tris gel (Invitrogen). After protein separation, gels were fixed in 50% methanol; 7% acetic acid, stained in SYPRO Ruby gel stain (Invitrogen), and washed in 10% methanol; 7% acetic acid. Gel images were captured on a Bio-Rad ChemiDoc MP imager.

### Example 1. Non-Primate AAV Chimeric Particles can be Produced and Purified Via Affinity Chromatography

Each virus was generated as described above by transfecting one 15 cm plate of 293T packaging cells with the following plasmids and quantities:

| | | |
|---|---|---|
| pAd Helper | 8 | μg |
| pAAV-UbC-Firefly Luciferase | 4 | μg |
| pRep Cap plasmid construct | 4 | μg |

Rep Cap plasmid constructs include:
pRep2 Cap AAV2 VP1 AAAV VP2 VP3
pRep2 Cap AAV2 VP1 Sea Lion VP2 VP3
pRep2 Cap AAV2 VP1 Bearded Dragon VP2 VP3

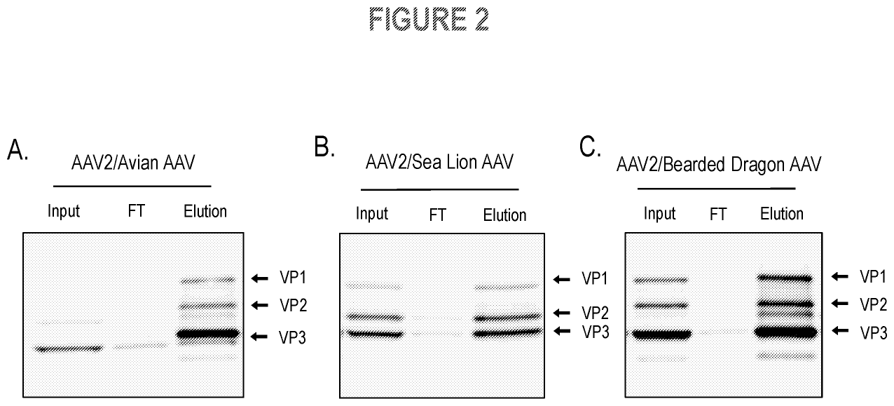

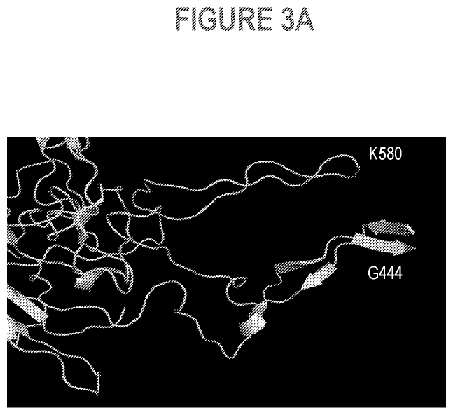

The crude virus preparations were then purified via affinity chromatography and the capsid proteins present in the input, flow-through (FT) and elution fractions were evaluated by Western Blotting (FIG. 2). Capsid proteins from all three viruses were present in the chromatography column input, reduced in the flow-through, and present in the elution fraction, suggesting that the affinity column chromatography approach used to purify primate-derived capsids such as AAV2 can also be utilized for the purification of non-primate AAVs.

In each of the following examples, the pRep2 Cap AAV2 VP1 AAAV VP2 VP3 plasmid comprising the rep gene of AAV2 and a chimeric cap gene encoding a chimeric VP1 capsid protein comprising the VP1-u region of AAV2 operably linked with the VP1/VP2 common region of the AAAV, an AAAV VP2 capsid protein, and an AAAV VP3 capsid protein is denoted by “pAAV R2Cap AAV2/AAAV.” The pRep2 Cap AAV2 VP1 Sea Lion VP2 VP3 plasmid comprising the rep gene of AAV2 and a chimeric cap gene encoding a chimeric VP1 capsid protein comprising the VP1-u region of AAV2 operably linked with the VP1/VP2 common region of the sea lion AAV, the sea lion VP2 capsid protein, and the sea lion VP3 capsid protein is denoted by “pAAV R2Cap AAV2/Sea Lion.” The pRep2 Cap AAV2 VP1 Bearded Dragon VP2 VP3 plasmid comprising the rep gene of AAV2 and a chimeric cap gene encoding a chimeric VP1 capsid protein comprising the VP1-u region of AAV2 operably linked with the VP1/VP2 common region of the Bearded Dragon AAV, the Bearded Dragon VP2 capsid protein, and the Bearded Dragon VP3 capsid protein is denoted by “pAAV R2Cap AAV2/Bearded Dragon.”

### Example 2. Peptide Insertions into the VP3 Region of Avian AAV Capsids are Well-Tolerated and can Mediate Covalent Attachment of Antibodies to Viral Particles Via SpyCatcher-SpyTag

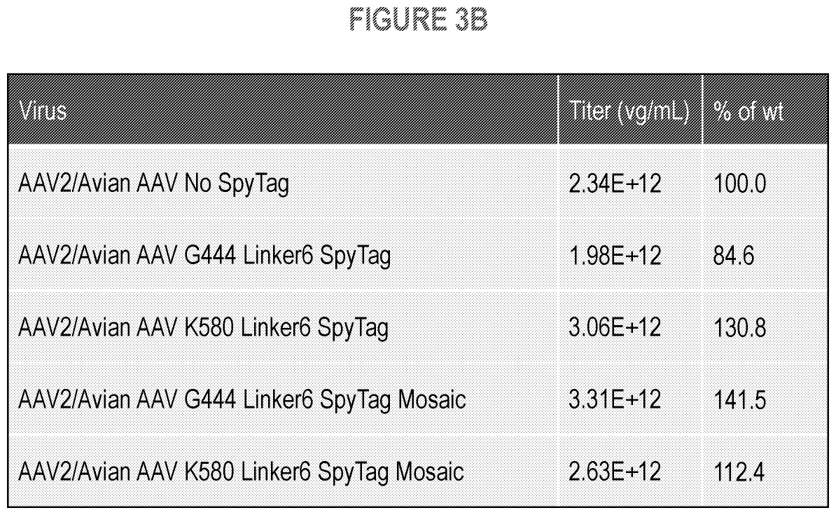

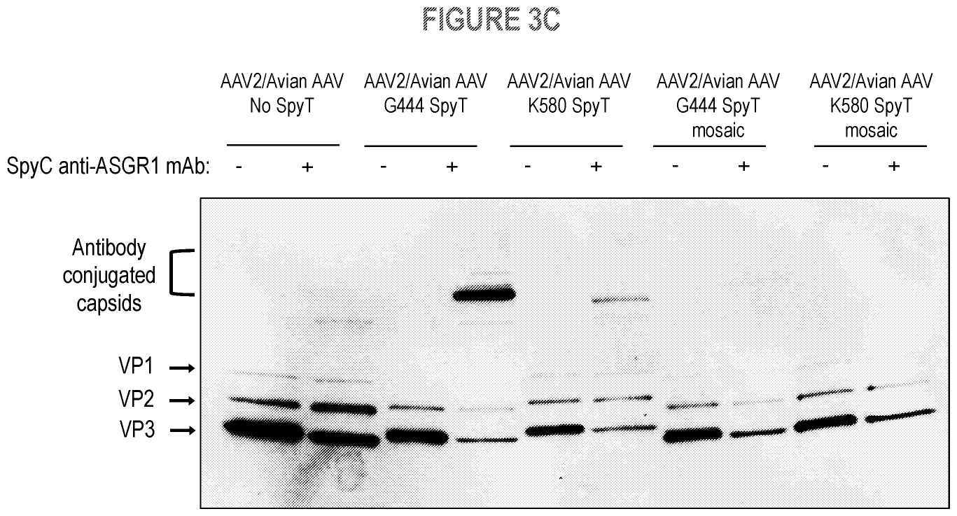

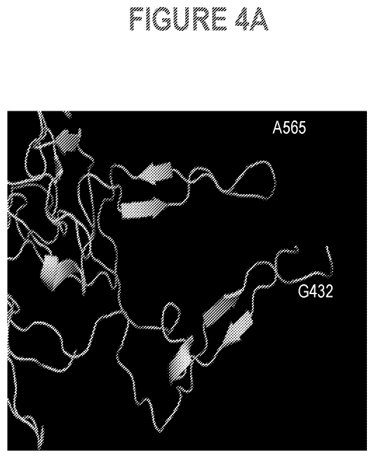

Potential peptide insertion sites within putative variable loop IV and variable loop VIII of Avian AAV were predicted using PyMol modeling (FIG. 3A) and the corresponding SpyTag insertion constructs were cloned. Each virus was generated as described above by transfecting one 15 cm plate of 293T packaging cells with the following plasmids and quantities:

| | | |
|---|---|---|
| pAd Helper | 16 | μg |
| pAAV-CAG-GFP | 8 | μg |
| pAAV R2CapX | 8 | μg |

pAAV R2CapX constructs include:
pAAV R2Cap AAV2/AAAV No SpyTag
pAAV R2Cap AAV2/AAAV G444 Linker6 SpyTag
pAAV R2Cap AAV2/AAAV K580 Linker6 SpyTag

or

| | | |
|---|---|---|
| pAd Helper | 16 | μg |
| pAAV-CAG-GFP | 8 | μg |
| pAAV R2Cap AAV2/AAAV No SpyTag | 6.7 | μg |
| WITH | | μg |
| pAAV R2Cap AAV2/AAAV G444 Linker6 SpyTag | 1.3 | μg |
| OR | | μg |
| pAAV R2Cap AAV2/AAAV K580 Linker6 SpyTag | 1.3 | μg |

Chimeric AAV2/AAAV particles lacking SpyTag insertions as well as chimeric AAV2/AAAV particles bearing SpyTag insertions at various positions within the capsid as listed above were packaged with ITR2-containing AAV genomes. In an effort to understand whether chimeric AAV2/AAAV capsids can be successfully formed and packaged with genomes containing AAV2 ITRs, quantitative PCR was performed to measure the titer (number of genomes per milliliter or vg/mL) of chimeric AAV2/AAAV particles bearing SpyTag insertions relative to chimeric AAV2/AAAV particles lacking SpyTag insertions (FIG. 3B). The measured titers demonstrate that chimeric AAV2/AAAV particles can be packaged with AAV2 ITR genomes. The titer of chimeric AAV2/AAAV particles was similar between chimeric AAV2/AAAV particles lacking SpyTag insertions and chimeric AAV2/AAAV particles bearing SpyTag insertions.

Next, chimeric AAV2/AAAV particles lacking SpyTag insertions and chimeric AAV2/AAAV particles bearing SpyTag insertions with and without a SpyCatcher-tagged antibody that binds to ASGR1. The reaction between SpyTagged chimeric AAV2/AAAV proteins VP1, VP2 and VP3 and SpyCatcher-tagged anti-ASGR1 heavy chains was monitored by Western blotting; SpyTagged capsid proteins that have reacted with SpyCatcher-tagged antibody exhibit an increase in size by SDS-PAGE. In the presence of SpyCatcher-tagged anti-ASGR1 mAb, SpyTagged chimeric AAV2/AAAV capsid proteins displayed an increase in apparent size by Western blotting compared to the SpyTagged AAAV capsid proteins alone (FIG. 3C). This indicated that SpyTagged chimeric AAV2/AAAV particles were able to successfully form a covalent bond with SpyCatcher-tagged anti-ASGR1 mAbs.

### Example 3. Peptide Insertions into the VP3 Region of Sea Lion AAV Capsids are Well-Tolerated and can Mediate Covalent Attachment of Antibodies to Viral Particles Via SpyCatcher-SpyTag

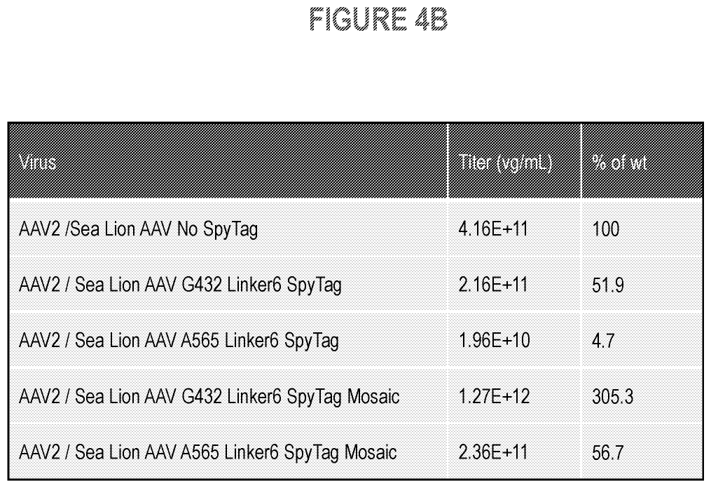

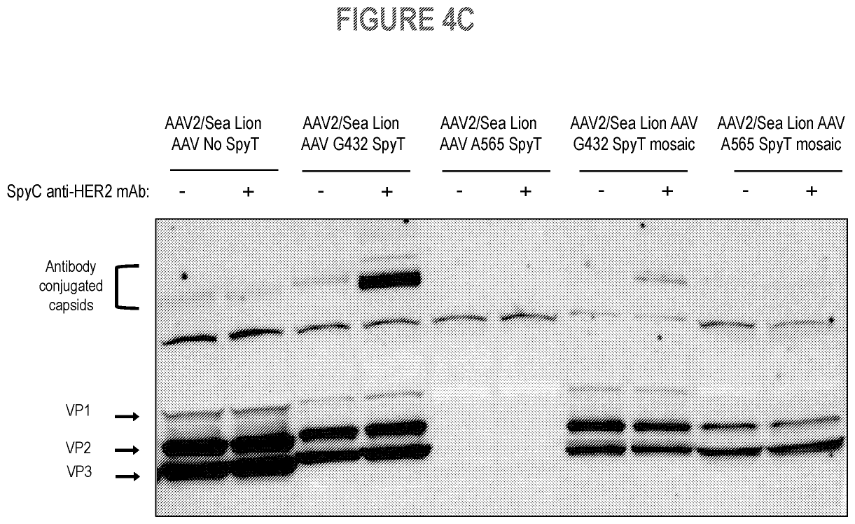

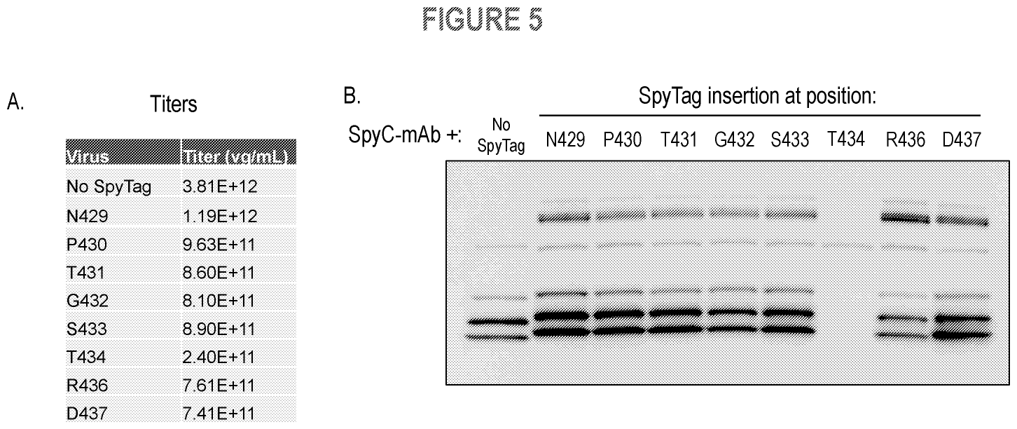

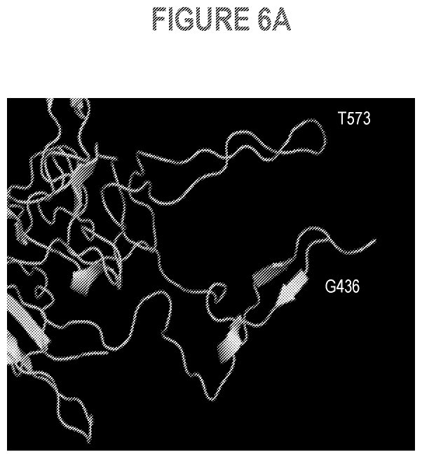

Potential peptide insertion sites within putative variable loop IV and variable loop VIII of Sea Lion AAV were predicted using PyMol modeling (FIG. 4A) and the corresponding SpyTag insertion constructs were cloned. Each virus was generated as described above by transfecting one 15 cm plate of 293T packaging cells with the following plasmids and quantities:

| | | |
|---|---|---|
| pAd Helper | 8 | μg |
| pAAV-CAG-GFP | 4 | μg |
| pAAV R2CapX | 4 | μg |

pAAV R2CapX constructs include:
pAAV R2Cap AAV2/Sea Lion No SpyTag
pAAV R2Cap AAV2/Sea Lion N429 Linker6 SpyTag
pAAV R2Cap AAV2/Sea Lion P430 Linker6 SpyTag
pAAV R2Cap AAV2/Sea Lion T431 Linker6 SpyTag
pAAV R2Cap AAV2/Sea Lion G432 Linker6 SpyTag
pAAV R2Cap AAV2/Sea Lion S433 Linker6 SpyTag
pAAV R2Cap AAV2/Sea Lion T434 Linker6 SpyTag
pAAV R2Cap AAV2/Sea Lion R436 Linker6 SpyTag
pAAV R2Cap AAV2/Sea Lion D437 Linker6 SpyTag
pAAV R2Cap AAV2/Sea Lion A565 Linker6 SpyTag

or

| | | |
|---|---|---|
| pAd Helper | 8 | μg |
| pAAV-CMV-GFP | 4 | μg |
| pAAV R2Cap AAV2/Sea Lion No SpyTa | 3.3 | μg |
| WITH | | |
| pAAV R2Cap AAV2/Sea Lion G432 Linker6 SpyTag | 0.7 | μg |
| OR | | |
| pAAV R2Cap AAV2/Sea Lion A565 Linker6 SpyTag | 0.7 | μg |

Chimeric AAV2/Sea Lion AAV particles lacking SpyTag insertions as well as chimeric AAV2/Sea Lion AAV particles bearing SpyTag insertions at various positions within the capsid as listed above were packaged with ITR2-containing AAV genomes. In an effort to understand whether chimeric AAV2/Sea Lion AAV capsids can be successfully formed and packaged with genomes containing AAV2 ITRs, quantitative PCR was performed to measure the titer (number of genomes per milliliter or vg/mL) of chimeric AAV2/Sea Lion AAV particles bearing SpyTag insertions relative to chimeric AAV2/Sea Lion AAV particles lacking SpyTag insertions (FIG. 4B, 5A). The measured titers demonstrate that chimeric AAV2/Sea Lion AAV particles lacking SpyTag insertions can be packaged with AAV2 ITR genomes. The titer was similar between chimeric AAV2/Sea Lion AAV particles lacking SpyTag and chimeric AAV2/Sea Lion AAV particles bearing a SpyTag insertion in positions N429, P430, T431, G432, S433, R436, and D437, but SpyTag insertions in position T434 or position A565 were not tolerated by chimeric AAV2/Sea Lion AAV particles and produced poor titers.

Next, we incubated the chimeric AAV2/Sea Lion AAV particles lacking SpyTag and chimeric AAV2/Sea Lion AAV particles bearing SpyTag insertions with and without a SpyCatcher-tagged antibody that binds to HER2. The reaction between SpyTagged chimeric AAV2/Sea Lion AAV proteins VP1, VP2 and VP3 and SpyCatcher-tagged anti-HER2 heavy chains was monitored by Western blotting; SpyTagged capsid proteins that have reacted with SpyCatcher-tagged antibody exhibit an increase in size by SDS-PAGE. In the presence of SpyCatcher-tagged anti-HER2 mAb, all detectable SpyTagged chimeric AAV2/Sea Lion AAV capsid proteins displayed an increase in apparent size by Western blotting compared to the SpyTagged chimeric AAV2/Sea Lion AAV capsid proteins alone (FIG. 4C, 5B). This indicated that SpyTagged chimeric AAV2/Sea Lion AAV particles were able to successfully form a covalent bond with SpyCatcher-tagged anti-HER2 mAbs. The SpyTagged chimeric AAV2/Sea Lion AAV particles with low measured titers (SpyTag insertions at T434 and A565) did not have any detectable protein on the Western blot (FIG. 4C, 5B).

### Example 4. Peptide Insertions into the VP3 Region of Bearded Dragon AAV Capsids are Well-Tolerated and can Mediate Covalent Attachment of Antibodies to Viral Particles Via SpyCatcher-SpyTag

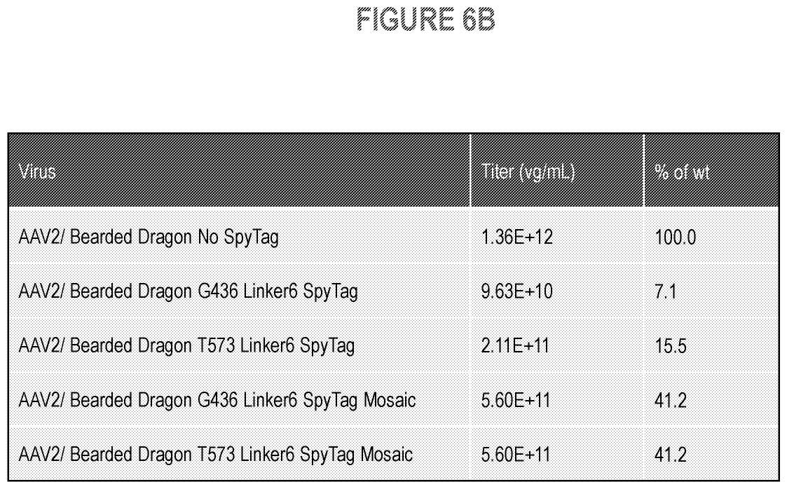

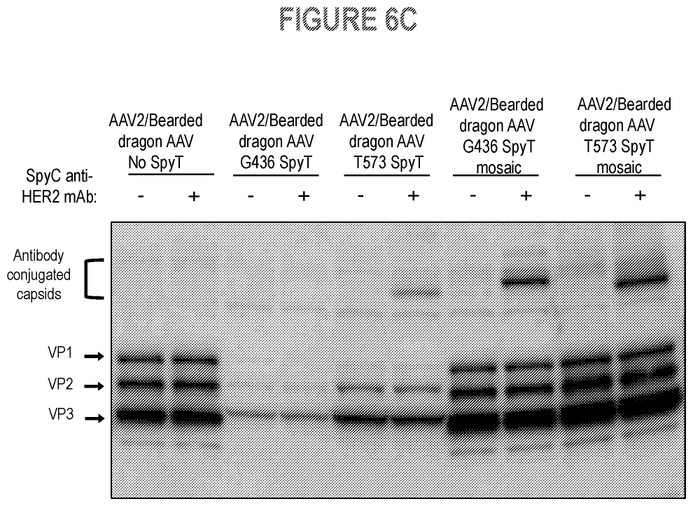

Potential peptide insertion sites within putative variable loop IV and variable loop VIII of Avian AAV were predicted using PyMol modeling (FIG. 6A) and the corresponding SpyTag insertion constructs were cloned. Each virus was generated as described above by transfecting one 15 cm plate of 293T packaging cells with the following plasmids and quantities:

| | |
|---|---|
| pAd Helper | 8 μg |
| pAAV-CMV-GFP | 4 μg |
| pAAV R2CapX | 4 μg |

pAAV R2CapX constructs include:
pAAV R2Cap AAV2/Bearded Dragon No SpyTag
pAAV R2Cap AAV2/Bearded Dragon G436 Linker6 SpyTag
pAAV R2Cap AAV2/Bearded Dragon T573 Linker6 SpyTag

or

| | |
|---|---|
| pAd Helper | 8 μg |
| pAAV-CMV-GFP | 4 μg |
| pAAV R2Cap AAV2/Bearded Dragon No SpyTag | 3.3 ug |
| WITH | |
| pAAV R2Cap AAV2/Bearded Dragon G436 Linker6 SpyTag | 0.7 μg |
| OR | |
| pAAV R2Cap AAV2/Bearded Dragon T573 Linker6 SpyTag | 0.7 μg |

Chimeric AAV2/Bearded Dragon AAV particles lacking SpyTag well as chimeric AAV2/Bearded Dragon AAV particles bearing SpyTag insertions at various positions within the capsid as listed above were packaged with ITR2-containing AAV genomes. In an effort to understand whether chimeric AAV2/Bearded Dragon capsids can be successfully formed and packaged with genomes containing AAV2 ITRs, quantitative PCR was performed to measure the titer (number of genomes per milliliter or vg/mL) of chimeric AAV2/Bearded Dragon AAV particles bearing SpyTag insertions relative to chimeric AAV2/Bearded Dragon AAV particles lacking SpyTag (FIG. 6B). The measured titers demonstrate that chimeric AAV2/Bearded Dragon AAV particles can be packaged with AAV2 ITR genomes. The titer of chimeric AAV2/Bearded Dragon AAV particles bearing SpyTag insertions was less than chimeric AAV2/Bearded Dragon AAV particles lacking SpyTag.

Next, chimeric AAV2/Bearded Dragon AAV particles lacking SpyTag and chimeric AAV2/Bearded Dragon AAV particles bearing SpyTag insertions were incubated with and without a SpyCatcher-tagged antibody that binds to HER2. The reaction between SpyTagged chimeric AAV2/Bearded Dragon AAV proteins VP1, VP2 and VP3 and SpyCatcher-tagged anti-HER2 heavy chains was monitored by Western blotting; SpyTagged capsid proteins that have reacted with SpyCatcher-tagged antibody exhibit an increase in size by SDS-PAGE. In the presence of SpyCatcher-tagged anti-HER2 mAb, SpyTagged chimeric AAV2/Bearded Dragon AAV capsid proteins displayed an increase in apparent size by Western blotting compared to the SpyTagged chimeric AAV2/Bearded Dragon AAV capsid proteins alone (FIG. 6C). This indicated that SpyTagged chimeric AAV2/Bearded Dragon AAV particles were able to successfully form a covalent bond with SpyCatcher-tagged anti-HER2 mAbs.

### Example 5. Conjugation of an Antibody to a Peptide Inserted into the Avian AAV Capsid at Residue G444 or K580 Directs Antigen-Specific Targeting In Vitro

Each virus was generated as described above by transfecting one 15 cm plate of 293T packaging cells with the following plasmids and quantities:

| | |
|---|---|
| pAd Helper | 16 μg |
| pAAV-CAG-GFP | 8 μg |
| pAAV R2CapX | 8 μg |

pAAV R2CapX constructs include:
pAAV R2Cap AAV2/AAAV No SpyTag
pAAV R2Cap AAV2/AAAV G444 Linker6 SpyTag
pAAV R2Cap AAV2/AAAV K580 Linker6 SpyTag

WITH OR WITHOUT

| | |
|---|---|
| SpyCatcher-fused Vh heavy chain plasmid | 1.5 μg |
| Vk light chain plasmid | 3 μg |

Chimeric AAV2/Avian AAV particles lacking SpyTag as well as chimeric AAV2/Avian AAV particles bearing SpyTag insertions at various positions within the capsid as listed above, were produced in the presence or absence of the antibody heavy and light chains encoding either SpyCatcher-anti-GLP1R, an antibody that binds GLP1R and is fused to SpyCatcher at the C-terminus of the heavy chain, SpyCatcher-Herceptin, an antibody that binds HER2 and is fused to SpyCatcher at the C-terminus of the heavy chain, or SpyCatcher-anti-ASGR1, an antibody that binds ASGR1 and is fused to SpyCatcher at the C-terminus of the heavy chain. Cells infected with viral particles as described above were evaluated by flow cytometric analysis to monitor transduction. Chimeric AAV2/AAAV conjugated to the HER2-targeting antibody specifically infected HER2+ cells, (FIG. 7A) and chimeric AAV2/AAAV conjugated to the control non-targeting anti-GLP1R antibody displayed little background infection in any cell type (FIG. 7A, 7B). Chimeric AAV2/AAAV conjugated to the ASGR1-targeting antibody specifically infected ASGR1+ cells, and displayed little background infection of ASGR1− cells (FIG. 7B).

### Example 6. Conjugation of an Antibody to a Peptide Inserted into the Sea Lion AAV Capsid at Residue G432 Directs Antigen-Specific Targeting In Vitro

Each virus was generated as described above by transfecting one 15 cm plate of 293T packaging cells with the following plasmids and quantities:

| | |
|---|---|
| pAd Helper | 16 μg |
| pAAV-CAG-GFP | 8 μg |
| pAAV R2Cap AAV2/Sea Lion No SpyTag | 8 ug |
| or | |
| pAd Helper | 16 μg |
| pAAV-CAG-GFP | 8 μg |
| pAAV R2Cap AAV2/Sea Lion No SpyTag | 4 ug |
| WITH | |
| pAAV R2CapX | 4 ug |

pAAV R2CapX constructs include:
pAAV R2Cap AAV2/Sea Lion N429 Linker6 SpyTag
pAAV R2Cap AAV2/Sea Lion P430 Linker6 SpyTag
pAAV R2Cap AAV2/Sea Lion T431 Linker6 SpyTag
pAAV R2Cap AAV2/Sea Lion G432 Linker6 SpyTag
pAAV R2Cap AAV2/Sea Lion S433 Linker6 SpyTag
pAAV R2Cap AAV2/Sea Lion R436 Linker6 SpyTag
pAAV R2Cap AAV2/Sea Lion D437 Linker6 SpyTag

WITH OR WITHOUT

| | |
|---|---|
| SpyCatcher-fused Vh heavy chain plasmid | 1.5 μg |
| Vk light chain plasmid | 3 μg |

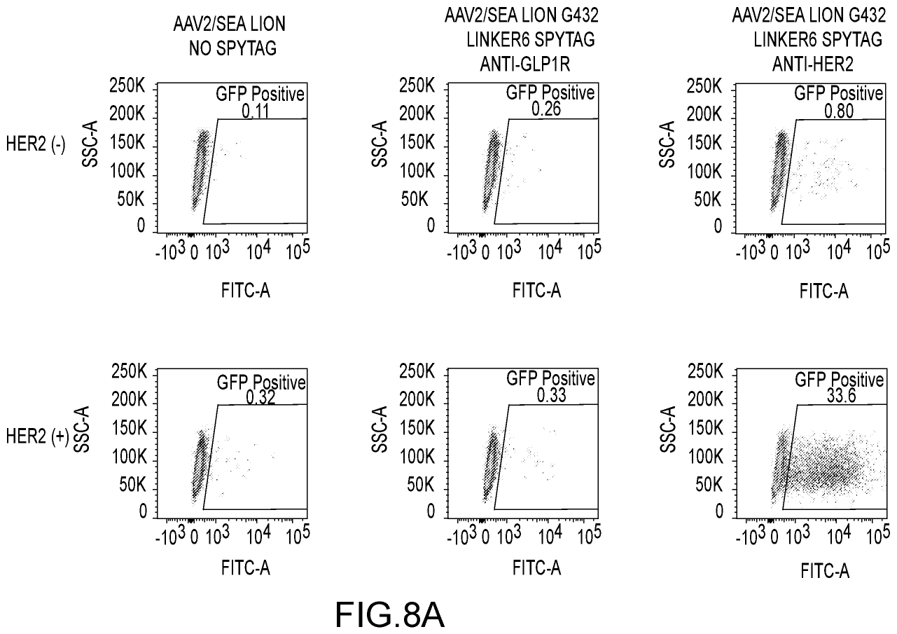

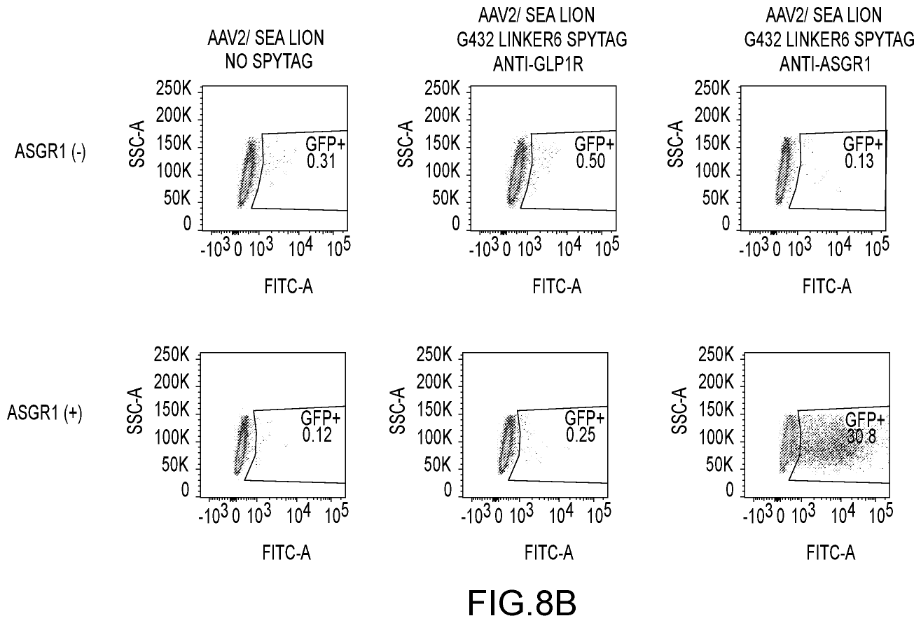

Chimeric AAV2/Sea Lion AAV particles lacking SpyTag as well as chimeric AAV2/Sea Lion AAV particles bearing a SpyTag insertion at position G432 within the capsid as listed above, were produced in the presence or absence of the antibody heavy and light chains encoding either SpyCatcher-anti-GLP1R, an antibody that binds GLP1R and is fused to SpyCatcher at the C-terminus of the heavy chain, SpyCatcher-Herceptin, an antibody that binds HER2 and is fused to SpyCatcher at the C-terminus of the heavy chain, or SpyCatcher-anti-ASGR1, an antibody that binds ASGR1 and is fused to SpyCatcher at the C-terminus of the heavy chain. Cells infected with viral particles as described above were evaluated by flow cytometric analysis to monitor transduction. Chimeric AAV2/Sea Lion AAV bearing a SpyTag at G432 and conjugated to the HER2-targeting antibody specifically infected HER2+ cells, and displayed little background infection of HER2− cells (FIG. 8A), while chimeric AAV2/AAAV conjugated to the control non-targeting anti-GLP1R antibody displayed little background infection in any cell type (FIG. 8A, 8B). Chimeric AAV2/Sea Lion AAV bearing a SpyTag at G432 and conjugated to the ASGR1-targeting antibody specifically infected ASGR1+ cells, and displayed little background infection of ASGR1− cells (FIG. 8B).

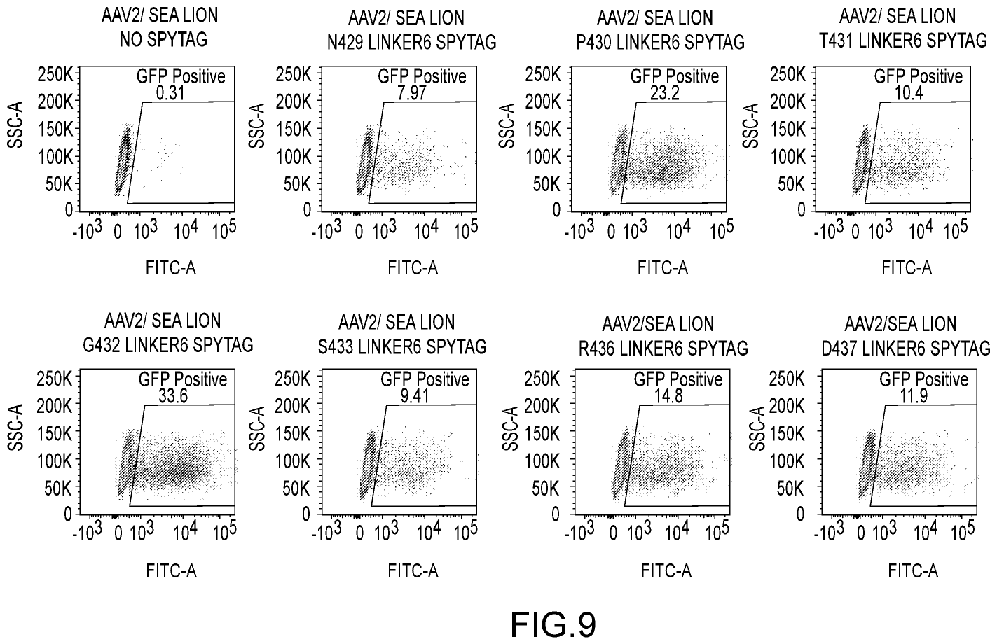

Chimeric AAV2/Sea Lion AAV particles lacking SpyTag as well as a panel of chimeric AAV2/Sea Lion AAV particles bearing SpyTag insertions at various positions within predicted variable loop 4 of the capsid as listed above, were produced in the presence or absence of the antibody heavy and light chains encoding SpyCatcher-Herceptin, an antibody that binds HER2 and is fused to SpyCatcher at the C-terminus of the heavy chain. Cells infected with viral particles as described above were evaluated by flow cytometric analysis to monitor transduction. Chimeric AAV2/Sea Lion AAV particles bearing SpyTag insertions at many different positions within the capsid and conjugated to the HER2-targeting antibody specifically infected HER2+ cells, and chimeric AAV2/Sea Lion AAV particles without a SpyTag displayed little background infection of HER2+ cells (FIG. 9). This suggests that multiple sites within the chimeric AAV2/Sea Lion AAV capsid are amenable to SpyTag insertion and retargeting using the SpyCatcher-fused antibody approach.

### Example 7. Conjugation of an Antibody to a Peptide Inserted into the Bearded Dragon AAV Capsid at Residue G436 or T573 Directs Antigen-Specific Targeting In Vitro

Each virus was generated as described above by transfecting one 15 cm plate of 293T packaging cells with the following plasmids and quantities:

| | |
|---|---|
| pAd Helper | 8 μg |
| pAAV-CMV-GFP | 4 μg |
| pAAV R2CapX | 4 μg |

pAAV R2CapX constructs include:
pAAV R2Cap AAV2/Bearded Dragon No SpyTag
pAAV R2Cap AAV2/Bearded Dragon G436 Linker6 SpyTag
pAAV R2Cap AAV2/Bearded Dragon T573 Linker6 SpyTag

or

| | |
|---|---|
| pAd Helper | 8 μg |
| pAAV-CMV-GFP | 4 μg |
| pAAV R2Cap AAV2/Bearded Dragon No SpyTag | 3.4 ug |
| WITH | |
| pAAV R2Cap AAV2/Bearded Dragon G436 Linker6 SpyTag | 0.6 μg |
| OR | |
| pAAV R2Cap AAV2/Bearded Dragon T573 Linker6 SpyTag | 0.6 μg |
| WITH OR WITHOUT | |
| SpyCatcher-fused Vh heavy chain plasmid | 1.5 μg |
| Vk light chain plasmid | 3 ug |

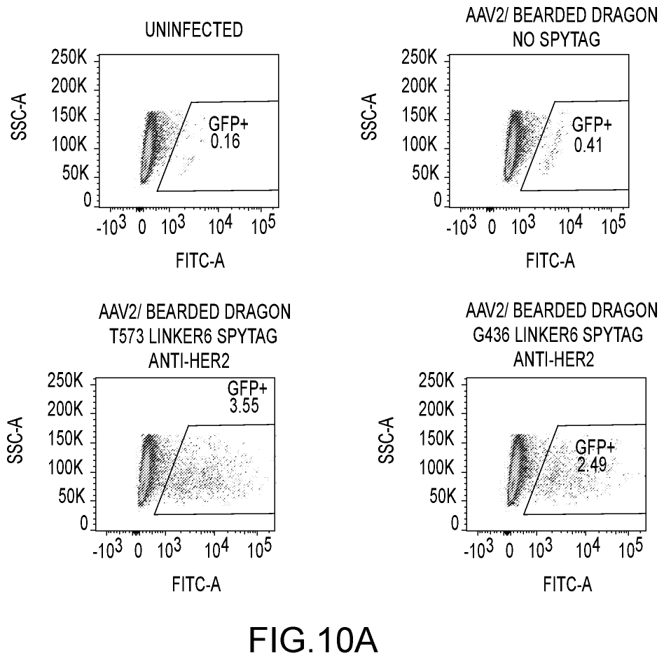

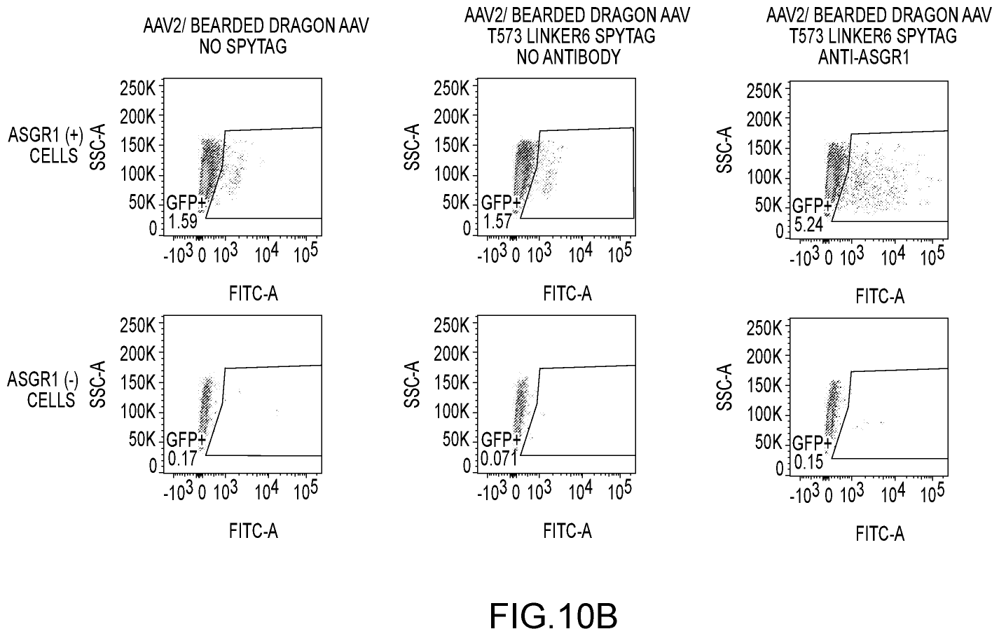

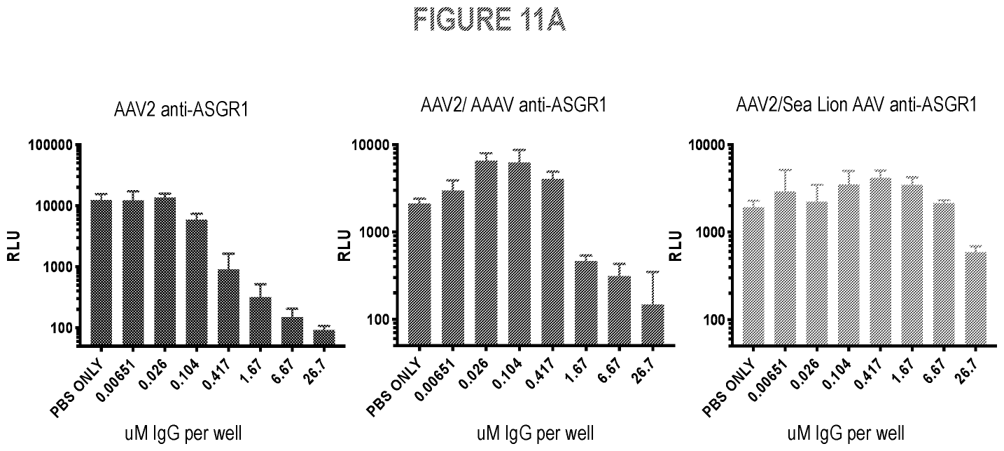

Chimeric AAV2/Bearded Dragon AAV particles lacking SpyTag as well as chimeric AAV2/Bearded Dragon AAV particles bearing SpyTag insertions at various positions within the capsid as listed above, were produced in the presence or absence of the antibody heavy and light chains encoding either SpyCatcher-Herceptin, an antibody that binds HER2 and is fused to SpyCatcher at the C-terminus of the heavy chain, or SpyCatcher-anti-ASGR1, an antibody that binds ASGR1 and is fused to SpyCatcher at the C-terminus of the heavy chain. Cells infected with viral particles as described above were evaluated by flow cytometric analysis to monitor transduction. Chimeric AAV2/Bearded Dragon AAV conjugated to the HER2-targeting antibody infected HER2+ cells (FIG. 10A). Chimeric AAV2/Bearded Dragon AAV conjugated to the ASGR1-targeting antibody specifically infected ASGR1+ cells, and displayed little background infection of ASGR1− cells (FIG. 10B).

### Example 8. Antibody-Conjugated Avian AAV and Sea Lion AAV can Infect Cells in the Presence of Higher Levels of Purified Human Immunoglobulins than AAV2

To probe the sensitivity of chimeric AAV2/AAAV and chimeric AAV2/Sea Lion AAV particles to neutralization by antibodies found in human serum, we performed a neutralization assay in which AAV particles are incubated with increasing amounts of IgG (hIgG) prepared from pooled serum samples from tens to thousands of human donors and represents a cross-section of immunoglobulins in the human population.

Each virus was generated as described above by transfecting 15 cm plates of 293T packaging cells with the following plasmids and quantities:

| | |
|---|---|
| pAd Helper | 16 μg |
| pAAV-CMV-Nanoluc | 8 μg |
| pAnti-hASGR1 SpyCatcher Vh | 3 μg |
| pAnti-hASGR1 Vk | 6 μg |
| WITH EITHER: | |
| pAAV R2C2 N587 Myc | 7.5 μg |
| pAAV R2C2 G453 Linker10 SpyTag | 0.5 μg |
| OR: | |
| pAAV R2Cap AAV2/AAAV No SpyTag | 5 μg |
| pAAV R2Cap AAV2/AAAV K580 Linker6 SpyTag | 3 μg |
| OR: | |
| pAAV R2Cap AAV2/Sea Lion No SpyTag | 5 μg |
| pAAV R2Cap AAV2/Sea Lion G432 Linker10 SpyTag | 3 μg |

Mosaic AAV2, mosaic chimeric AAV2/AAAV particles bearing SpyTag insertions within the capsid, and mosaic chimeric AAV2/Sea Lion AAV particles bearing SpyTag insertions within the capsid as listed above, were produced in the presence of the antibody heavy and light chains encoding SpyCatcher-anti-ASGR1, an antibody that binds hASGR1 and is fused to SpyCatcher at the C-terminus of the heavy chain. The particles were then incubated in the presence of increasing concentrations of hIgG for 30 minutes at 37 C, then the mixture of viral particles and hIgG was added to cells expressing hASGR1. Cells infected with viral particles as described above were evaluated by Nanoglo Luciferase assay to monitor transduction.

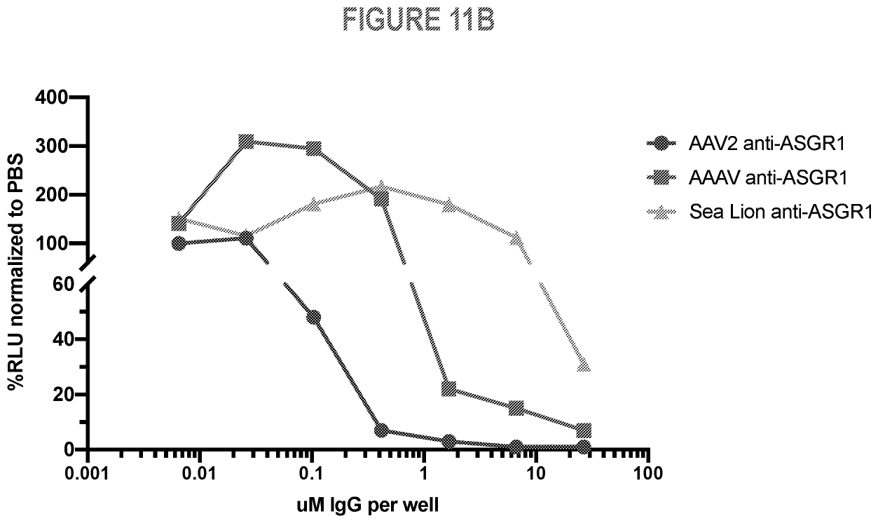

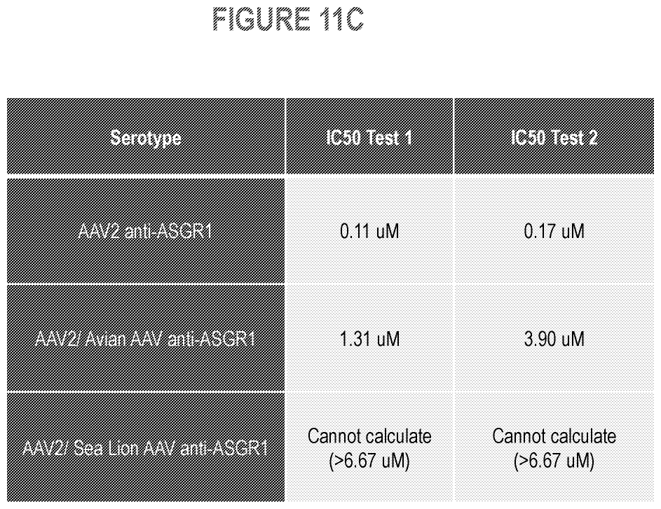

FIG. 11A shows the raw Nanoluc luciferase activity of ASGR1+ cells infected with the indicated viral particles in the presence of the indicated concentration of hIgG. Compared to mosaic AAV2 particles conjugated to anti-ASGR1 antibodies, a higher amount of hIgG is required to inhibit infection of mosaic chimeric AAV2/AAAV particles conjugated to anti-ASGR1 antibodies, and an even higher amount of hIgG is required to inhibit the infection of mosaic chimeric AAV2/Sea Lion AAV particles conjugated to anti-ASGR1 antibodies. FIG. 11B shows the Nanoluc luciferase data from FIG. 11A normalized to the PBS only (no hIgG) condition for each AAV serotype, in order to compare the three AAV serotypes to one another directly. Higher concentrations of hIgG are required to reduce the Nanoluc luciferase expression of both mosaic chimeric AAV2/AAAV particles conjugated to anti-ASGR1 antibodies and mosaic chimeric AAV2/Sea Lion AAV particles conjugated to anti-ASGR1 antibodies. The concentration of hIgG per well that is required to inhibit the infection by 50% (IC50 values) for two independent experiments is shown in FIG. 11C. The hIgG IC50 values for mosaic chimeric AAV2/AAAV particles conjugated to anti-ASGR1 antibodies and the hIgG IC50 values for mosaic chimeric AAV2/Sea Lion AAV particles conjugated to anti-ASGR1 antibodies are much greater than the hIgG IC50 values for mosaic AAV2 particles conjugated to anti-ASGR1 antibodies. In particular, mosaic chimeric AAV2/Sea Lion AAV particles appear to be unaffected by human antibodies at all but the highest concentrations of hIgG tested.

### Example 9. Avian AAV can be Retargeted to ASGR1 In Vivo

To determine whether the viral particles conjugated to antibodies specific to ASGR1 could be retargeted to liver cells expressing hASGR1 in vivo, mice genetically modified such that their liver cells express hASGR1 on a C57BL/6 background were injected intravenously with mosaic chimeric AAV2/AAAV viral particles carrying a firefly luciferase reporter gene and conjugated via SpyCatcher-SpyTag either to an antibody specific to hASGR1 or a control antibody targeting human GLP1R. A mouse injected with phosphate buffered saline (PBS) served as an additional control.

Each virus was generated as described above by transfecting 15 cm plates of 293T packaging cells with the following plasmids and quantities:

Avian AAV Anti-Human GLP1R/anti-Human ASGR1 Luciferase

| | |
|---|---|
| pAd Helper | 16 μg |
| pAAV-UbC-Firefly Luciferase | 8 μg |
| pAAV R2Cap AAV2/AAAV No SpyTag | 5 μg |
| pAAV R2Cap AAV2/AAAV K580 Linker6 SpyTag | 3 μg |
| WITH or WITHOUT | |
| pAnti-hGLP1R or Anti-hASGR1 SpyCatcher Vh | 2.5 μg |
| pAnti-hGLP1R or Anti-hASGR1 Vk | 5 μg |

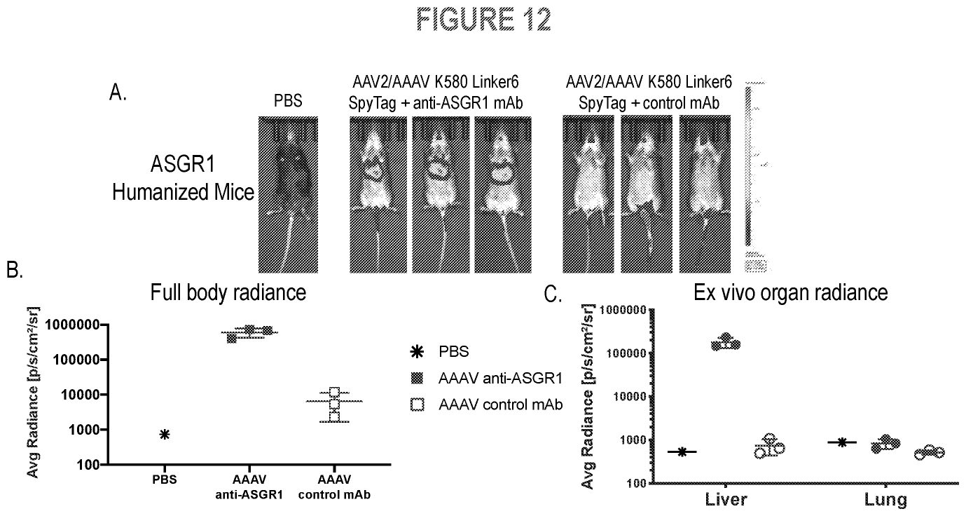

FIG. 12 shows luminescence of animals 33 days post-injection either with PBS, or with mosaic chimeric AAV2/AAAV viral particles as described above conjugated to antibodies targeting hASGR1 or hGLP1R as a non-targeting control. Live animals were anesthetized using isoflurane, injected with a Luciferin substrate and imaged 10 minutes later using the IVIS Spectrum In Vivo Imaging System (PerkinElmer). FIG. 12A shows that infection with the mosaic chimeric AAV2/AAAV-SpyTag-SpyCatcher-Vh complexes was detected only in the liver of hASGR1-expressing mice injected with hASGR1-retargeted mosaic chimeric AAV2/AAAV and was not detected in the liver of hASGR1-expressing mice injected with PBS or control non-targeting hGLP1R-retargeted mosaic chimeric AAV2/AAAV. The average radiance of the Firefly Luciferase signal that was detected from the live mice using the IVIS Spectrum In Vivo Imaging System (PerkinElmer) is quantified in FIG. 12B, and the average radiance of individual organs imaged ex-vivo following dissection of the infected mice is quantified in FIG. 12C. These figures demonstrate that mosaic chimeric AAV2/AAAV specifically transduces the liver of hASGR1-expressing mice only when conjugated to a hASGR1-specific antibody.

### Example 10. Sea Lion AAV Transduces the Liver and Lung In Vivo in Mice

To determine whether the viral particles conjugated to antibodies specific to ASGR1 could be retargeted to liver cells expressing hASGR1 in vivo, mice genetically modified such that their liver cells express hASGR1 on a C57BL/6 background were injected intravenously with mosaic chimeric AAV2/Sea Lion AAV viral particles carrying a firefly luciferase reporter gene and conjugated via SpyCatcher-SpyTag either to an antibody specific to hASGR1 or a control antibody targeting human GLP1R. A mouse injected with phosphate buffered saline (PBS) served as an additional control.

Each virus was generated as described above by transfecting 15 cm plates of 293T packaging cells with the following plasmids and quantities:

Sea Lion AAV Anti-Human GLP1R/anti-Human ASGR1 Luciferase

| | |
|---|---|
| pAd Helper | 16 μg |
| pAAV-UbC-Firefly Luciferase | 8 μg |
| pAAV R2Cap AAV2/Sea Lion No SpyTag | 5 μg |
| pAAV R2Cap AAV2/Sea Lion G432 Linker10 SpyTag | 3 ug |
| WITH or WITHOUT | |
| pAnti-hGLP1R or Anti-hASGR1 SpyCatcher Vh | 2.5 μg |
| pAnti-hGLP1R or Anti-hASGR1 Vk | 5 μg |

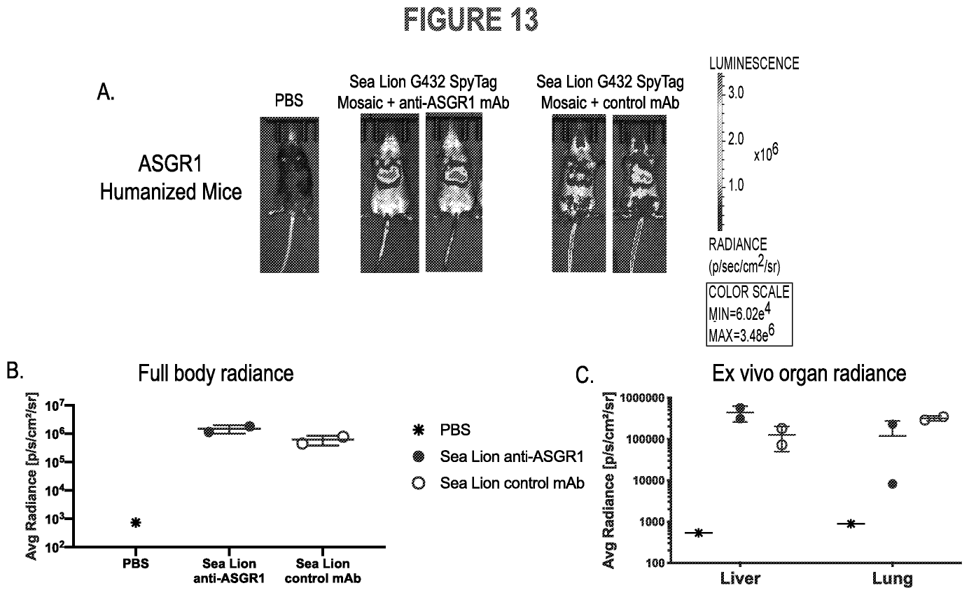

FIG. 13 shows luminescence of animals 33 days post-injection either with PBS, or with mosaic chimeric AAV2/Sea Lion AAV viral particles as described above conjugated to antibodies targeting hASGR1 or hGLP1R as a non-targeting control. Live animals were anesthetized using isoflurane, injected with a Luciferin substrate and imaged 10 minutes later using the IVIS Spectrum In Vivo Imaging System (PerkinElmer). FIG. 13A shows that infection with the mosaic chimeric AAV2/Sea Lion AAV-SpyTag-SpyCatcher-Vh complexes was detected in hASGR1-expressing mice injected with both hASGR1-retargeted chimeric AAV2/Sea Lion AAV and the control non-targeting hGLP1R-retargeted mosaic chimeric AAV2/Sea Lion AAV. This suggests that chimeric AAV2/Sea Lion AAV is able to naturally transduce the mouse liver and other organs without the aid of a retargeting antibody. The average radiance of the Firefly Luciferase signal that was detected from the live mice using the IVIS Spectrum In Vivo Imaging System (PerkinElmer) is quantified in FIG. 13B, and the average radiance of individual organs imaged ex-vivo following dissection of the infected mice is quantified in FIG. 13C. These figures demonstrate that mosaic chimeric AAV2/Sea Lion AAV transduces the liver and lungs of hASGR1-expressing mice when conjugated to a hASGR1-specific antibody or a non-targeting control hGLP1R-specific control antibody.

### Example 11. Chimeric AAV2/Sea Lion AAV Displays Some Natural Tropism in the Mouse Inner Ear

Each virus was generated as described above by transfecting 15 cm plates of 293T packaging cells with the following plasmids and quantities:

| | |
|---|---|
| pAd Helper | 16 μg |
| pAAV-CAG-GFP | 8 μg |
| pAAV R2Cap AAV2/Sea Lion No SpyTag | 8 μg |

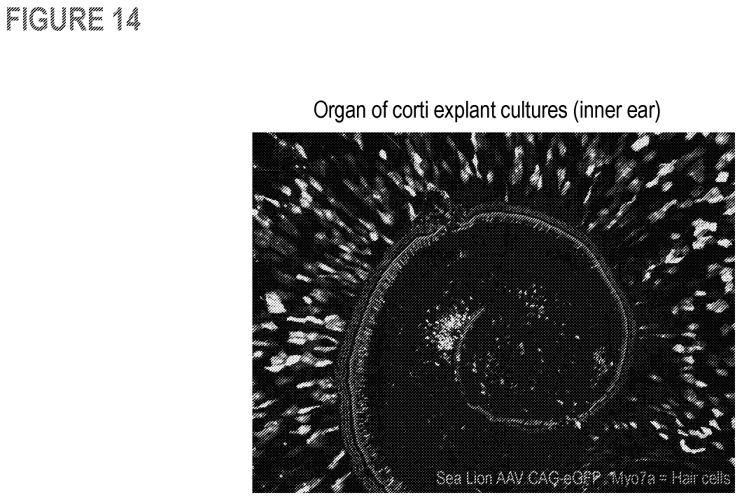

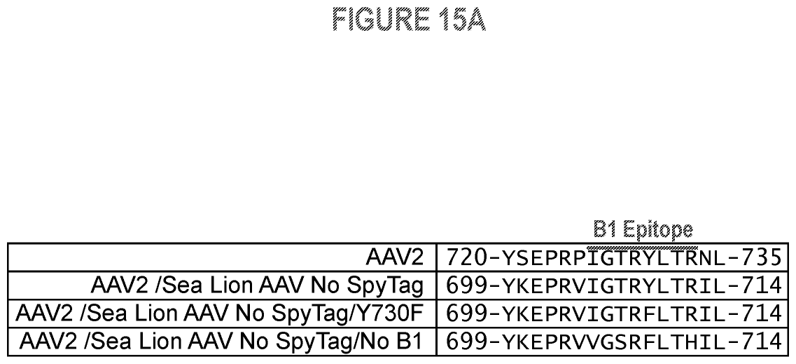

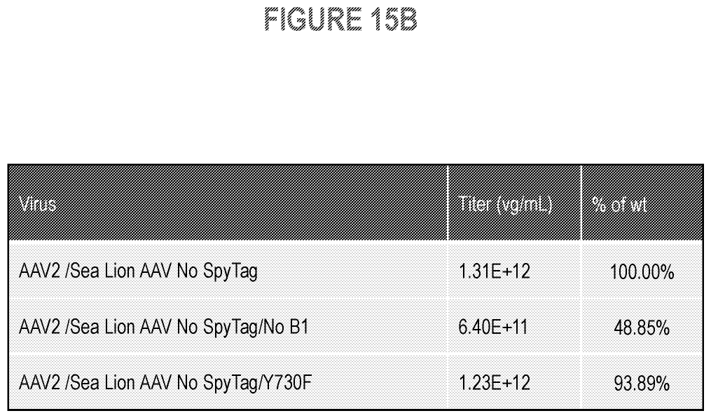

To determine whether chimeric AAV2/Sea Lion AAV has natural tropism for particular tissues in the mouse, the organ of corti was dissected from a neonatal mouse, cultured ex vivo, then AAV2/Sea Lion AAV virus particles were used to infect the culture. Three days post-infection, cochlear hair cells were stained red with Myo7a, and the virus expressed GFP as a marker of transduction. Robust transduction of multiple cell types was observed (FIG. 14) suggesting that chimeric AAV2/Sea Lion AAV particles are naturally able to transduce the inner ear.

### Example 12. Replacement of B1 Epitope of Chimeric AAV2/Sea Lion AAV Capsids Partially or Entirely with Homologous Sequence from Sea Lion AAV is Well-Tolerated and Enhances Transduction Efficiency

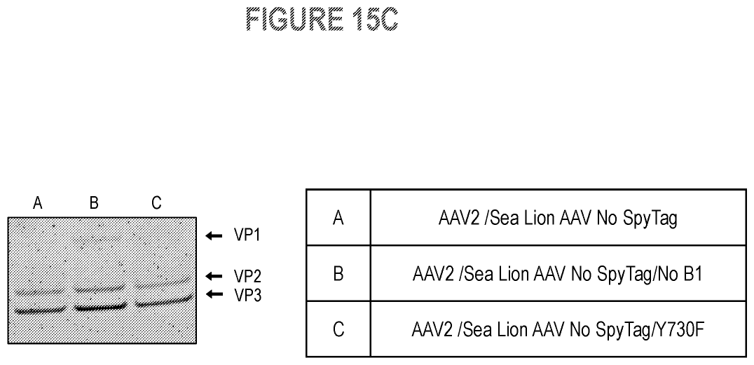

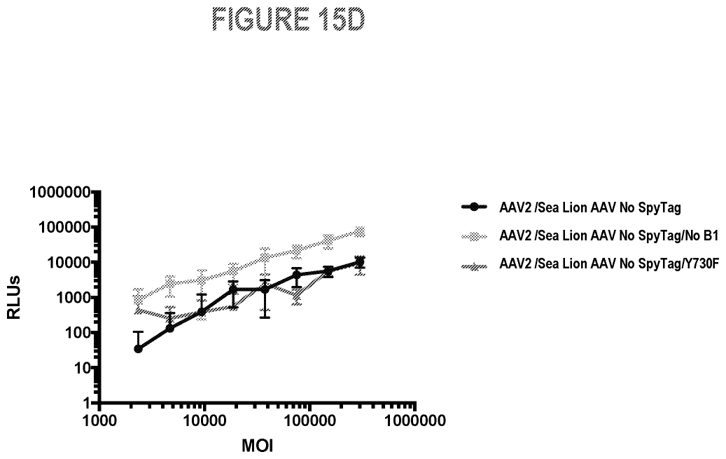

Modifications were made within the B1 epitope of AAV2/Sea Lion chimeras (FIG. 15A) and the B1 epitope sequence was replaced at Y730 or from I705 to H712 with homologous Sea Lion capsid sequence. Each virus was generated as described above by transfecting five, ten, or twenty 15 cm plates of HEK 293T packaging cells with the following plasmids and quantities per plate:

| | |
|---|---|
| pAd Helper | 12 μg |
| pAAV-CMV-X | 6 μg |
| pAAV-CMV-X constructs include: | |
| pAAV-CMV-NanoLuc Luciferase | |
| pAAV-CMV-Firefly Luciferase | |
| pAAV R2CapX | 10 μg |

pAAV R2CapX constructs include:
pAAV R2Cap AAV2/Sea Lion AAV No SpyTag
pAAV R2Cap AAV2/Sea Lion AAV No SpyTag/Y730F
pAAV R2Cap AAV2/Sea Lion AAV No SpyTag/No B1

Chimeric AAV2/Sea Lion AAV particles lacking SpyTag insertions and containing modification of B1 epitope sequence within the capsid as listed above were packaged with ITR2-containing AAV genomes. In an effort to understand whether chimeric AAV2/Sea Lion capsids with modified B1 epitopes can be successfully formed and packaged with genomes containing AAV2 ITRs, quantitative PCR was performed to measure the titer (number of vector genomes per milliliter or vg/mL) of chimeric AAV2/Sea Lion AAV particles with B1 epitope relative to chimeric AAV2/Sea Lion AAV particles with B1 epitope modifications (FIG. 15B). The measured titers demonstrate that chimeric AAV2/Sea Lion AAV particles with B1 epitope modifications can be packaged with AAV2 ITR genomes. The titer of chimeric AAV2/Sea Lion AAV particles was similar between chimeric AAV2/Sea Lion particles with or without B1 epitope modifications.

The presence of VP1, VP2, and VP3 assembled into chimeric AAV2/Sea Lion AAV particles with modified B1 epitopes we monitored by SDS-PAGE and protein staining. The results indicated that the proportion of VP1 expression relative to VP2 and VP3 was greater in the AAV2/Sea Lion AAV viral particles in which the B1 epitope had been entirely replaced with homologous Sea Lion AAV capsid sequence than in AAV2/Sea Lion AAV viral particles with the B1 epitope or a Y730F mutation in the B1 epitope (FIG. 15C). This observation correlated with an increase in transduction efficiency of HEK 293T cells as determined by NanoLuc Luciferase activity (FIG. 15D).

To determine if the increase in transduction efficiency for AAV2/Sea Lion AAV particles with the B1 epitope replaced entirely with the AAV Sea Lion sequence was reproduced in vivo, C57BL/6 mice were injected intravenously with chimeric AAV2/Sea Lion particles carrying a firefly luciferase reporter gene. A mouse injected with phosphate buffered saline (PBS) served as an additional control.

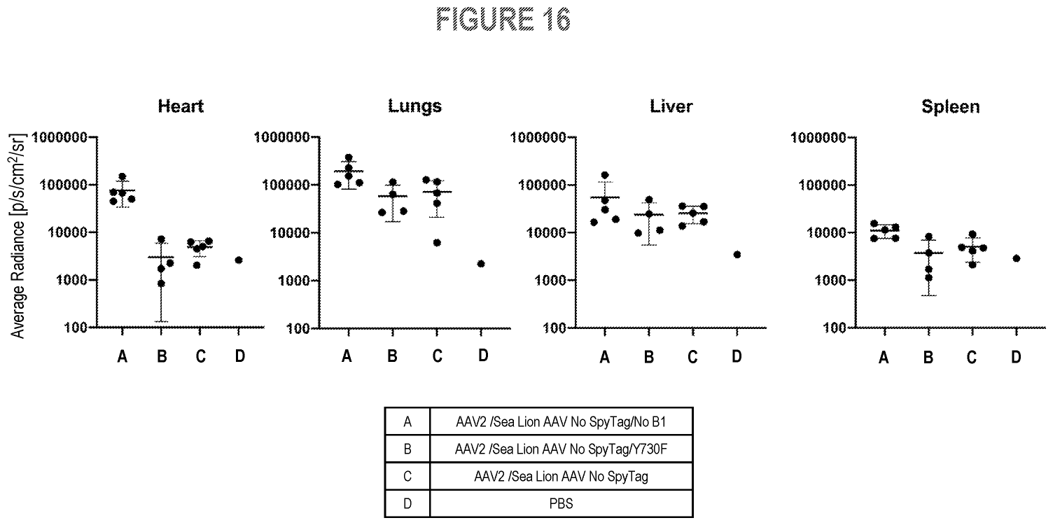

FIG. 16 shows luminescence data of animals 34 days post-injection with either PBS or chimeric AAV2/Sea Lion viral particles as described above. Live animals were anesthetized using isoflurane, injected with a Luciferin substrate and imaged 10 minutes later using the IVIS Spectrum In Vivo Imaging System (PerkinElmer). The average radiance of individual organs imaged ex-vivo following dissection of the infected mice is quantified. The Figure shows that chimeric AAV2/Sea Lion AAVs transduce the liver and lungs. AAV2/Sea Lion AAVs containing modifications of the B1 epitope in which it is entirely replaced with homologous Sea Lion AAV capsid sequence have expanded tropism to the heart and slightly improved transduction in liver and lungs.

### Example 13. Adjustment of Chimeric Interface Between AAV2 and Sea Lion AAV Capsid Sequences or Use of Capsid Sequences Entirely Composed Those from AAV Sea Lion is Well-Tolerated

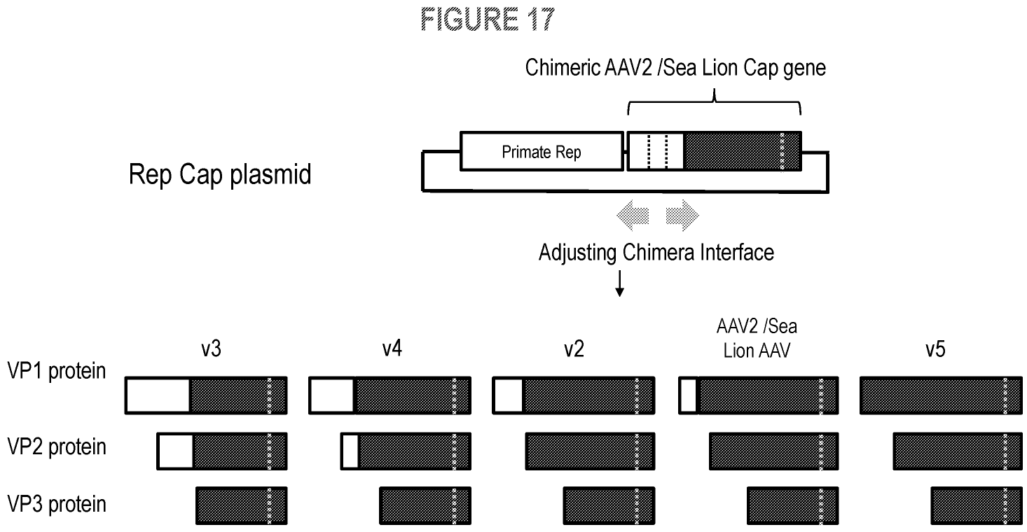

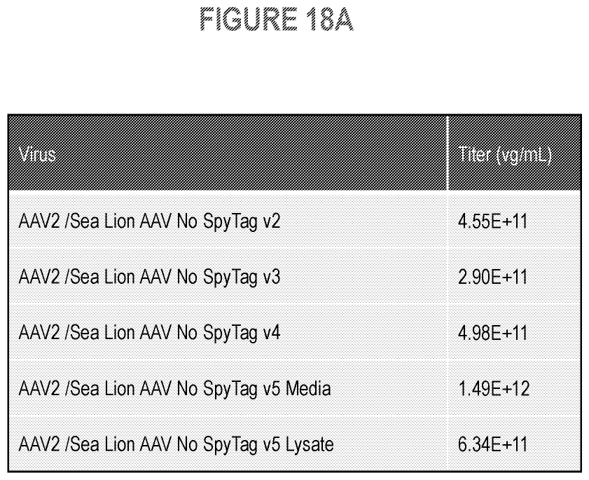

Modifications were made to adjust the chimeric interface between AAV2 and Sea Lion capsid sequences to generate transcripts in which the VP1 and VP2 unique sequences were derived from either AAV2 or Sea Lion AAV or a combination thereof (FIG. 17). Each virus was generated as described above by transfecting five, ten, or twenty 15 cm plate of HEK 293T packaging cells with the following plasmids and quantities per plate:

| | |
|---|---|
| pAd Helper | 12 μg |
| pAAV-CMV-NanoLuc Luciferase | 6 μg |
| pAAV R2CapX | 10 μg |

pAAV R2CapX constructs include:
pAAV R2Cap AAV2/Sea Lion AAV No SpyTag
pAAV R2Cap AAV2/Sea Lion AAV No SpyTag v2
pAAV R2Cap AAV2/Sea Lion AAV No SpyTag v3
pAAV R2Cap AAV2/Sea Lion AAV No SpyTag v4
pAAV R2Cap AAV2/Sea Lion AAV No SpyTag v5

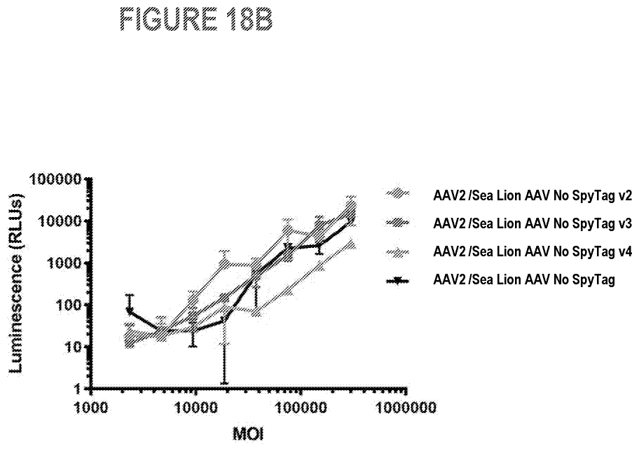

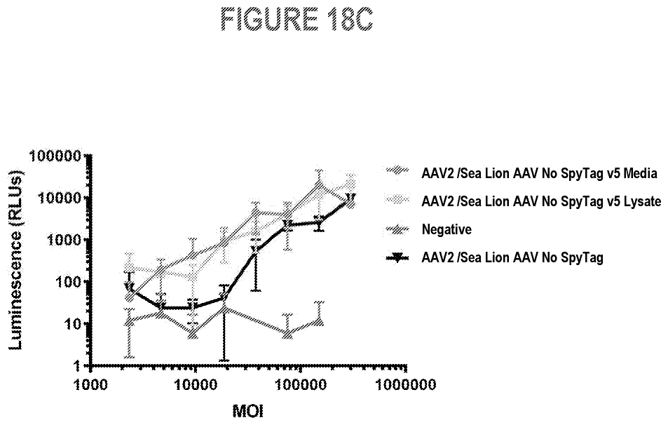

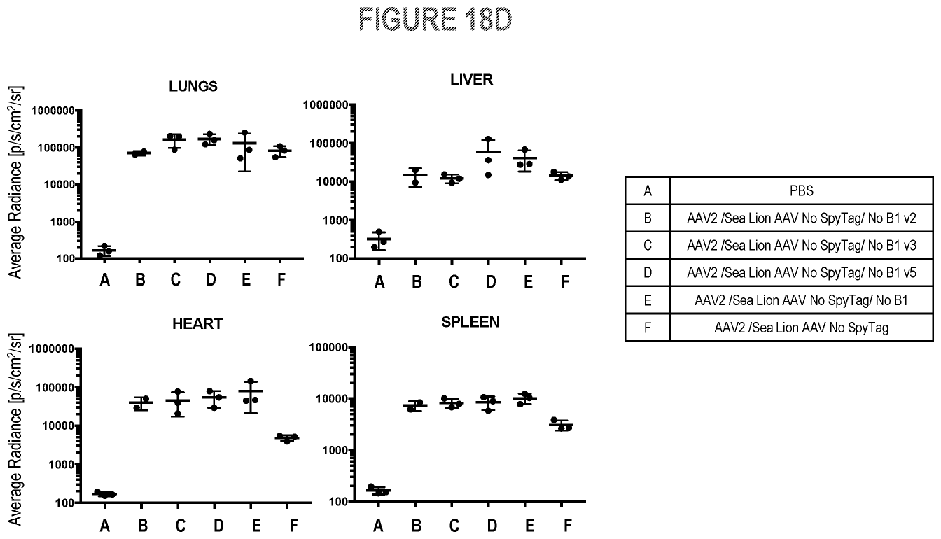

Chimeric AAV2/Sea Lion AAV particles with alternative interfaces between chimeric AAV2 and AAV Sea Lion capsid sequences as listed above were packaged with ITR2-containing AAV genomes. In an effort to understand whether these alternative chimeric AAV2/Sea Lion capsids can be successfully formed and packaged with genomes containing AAV2 ITRs, quantitative PCR was performed to measure the titer (number of vector genomes per milliliter or vg/mL) of viral particles purified from either the lysate or supernatant (FIG. 18A). The measured titers demonstrate that chimeric AAV2/Sea Lion AAV particles with alternative chimeric interfaces can be packaged with AAV2 ITR genomes. The titer of chimeric AAV2/Sea Lion AAV particles with exception of v3 was similar between chimeric AAV2/Sea Lion particles purified from lysate. The titer of AAV2/Sea Lion AAV No SpyTag v5 particles purified from media was slightly higher than the titer purified from the lysate. Transduction efficiency of HEK 293T cells as determined by NanoLuc Luciferase activity of chimeric AAV2/Sea Lion AAV particles was comparable between alternative interface locations (FIGS. 18B and 18C).

The B1 epitope sequence was modified to entirely homologous Sea Lion capsid sequence in AAV2/Sea Lion chimeras with alternative interface sites to determine if the B1 modifications would similarly enhance transduction as it had done for AAV2/Sea Lion AAV No SpyTag. Each virus was generated as described above by transfecting five, ten, or twenty 15 cm plate of HEK 293T packaging cells with the following plasmids and quantities per plate:

| | |
|---|---|
| pAd Helper | 12 μg |
| pAAV-CMV-Firefly Luciferase | 6 μg |
| pAAV R2CapX | 10 μg |

pAAV R2CapX constructs include:
pAAV R2Cap AAV2/Sea Lion AAV No SpyTag
pAAV R2Cap AAV2/Sea Lion AAV No SpyTag/No B1 v2
pAAV R2Cap AAV2/Sea Lion AAV No SpyTag/No B1 v3
pAAV R2Cap AAV2/Sea Lion AAV No SpyTag/No B1 v4
pAAV R2Cap AAV2/Sea Lion AAV No SpyTag/No B1 v5

To determine if the increase in transduction efficiency for alternative chimeric AAV2/Sea Lion AAV particles with the B1 epitope replaced entirely with the AAV Sea Lion sequence resulted in enhanced transduction in vivo, C57BL/6 mice were injected intravenously with chimeric AAV2/Sea Lion particles carrying a firefly luciferase reporter gene. A mouse injected with phosphate buffered saline (PBS) served as an additional control.

FIG. 18D shows luminescence data of animals 46 days post-injection with either PBS or chimeric AAV2/Sea Lion viral particles as described above. Live animals were anesthetized using isoflurane, injected with a Luciferin substrate and imaged 10 minutes later using the IVIS Spectrum In Vivo Imaging System (PerkinElmer). The radiance of individual organs imaged ex-vivo following dissection of the infected mice is quantified. The Figure shows that chimeric AAV2/Sea Lion AAVs transduce the liver, lungs, and heart. The capacity of AAV2/Sea Lion AAV No SpyTag/No B1 v5 (SEQ ID NO:71) to transduce tissues indicates that in addition to the chimeric AAV2/Sea Lion AAV capsid particles, an AAV capsid particle comprised entirely of Sea Lion capsids (non-chimeric capsid proteins) can function as a gene transfer vector.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims. Although any methods and materials similar or equivalent to those described herein can be used in the practice or testing of the present invention, some preferred methods and materials are now described. All publications cited herein are incorporated herein by reference to describe in their entirety. Unless defined otherwise, all technical and scientific terms used herein have the same meaning as commonly understood by one of ordinary skill in the art to which this invention belongs.

What is claimed is:

1. A recombinant AAV viral particle comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,
wherein at least one of: the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein, comprises an amino acid sequence having significant sequence identity, e.g., at least 95% identity, to the amino acid sequence of a capsid protein of a non-primate animal AAV or portion thereof, or a remote AAV or a portion thereof,
wherein
I. at least one of the AAV VP1, VP2, and VP3 capsid proteins comprises a modification selected from the group consisting of:
(a) a first member of a protein:protein binding pair, wherein the protein:protein binding pair directs the tropism of the AAV viral particle,
(b) a detectable label,
(c) a point mutation, preferably wherein the point mutation reduces the natural tropism of the AAV viral particle and/or creates a detectable label,
(d) a chimeric amino acid sequence, and
(e) any combination of (a), (b), (c), and (d), and/or
II. the ITR sequence, or portion thereof, comprises a nucleic acid sequence having significant sequence identity, e.g., at least 95% identity, to the ITR sequence of a second AAV, wherein the second AAV is not the same as the non-primate animal AAV or the remote AAV, and
wherein the recombinant AAV viral particle is capable of infecting a mammalian host, preferably a primate host.

2. A recombinant AAV viral particle comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,
wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a non-primate animal AAV or portion thereof, and
wherein at least one of the AAV VP1, VP2, and VP3 capsid proteins comprises a modification selected from the group consisting of:
(a) a first member of a protein:protein binding pair, wherein the protein:protein binding pair directs the tropism of the AAV viral particle,
(b) a detectable label,
(c) a point mutation, preferably wherein the point mutation reduces the natural tropism of the AAV viral particle and/or creates a detectable label,
(d) a chimeric amino acid sequence, and
(e) any combination of (a), (b), (c), and (d),
wherein the entire ITR sequence or a portion of the ITR sequence comprises a nucleic acid sequence having significant sequence identity, e.g., at least 95% identity, to the ITR of the non-primate animal AAV, optionally wherein the ITR sequence comprises a chimeric nucleic acid sequence, and wherein a portion of the chimeric nucleic acid sequence having significant sequence identity, e.g., at least 95% identity, to the ITR of the non-primate AAV is operably linked to a portion of the chimeric nucleic acid sequence having significant sequence identity, e.g., at least 95% identity, to the ITR of a second AAV, wherein the second AAV is not the same as the non-primate animal AAV, and
wherein the recombinant AAV viral particle is capable of infecting a mammalian host preferably a primate host.

3. A recombinant AAV viral particle comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,
wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises an amino acid sequence having significant sequence identity, e.g., at least 95% identity, to the amino acid sequence of a capsid protein of a non-primate animal AAV or portion thereof, optionally wherein at least one of the AAV VP1, VP2, and VP3 capsid proteins comprises a modification selected from the group consisting of:
(a) a first member of a protein:protein binding pair, wherein the protein:protein binding pair directs the tropism of the AAV viral particle,
(b) a detectable label,
(c) a point mutation, preferably wherein the point mutation reduces the natural tropism of the AAV viral particle and/or creates a detectable label, and
(d) any combination of (a)-(c),
wherein the ITR sequence, or portion thereof, comprises a nucleic acid sequence having significant sequence identity, e.g., at least 95% identity, to the ITR sequence of a second AAV, wherein the second AAV is not the same as the non-primate animal AAV, and
wherein the recombinant AAV viral particle is capable of infecting a mammalian host preferably a primate host.

4. A recombinant AAV viral particle comprising (i) an AAV capsid comprising AAV VP1, VP2, and VP3 capsid proteins, and (ii) packaged within the AAV capsid, a nucleic acid sequence comprising an AAV Inverted Terminal Repeat (ITR) sequence,
wherein at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprises a chimeric amino acid sequence comprising (A) an amino acid sequence having significant sequence identity, e.g., at least 95% identity, to the amino acid sequence of a non-primate animal AAV capsid protein, or a portion thereof, operably linked to (B) an amino acid sequence having significant sequence identity, e.g., at least 95% identity, to the amino acid sequence of a second AAV capsid protein, or a portion thereof,
wherein the second AAV is not the same as the non-primate animal AAV,
wherein the recombinant AAV viral particle is capable of infecting a mammalian host, preferably a primate host, and optionally
wherein the at least one of the AAV VP1 capsid protein, any portion of the AAV VP1 capsid protein, the AAV VP2 capsid protein, any portion of the AAV VP2 capsid protein, the AAV VP3 capsid protein, and any portion of the AAV VP3 capsid protein comprising a chimeric amino acid sequence further comprises a modification selected from the group consisting of
(a) a first member of a protein:protein binding pair,
(b) a detectable label, and
(c) a combination of (a) and (b).

5. The recombinant AAV particle of any one of the preceding claims, wherein the protein:protein binding pair is selected from SpyTag:SpyCatcher, SpyTag:KTag, Isopeptag:pilin-C, SnoopTag:SnoopCatcher, and SpyTag002:SpyCatcher002.

6. The recombinant AAV particle of any one of the preceding claims, wherein the first member of a protein:protein binding pair comprises c-myc comprising a sequence set forth as SEQ ID NO:44.

7. The recombinant AAV particle of any one of the preceding claims, wherein the detectable label comprises the B1 epitope comprising an amino acid sequence of IGTRYLTR (SEQ ID NO: 45).

8. The recombinant AAV viral particle of any one of the preceding claims, wherein the capsid protein or portion thereof comprises an amino acid sequence having significant sequence identity, e.g., at least 95% identity, to an amino acid sequence of a VP3 capsid protein of the non-primate animal AAV.

9. The recombinant AAV viral particle of any one of the preceding claims, wherein the capsid protein or portion thereof comprises an amino acid sequence having significant sequence identity, e.g., at least 95% identity, to an amino acid sequence of a VP2 capsid protein of the non-primate animal AAV.

10. The recombinant AAV viral particle of any one of the preceding claims, wherein the capsid protein or portion thereof comprises an amino acid sequence having significant sequence identity, e.g., at least 95% identity, to an amino acid sequence of a VP1 capsid protein of the non-primate animal AAV.

11. The recombinant AAV viral particle of any one of the preceding claims, wherein
(i) the VP1 capsid protein comprises either
a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV, or
an amino acid sequence having significant sequence identity, e.g., at least 95% identity, to the VP1 capsid protein of the non-primate animal AAV,
(ii) the VP2 capsid protein comprises either
a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises at least 95% identity to the VP3 region of the non-primate animal AAV, or
an amino acid sequence having significant sequence identity, e.g., at least 95% identity, to the VP2 capsid protein of the non-primate animal AAV, and
(iii) the VP3 capsid protein comprising an amino acid sequence having significant sequence identity, e.g., at least 95% identity, to an amino acid sequence of the VP3 capsid protein of the non-primate animal AAV.

12. The recombinant AAV viral particle of any one of the preceding claims, wherein
(i) the VP1 capsid protein comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV,
(ii) the VP2 capsid protein comprises a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises at least 95% identity to the VP3 region of the non-primate animal AAV, and
(iii) the VP3 capsid protein comprises an amino acid sequence having significant sequence identity, e.g., at least 95% identity, to an amino acid sequence of the VP3 capsid protein of the non-primate animal AAV.

13. The recombinant AAV viral particle of any one of the preceding claims, wherein
(i) the AAV VP1 capsid protein comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV,
(ii) the VP2 capsid protein comprises an amino acid sequence having significant sequence identity, e.g., at least 95% identity, to an amino acid sequence of the VP2 capsid protein of the non-primate animal AAV, and
(iii) the VP3 capsid protein comprises an amino acid sequence having significant sequence identity, e.g., at least 95% identity, to the amino acid sequence of a VP3 capsid protein of the non-primate animal AAV.

14. The recombinant AAV viral particle of any one of the preceding claims, wherein
(i) the VP1 capsid protein comprises an amino acid sequence having significant sequence identity, e.g., at least 95% identity, to an amino acid sequence of the VP1 capsid protein of the non-primate animal AAV,
(ii) the VP2 capsid protein comprises an amino acid sequence having significant sequence identity, e.g., at least 95% identity, to an amino acid sequence of the VP2 capsid protein of the non-primate animal AAV, and
(iii) the VP3 capsid protein comprises an amino acid sequence having significant sequence identity, e.g., at least 95% identity, to an amino acid sequence of the VP3 capsid protein of the non-primate animal AAV.

15. The recombinant AAV viral particle of any one of the preceding claims, wherein the second AAV is a primate AAV or a combination of primate AAVs.

16. The recombinant AAV viral particle of any one of the preceding claims, wherein the second AAV is a selected from the group consisting of AAV1, AAV2, AAV3, AAV4, AAV5, AAV6, AAV7, AAV8, AAV9, and a combination thereof.

17. The recombinant AAV viral particle of any one of the preceding claims, wherein the second AAV is AAV2.

18. The recombinant AAV viral particle of any one of the preceding claims, wherein the non-primate animal AAV is a non-primate AAV selected from the group listed in Table 2.

19. The recombinant AAV viral particle of any one of the preceding claims, wherein the non-primate animal AAV is an avian AAV (AAAV), a sea lion AAV or a bearded dragon AAV.

20. The recombinant AAV viral particle of any one of the preceding claims, wherein the non-primate animal AAV an AAAV.

21. The recombinant AAV viral particle of any one of the preceding claims, wherein the modification is at position I444 or I580 of a VP1 capsid protein of AAAV.

22. The recombinant AAV viral particle of any one of claims 1-19, wherein the non-primate animal AAV is a squamate AAV.

23. The recombinant AAV viral particle of any one of claims 1-19 and 22, wherein the squamate AAV is a bearded dragon AAV.

24. The recombinant AAV viral particle of any one of claims 1-19 and 22-23, wherein the modification is at position I573 or I436 of a VP1 capsid protein of a bearded dragon AAV.

25. The recombinant AAV viral particle of any one of claims 1-19, wherein the non-primate animal AAV is a mammalian AAV.

26. The recombinant AAV viral particle of any one of claims 1-19 and 25, wherein the mammalian AAV is a sea lion AAV.

27. The recombinant AAV viral particle of any one of claims 1-19 and 25-26, wherein modification is at position selected from the group consisting of I429, I430, I431, I432, I433, I434, I436, I437, and A565 of a VP1 of a sea lion AAV.

28. The recombinant AAV viral particle of any one of the preceding claims, wherein the VP3 capsid protein is modified to comprise
(a) at least a first member of a protein:protein binding pair, optionally wherein the protein:protein binding pair is selected from the group consisting of SpyTag:SpyCatcher, SpyTag:KTag, Isopeptag:pilin-C, SnoopTag:SnoopCatcher, and SpyTag002:SpyCatcher002
(b) a detectable label, optionally wherein the detectable label comprises the amino acid sequence set forth as SEQ ID NO: 44 or the amino acid sequence set forth as SEQ ID NO:45,
(c) a point mutation, or
(d) any combination of (a), (b), and (c).

29. The recombinant AAV viral particle of claim 28, wherein the VP3 capsid protein is modified to comprise
(a) at least a SpyTag comprising an amino acid sequence set forth as SEQ ID NO:42 and/or
(b) a detectable label comprising amino acid sequence set forth SEQ ID NO:45.

30. The recombinant AAV viral particle of any one of the preceding claims, comprising a first and/or second linker operably linking a first member of a protein:protein binding pair and/or a detectable label to a capsid protein of the capsid of said AAV particle.

31. The recombinant AAV viral particle of claim 30, wherein the first and second linker are not identical.

32. The recombinant AAV viral particle of claim 30, wherein the first and second linker are identical.

33. The recombinant AAV viral particle of any one of claims 30-32, wherein the first and/or second linkers is 10 amino acids in length.

34. The recombinant AAV viral particle of any one of the preceding claims, comprising a first member of a protein:protein binding pair and/or a detectable label operably linked to a variable region of a capsid protein of the capsid of said AAV particle.

35. The recombinant AAV viral particle of any one of the preceding claims, comprising a capsid protein comprising an amino acid sequence selected from the group consisting of
(a) an amino acid sequence set forth as SEQ ID NO:2,
(b) an amino acid sequence set forth as SEQ ID NO:4,
(c) an amino acid sequence set forth as SEQ ID NO:6,
(d) an amino acid sequence set forth as SEQ ID NO:8,
(e) an amino acid sequence set forth as SEQ ID NO:10,
(f) an amino acid sequence set forth as SEQ ID NO:12,
(g) an amino acid sequence set forth as SEQ ID NO:14,
(h) an amino acid sequence set forth as SEQ ID NO:16,
(i) an amino acid sequence set forth as SEQ ID NO:18,
(j) an amino acid sequence set forth as SEQ ID NO:20,
(k) an amino acid sequence set forth as SEQ ID NO:22,
(l) an amino acid sequence set forth as SEQ ID NO:24,
(m) an amino acid sequence set forth as SEQ ID NO:26,
(n) an amino acid sequence set forth as SEQ ID NO:28,
(o) an amino acid sequence set forth as SEQ ID NO:30,
(p) an amino acid sequence set forth as SEQ ID NO:32,
(q) an amino acid sequence set forth as SEQ ID NO:34,
(r) an amino acid sequence set forth as SEQ ID NO:36,
(s) the amino acid sequence set forth as SEQ ID NO:53,
(t) the amino acid sequence set forth as SEQ ID NO:55,
(u) the amino acid sequence set forth as SEQ ID NO:57,
(v) the amino acid sequence set forth as SEQ ID NO:59,
(w) the amino acid sequence set forth as SEQ ID NO:61,
(x) the amino acid sequence set forth as SEQ ID NO:63,
(y) the amino acid sequence set forth as SEQ ID NO:65,
(z) the amino acid sequence set forth as SEQ ID NO:67,
(aa) the amino acid sequence set forth as SEQ ID NO:69,
(bb) the amino acid sequence set forth as SEQ ID NO:71,
(cc) an amino acid sequence having significant sequence identity, e.g., at least 95% identity, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71, and
(dd) an amino acid sequence of any VP2 and/or VP3 portions of the amino acid sequences set forth in any of (a)-(cc).

36. The recombinant AAV viral particle of any one of the preceding claims, further comprising a reference capsid protein such that the capsid is a mosaic capsid.

37. The recombinant AAV viral particle of any one of the preceding claims, wherein the VP3 capsid protein comprises a modification with a first member of a protein:protein binding pair, wherein the capsid further comprises a reference VP3 capsid protein lacking the first member of a protein:protein binding pair.

38. An adeno-associated virus (AAV) capsid protein comprising an amino acid sequence, wherein the amino acid sequence or a portion thereof has significant sequence identity, e.g., at least 95% identity, to an amino acid sequence of a capsid protein of a non-primate animal AAV or a portion thereof, or a remote AAV or a portion thereof,
wherein the AAV capsid protein is selected from the group consisting of
(a) a chimeric AAV VP1 capsid protein, optionally wherein the chimeric AAV VP1 capsid protein modified to comprise at least a first member of a protein:protein binding pair, a detectable label and/or a point mutation that reduces the natural tropism of the AAV viral particle and/or creates a detectable label,
(b) a non-chimeric AAV VP1 capsid protein of the non-primate animal AAV or remote AAV modified to comprise at least a first member of a protein:protein binding pair, a detectable label and/or a point mutation that reduces the natural tropism of the AAV viral particle and/or creates a detectable label,
(c) a chimeric VP2 capsid protein, optionally wherein the chimeric AAV VP2 capsid protein is modified to comprise at least a first member of a protein:protein binding pair, a detectable label and/or a point mutation that reduces the natural tropism of the AAV viral particle and/or creates a detectable label,
(d) a non-chimeric AAV VP2 capsid protein of the non-primate animal AAV or remote AAV modified to comprise at least a first member of a protein:protein binding pair, a detectable label and/or a point mutation that reduces the natural tropism of the AAV viral particle and/or creates a detectable label,
(e) a chimeric AAV VP3 capsid protein modified to comprise at least a first member of a protein:protein binding pair, a detectable label and/or a point mutation that reduces the natural tropism of the AAV viral particle and/or creates a detectable label, and
(f) a non-chimeric AAV VP3 capsid protein of the non-human AAV or remote AAV modified to comprise at least a first member of a protein:protein binding pair, a detectable label and/or a point mutation that reduces the natural tropism of the AAV viral particle and/or creates a detectable label.

39. The AAV capsid protein of claim 38, wherein the first member of a protein:protein binding pair is flanked by a first and/or second linker that link(s) the a first member of a protein:protein binding pair to the capsid protein, and wherein the first and/or second linker is each independently at least one amino acid in length.

40. The AAV capsid protein of claim 39, wherein the first and second linkers are not identical.

41. The AAV capsid protein of claim 39, wherein the first and second linkers are identical and are 10 amino acids in length.

42. The AAV capsid protein of any one of claims 38-41, wherein the capsid protein further comprises a second cognate member of the protein:protein binding pair, optionally wherein the first and second members are bound by a covalent bond, optionally an isopeptide bond.

43. The AAV capsid protein of any one of claims 38-42, wherein the first member of a protein:protein binding pair comprises SpyTag.

44. The AAV capsid protein of claim 42 or claim 43, wherein the second cognate member comprises SpyCatcher.

45. The AAV capsid protein of any one of claims 42-44, wherein the second cognate member comprises KTag.

46. The AAV capsid protein of claim 42, wherein the first member is KTag and the second cognate member comprises SpyTag.

47. The AAV capsid protein of claim 42, wherein the first member is SnoopTag and the second cognate member comprises SnoopCatcher.

48. The AAV capsid protein of claim 42, wherein the first member is isopeptag and the second cognate member comprises Pilin-C.

49. The AAV capsid protein of claim 42, wherein the first member is SpyTag002 and the second cognate member comprises SpyCatcher002.

50. The AAV capsid protein of any one of claims 42-49, wherein the second member is operably linked to a targeting ligand, optionally wherein the targeting ligand is a binding moiety.

51. The AAV capsid protein of claim 50, wherein the binding moiety is an antibody, or a portion thereof.

52. The AAV capsid protein of claim 51, wherein the antibody, or portion thereof, is fused to SpyCatcher.

53. The AAV capsid protein of claim 51 or claim 52, wherein the antibody, or portion thereof, is fused to a linker at the C-terminus, and the linker is fused to SpyCatcher at the linker's C-terminus.

54. The AAV capsid protein of claim 53, wherein the linker comprises a sequence set forth as SEQ ID NO:49 (GSGESG).

55. The AAV capsid protein of any one of claims 38-54, wherein the detectable label comprises a B1 epitope comprising an amino acid sequence set forth as SEQ ID NO:45.

56. The AAV capsid protein of any one of claims 38-55, wherein said non-primate animal AAV is a non-primate AAV listed in Table 2.

57. The AAV capsid protein of any one of claims 38-56, wherein the non-primate AAV is an avian AAV (AAAV), a sea lion AAV or a bearded dragon AAV.

58. The AAV capsid protein of any one of claims 38-57, wherein the non-primate animal AAV is an AAAV.

59. The AAV capsid protein of any one of claims 38-58, wherein the modification is at position I444 or I580 of a VP1 capsid protein of AAAV.

60. The AAV capsid protein of any one of claims 38-56, wherein the non-primate animal AAV is a squamate AAV.

61. The AAV capsid protein of any one of claims 38-56 and 60, wherein the squamate AAV is a bearded dragon AAV.

62. The AAV capsid protein of any one of claims 38-56 and 60-61, wherein the modification is at position I573 or I436 of a VP1 capsid protein of a bearded dragon AAV.

63. The AAV capsid protein of any one of claims 38-56, wherein the non-primate animal AAV is a mammalian AAV.

64. The AAV capsid protein of any one of claims 38-56 and 63, wherein the mammalian AAV is a sea lion AAV.

65. The AAV capsid protein of any one of claims 38-56 and 63-64, wherein modification is at position selected from the group consisting of I429, I430, I431, I432, I433, I434, I436, I437, and A565 of a VP1 of a sea lion AAV.

66. The AAV capsid protein of any one of claims 38-65, comprising an amino acid sequence selected from the group consisting of
(a) an amino acid sequence set forth as SEQ ID NO:2,
(b) an amino acid sequence set forth as SEQ ID NO:4,
(c) an amino acid sequence set forth as SEQ ID NO:6,
(d) an amino acid sequence set forth as SEQ ID NO:8,
(e) an amino acid sequence set forth as SEQ ID NO:10,
(f) an amino acid sequence set forth as SEQ ID NO:12,
(g) an amino acid sequence set forth as SEQ ID NO:14,
(h) an amino acid sequence set forth as SEQ ID NO:16,
(i) an amino acid sequence set forth as SEQ ID NO:18,
(j) an amino acid sequence set forth as SEQ ID NO:20,
(k) an amino acid sequence set forth as SEQ ID NO:22,
(l) an amino acid sequence set forth as SEQ ID NO:24,
(m) an amino acid sequence set forth as SEQ ID NO:26,
(n) an amino acid sequence set forth as SEQ ID NO:28,
(o) an amino acid sequence set forth as SEQ ID NO:30,
(p) an amino acid sequence set forth as SEQ ID NO:32,
(q) an amino acid sequence set forth as SEQ ID NO:34,
(r) an amino acid sequence set forth as SEQ ID NO:36,
(s) the amino acid sequence set forth as SEQ ID NO:53,
(t) the amino acid sequence set forth as SEQ ID NO:55,
(u) the amino acid sequence set forth as SEQ ID NO:57,
(v) the amino acid sequence set forth as SEQ ID NO:59,
(w) the amino acid sequence set forth as SEQ ID NO:61,
(x) the amino acid sequence set forth as SEQ ID NO:63,
(y) the amino acid sequence set forth as SEQ ID NO:65,
(z) the amino acid sequence set forth as SEQ ID NO:67,
(aa) the amino acid sequence set forth as SEQ ID NO:69,
(bb) the amino acid sequence set forth as SEQ ID NO:71,
(cc) an amino acid sequence having significant sequence identity, e.g., at least 95% identity, to SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24, SEQ ID NO:26, SEQ ID NO:28, SEQ ID NO:30, SEQ ID NO:32, SEQ ID NO:34, SEQ ID NO:36, SEQ ID NO:53, SEQ ID NO:55, SEQ ID NO:57, SEQ ID NO:59, SEQ ID NO:61, SEQ ID NO:63, SEQ ID NO:65, SEQ ID NO:67, SEQ ID NO:69, or SEQ ID NO:71, and
(dd) an amino acid sequence of any VP2 and/or VP3 portions of the amino acid sequences set forth in any of (a)-(cc).

67. The AAV capsid protein of any one of 38-66, wherein the first member of a protein:protein binding pair comprises a detectable label.

68. The AAV capsid protein of claim 67, wherein the detectable label comprises c-myc (SEQ ID NO:44).

69. An AAV particle comprising the AAV capsid protein of any one of claims 38-68.

70. A nucleic acid molecule comprising a cap gene encoding the AAV capsid protein of any one of claims 38-68.

71. A nucleic acid molecule comprising an AAV cap gene that encodes an AAV VP1 capsid protein, an AAV VP2 capsid protein and/or an AAV VP3 capsid protein,
wherein the AAV cap gene, or portion thereof, comprises a nucleic acid sequence having significant sequence identity, e.g., at least 95% identity, to the nucleic acid sequence of a cap gene of a non-primate animal AAV or portion thereof, or a remote AAV or a portion thereof, and
wherein the AAV cap gene is further modified to comprise
(a) a nucleotide sequence encoding a first member of a protein:protein binding pair,
(b) a nucleotide sequence encoding a detectable label,
(c) a point mutation,
(d) a chimeric nucleotide sequence, or
(e) any combination of (a), (b), (c), and (d).

72. A nucleic acid molecule comprising an AAV rep gene and an AAV cap gene,
wherein the entire AAV cap gene comprises a first nucleic acid sequence having significant sequence identity, e.g., at least 95% identity, to the nucleic acid sequence of a cap gene of a non-primate animal AAV or a remote AAV, and
wherein the AAV rep gene, or portion thereof, comprises a second nucleic acid sequence having significant sequence identity, e.g., at least 95% identity, to the nucleic acid sequence of a rep gene of a second AAV, or portion thereof
wherein the non-primate animal AAV is not identical to the second AAV.

73. The nucleic acid molecule of any one of claims 70-72, wherein the cap gene is operably linked to a promoter.

74. The nucleic acid molecule of claim 73, wherein said promoter directs the expression of the capsid protein(s) in a packaging cell.

75. The nucleic acid molecule of claim 73 or claim 74, wherein the promoter is selected from p40, SV40, EF, CMV, B19p6, and CAG.

76. The nucleic acid molecule of any one of claims 70-75, wherein the AAV cap gene is modified to comprise
(a) a nucleotide sequence encoding at least a first member of a protein:protein binding pair,
(b) a nucleotide sequence encoding a detectable label, and/or
(c) a nucleotide sequence encoding a point mutation.

77. The nucleic acid molecule of claim 76, wherein the protein:protein binding pair is selected from SpyTag:SpyCatcher, SpyTag:KTag, Isopeptag:pilin-C, SnoopTag:SnoopCatcher, and SpyTag002:SpyCatcher002.

78. The nucleic acid molecule of claim 76, wherein the first member of a protein:protein binding pair comprises c-myc comprising a sequence set forth as SEQ ID NO:44.

79. The nucleic acid molecule of any one of claims 76-78, wherein the detectable label comprises the B1 epitope comprising an amino acid sequence of IGTRYLTR (SEQ ID NO: 45).

80. The nucleic acid molecule of any one of claims 70-79, wherein the cap gene encodes a VP3 capsid protein, wherein the VP3 capsid protein, or portion thereof, comprises an amino acid sequence having significant sequence identity, e.g., at least 95% identity, to the amino acid sequence of a VP3 capsid protein of the non-primate animal AAV.

81. The nucleic acid molecule of any one of claims 70-80, wherein the cap gene encodes a VP2 capsid protein, wherein the VP2 capsid protein, or portion thereof, comprises an amino acid sequence having significant sequence identity, e.g., at least 95% identity, to the amino acid sequence of a VP2 capsid protein of the non-primate animal AAV.

82. The nucleic acid molecule of any one of claims 70-81, wherein the cap gene encodes a VP1 capsid protein, wherein the VP1 capsid protein, or portion thereof, comprises an amino acid sequence having significant sequence identity, e.g., at least 95% identity, to the amino acid sequence of a VP1 capsid protein of the non-primate animal AAV.

83. The nucleic acid molecule of any one of claims 70-82, wherein cap gene encodes
(i) a VP1 capsid protein that comprises either
a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV, or
an amino acid sequence having significant sequence identity, e.g., at least 95% identity, to the VP1 capsid protein of the non-primate animal AAV,
(ii) a VP2 capsid protein that comprises either
a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises an amino acid sequence having significant sequence identity, e.g., at least 95% identity to the VP3 region of the non-primate animal AAV, or
an amino acid sequence having significant sequence identity, e.g., at least 95% identity, to the VP2 capsid protein of the non-primate animal AAV, and/or
(iii) a VP3 capsid protein comprising an amino acid sequence having significant sequence identity, e.g., at least 95% identity, to an amino acid sequence of the VP3 capsid protein of the non-primate animal AAV.

84. The nucleic acid molecule of any one of claims 70-83, wherein cap gene encodes
(i) a VP1 capsid protein that comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV,
(ii) a VP2 capsid protein that comprises a chimeric amino acid sequence, optionally wherein the VP1/VP2 common region of the chimeric AAV VP2 capsid protein comprises an amino acid sequence having significant sequence identity, e.g., at least 95% identity, to the amino acid sequence of the VP1/VP2 common region of a second AAV and wherein the VP3 region of the chimeric VP2 capsid protein comprises an amino acid sequence having significant sequence identity, e.g., at least 95% identity, to the amino acid sequence of a VP3 region of the non-primate animal AAV, and/or
(iii) a VP3 capsid protein comprising an amino acid sequence having significant sequence identity, e.g., at least 95% identity, to an amino acid sequence of the VP3 capsid protein of the non-primate animal AAV.

85. The nucleic acid molecule of any one of claims 70-84, wherein cap gene encodes
(i) a VP1 capsid protein that comprises a chimeric amino acid sequence, optionally wherein the VP1-unique region (VP1-u) of the chimeric AAV VP1 capsid protein comprises an amino acid sequence having significant sequence identity, e.g., at least 95% identity, to the amino acid sequence of the VP1-u of a second AAV and wherein the VP1/VP2 common region and the VP3 region of the chimeric AAV VP1 capsid comprises an amino acid sequence having significant sequence identity, e.g., at least 95% identity, to the amino acid sequence of the VP1/VP2 comment region and VP3 region of the non-primate animal AAV,
(ii) a VP2 capsid protein that comprises an amino acid sequence having significant sequence identity, e.g., at least 95% identity, to the VP2 capsid protein of the non-primate animal AAV, and
(iii) a VP3 capsid protein comprising an amino acid sequence having significant sequence identity, e.g., at least 95% identity, to an amino acid sequence of the VP3 capsid protein of the non-primate animal AAV.

86. The nucleic acid molecule of any one of claims 70-83, wherein cap gene encodes
(i) a VP1 capsid protein comprising an amino acid sequence having significant sequence identity, e.g., at least 95% identity, to the VP1 capsid protein of the non-primate animal AAV,
(ii) a VP2 capsid protein that comprises an amino acid sequence having significant sequence identity, e.g., at least 95% identity, to the VP2 capsid protein of the non-primate animal AAV, and
(iii) a VP3 capsid protein comprising an amino acid sequence having significant sequence identity, e.g., at least 95% identity, to an amino acid sequence of the VP3 capsid protein of the non-primate animal AAV.

87. The nucleic acid molecule of any one of claims 70-86, wherein the second AAV is a primate AAV or a combination of primate AAVs.

88. The nucleic acid molecule of any one of claims 70-87, wherein the second AAV is a selected from the group consisting of AAV1, AAV2, AAV3, AAV4, AAV5, AAV6, AAV7, AAV8, AAV9, and a combination thereof.

89. The nucleic acid molecule of any one of claims 70-88, wherein the second AAV is AAV2.

90. The nucleic acid molecule of any one of claims 70-89, wherein the non-primate animal AAV is a non-primate AAV selected from the non-primate AAV listed in Table 2.

91. The nucleic acid molecule of any one of claims 70-90, wherein the non-primate AAV is an avian AAV, a sea lion AAV or a bearded dragon AAV.

92. The nucleic acid molecule of any one of claims 70-91, wherein the non-primate animal AAV is an AAAV.

93. The nucleic acid molecule of any one of claims 70-92, wherein the modification is at a codon encoding position I444 or I580 of a VP1 capsid protein of AAAV.

94. The nucleic acid molecule of any one of claims 70-91, wherein the non-primate animal AAV is a squamate AAV.

95. The nucleic acid molecule of any one of claims 70-91 and 94, wherein the squamate AAV is a bearded dragon AAV.

96. The nucleic acid molecule of any one of claims 70-91 and 94-95, wherein the modification is at a codon encoding position I573 or I436 of a VP1 capsid protein of a bearded dragon AAV.

97. The nucleic acid molecule of any one of claims 70-91, wherein the non-primate animal AAV is a mammalian AAV.

98. The nucleic acid molecule of any one of claims 70-91 and 97, wherein the mammalian AAV is a sea lion AAV.

99. The nucleic acid molecule of any one of claims 70-91 and 97-98, wherein modification is at position selected from the group consisting of I429, I430, I431, I432, I433, I434, I436, I437, and A565 of a VP1 of a sea lion AAV.

100. The nucleic acid molecule of any one of claims 70-99, comprising a nucleotide sequence selected from the group consisting of
(a) the nucleotide sequence set forth as SEQ ID NO:1,
(b) the nucleotide sequence set forth as SEQ ID NO:3,
(c) the nucleotide sequence set forth as SEQ ID NO:5,
(d) the nucleotide sequence set forth as SEQ ID NO:7,
(e) the nucleotide sequence set forth as SEQ ID NO:9,
(f) the nucleotide sequence set forth as SEQ ID NO:11,
(g) the nucleotide sequence set forth as SEQ ID NO:13,
(h) the nucleotide sequence set forth as SEQ ID NO:15,
(i) the nucleotide sequence set forth as SEQ ID NO:17,
(j) the nucleotide sequence set forth as SEQ ID NO:19,
(k) the nucleotide sequence set forth as SEQ ID NO:21,
(l) the nucleotide sequence set forth as SEQ ID NO:23,
(m) the nucleotide sequence set forth as SEQ ID NO:25,
(n) the nucleotide sequence set forth as SEQ ID NO:27,
(o) the nucleotide sequence set forth as SEQ ID NO:29,
(p) the nucleotide sequence set forth as SEQ ID NO:31,
(q) the nucleotide sequence set forth as SEQ ID NO:33,
(r) the nucleotide sequence set forth as SEQ ID NO:35,
(s) the nucleotide sequence set forth as SEQ ID NO:52,
(t) the nucleotide sequence set forth as SEQ ID NO:54
(u) the nucleotide sequence set forth as SEQ ID NO:56
(v) the nucleotide sequence set forth as SEQ ID NO:58
(w) the nucleotide sequence set forth as SEQ ID NO:60
(x) the nucleotide sequence set forth as SEQ ID NO:62
(y) the nucleotide sequence set forth as SEQ ID NO:64
(z) the nucleotide sequence set forth as SEQ ID NO:66
(aa) the nucleotide sequence set forth as SEQ ID NO:68
(bb) the nucleotide sequence set forth as SEQ ID NO:70
(cc) a nucleotide sequence having significant sequence identity, e.g., at least 95% identity, to the nucleotide sequence set forth as SEQ ID NO: 1, 3, 5, 7, 9, 11, 13, 15, 17, 19, 21, 23, 25, 27, 29, 31, 33, 35, 52, 54, 56, 58, 60, 62, 64, 66, 68, 70, or any thereof encoding a VP2 capsid,
(dd) any portion of the nucleotide sequence of (a)-(s) encoding a VP2 capsid protein and/or a VP3 capsid protein.

101. The nucleic acid molecule of any one of claims 70-100, further comprising an AAV rep gene that encodes one or more AAV Rep proteins and is operably linked to a promoter.

102. The nucleic acid molecule of claim 101, wherein the promoter is selected from the group consisting of p5, p19 SV40, EF, CMV, B19p6, and CAG.

103. The nucleic acid molecule of claim 101 or claim 102, wherein the one or more Rep proteins are selected from Rep78, Rep68, Rep52 and Rep40.

104. The nucleic acid molecule of any one of claims 101-103, wherein the one or more Rep proteins comprises Rep78.

105. The nucleic acid molecule of any one of claims 101-104, wherein the one or more Rep proteins are primate animal AAV Rep proteins.

106. An AAV capsid protein comprising an amino acid sequence encoded by the nucleic acid molecule of one of claims 70-105.

107. An AAV particle comprising the capsid protein of claim 106.

108. A packaging cell for producing AAV particles comprising the nucleic acid molecule of any one of claims 70-105.

109. The packaging cell of claim 108, comprising a nucleic acid molecule comprising a rep gene encoding one or more AAV Rep proteins, wherein said rep gene is operably linked to a promoter, optionally wherein the rep gene and the cap gene are of two different AAV.

110. The packaging cell of claim 109, wherein the promoter operably linked to the rep gene directs the expression of the Rep protein(s) in the packaging cell.

111. The packaging cell of claim 109 or claim 110, wherein the promoter is selected from p5, p19 SV40, EF, CMV, B19p6, and CAG.

112. The packaging cell of any one of claims 109-111, wherein the one or more Rep proteins are selected from Rep78, Rep68, Rep52 and Rep40.

113. The packaging cell of any one of claims 109-112, wherein the one or more Rep proteins comprises Rep78.

114. The packaging cell of any one of claims 109-113, wherein the one or more Rep proteins are primate animal AAV Rep proteins.

115. The packaging cell of any one of claims 109-114, wherein the one or more Rep proteins are non-primate animal AAV Rep proteins.

116. The packaging cell of any one of claims 109-115, further comprising a nucleic acid molecule comprising a nucleotide sequence of a nucleotide of interest flanked on at least one side by at least one AAV inverted terminal repeat (ITR) that is recognized by the one or more Rep proteins.

117. The packaging cell of claim 116, wherein the nucleotide is flanked on the other side by a second ITR of the same AAV as the at least one ITR.

118. The packaging cell of claim 117, wherein the nucleotide is flanked on the other side by a second ITR, wherein the second ITR and the at least one ITR are of different AAV.

119. The packaging cell of any one of claims 116-118, wherein the nucleotide of interest is a reporter gene.

120. The packaging cell of claim 119, wherein the reporter gene encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

121. The packaging cell of any one of claims 116-118, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

122. The packaging cell of any one of claims 108-121, further comprising a nucleotide sequence encoding a reference capsid protein.

123. A method of producing a viral particle comprising culturing a packaging cell according to any one of claims 108-122 in conditions sufficient for the production of viral particles.

124. The method of claim 123, wherein the packaging cell further comprises a helper plasmid and/or a transfer plasmid comprising a nucleotide of interest.

125. The method of claim 123 or claim 124, further comprising one or more of the following steps:
a. clearing cell debris,
b. treating the supernatant containing viral particles with Benzonase or DNase I and MgCl2,
c. concentrating viral particles,
d. purifying the viral particles, and
e. any combination of a.-d,
optionally wherein the viral particles are self-complementary adeno-associated viral particles and/or isolated from culture supernatant.

126. An AAV particle made according to the method of any one of claims 123-125.

127. An AAV particle according to any one of claims 1-37, 69, 107, and 126, comprising a nucleotide of interest.

128. The AAV particle of claim 127, wherein the nucleotide of interest is a reporter gene.

129. The AAV particle of claim 128, wherein the reporter gene encodes β-galactosidase, green fluorescent protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof.

130. The AAV particle of claim 127, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.

131. A pharmaceutical composition comprising (a) an AAV particle according to any one of claims 1-37, 69, 107, and 126-130, an AAV particle comprising an AAV capsid protein according to any one of claims 38-68 and 106, or an AAV particle made according to the method of any one of claims 123-125, and (b) a pharmaceutically acceptable carrier or excipient.

132. A method of delivering a nucleotide of interest to a target cell comprising contacting the target cell with (a) the AAV particle according to any one of claims 1-37, 69, 107, and 126-130, the AAV particle comprising an AAV capsid protein according to any one of claims 38-68 and 106, or the AAV particle made according to the method of any one of claims 123-125, or (b) the composition of claim 131.

133. The method of claim 132, wherein the capsid of the AAV particle comprises a targeting ligand that specifically binds a protein expressed on the surface the target cell.

134. The method of claim 132 or claim 133, wherein the contacting is performed ex vivo.

135. The method of claim 132 or claim 133, wherein the target cell is in a subject.

136. The method of claim 135, wherein the subject is a primate animal, preferably a human.

137. The method of any one of claims 132-136, wherein the target cell is a human cell.

138. The method of any one of claims 132-137, wherein the nucleotide of interest encodes a therapeutic protein, a suicide gene, an antibody or a fragment thereof, a CRISPR/Cas system or a portion(s) thereof, an antisense oligonucleotide, a ribozyme, an RNAi molecule, or a shRNA molecule.


SEQUENCE LISTING (73 sequences)

SEQ ID NO: 1 - DNA, 2190 bp, Artificial Sequence
  Cap AAV2 VP1 AAAV VP2 VP3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgggaccggggagaagcgtcccgaacgcgtcgacgactttttcccgaaaaagaagaaggccaagaccgagcaaggcaaagcccctgctcaaacgggcgaagaccccggagaaggaacctcttccaacgctggatcaagcgccccctctagtgtgggatcatctgtcatggctgaaggaggtggcggtccaatgggcgatgcaggccaaggtgccgacggagtgggcaattcctcgggaaattggcattgcgattcccaatggctggacaacggagtcgttacccgaaccactcgaacctgggtcctgcccagctacaacaaccacttgtacaagcggatccaaggaccgggaggaaccgaccccaacaataaattctttggattcagcaccccctgggggtactttgactacaaccgattccactgccacttctccccccgagactggcaacgactcatcaacaacaactggggcatccgacccaaagcgatgcgctttagactctttaacatccaggttaaagaagtcactgtccaagactccaacaccaccatcgccaacaacctcaccagcacggtccaagtctttgcggacaaggactaccagctgccgtacgtcctcggatcggctacagagggcaccttcccgccgttcccagcggatatctacacgatcccgcagtatggttactgcacgctaaactacaacaacgaggcggtggatcgttcggccttctactgtctagactatttcccctcagacatgctgcggacaggaaataactttgaattcacttacacgttcgaggacgttcctttccatagcatgtttgctcacaaccagacgctagaccggctgatgaatcctctcgtcgatcagtacctgtgggctttcagttccgtcagccaaacaggctcgtctggacgggcactcaattattcacgcgcgaccaaaaccaatatggcaacccagtacagaaactggttacctggacccttcgtccgggatcagcaaatctttacgggggctagcaacatcacccaaaacaacgtgttcaacgtttgggataaaggcaagcagtgggtgatagacaatcggatcaatatgatgcagcccggccctgcagcagcgaccacctttagcggagaacccgaccgtcaagccatgcaaaacacgctggcctttagtcggacggtctacgaccagacaaccagtacgaccgatcgtaaccagttgctcattaccaacgaagatgaaatcagacccaccaactcggtcggcatcgacacgtggggagtagttcccaacaacaaccagtccaaggtgaccgccggcactcgcgcggccatcaacaaccaaggggcgcttcccgggatggtgtggcaaaacagagacatttacctccaaggacccatttgggccaaaatccccgacacagacaatcacttccatccgtccccgcttattggcgggtttggctgcaagcatccccctccccagattttcattaaaaacacacccgtcccggccaacccttcggaaacgttccagacggccaaggtggcctccttcatcaaccagtactcgaccggacagtgcaccgtcgaaatcttttgggaactcaagaaggaaacctccaagcgctggaaccccgaaatccagttcacctccaactttggcaacgcggccgacatccagtttgctgtctccgacaccggatcctattccgaacctcgtcccattggcaccagatacctgactcgtcctctgtaa

SEQ ID NO: 2 - protein, 729 aa, Artificial Sequence
  Cap AAV2 VP1 AAAV VP2 VP3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTRPL

SEQ ID NO: 3 - DNA, 2145 bp, Artificial Sequence
  Cap AAV2 VP1 Sea Lion VP2 VP3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 4 - protein, 714 aa, Artificial Sequence
  Cap AAV2 VP1 Sea Lion VP2 VP3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 5 - DNA, 2187 bp, Artificial Sequence
  Cap AAV2 VP1 Dragon VP2 VP3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgaaaagcgaaagacacccgaagagtggttagctcaagaaaagactccaaccaaacaaaggttccagataccagctccaggacaatctggatcagattctccttccacctcaggatccggcggtactgcaggctccagttctagcgcatcaaatacaatggctcaaggaggtggcggaccaatggcagacgataaccaaggcgccgagggagtgggtaatgcctcgggagattggcattgcgatacccaatggctgggcgaccacgtcattacaaaatctaccagaacttgggttctgccctcttacgggaatcatctctactcgcccatcaactttgatggaaccacagggaacggaacccaagccgcttactgcggatacgctaccccctgggcctactttgactttaaccgattccactgccacttttccccccgagactggcaaagactcattaacaaccataccggaatacgaccagtcggactcaaattcaagctgttcaacatccaggtcaaggaaatcacagtacaagattcgaccaaaacgatcgccaacaatctcaccagcaccgtacaggtctttgcggacacggagcaccagctcccgtacgtattaggaaatgccacgcagggcacgtttcctccctttccggctgaagtctttcagttgcctcagtacggctactgtaccatccaaggccctcagggtaagtttaccgacagaagtgccttctactgcttggagtactttccttccaagatgctgagaacggggaataactttgaatttacttacaagttcgagaaggttcccttccattctggctgggctcaaagccagtcgttggatcgattgatgaatcccttgatcccacagtacctgttggccgattatggtacgaccgccagtagtgccataacatactatcggccaaacagcacagacttgagctggtactttaaaaactggctgcctgggccagtggaaaggcgacagcaaatcaattctgaagactcgacgaaaaaccatgccaatttgaacgggaatgcacacaccaacaaatacagcatccagcacagacaaaccaagatgatgccaggggtggctctcagtagtaaatacacaggtgctgctgaaggaacgtcgttactgaacggggtattgacctttgataagatcgccaatgacaatactaacatcacggatacaaataatgtcaatcggacgatcgaagatgaaattcaaggtaccaatccttatggaaacgacgtacccattaccgtcgctgtcaatacacaaaacgcgaccacttctcctaccatgcaaaacagcagcacgtatgaactattacctggtgctgtctggtccaacagagatatttacctgcaaggtcccatctgggctaaaatacccgataccgatggacactttcacccgtctccacaaatgggtggtttcggacttaaaaacccaccaccgatgatcctcatcaaaaacacacccgttcccgccgatccaccaacgaccttcaaaccgactccgatgaacagcttcatcagcgaatacagtaccggtcaggtgactgtagagatgctgtgggaggtccggaaagaagactccaagagatggaacccggaaatccagtttacctccaattttggattgtccgatcctcagatcgacggtataccctttggtattaccaaattgggtaattacgaggaaccacgtcctattggcaccagatacctgactcgtcatttgtaa

SEQ ID NO: 6 - protein, 728 aa, Artificial Sequence
  Cap AAV2 VP1 Dragon VP2 VP3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGTRYLTRHL

SEQ ID NO: 7 - DNA, 2265 bp, Artificial Sequence
  Cap AAV2 VP1 AAAV VP2 VP3 G444 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgggaccggggagaagcgtcccgaacgcgtcgacgactttttcccgaaaaagaagaaggccaagaccgagcaaggcaaagcccctgctcaaacgggcgaagaccccggagaaggaacctcttccaacgctggatcaagcgccccctctagtgtgggatcatctgtcatggctgaaggaggtggcggtccaatgggcgatgcaggccaaggtgccgacggagtgggcaattcctcgggaaattggcattgcgattcccaatggctggacaacggagtcgttacccgaaccactcgaacctgggtcctgcccagctacaacaaccacttgtacaagcggatccaaggaccgggaggaaccgaccccaacaataaattctttggattcagcaccccctgggggtactttgactacaaccgattccactgccacttctccccccgagactggcaacgactcatcaacaacaactggggcatccgacccaaagcgatgcgctttagactctttaacatccaggttaaagaagtcactgtccaagactccaacaccaccatcgccaacaacctcaccagcacggtccaagtctttgcggacaaggactaccagctgccgtacgtcctcggatcggctacagagggcaccttcccgccgttcccagcggatatctacacgatcccgcagtatggttactgcacgctaaactacaacaacgaggcggtggatcgttcggccttctactgtctagactatttcccctcagacatgctgcggacaggaaataactttgaattcacttacacgttcgaggacgttcctttccatagcatgtttgctcacaaccagacgctagaccggctgatgaatcctctcgtcgatcagtacctgtgggctttcagttccgtcagccaaacaggcggcctgagcggcagcggcgcccacatcgtgatggtggacgcctacaagccgacgaagggcctgagcggcagcggctcgtctggacgggcactcaattattcacgcgcgaccaaaaccaatatggcaacccagtacagaaactggttacctggacccttcgtccgggatcagcaaatctttacgggggctagcaacatcacccaaaacaacgtgttcaacgtttgggataaaggcaagcagtgggtgatagacaatcggatcaatatgatgcagcccggccctgcagcagcgaccacctttagcggagaacccgaccgtcaagccatgcaaaacacgctggcctttagtcggacggtctacgaccagacaaccagtacgaccgatcgtaaccagttgctcattaccaacgaagatgaaatcagacccaccaactcggtcggcatcgacacgtggggagtagttcccaacaacaaccagtccaaggtgaccgccggcactcgcgcggccatcaacaaccaaggggcgcttcccgggatggtgtggcaaaacagagacatttacctccaaggacccatttgggccaaaatccccgacacagacaatcacttccatccgtccccgcttattggcgggtttggctgcaagcatccccctccccagattttcattaaaaacacacccgtcccggccaacccttcggaaacgttccagacggccaaggtggcctccttcatcaaccagtactcgaccggacagtgcaccgtcgaaatcttttgggaactcaagaaggaaacctccaagcgctggaaccccgaaatccagttcacctccaactttggcaacgcggccgacatccagtttgctgtctccgacaccggatcctattccgaacctcgtcccattggcaccagatacctgactcgtcctctgtaa

SEQ ID NO: 8 - protein, 754 aa, Artificial Sequence
  Cap AAV2 VP1 AAAV VP2 VP3 G444 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGGLSGSGAHIVMVDAYKPTKGLSGSGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTRPL

SEQ ID NO: 9 - DNA, 2265 bp, Artificial Sequence
  Cap AAV2 VP1 AAAV VP2 VP3 K580 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgggaccggggagaagcgtcccgaacgcgtcgacgactttttcccgaaaaagaagaaggccaagaccgagcaaggcaaagcccctgctcaaacgggcgaagaccccggagaaggaacctcttccaacgctggatcaagcgccccctctagtgtgggatcatctgtcatggctgaaggaggtggcggtccaatgggcgatgcaggccaaggtgccgacggagtgggcaattcctcgggaaattggcattgcgattcccaatggctggacaacggagtcgttacccgaaccactcgaacctgggtcctgcccagctacaacaaccacttgtacaagcggatccaaggaccgggaggaaccgaccccaacaataaattctttggattcagcaccccctgggggtactttgactacaaccgattccactgccacttctccccccgagactggcaacgactcatcaacaacaactggggcatccgacccaaagcgatgcgctttagactctttaacatccaggttaaagaagtcactgtccaagactccaacaccaccatcgccaacaacctcaccagcacggtccaagtctttgcggacaaggactaccagctgccgtacgtcctcggatcggctacagagggcaccttcccgccgttcccagcggatatctacacgatcccgcagtatggttactgcacgctaaactacaacaacgaggcggtggatcgttcggccttctactgtctagactatttcccctcagacatgctgcggacaggaaataactttgaattcacttacacgttcgaggacgttcctttccatagcatgtttgctcacaaccagacgctagaccggctgatgaatcctctcgtcgatcagtacctgtgggctttcagttccgtcagccaaacaggctcgtctggacgggcactcaattattcacgcgcgaccaaaaccaatatggcaacccagtacagaaactggttacctggacccttcgtccgggatcagcaaatctttacgggggctagcaacatcacccaaaacaacgtgttcaacgtttgggataaaggcaagcagtgggtgatagacaatcggatcaatatgatgcagcccggccctgcagcagcgaccacctttagcggagaacccgaccgtcaagccatgcaaaacacgctggcctttagtcggacggtctacgaccagacaaccagtacgaccgatcgtaaccagttgctcattaccaacgaagatgaaatcagacccaccaactcggtcggcatcgacacgtggggagtagttcccaacaacaaccagtccaagggcctgagcggcagcggcgcccacatcgtgatggtggacgcctacaagccgacgaagggcctgagcggcagcggcgtgaccgccggcactcgcgcggccatcaacaaccaaggggcgcttcccgggatggtgtggcaaaacagagacatttacctccaaggacccatttgggccaaaatccccgacacagacaatcacttccatccgtccccgcttattggcgggtttggctgcaagcatccccctccccagattttcattaaaaacacacccgtcccggccaacccttcggaaacgttccagacggccaaggtggcctccttcatcaaccagtactcgaccggacagtgcaccgtcgaaatcttttgggaactcaagaaggaaacctccaagcgctggaaccccgaaatccagttcacctccaactttggcaacgcggccgacatccagtttgctgtctccgacaccggatcctattccgaacctcgtcccattggcaccagatacctgactcgtcctctgtaa

SEQ ID NO: 10 - protein, 754 aa, Artificial Sequence
  Cap AAV2 VP1 AAAV VP2 VP3 K580 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAGTGEKRPERVDDFFPKKKKAKTEQGKAPAQTGEDPGEGTSSNAGSSAPSSVGSSVMAEGGGGPMGDAGQGADGVGNSSGNWHCDSQWLDNGVVTRTTRTWVLPSYNNHLYKRIQGPGGTDPNNKFFGFSTPWGYFDYNRFHCHFSPRDWQRLINNNWGIRPKAMRFRLFNIQVKEVTVQDSNTTIANNLTSTVQVFADKDYQLPYVLGSATEGTFPPFPADIYTIPQYGYCTLNYNNEAVDRSAFYCLDYFPSDMLRTGNNFEFTYTFEDVPFHSMFAHNQTLDRLMNPLVDQYLWAFSSVSQTGSSGRALNYSRATKTNMATQYRNWLPGPFVRDQQIFTGASNITQNNVFNVWDKGKQWVIDNRINMMQPGPAAATTFSGEPDRQAMQNTLAFSRTVYDQTTSTTDRNQLLITNEDEIRPTNSVGIDTWGVVPNNNQSKGLSGSGAHIVMVDAYKPTKGLSGSGVTAGTRAAINNQGALPGMVWQNRDIYLQGPIWAKIPDTDNHFHPSPLIGGFGCKHPPPQIFIKNTPVPANPSETFQTAKVASFINQYSTGQCTVEIFWELKKETSKRWNPEIQFTSNFGNAADIQFAVSDTGSYSEPRPIGTRYLTRPL

SEQ ID NO: 11 - DNA, 2220 bp, Artificial Sequence
  Cap AAV2 VP1 Sea Lion VP2 VP3 G432 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggaggcctgagcggcagcggcgcccacatcgtgatggtggacgcctacaagccgacgaagggcctgagcggcagcggctcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 12 - protein, 739 aa, Artificial Sequence
  Cap AAV2 VP1 Sea Lion VP2 VP3 G432 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGGLSGSGAHIVMVDAYKPTKGLSGSGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 13 - DNA, 2220 bp, Artificial Sequence
  Cap AAV2 VP1 Sea Lion VP2 VP3 A565 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaggcctgagcggcagcggcgcccacatcgtgatggtggacgcctacaagccgacgaagggcctgagcggcagcggcacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 14 - protein, 739 aa, Artificial Sequence
  Cap AAV2 VP1 Sea Lion VP2 VP3 A565 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNAGLSGSGAHIVMVDAYKPTKGLSGSGTTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 15 - DNA, 2220 bp, Artificial Sequence
  Cap AAV2 VP1 Sea Lion VP2 VP3 N429 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacggcctgagcggcagcggcgcccacatcgtgatggtggacgcctacaagccgacgaagggcctgagcggcagcggcccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 16 - protein, 739 aa, Artificial Sequence
  Cap AAV2 VP1 Sea Lion VP2 VP3 N429 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNGLSGSGAHIVMVDAYKPTKGLSGSGPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 17 - DNA, 2220 bp, Artificial Sequence
  Cap AAV2 VP1 Sea Lion VP2 VP3 P430 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgggcctgagcggcagcggcgcccacatcgtgatggtggacgcctacaagccgacgaagggcctgagcggcagcggcactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 18 - protein, 739 aa, Artificial Sequence
  Cap AAV2 VP1 Sea Lion VP2 VP3 P430 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPGLSGSGAHIVMVDAYKPTKGLSGSGTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 19 - DNA, 2220 bp, Artificial Sequence
  Cap AAV2 VP1 Sea Lion VP2 VP3 T431 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggcctgagcggcagcggcgcccacatcgtgatggtggacgcctacaagccgacgaagggcctgagcggcagcggcggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 20 - protein, 739 aa, Artificial Sequence
  Cap AAV2 VP1 Sea Lion VP2 VP3 T431 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGLSGSGAHIVMVDAYKPTKGLSGSGGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 21 - DNA, 2220 bp, Artificial Sequence
  Cap AAV2 VP1 Sea Lion VP2 VP3 S433 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaggcctgagcggcagcggcgcccacatcgtgatggtggacgcctacaagccgacgaagggcctgagcggcagcggcacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 22 - protein, 739 aa, Artificial Sequence
  Cap AAV2 VP1 Sea Lion VP2 VP3 S433 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSGLSGSGAHIVMVDAYKPTKGLSGSGTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 23 - DNA, 2220 bp, Artificial Sequence
  Cap AAV2 VP1 Sea Lion VP2 VP3 T434 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaggcctgagcggcagcggcgcccacatcgtgatggtggacgcctacaagccgacgaagggcctgagcggcagcggcacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 24 - protein, 739 aa, Artificial Sequence
  Cap AAV2 VP1 Sea Lion VP2 VP3 T434 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTGLSGSGAHIVMVDAYKPTKGLSGSGTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 25 - DNA, 2220 bp, Artificial Sequence
  Cap AAV2 VP1 Sea Lion VP2 VP3 T435 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgggcctgagcggcagcggcgcccacatcgtgatggtggacgcctacaagccgacgaagggcctgagcggcagcggcagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 26 - protein, 739 aa, Artificial Sequence
  Cap AAV2 VP1 Sea Lion VP2 VP3 T435 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTGLSGSGAHIVMVDAYKPTKGLSGSGRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 27 - DNA, 2220 bp, Artificial Sequence
  Cap AAV2 VP1 Sea Lion VP2 VP3 R436 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagaggcctgagcggcagcggcgcccacatcgtgatggtggacgcctacaagccgacgaagggcctgagcggcagcggcgacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 28 - protein, 739 aa, Artificial Sequence
  Cap AAV2 VP1 Sea Lion VP2 VP3 R436 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRGLSGSGAHIVMVDAYKPTKGLSGSGDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 29 - DNA, 2220 bp, Artificial Sequence
  Cap AAV2 VP1 Sea Lion VP2 VP3 D437 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacggcctgagcggcagcggcgcccacatcgtgatggtggacgcctacaagccgacgaagggcctgagcggcagcggcttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 30 - protein, 739 aa, Artificial Sequence
  Cap AAV2 VP1 Sea Lion VP2 VP3 D437 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDGLSGSGAHIVMVDAYKPTKGLSGSGLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 31 - DNA, 2244 bp, Artificial Sequence
  Cap AAV2 VP1 Sea Lion VP2 VP3 G432 Linker10 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggaggcctggggagcggcggaggcctgagcggcgcccacatcgtgatggtggacgcctacaagccgacgaagggcctgagcggcggcagcggcctggggggctcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 32 - protein, 747 aa, Artificial Sequence
  Cap AAV2 VP1 Sea Lion VP2 VP3 G432 Linker10 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGGLGSGGGLSGAHIVMVDAYKPTKGLSGGSGLGGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 33 - DNA, 2262 bp, Artificial Sequence
  Cap AAV2 VP1 Dragon VP2 VP3 G436 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgaaaagcgaaagacacccgaagagtggttagctcaagaaaagactccaaccaaacaaaggttccagataccagctccaggacaatctggatcagattctccttccacctcaggatccggcggtactgcaggctccagttctagcgcatcaaatacaatggctcaaggaggtggcggaccaatggcagacgataaccaaggcgccgagggagtgggtaatgcctcgggagattggcattgcgatacccaatggctgggcgaccacgtcattacaaaatctaccagaacttgggttctgccctcttacgggaatcatctctactcgcccatcaactttgatggaaccacagggaacggaacccaagccgcttactgcggatacgctaccccctgggcctactttgactttaaccgattccactgccacttttccccccgagactggcaaagactcattaacaaccataccggaatacgaccagtcggactcaaattcaagctgttcaacatccaggtcaaggaaatcacagtacaagattcgaccaaaacgatcgccaacaatctcaccagcaccgtacaggtctttgcggacacggagcaccagctcccgtacgtattaggaaatgccacgcagggcacgtttcctccctttccggctgaagtctttcagttgcctcagtacggctactgtaccatccaaggccctcagggtaagtttaccgacagaagtgccttctactgcttggagtactttccttccaagatgctgagaacggggaataactttgaatttacttacaagttcgagaaggttcccttccattctggctgggctcaaagccagtcgttggatcgattgatgaatcccttgatcccacagtacctgttggccgattatggtggcctgagcggcagcggcgcccacatcgtgatggtggacgcctacaagccgacgaagggcctgagcggcagcggcacgaccgccagtagtgccataacatactatcggccaaacagcacagacttgagctggtactttaaaaactggctgcctgggccagtggaaaggcgacagcaaatcaattctgaagactcgacgaaaaaccatgccaatttgaacgggaatgcacacaccaacaaatacagcatccagcacagacaaaccaagatgatgccaggggtggctctcagtagtaaatacacaggtgctgctgaaggaacgtcgttactgaacggggtattgacctttgataagatcgccaatgacaatactaacatcacggatacaaataatgtcaatcggacgatcgaagatgaaattcaaggtaccaatccttatggaaacgacgtacccattaccgtcgctgtcaatacacaaaacgcgaccacttctcctaccatgcaaaacagcagcacgtatgaactattacctggtgctgtctggtccaacagagatatttacctgcaaggtcccatctgggctaaaatacccgataccgatggacactttcacccgtctccacaaatgggtggtttcggacttaaaaacccaccaccgatgatcctcatcaaaaacacacccgttcccgccgatccaccaacgaccttcaaaccgactccgatgaacagcttcatcagcgaatacagtaccggtcaggtgactgtagagatgctgtgggaggtccggaaagaagactccaagagatggaacccggaaatccagtttacctccaattttggattgtccgatcctcagatcgacggtataccctttggtattaccaaattgggtaattacgaggaaccacgtcctattggcaccagatacctgactcgtcatttgtaa

SEQ ID NO: 34 - protein, 753 aa, Artificial Sequence
  Cap AAV2 VP1 Dragon VP2 VP3 G436 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGGLSGSGAHIVMVDAYKPTKGLSGSGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGTRYLTRHL

SEQ ID NO: 35 - DNA, 2262 bp, Artificial Sequence
  Cap AAV2 VP1 Dragon VP2 VP3 T573 Linker6 SpyTag
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggccgaaaagcgaaagacacccgaagagtggttagctcaagaaaagactccaaccaaacaaaggttccagataccagctccaggacaatctggatcagattctccttccacctcaggatccggcggtactgcaggctccagttctagcgcatcaaatacaatggctcaaggaggtggcggaccaatggcagacgataaccaaggcgccgagggagtgggtaatgcctcgggagattggcattgcgatacccaatggctgggcgaccacgtcattacaaaatctaccagaacttgggttctgccctcttacgggaatcatctctactcgcccatcaactttgatggaaccacagggaacggaacccaagccgcttactgcggatacgctaccccctgggcctactttgactttaaccgattccactgccacttttccccccgagactggcaaagactcattaacaaccataccggaatacgaccagtcggactcaaattcaagctgttcaacatccaggtcaaggaaatcacagtacaagattcgaccaaaacgatcgccaacaatctcaccagcaccgtacaggtctttgcggacacggagcaccagctcccgtacgtattaggaaatgccacgcagggcacgtttcctccctttccggctgaagtctttcagttgcctcagtacggctactgtaccatccaaggccctcagggtaagtttaccgacagaagtgccttctactgcttggagtactttccttccaagatgctgagaacggggaataactttgaatttacttacaagttcgagaaggttcccttccattctggctgggctcaaagccagtcgttggatcgattgatgaatcccttgatcccacagtacctgttggccgattatggtacgaccgccagtagtgccataacatactatcggccaaacagcacagacttgagctggtactttaaaaactggctgcctgggccagtggaaaggcgacagcaaatcaattctgaagactcgacgaaaaaccatgccaatttgaacgggaatgcacacaccaacaaatacagcatccagcacagacaaaccaagatgatgccaggggtggctctcagtagtaaatacacaggtgctgctgaaggaacgtcgttactgaacggggtattgacctttgataagatcgccaatgacaatactaacatcacggatacaaataatgtcaatcggacgatcgaagatgaaattcaaggtaccaatccttatggaaacgacgtacccattaccgtcgctgtcaatacacaaaacgcgaccggcctgagcggcagcggcgcccacatcgtgatggtggacgcctacaagccgacgaagggcctgagcggcagcggcacttctcctaccatgcaaaacagcagcacgtatgaactattacctggtgctgtctggtccaacagagatatttacctgcaaggtcccatctgggctaaaatacccgataccgatggacactttcacccgtctccacaaatgggtggtttcggacttaaaaacccaccaccgatgatcctcatcaaaaacacacccgttcccgccgatccaccaacgaccttcaaaccgactccgatgaacagcttcatcagcgaatacagtaccggtcaggtgactgtagagatgctgtgggaggtccggaaagaagactccaagagatggaacccggaaatccagtttacctccaattttggattgtccgatcctcagatcgacggtataccctttggtattaccaaattgggtaattacgaggaaccacgtcctattggcaccagatacctgactcgtcatttgtaa

SEQ ID NO: 36 - protein, 753 aa, Artificial Sequence
  Cap AAV2 VP1 Dragon VP2 VP3 T573 Linker6 SpyTag
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAEKRKTPEEWLAQEKTPTKQRFQIPAPGQSGSDSPSTSGSGGTAGSSSSASNTMAQGGGGPMADDNQGAEGVGNASGDWHCDTQWLGDHVITKSTRTWVLPSYGNHLYSPINFDGTTGNGTQAAYCGYATPWAYFDFNRFHCHFSPRDWQRLINNHTGIRPVGLKFKLFNIQVKEITVQDSTKTIANNLTSTVQVFADTEHQLPYVLGNATQGTFPPFPAEVFQLPQYGYCTIQGPQGKFTDRSAFYCLEYFPSKMLRTGNNFEFTYKFEKVPFHSGWAQSQSLDRLMNPLIPQYLLADYGTTASSAITYYRPNSTDLSWYFKNWLPGPVERRQQINSEDSTKNHANLNGNAHTNKYSIQHRQTKMMPGVALSSKYTGAAEGTSLLNGVLTFDKIANDNTNITDTNNVNRTIEDEIQGTNPYGNDVPITVAVNTQNATGLSGSGAHIVMVDAYKPTKGLSGSGTSPTMQNSSTYELLPGAVWSNRDIYLQGPIWAKIPDTDGHFHPSPQMGGFGLKNPPPMILIKNTPVPADPPTTFKPTPMNSFISEYSTGQVTVEMLWEVRKEDSKRWNPEIQFTSNFGLSDPQIDGIPFGITKLGNYEEPRPIGTRYLTRHL

SEQ ID NO: 37 - protein, 4 aa, Artificial Sequence
  4 linker
GLSG

SEQ ID NO: 38 - protein, 6 aa, Artificial Sequence
  6 linker
GLSGSG

SEQ ID NO: 39 - protein, 8 aa, Artificial Sequence
  8 linker
GLSGLSGS

SEQ ID NO: 40 - protein, 10 aa, Artificial Sequence
  10 linker
GLSGLSGLSG

SEQ ID NO: 41 - protein, 10 aa, Artificial Sequence
  10 linker
GLSGGSGLSG

SEQ ID NO: 42 - protein, 13 aa, Artificial Sequence
  SpyTag
AHIVMVDAYKPTK

SEQ ID NO: 43 - protein, 113 aa, Artificial Sequence
  Spycatcher
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 44 - protein, 10 aa, Artificial Sequence
  c-myc
EQKLISEEDL

SEQ ID NO: 45 - protein, 8 aa, Artificial Sequence
  B1
IGTRYLTR

SEQ ID NO: 46 - DNA, 1341 bp, Artificial Sequence
  Human IgG4 heavy chain - linker - SpyCatcher
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaaggaagcggcgaaagcggcgtggataccctgtccggactgagcagtgagcaaggccagtccggagatatgacaattgaagaagatagcgccacccatattaaattctccaaaagagatgaggacggcaaagagctggctggagcaacaatggagctgagagattcctctggaaagactattagtacatggatctctgatggccaagtgaaagatttctatctgtatccaggaaagtacacatttgtcgaaaccgctgcaccagacggatatgaggtggctacagctattacctttacagtgaatgagcaaggacaggtgactgttaatggcaaagctactaaaggagacgctcatatttaa

SEQ ID NO: 47 - protein, 446 aa, Artificial Sequence
  Human IgG4 heavy chain - linker - SpyCatcher
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGSGESGVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 48 - DNA, 18 bp, Artificial Sequence
  IgG-SpyCatcher Linker
ggaagcggcgaaagcggc

SEQ ID NO: 49 - protein, 6 aa, Artificial Sequence
  IgG-SpyCatcher Linker
GSGESG

SEQ ID NO: 50 - DNA, 981 bp, Artificial Sequence
  hIgG4
gcctccaccaagggcccatcggtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccctgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctcaccgtggacaagagcaggtggcaggaggggaatgtcttctcatgctccgtgatgcatgaggctctgcacaaccactacacacagaagtccctctccctgtctctgggtaaa

SEQ ID NO: 51 - protein, 327 aa, Artificial Sequence
  hIgG4
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 52 - DNA, 2145 bp, Artificial Sequence
  Cap AAV2 VP1 Sea Lion VP2 VP3 Y730F
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagattcctgactcgtatactataa

SEQ ID NO: 53 - protein, 714 aa, Artificial Sequence
  Cap AAV2 VP1 Sea Lion VP2 VP3 Y730F
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRFLTRIL

SEQ ID NO: 54 - DNA, 2145 bp, Artificial Sequence
  Cap AAV2 VP1 Sea Lion VP2 VP3 B1 Replaced
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaaccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 55 - protein, 714 aa, Artificial Sequence
  Cap AAV2 VP1 Sea Lion VP2 VP3 B1 Replaced
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 56 - DNA, 2145 bp, Artificial Sequence
  Cap AAV2 VP1 Sea Lion VP2 VP3 v2
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 57 - protein, 714 aa, Artificial Sequence
  Cap AAV2 VP1 Sea Lion VP2 VP3 v2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 58 - DNA, 2145 bp, Artificial Sequence
  Cap AAV2 VP1 Sea Lion VP2 VP3 v3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 59 - protein, 714 aa, Artificial Sequence
  Cap AAV2 VP1 Sea Lion VP2 VP3 v3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 60 - DNA, 2145 bp, Artificial Sequence
  Cap AAV2 VP1 Sea Lion VP2 VP3 v4
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 61 - protein, 714 aa, Artificial Sequence
  Cap AAV2 VP1 Sea Lion VP2 VP3 v4
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 62 - DNA, 2157 bp, Artificial Sequence
  Cap AAV2 VP1 Sea Lion VP2 VP3 v5
atgtcttctttgtttaaagagtacctgcagagtaccggactggtgggtatccagtcgggagcgccgaagccaaaggccggccagcagaagcaggatacagggtcctttgagtggaaaaagaaggaggacacagccagagggctggtccttcccggttacaagtacctcgggcccttcaacggtctagagcggggcgagcctgtgaatgccgcggacgccgcggcccagcgacacgaccgacagtacgatcgtattctacaacaagggggtaatccatacctcacgtacaaccacgccgaccgagagttccaggaggagctccagtctgacgagtcttttgggggaaatcttggcaaggctgtgtttcaggccaagaagcgaatcctagagcccctcggtctggttgaagaggatccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtgattggcaccagatacctgactcgtatactataa

SEQ ID NO: 63 - protein, 718 aa, Artificial Sequence
  Cap AAV2 VP1 Sea Lion VP2 VP3 v5
MSSLFKEYLQSTGLVGIQSGAPKPKAGQQKQDTGSFEWKKKEDTARGLVLPGYKYLGPFNGLERGEPVNAADAAAQRHDRQYDRILQQGGNPYLTYNHADREFQEELQSDESFGGNLGKAVFQAKKRILEPLGLVEEDPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVIGTRYLTRIL

SEQ ID NO: 64 - DNA, 2145 bp, Artificial Sequence
  Cap AAV2 VP1 Sea Lion VP2 VP3 v2 B1 Replaced
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 65 - protein, 714 aa, Artificial Sequence
  Cap AAV2 VP1 Sea Lion VP2 VP3 v2 B1 Replaced
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 66 - DNA, 2145 bp, Artificial Sequence
  Cap AAV2 VP1 Sea Lion VP2 VP3 v3 B1 Replaced
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 67 - protein, 714 aa, Artificial Sequence
  Cap AAV2 VP1 Sea Lion VP2 VP3 v3 B1 Replaced
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 68 - DNA, 2145 bp, Artificial Sequence
  Cap AAV2 VP1 Sea Lion VP2 VP3 v4 B1 Replaced
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 69 - protein, 714 aa, Artificial Sequence
  Cap AAV2 VP1 Sea Lion VP2 VP3 v4 B1 Replaced
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 70 - DNA, 2157 bp, Artificial Sequence
  Cap AAV2 VP1 Sea Lion VP2 VP3 v5 B1 Replaced
atgtcttctttgtttaaagagtacctgcagagtaccggactggtgggtatccagtcgggagcgccgaagccaaaggccggccagcagaagcaggatacagggtcctttgagtggaaaaagaaggaggacacagccagagggctggtccttcccggttacaagtacctcgggcccttcaacggtctagagcggggcgagcctgtgaatgccgcggacgccgcggcccagcgacacgaccgacagtacgatcgtattctacaacaagggggtaatccatacctcacgtacaaccacgccgaccgagagttccaggaggagctccagtctgacgagtcttttgggggaaatcttggcaaggctgtgtttcaggccaagaagcgaatcctagagcccctcggtctggttgaagaggatccctctcagccagattcgtcctcggacaacacggcgccacccgtgaaaaagtcccgtctcgaagaagcccagcccattcaaagtccagacgtttccagcagcactggcggaggaattgccgacgtcatgtctggagatgctgaaatggctgcagtgggcgggggagcaccgggcgtcgacggccagggtgccgagggagtgggtacttcctcgggtaattggcattgcgattcccagtggtcagaaggacacgtcagaaccaccagcaccagaacctgggtgttgcccagctacaacaaccacctgtataaacggcttggaagtagcgcacaatccaatacctacaacggattctccaccccctggggatacctcgacttcaatagatggcactgtcacttcagtcctcggaactggcaacgtctcatcaacaacaactggggcatcagaccaaaaagacttaatgttaaattgttcaacatacaagtcaaagaggtcacgacggaaggggggacgacgaccgtcgccaataaccttaccagcacgattcaggtgtttgcggacaacgcgtacgaactcccgtatgttgtcgacgcgggtcacgagggggcattgccgccgttcccaaacgacgtgtttatgattccccaatacggatactgcgggctggtgtctggtcaaagtcaggctcagtcggacttgtgttcgttttattgcctggagtattttccatcccagatgctgagaacaggaaacaattttgaaatgaatttcaggtttgaagacgttccatttcactccatgtacgcccacagccagtctctagacaggctcatgaatcctttaattgatcagtatctgtggagtctaaaacaaacaggaaacccgactggatcaacaacgagagacttaaaatttatcaagaacaaagttcccaattttgcacattatggaaaaaattggcttcctggaccttttattagacaacaggggtggacaacacaaaatattaataatagtgttgttaattttaatgacatgctgggaaaaaattcgacatttactttggacactagatggagttcattagcgcctggtccgtgtatgggggatgacggacgaactccatccaccaccaagttctcaaatgctcagctcatgtttggatctggaacacaacccaccgaaggcggtgaagatgctgtacatattacatccgagtcggaggtcaaggcaaccaacccaactgcaatcgatgaatacggacgagtggccgataatacgcaaaatgcaacaaccgccccaaccacagtgggaaatgctgcaatgggggccatgcctgggatggtgtggcaagatagggatatctatcttcaaggacccatctggggaaaaataccccatacagacggacattttcatccgtctcctctcatgggggggtttggatacagaaaaccccctccgcaaatttttattaaaaacacccccgttcctggaaatccggcaactacattttctccaaatagaataaacaatttcattactcaatactcaacgggacaagtgaccgttactattgactgggagctgcaaaaggaaaactcaaagagatggaacccagaagtacaatttacatcaaattttggcacggtcgattcactaaactgggcaccggacaacgcgggaaactacaaagaaccaagggtggtggggtctcgattccttacgcatatactataa

SEQ ID NO: 71 - protein, 718 aa, Artificial Sequence
  Cap AAV2 VP1 Sea Lion VP2 VP3 v5 B1 Replaced
MSSLFKEYLQSTGLVGIQSGAPKPKAGQQKQDTGSFEWKKKEDTARGLVLPGYKYLGPFNGLERGEPVNAADAAAQRHDRQYDRILQQGGNPYLTYNHADREFQEELQSDESFGGNLGKAVFQAKKRILEPLGLVEEDPSQPDSSSDNTAPPVKKSRLEEAQPIQSPDVSSSTGGGIADVMSGDAEMAAVGGGAPGVDGQGAEGVGTSSGNWHCDSQWSEGHVRTTSTRTWVLPSYNNHLYKRLGSSAQSNTYNGFSTPWGYLDFNRWHCHFSPRNWQRLINNNWGIRPKRLNVKLFNIQVKEVTTEGGTTTVANNLTSTIQVFADNAYELPYVVDAGHEGALPPFPNDVFMIPQYGYCGLVSGQSQAQSDLCSFYCLEYFPSQMLRTGNNFEMNFRFEDVPFHSMYAHSQSLDRLMNPLIDQYLWSLKQTGNPTGSTTRDLKFIKNKVPNFAHYGKNWLPGPFIRQQGWTTQNINNSVVNFNDMLGKNSTFTLDTRWSSLAPGPCMGDDGRTPSTTKFSNAQLMFGSGTQPTEGGEDAVHITSESEVKATNPTAIDEYGRVADNTQNATTAPTTVGNAAMGAMPGMVWQDRDIYLQGPIWGKIPHTDGHFHPSPLMGGFGYRKPPPQIFIKNTPVPGNPATTFSPNRINNFITQYSTGQVTVTIDWELQKENSKRWNPEVQFTSNFGTVDSLNWAPDNAGNYKEPRVVGSRFLTHIL

SEQ ID NO: 72 - protein, 10 aa, Artificial Sequence
  KTag
ATHIKFSKRD

SEQ ID NO: 73 - protein, 14 aa, Artificial Sequence
  SpyTag002
VPTIVMVDAYKRYK